SEQUENCE LISTING (30 sequences)

SEQ ID NO: 1 - DNA, 20 bp, Artificial Sequence
  5′ junction EE-GM4
  misc_feature (1)..(10): part of T-DNA 5′ flanking region
  misc_feature (11)..(20): part of T-DNA
ttaggcccaggcgctctatc

SEQ ID NO: 2 - DNA, 20 bp, Artificial Sequence
  3′ junction EE-GM4
  misc_feature (1)..(10): part of T-DNA
  misc_feature (11)..(20): part of T-DNA 3′ flanking region
tcacatcaatccatatttaa

SEQ ID NO: 3 - DNA, 40 bp, Artificial Sequence
  EE-GM4 5′ junction
  misc_feature (1)..(20): part of T-DNA 5′ flanking region
  misc_feature (21)..(40): part of T-DNA
ttttagaaagttaggcccaggcgctctatcatagctataa

SEQ ID NO: 4 - DNA, 40 bp, Artificial Sequence
  EE-GM4 3′ junction
  misc_feature (1)..(20): part of T-DNA
  misc_feature (21)..(40): part of T-DNA 3′ flanking region
ccaccagttatcacatcaatccatatttaaagttaaaaca

SEQ ID NO: 5 - DNA, 398 bp, Artificial Sequence
  EE-GM4 5′ region
  misc_feature (1)..(227): part of T-DNA 5′ flanking region
  misc_feature (228)..(398): part of T-DNA region
aaattgaaactgttaaaggccgcttgagccgacttctagttatttattgttaattacatcattttgtttgttacttttcttcatatttctatgtcaatatttaatataatcttcatctgcaggggaaagctaggatactttattggttatataattcagtgtgcaaaaagatattgacaaatactactttgttaagtttagacccctttttagaaagttaggcccaggcgctctatcatagctataaacctattcagcacaatgggctcgagggcgatcgctacgggaactcgagaaggatccttaagcttctagttctagagcggccgctcgaggaattctggattttagtactggattttggttttaggaattagaaattttattgatagaagtat

SEQ ID NO: 6 - DNA, 501 bp, Artificial Sequence
  EE-GM4 3′ region
  misc_feature (1)..(253): part of T-DNA region
  misc_feature (254)..(501): part of T-DNA 3′ flanking region
gggaccactgtcggtagaggcatcttgaacgatagcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccagttatcacatcaatccatatttaaagttaaaacaaataattctaaaaaaaattgattggttttgatcgactcagttctgttttgattagatctataaatttaaactcatgatccaatctatatatgaatgattttgattggtttgattcgattttgatcattaaatacataacctaactcaaactgttcagatcgatttaagtcaattcatattcgttgtcctgtatcaaaaaatacccgtactcttgtttgttgttttcct

SEQ ID NO: 7 - DNA, 3558 bp, Artificial Sequence
  cry14Ab-1.b coding sequence
  CDS (1)..(3558)
atggattgcaaccttcagtcccagcagaacattccatacaacgtgctcgctattccagtttctaacgtgaactcccttactgataccgtgggtgatcttaagaaggcttgggaagagttccaaaagaccggatctttctctcttactgctctccaacagggattctctgcttctcaaggtggaaccttcaactaccttacccttctccagtctggaatttctcttgctggatccttcgttccaggtggaactttcgtggctccaatcatcaacatggtgattggatggctttggccacacaagaacaagaacgctgataccgagaacctcattaacctcatcgattccgagattcagaagcagcttaacaaggctcttctcgatgctgataggaacgagtggtcctcttaccttgagtccatcttcgattcctccaacaacctcaacggtgctattgtggatgctcagtggagtggaactgttaacactaccaacaggacccttagaaacccaaccgagtccgattacaccaacgttgtgaccaacttcattgctgctgatggcgatattgccaacaacgagaaccacatcatgaacggaaacttcgatgttgctgctgctccatacttcgttattggagctaccgctagattcgctgctatgcaatcctacatcaagttctgcaacgcttggattgacaaagtgggactttccgatgctcaacttactacccagaaggctaaccttgataggaccaagcagaacatgaggaacgctatccttaactacacccagcaggttatgaaggtgttcaaggactccaagaacatgccaaccattggcaccaacaagttctctgtggacacctacaacgtgtacatcaagggcatgaccttgaacgtgctcgatattgtggctatttggccatccctttacccagatgattacacctctcagactgctcttgagcaaactagggtgaccttctctaacatggtgggtcaagaagaaggtactgacggatctctcaggatctacaacaccttcgactcattctcttaccagcactccccaatcccaaacaacaacgtgaacctcatctcctactacaacgacgagcttcagaaccttgagcttggagtttacaccccaccaaagaagggatctggatactcttacccatacggcttcgtgcttaactacgccaactccaagtacaagtacggcgattctaacgatccagagtctcttggaggactttctaccctttccgctccaattcaacaggttaacgctgctacccagaactctaagtacctcgatggcgagattcttaacggaattggagcttcccttccaggatattgcactactggatgctctccaactgaaccaccattctcttgcacttctaccgctaacggatacaaggcttcttgcaacccatctgacaccaaccagaagatcaacgctctttacccattcactcaggctaacgtgaagggaaacaccggaaagcttggagttcttgcttctctcgtgtcctacgatctcaacccaaagaacgtgttcggagagcttgattccgataccaacaacgtgattctcaagggaattccagctgagaagggctatttcccaaacaacgctaggccaaccgttgtgaaagagtggattaacggcgcttctgctgttccacttgattctggcaacacccttttcatgaccgctactaaccttactgctacccagtacaggattaggatcagatacgccaacccaaactccaacacccaaatcggagttaggattacccagaacggatcccttatttcttcttccaacctcaccctttactctaccaccgacatgaacaacacccttccacttaacgtgtacgtgattggagagaacggaaactacacccttcaggacctttacaacaccaccaacgtgctttctaccggtgatattaccctccaaatcaccggtggagatcagaagattttcatcgacaggatcgagttcgttccaactatgccagttccaggcaacactaacaacaacaacggaaacaacaatggcaacaataacccaccacatcatgtgtgtgctattgctggaactcagcagtcttgttctggaccaccaaagttcgagcaagtgtccgatcttgagaagattaccacccaggtgtacatgcttttcaagtcctccccatacgaagaacttgctcttgaggtgtcctcttaccagatttcccaagtggctcttaaggtgatggctctctccgatgaacttttctgcgaagagaagaacgtgcttaggaagcttgtgaacaaggccaagcaacttcttgaggcttccaaccttcttgttggaggcaacttcgagactactcagaactgggtgttgggaactaacgcctacatcaactacgattccttcctcttcaacggtaactacctttctcttcagccagcttctggattcttcacctcctacgcctaccaaaagattgatgagtccacccttaagccatacaccaggtacaaggtgtcaggattcattggacagtctaaccaggtggagcttatcatttccagatacggcaaagagatcgacaagatcctcaacgttccatatgctggaccacttccaattaccgctgatgcttccattacttgctgcgctccagaaattggacaatgcgacggcgaacagtctgattctcacttcttcaactactccatcgatgtgggtgctcttcatccagaactcaacccaggaattgagatcggactcaagatcgttcagtccaacggttacatcaccatttccaacctcgagatcattgaggaaaggccacttaccgagatggaaatccaggctgtgaataggaagaaccagaagtgggagagggaaaagcttcttgagtgcgcttctatttctgagcttctccagcctatcatcaaccagattgactccctcttcaaggatggaaactggtacaacgatatccttccacatgtgacctaccaggacctcaagaacattatcatcccagagcttccaaagcttaagcactggttcattgagaacttgcctggtgagtaccatgagatcgagcagaagatgaaggaagctctcaagtacgctttcacccagcttgatgagaagaacctcattcacaacggacatttcaccaccaacctcattgattggcaagttgagggtgatgctcagatgaaggtgttggagaacgatgctcttgctcttcagctcttcaactgggatgcttctgcttcccagtccattaacatcctcgagttcgatgaggataaggcttacaagcttagggtttacgctcaaggatctggaactatccagttcggaaactgcgaagatgaggccattcagttcaacaccaacagcttcatctaccaagagaagatcgtgtacttcgataccccatctgtgaaccttcacattcagtctgagggatccgagttcattgtgtcctccatcgatctcattgagctttccgacgaccagtga

SEQ ID NO: 8 - protein, 1185 aa, Artificial Sequence
  Synthetic Construct
MDCNLQSQQNIPYNVLAIPVSNVNSLTDTVGDLKKAWEEFQKTGSFSLTALQQGFSASQGGTFNYLTLLQSGISLAGSFVPGGTFVAPIINMVIGWLWPHKNKNADTENLINLIDSEIQKQLNKALLDADRNEWSSYLESIFDSSNNLNGAIVDAQWSGTVNTTNRTLRNPTESDYTNVVTNFIAADGDIANNENHIMNGNFDVAAAPYFVIGATARFAAMQSYIKFCNAWIDKVGLSDAQLTTQKANLDRTKQNMRNAILNYTQQVMKVFKDSKNMPTIGTNKFSVDTYNVYIKGMTLNVLDIVAIWPSLYPDDYTSQTALEQTRVTFSNMVGQEEGTDGSLRIYNTFDSFSYQHSPIPNNNVNLISYYNDELQNLELGVYTPPKKGSGYSYPYGFVLNYANSKYKYGDSNDPESLGGLSTLSAPIQQVNAATQNSKYLDGEILNGIGASLPGYCTTGCSPTEPPFSCTSTANGYKASCNPSDTNQKINALYPFTQANVKGNTGKLGVLASLVSYDLNPKNVFGELDSDTNNVILKGIPAEKGYFPNNARPTVVKEWINGASAVPLDSGNTLFMTATNLTATQYRIRIRYANPNSNTQIGVRITQNGSLISSSNLTLYSTTDMNNTLPLNVYVIGENGNYTLQDLYNTTNVLSTGDITLQITGGDQKIFIDRIEFVPTMPVPGNTNNNNGNNNGNNNPPHHVCAIAGTQQSCSGPPKFEQVSDLEKITTQVYMLFKSSPYEELALEVSSYQISQVALKVMALSDELFCEEKNVLRKLVNKAKQLLEASNLLVGGNFETTQNWVLGTNAYINYDSFLFNGNYLSLQPASGFFTSYAYQKIDESTLKPYTRYKVSGFIGQSNQVELIISRYGKEIDKILNVPYAGPLPITADASITCCAPEIGQCDGEQSDSHFFNYSIDVGALHPELNPGIEIGLKIVQSNGYITISNLEIIEERPLTEMEIQAVNRKNQKWEREKLLECASISELLQPIINQIDSLFKDGNWYNDILPHVTYQDLKNIIIPELPKLKHWFIENLPGEYHEIEQKMKEALKYAFTQLDEKNLIHNGHFTTNLIDWQVEGDAQMKVLENDALALQLFNWDASASQSINILEFDEDKAYKLRVYAQGSGTIQFGNCEDEAIQFNTNSFIYQEKIVYFDTPSVNLHIQSEGSEFIVSSIDLIELSDDQ

SEQ ID NO: 9 - DNA, 1077 bp, Artificial Sequence
  hppdPf-4Pa coding sequence
  CDS (1)..(1077)
atggcagatttatatgaaaacccaatgggactcatgggcttcgagtttattgagtttgcttctcctactcctggaactcttgaacctatcttcgaaataatgggttttacaaaggtcgctacccacaggtctaagaacgttcatctgtacagacaaggagagataaatctaatcctgaacaatgagccaaacagcattgcttcctactttgccgctgaacatggtccatcagtgtgtggaatggcctttagggttaaagatagccagaaggcatataatcgtgctttggaactgggagcccaaccaattcacattgatactgggccaatggaacttaaccttccagccataaagggaattggaggtgctcctttgtatcttattgaccgcttcggagagggctcttccatttacgatatcgacttcgtttacttggaaggtgtcgaacgcaatccagttggagctggtttgaaagtgatcgatcaccttacccacaatgtatatagaggaaggatggtgtattgggctaacttctatgagaaactcttcaactttagagaggcaaggtatttcgacattaagggagaatacacaggtctaacttctaaagctatgtcagcaccagacggaatgattaggattcctcttaatgaggaaagttctaagggtgctggacaaatcgaagagttccttatgcagtttaacggtgagggaatccaacacgttgcttttttgacagacgatcttgtcaagacttgggatgctctgaagaaaattggaatgaggtttatgactgcacctcccgatacctattacgaaatgctcgaaggacgacttccagatcacggtgaacccgttgaccagctccaagctagaggtatactacttgatggaagttctgtggaaggagataagaggttgcttctgcagattttttccgagacactaatgggtccagttttctttgagtttattcagcgtaaaggagatgacggctttggcccatggaactttgcgcaacttttcgaaagtattgagcgtgaccaagttcgtagaggtgttcttactgctgattga

SEQ ID NO: 10 - protein, 358 aa, Artificial Sequence
  Synthetic Construct
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGPWNFAQLFESIERDQVRRGVLTAD

SEQ ID NO: 11 - DNA, 8068 bp, Artificial Sequence
  transforming plasmid pSZ8832 - sequence between T-DNA borders
  misc_feature (1)..(130): polylinker
  misc_feature (131)..(400): 3′ untranslated region of 35S transcript (counter clockwise)
  misc_feature (401)..(411): polylinker
  misc_feature (412)..(3969): cry14Ab-1.b coding sequence (counter clockwise)
  misc_feature (3970)..(5276): ubiquitin-10 promoter (counter clockwise)
  misc_feature (5277)..(5381): polylinker
  misc_feature (5382)..(5576): 3′ untranslated region 35S transcript (counter clockwise)
  misc_feature (5577)..(5588): polylinker
  misc_feature (5589)..(6665): hppdPf-4Pa coding sequence (counter clockwise)
  misc_feature (6666)..(7037): TPotpY-1Pf optimized transit peptide (counter clockwise)
  misc_feature (7038)..(7058): polylinker
  misc_feature (7059)..(7185): Tobacco Etch Virus genomic RNA leader sequence (counter clockwise)
  misc_feature (7186)..(7191): polylinker sequence
  misc_feature (7192)..(7941): double enhanced promoter region of CaMV 35S transcript (counter clockwise)
  misc_feature (7942)..(8068): polylinker sequence
gtactgggcccttgtggcgctctatcatagctataaacctattcagcacaatgggctcgagggcgatcgctacgggaactcgagaaggatccttaagcttctagttctagagcggccgctcgaggaattctggattttagtactggattttggttttaggaattagaaattttattgatagaagtattttacaaatacaaatacatactaagggtttcttatatgctcaacacgtgagcgaaaccctataagaaccctaattcccttatctgggaactactcacacattattatggagaaaatagagagagatagatttgtagagagagactggtgatttcagcgtgtcctctccaaatgaaatgaacttccttatatagaggaagggtcttgcgaaggaggcgcgcctcatcactggtcgtcggaaagctcaatgagatcgatggaggacacaatgaactcggatccctcagactgaatgtgaaggttcacagatggggtatcgaagtacacgatcttctcttggtagatgaagctgttggtgttgaactgaatggcctcatcttcgcagtttccgaactggatagttccagatccttgagcgtaaaccctaagcttgtaagccttatcctcatcgaactcgaggatgttaatggactgggaagcagaagcatcccagttgaagagctgaagagcaagagcatcgttctccaacaccttcatctgagcatcaccctcaacttgccaatcaatgaggttggtggtgaaatgtccgttgtgaatgaggttcttctcatcaagctgggtgaaagcgtacttgagagcttccttcatcttctgctcgatctcatggtactcaccaggcaagttctcaatgaaccagtgcttaagctttggaagctctgggatgataatgttcttgaggtcctggtaggtcacatgtggaaggatatcgttgtaccagtttccatccttgaagagggagtcaatctggttgatgataggctggagaagctcagaaatagaagcgcactcaagaagcttttccctctcccacttctggttcttcctattcacagcctggatttccatctcggtaagtggcctttcctcaatgatctcgaggttggaaatggtgatgtaaccgttggactgaacgatcttgagtccgatctcaattcctgggttgagttctggatgaagagcacccacatcgatggagtagttgaagaagtgagaatcagactgttcgccgtcgcattgtccaatttctggagcgcagcaagtaatggaagcatcagcggtaattggaagtggtccagcatatggaacgttgaggatcttgtcgatctctttgccgtatctggaaatgataagctccacctggttagactgtccaatgaatcctgacaccttgtacctggtgtatggcttaagggtggactcatcaatcttttggtaggcgtaggaggtgaagaatccagaagctggctgaagagaaaggtagttaccgttgaagaggaaggaatcgtagttgatgtaggcgttagttcccaacacccagttctgagtagtctcgaagttgcctccaacaagaaggttggaagcctcaagaagttgcttggccttgttcacaagcttcctaagcacgttcttctcttcgcagaaaagttcatcggagagagccatcaccttaagagccacttgggaaatctggtaagaggacacctcaagagcaagttcttcgtatggggaggacttgaaaagcatgtacacctgggtggtaatcttctcaagatcggacacttgctcgaactttggtggtccagaacaagactgctgagttccagcaatagcacacacatgatgtggtgggttattgttgccattgttgtttccgttgttgttgttagtgttgcctggaactggcatagttggaacgaactcgatcctgtcgatgaaaatcttctgatctccaccggtgatttggagggtaatatcaccggtagaaagcacgttggtggtgttgtaaaggtcctgaagggtgtagtttccgttctctccaatcacgtacacgttaagtggaagggtgttgttcatgtcggtggtagagtaaagggtgaggttggaagaagaaataagggatccgttctgggtaatcctaactccgatttgggtgttggagtttgggttggcgtatctgatcctaatcctgtactgggtagcagtaaggttagtagcggtcatgaaaagggtgttgccagaatcaagtggaacagcagaagcgccgttaatccactctttcacaacggttggcctagcgttgtttgggaaatagcccttctcagctggaattcccttgagaatcacgttgttggtatcggaatcaagctctccgaacacgttctttgggttgagatcgtaggacacgagagaagcaagaactccaagctttccggtgtttcccttcacgttagcctgagtgaatgggtaaagagcgttgatcttctggttggtgtcagatgggttgcaagaagccttgtatccgttagcggtagaagtgcaagagaatggtggttcagttggagagcatccagtagtgcaatatcctggaagggaagctccaattccgttaagaatctcgccatcgaggtacttagagttctgggtagcagcgttaacctgttgaattggagcggaaagggtagaaagtcctccaagagactctggatcgttagaatcgccgtacttgtacttggagttggcgtagttaagcacgaagccgtatgggtaagagtatccagatcccttctttggtggggtgtaaactccaagctcaaggttctgaagctcgtcgttgtagtaggagatgaggttcacgttgttgtttgggattggggagtgctggtaagagaatgagtcgaaggtgttgtagatcctgagagatccgtcagtaccttcttcttgacccaccatgttagagaaggtcaccctagtttgctcaagagcagtctgagaggtgtaatcatctgggtaaagggatggccaaatagccacaatatcgagcacgttcaaggtcatgcccttgatgtacacgttgtaggtgtccacagagaacttgttggtgccaatggttggcatgttcttggagtccttgaacaccttcataacctgctgggtgtagttaaggatagcgttcctcatgttctgcttggtcctatcaaggttagccttctgggtagtaagttgagcatcggaaagtcccactttgtcaatccaagcgttgcagaacttgatgtaggattgcatagcagcgaatctagcggtagctccaataacgaagtatggagcagcagcaacatcgaagtttccgttcatgatgtggttctcgttgttggcaatatcgccatcagcagcaatgaagttggtcacaacgttggtgtaatcggactcggttgggtttctaagggtcctgttggtagtgttaacagttccactccactgagcatccacaatagcaccgttgaggttgttggaggaatcgaagatggactcaaggtaagaggaccactcgttcctatcagcatcgagaagagccttgttaagctgcttctgaatctcggaatcgatgaggttaatgaggttctcggtatcagcgttcttgttcttgtgtggccaaagccatccaatcaccatgttgatgattggagccacgaaagttccacctggaacgaaggatccagcaagagaaattccagactggagaagggtaaggtagttgaaggttccaccttgagaagcagagaatccctgttggagagcagtaagagagaaagatccggtcttttggaactcttcccaagccttcttaagatcacccacggtatcagtaagggagttcacgttagaaactggaatagcgagcacgttgtatggaatgttctgctgggactgaaggttgcaatccatctgttaatcagaaaaactcagattaatcgacaaattcgatcgcacaaactagaaactaacacctgatctagatagaaatcacaaatcgaagagtaattattcgacaaaactcaaattatttgaacaaatcggatgatatctatgaaaccctaatcgagaattaagatgatatctaacgatcaaacccagaaaatcgtcttcgatctaagattaacagaatctaaaccaaagaacatatacgaaattgggatcgaacgaaaacaaaatcgaagattttgagagaataaggaacacagaaatttaccttgatcacggtagagagaattgagagaaagtttttaagattttgagaaattgaaatctgaattgtgaagaagaagagctctttgggtattgttttatagaagaagaagaagaaaagacgaggacgactaggtcacgagaaagctaaggcggtgaagcaatagctaataataaaatgacacgtgtattgagcgttgtttacacgcaaagttgtttttggctaattgccttatttttaggttgaggaaaagtatttgtgctttgagttgataaacacgactcgtgtgtgccggctgcaaccactttgacgccgtttattactgactcgtcgacaaccacaatttctaacggtcgtcataagatccagccgttgagatttaacgatcgttacgatttatatttttttagcattatcgttttattttttaaatatacggtggagctgaaaattggcaataattgaaccgtgggtcccactgcattgaagcgtatttcgtattttctagaattcttcgtgctttatttcttttcctttttgtttttttttgccatttatctaatgcaagtgggcttataaaatcagtgaatttcttggaaaagtaacttctttatcgtataacatattgtgaaattatccatttcttttaattttttagtgttattggatatttttgtatgattattgatttgcataggataatgacttttgtatcaagttggtgaacaagtctcgttaaaaaaggcaagtggtttggtgactcgatttattcttgttatttaattcatatatcaatggatcttatttggggcctggtccatatttaacactcgtgttcagtccaatgaccaataatattttttcattaataacaatgtaacaagaatgatacacaaaacattctttgaataagttcgctatgaagaagggaacttatccggtcctagatcatcagttcatacaaacctccatagagttcaacatcttaaacaagaatatcctgatccccaaacaatgattaatagatctaagtcgacactaagctttaactagtttaggcctaatgaattccaggatccatactcgagatacccgggcctgcaggcctaggactggattttggttttaggaattagaaattttattgatagaagtattttacaaatacaaatacatactaagggtttcttatatgctcaacacatgagcgaaaccctataagaaccctaattcccttatctgggaactactcacacattattatagagagagatagatttgtagagagagactggtgatttcagcggacgtcgttaactcaatcagcagtaagaacacctctacgaacttggtcacgctcaatactttcgaaaagttgcgcaaagttccatgggccaaagccgtcatctcctttacgctgaataaactcaaagaaaactggacccattagtgtctcggaaaaaatctgcagaagcaacctcttatctccttccacagaacttccatcaagtagtatacctctagcttggagctggtcaacgggttcaccgtgatctggaagtcgtccttcgagcatttcgtaataggtatcgggaggtgcagtcataaacctcattccaattttcttcagagcatcccaagtcttgacaagatcgtctgtcaaaaaagcaacgtgttggattccctcaccgttaaactgcataaggaactcttcgatttgtccagcacccttagaactttcctcattaagaggaatcctaatcattccgtctggtgctgacatagctttagaagttagacctgtgtattctcccttaatgtcgaaataccttgcctctctaaagttgaagagtttctcatagaagttagcccaatacaccatccttcctctatatacattgtgggtaaggtgatcgatcactttcaaaccagctccaactggattgcgttcgacaccttccaagtaaacgaagtcgatatcgtaaatggaagagccctctccgaagcggtcaataagatacaaaggagcacctccaattccctttatggctggaaggttaagttccattggcccagtatcaatgtgaattggttgggctcccagttccaaagcacgattatatgccttctggctatctttaaccctaaaggccattccacacactgatggaccatgttcagcggcaaagtaggaagcaatgctgtttggctcattgttcaggattagatttatctctccttgtctgtacagatgaacgttcttagacctgtgggtagcgacctttgtaaaacccattatttcgaagataggttcaagagttccaggagtaggagaagcaaactcaataaactcgaagcccatgagtcccattgggttttcatataaatctgccatgcaccggatccttccgccgttgctgacgttgccgaggcttctggaggagcggcgggcgacggggaggctggcggtggacttgagcccctggaacggagcgacggcggtggccgacgaggccatcatcacggtgggcgccatagacagcggcggcaggtacgacagcgtctcgaacttcttgttgccgtaggccggccacacctgcatatattgaactcttccaccgttgctgggaagggtggagaagtcgttagccttcttggtggtggggaaggcggcgttggacttaaggccggtgaacggagccaccatgttggcctgagcaggggcggtccggctaacggtcgcaactgaggaggagatcgaagccattttttttttaattaacacgtgcgttcgtaaatggtgaaaattttcagaaaattgcttttgctttaaaagaaatgatttaaattgctgcaatagaagtagaatgcttgattgcttgagattcgtttgttttgtatatgttgtgttgagaatttattgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggtagaggcatcttgaacgatagcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccagttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccagttgactattcgctaccttaggaccgttatagttacgcccgggttagttagttagcgagcggcgaactaataactccgctctaccgaaagttacgataaacggtcgggtgcggagaaagaggtaatgaaatggca

SEQ ID NO: 12 - DNA, 31 bp, Artificial Sequence
  primer PRIM0937
gagactgtatctttgatatttttggagtaga

SEQ ID NO: 13 - DNA, 24 bp, Artificial Sequence
  primer PRIM0938
ctgagtcgatcaaaaccaatcaat

SEQ ID NO: 14 - DNA, 29 bp, Artificial Sequence
  probe TM1734
aagtgtgtcgtgctccaccagttatcaca

SEQ ID NO: 15 - DNA, 20 bp, Artificial Sequence
  primer SHA071
ccagcttcgccgcttccttc

SEQ ID NO: 16 - DNA, 23 bp, Artificial Sequence
  primer SHA072
gaaggcaagcccatctgcaagcc

SEQ ID NO: 17 - DNA, 24 bp, Artificial Sequence
  probe TM1428
cttcaccttctatgcccctgacac

SEQ ID NO: 18 - DNA, 37 bp, Artificial Sequence
  primer PRIM1652
gagaagtttcaatactaatagtatcaatactcagaat

SEQ ID NO: 19 - DNA, 19 bp, Artificial Sequence
  probe TM2084
cgagtattagccatattta

SEQ ID NO: 20 - DNA, 26 bp, Artificial Sequence
  primer PRIM0939
ccattgtgctgaataggtttatagct

SEQ ID NO: 21 - DNA, 31 bp, Artificial Sequence
  primer 0940
gacaaatactactttgttaagtttagacccc

SEQ ID NO: 22 - DNA, 30 bp, Artificial Sequence
  probe TM1735
tgatagagcgcctgggcctaactttctaaa

SEQ ID NO: 23 - DNA, 9749 bp, Artificial Sequence
  soybean event EE-GM4
  misc_feature (1)..(1058): part of T-DNA 5′ flanking region
  misc_feature (1059)..(8663): inserted T-DNA region
  misc_feature (8664)..(9749): part of 3′ flanking region
ctaaaccttccaacaattttctgggaatagcttgagcattggttcaatgatatcagcacgcattaagactaaaaataaaattattttactatcggcatagttgggttcattggcattagttagaatgtgtcggatacttgacattaaccaaaataactaataaagacttatctaagtcagcaatgcaacctgttgctgtgctatggctctagtgcaaactcgtgattatgctatagccaccttttgaggattattcttaattgattaaaaagatatctatgtgttataaattttgttgatattattttcaattcctatttatgaaaaaccttcatcctaagggactttagcatgacatttgatatgtcaacaaacatgtaagtctatgactctgagataagcttatagggggaatctgagcaactttctagttccaatttgtttctgaggtcatctttttacccaataataattctgatatttttttttatcgcatatatatatagtctatctagtgccttttttctgtgttgcacattcagtatctagaagtcactttggagcttggaccttgactcatctaagatccactgggtggactcgctataaaatcctcctagcggctagcacccatttctttcttatacaaaattcaaatttaaaattttatttaagaaaaaacaaattccataatatttagactaatagacattggtatatctatccaatttcttcaaagctttgattgatcatttatagtctttaaataataaaattatttttaaaatttctccaaaagtagattgatcaatttaacttataataatgattaaattgaaactgttaaaggccgcttgagccgacttctagttatttattgttaattacatcattttgtttgttacttttcttcatatttctatgtcaatatttaatataatcttcatctgcaggggaaagctaggatactttattggttatataattcagtgtgcaaaaagatattgacaaatactactttgttaagtttagacccctttttagaaagttaggcccaggcgctctatcatagctataaacctattcagcacaatgggctcgagggcgatcgctacgggaactcgagaaggatccttaagcttctagttctagagcggccgctcgaggaattctggattttagtactggattttggttttaggaattagaaattttattgatagaagtattttacaaatacaaatacatactaagggtttcttatatgctcaacacgtgagcgaaaccctataagaaccctaattcccttatctgggaactactcacacattattatggagaaaatagagagagatagatttgtagagagagactggtgatttcagcgtgtcctctccaaatgaaatgaacttccttatatagaggaagggtcttgcgaaggaggcgcgcctcatcactggtcgtcggaaagctcaatgagatcgatggaggacacaatgaactcggatccctcagactgaatgtgaaggttcacagatggggtatcgaagtacacgatcttctcttggtagatgaagctgttggtgttgaactgaatggcctcatcttcgcagtttccgaactggatagttccagatccttgagcgtaaaccctaagcttgtaagccttatcctcatcgaactcgaggatgttaatggactgggaagcagaagcatcccagttgaagagctgaagagcaagagcatcgttctccaacaccttcatctgagcatcaccctcaacttgccaatcaatgaggttggtggtgaaatgtccgttgtgaatgaggttcttctcatcaagctgggtgaaagcgtacttgagagcttccttcatcttctgctcgatctcatggtactcaccaggcaagttctcaatgaaccagtgcttaagctttggaagctctgggatgataatgttcttgaggtcctggtaggtcacatgtggaaggatatcgttgtaccagtttccatccttgaagagggagtcaatctggttgatgataggctggagaagctcagaaatagaagcgcactcaagaagcttttccctctcccacttctggttcttcctattcacagcctggatttccatctcggtaagtggcctttcctcaatgatctcgaggttggaaatggtgatgtaaccgttggactgaacgatcttgagtccgatctcaattcctgggttgagttctggatgaagagcacccacatcgatggagtagttgaagaagtgagaatcagactgttcgccgtcgcattgtccaatttctggagcgcagcaagtaatggaagcatcagcggtaattggaagtggtccagcatatggaacgttgaggatcttgtcgatctctttgccgtatctggaaatgataagctccacctggttagactgtccaatgaatcctgacaccttgtacctggtgtatggcttaagggtggactcatcaatcttttggtaggcgtaggaggtgaagaatccagaagctggctgaagagaaaggtagttaccgttgaagaggaaggaatcgtagttgatgtaggcgttagttcccaacacccagttctgagtagtctcgaagttgcctccaacaagaaggttggaagcctcaagaagttgcttggccttgttcacaagcttcctaagcacgttcttctcttcgcagaaaagttcatcggagagagccatcaccttaagagccacttgggaaatctggtaagaggacacctcaagagcaagttcttcgtatggggaggacttgaaaagcatgtacacctgggtggtaatcttctcaagatcggacacttgctcgaactttggtggtccagaacaagactgctgagttccagcaatagcacacacatgatgtggtgggttattgttgccattgttgtttccgttgttgttgttagtgttgcctggaactggcatagttggaacgaactcgatcctgtcgatgaaaatcttctgatctccaccggtgatttggagggtaatatcaccggtagaaagcacgttggtggtgttgtaaaggtcctgaagggtgtagtttccgttctctccaatcacgtacacgttaagtggaagggtgttgttcatgtcggtggtagagtaaagggtgaggttggaagaagaaataagggatccgttctgggtaatcctaactccgatttgggtgttggagtttgggttggcgtatctgatcctaatcctgtactgggtagcagtaaggttagtagcggtcatgaaaagggtgttgccagaatcaagtggaacagcagaagcgccgttaatccactctttcacaacggttggcctagcgttgtttgggaaatagcccttctcagctggaattcccttgagaatcacgttgttggtatcggaatcaagctctccgaacacgttctttgggttgagatcgtaggacacgagagaagcaagaactccaagctttccggtgtttcccttcacgttagcctgagtgaatgggtaaagagcgttgatcttctggttggtgtcagatgggttgcaagaagccttgtatccgttagcggtagaagtgcaagagaatggtggttcagttggagagcatccagtagtgcaatatcctggaagggaagctccaattccgttaagaatctcgccatcgaggtacttagagttctgggtagcagcgttaacctgttgaattggagcggaaagggtagaaagtcctccaagagactctggatcgttagaatcgccgtacttgtacttggagttggcgtagttaagcacgaagccgtatgggtaagagtatccagatcccttctttggtggggtgtaaactccaagctcaaggttctgaagctcgtcgttgtagtaggagatgaggttcacgttgttgtttgggattggggagtgctggtaagagaatgagtcgaaggtgttgtagatcctgagagatccgtcagtaccttcttcttgacccaccatgttagagaaggtcaccctagtttgctcaagagcagtctgagaggtgtaatcatctgggtaaagggatggccaaatagccacaatatcgagcacgttcaaggtcatgcccttgatgtacacgttgtaggtgtccacagagaacttgttggtgccaatggttggcatgttcttggagtccttgaacaccttcataacctgctgggtgtagttaaggatagcgttcctcatgttctgcttggtcctatcaaggttagccttctgggtagtaagttgagcatcggaaagtcccactttgtcaatccaagcgttgcagaacttgatgtaggattgcatagcagcgaatctagcggtagctccaataacgaagtatggagcagcagcaacatcgaagtttccgttcatgatgtggttctcgttgttggcaatatcgccatcagcagcaatgaagttggtcacaacgttggtgtaatcggactcggttgggtttctaagggtcctgttggtagtgttaacagttccactccactgagcatccacaatagcaccgttgaggttgttggaggaatcgaagatggactcaaggtaagaggaccactcgttcctatcagcatcgagaagagccttgttaagctgcttctgaatctcggaatcgatgaggttaatgaggttctcggtatcagcgttcttgttcttgtgtggccaaagccatccaatcaccatgttgatgattggagccacgaaagttccacctggaacgaaggatccagcaagagaaattccagactggagaagggtaaggtagttgaaggttccaccttgagaagcagagaatccctgttggagagcagtaagagagaaagatccggtcttttggaactcttcccaagccttcttaagatcacccacggtatcagtaagggagttcacgttagaaactggaatagcgagcacgttgtatggaatgttctgctgggactgaaggttgcaatccatctgttaatcagaaaaactcagattaatcgacaaattcgatcgcacaaactagaaactaacacctgatctagatagaaatcacaaatcgaagagtaattattcgacaaaactcaaattatttgaacaaatcggatgatatctatgaaaccctaatcgagaattaagatgatatctaacgatcaaacccagaaaatcgtcttcgatctaagattaacagaatctaaaccaaagaacatatacgaaattgggatcgaacgaaaacaaaatcgaagattttgagagaataaggaacacagaaatttaccttgatcacggtagagagaattgagagaaagtttttaagattttgagaaattgaaatctgaattgtgaagaagaagagctctttgggtattgttttatagaagaagaagaagaaaagacgaggacgactaggtcacgagaaagctaaggcggtgaagcaatagctaataataaaatgacacgtgtattgagcgttgtttacacgcaaagttgtttttggctaattgccttatttttaggttgaggaaaagtatttgtgctttgagttgataaacacgactcgtgtgtgccggctgcaaccactttgacgccgtttattactgactcgtcgacaaccacaatttctaacggtcgtcataagatccagccgttgagatttaacgatcgttacgatttatatttttttagcattatcgttttattttttaaatatacggtggagctgaaaattggcaataattgaaccgtgggtcccactgcattgaagcgtatttcgtattttctagaattcttcgtgctttatttcttttcctttttgtttttttttgccatttatctaatgcaagtgggcttataaaatcagtgaatttcttggaaaagtaacttctttatcgtataacatattgtgaaattatccatttcttttaattttttagtgttattggatatttttgtatgattattgatttgcataggataatgacttttgtatcaagttggtgaacaagtctcgttaaaaaaggcaagtggtttggtgactcgatttattcttgttatttaattcatatatcaatggatcttatttggggcctggtccatatttaacactcgtgttcagtccaatgaccaataatattttttcattaataacaatgtaacaagaatgatacacaaaacattctttgaataagttcgctatgaagaagggaacttatccggtcctagatcatcagttcatacaaacctccatagagttcaacatcttaaacaagaatatcctgatccccaaacaatgattaatagatctaagtcgacactaagctttaactagtttaggcctaatgaattccaggatccatactcgagatacccgggcctgcaggcctaggactggattttggttttaggaattagaaattttattgatagaagtattttacaaatacaaatacatactaagggtttcttatatgctcaacacatgagcgaaaccctataagaaccctaattcccttatctgggaactactcacacattattatagagagagatagatttgtagagagagactggtgatttcagcggacgtcgttaactcaatcagcagtaagaacacctctacgaacttggtcacgctcaatactttcgaaaagttgcgcaaagttccatgggccaaagccgtcatctcctttacgctgaataaactcaaagaaaactggacccattagtgtctcggaaaaaatctgcagaagcaacctcttatctccttccacagaacttccatcaagtagtatacctctagcttggagctggtcaacgggttcaccgtgatctggaagtcgtccttcgagcatttcgtaataggtatcgggaggtgcagtcataaacctcattccaattttcttcagagcatcccaagtcttgacaagatcgtctgtcaaaaaagcaacgtgttggattccctcaccgttaaactgcataaggaactcttcgatttgtccagcacccttagaactttcctcattaagaggaatcctaatcattccgtctggtgctgacatagctttagaagttagacctgtgtattctcccttaatgtcgaaataccttgcctctctaaagttgaagagtttctcatagaagttagcccaatacaccatccttcctctatatacattgtgggtaaggtgatcgatcactttcaaaccagctccaactggattgcgttcgacaccttccaagtaaacgaagtcgatatcgtaaatggaagagccctctccgaagcggtcaataagatacaaaggagcacctccaattccctttatggctggaaggttaagttccattggcccagtatcaatgtgaattggttgggctcccagttccaaagcacgattatatgccttctggctatctttaaccctaaaggccattccacacactgatggaccatgttcagcggcaaagtaggaagcaatgctgtttggctcattgttcaggattagatttatctctccttgtctgtacagatgaacgttcttagacctgtgggtagcgacctttgtaaaacccattatttcgaagataggttcaagagttccaggagtaggagaagcaaactcaataaactcgaagcccatgagtcccattgggttttcatataaatctgccatgcaccggatccttccgccgttgctgacgttgccgaggcttctggaggagcggcgggcgacggggaggctggcggtggacttgagcccctggaacggagcgacggcggtggccgacgaggccatcatcacggtgggcgccatagacagcggcggcaggtacgacagcgtctcgaacttcttgttgccgtaggccggccacacctgcatatattgaactcttccaccgttgctgggaagggtggagaagtcgttagccttcttggtggtggggaaggcggcgttggacttaaggccggtgaacggagccaccatgttggcctgagcaggggcggtccggctaacggtcgcaactgaggaggagatcgaagccattttttttttaattaacacgtgcgttcgtaaatggtgaaaattttcagaaaattgcttttgctttaaaagaaatgatttaaattgctgcaatagaagtagaatgcttgattgcttgagattcgtttgttttgtatatgttgtgttgagaatttattgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggtagaggcatcttgaacgatagcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccagttatcacatcaatccatatttaaagttaaaacaaataattctaaaaaaaattgattggttttgatcgactcagttctgttttgattagatctataaatttaaactcatgatccaatctatatatgaatgattttgattggtttgattcgattttgatcattaaatacataacctaactcaaactgttcagatcgatttaagtcaattcatattcgttgtcctgtatcaaaaaatacccgtactcttgtttgttgttttcctgaagggaagggagcatcaaccccgttgatctctccaatctaaggaaaaaatgatgagaaaagaaacgatttgccttaccaagtattatttattttaattttatgttactatatcataaagataaaaaaaaacatttaatacaattatattttttattctcccgttcatccaattaaataagtagaggaataacttgacttttctttatcttcattctcctttttatttttttcaatttagataatttaaaatctacgtttatttttctttcttctaattatttctcctctatttattttcactacaaaacaaaaagttgaaagagacgaacaacaatgcacgtagctatagctgctagtaacaggtccccacagagacgaaaagttgatgcaaatccctccagcagcagattgtaattgtattgtgttgtcccttcctcgctccgtgagcctcgcttgctgccaatgccatttgtttttctcctttctatttctagcaagtagcaacactgtttatttggcagctcacgattttaacctaactcaaacctagctgatattataattcattcaatattttatcacacttaacccacccaactaataaatgagccacaaccggaccaatccttatttaaacaaaatacacaaatatttatgtcatatttttaaaataaattaaatcattaaaataacacgttttaatttaaatcaaaagatcttataaagcaggctaattacttaacttaatcatgaaatataacccaatcttataaagcacaggcttattattttcaagacatgccaatcttggggatcgtaatttctagggtagtgat

SEQ ID NO: 24 - DNA, 1229 bp, Artificial Sequence
  EE-GM4 5′ junction
  misc_feature (1)..(1058): part of T-DNA 5′ flanking region
  misc_feature (1059)..(1229): part of T-DNA
ctaaaccttccaacaattttctgggaatagcttgagcattggttcaatgatatcagcacgcattaagactaaaaataaaattattttactatcggcatagttgggttcattggcattagttagaatgtgtcggatacttgacattaaccaaaataactaataaagacttatctaagtcagcaatgcaacctgttgctgtgctatggctctagtgcaaactcgtgattatgctatagccaccttttgaggattattcttaattgattaaaaagatatctatgtgttataaattttgttgatattattttcaattcctatttatgaaaaaccttcatcctaagggactttagcatgacatttgatatgtcaacaaacatgtaagtctatgactctgagataagcttatagggggaatctgagcaactttctagttccaatttgtttctgaggtcatctttttacccaataataattctgatatttttttttatcgcatatatatatagtctatctagtgccttttttctgtgttgcacattcagtatctagaagtcactttggagcttggaccttgactcatctaagatccactgggtggactcgctataaaatcctcctagcggctagcacccatttctttcttatacaaaattcaaatttaaaattttatttaagaaaaaacaaattccataatatttagactaatagacattggtatatctatccaatttcttcaaagctttgattgatcatttatagtctttaaataataaaattatttttaaaatttctccaaaagtagattgatcaatttaacttataataatgattaaattgaaactgttaaaggccgcttgagccgacttctagttatttattgttaattacatcattttgtttgttacttttcttcatatttctatgtcaatatttaatataatcttcatctgcaggggaaagctaggatactttattggttatataattcagtgtgcaaaaagatattgacaaatactactttgttaagtttagacccctttttagaaagttaggcccaggcgctctatcatagctataaacctattcagcacaatgggctcgagggcgatcgctacgggaactcgagaaggatccttaagcttctagttctagagcggccgctcgaggaattctggattttagtactggattttggttttaggaattagaaattttattgatagaagtat

SEQ ID NO: 25 - DNA, 1339 bp, Artificial Sequence
  EE-GM4 3′ junction
  misc_feature (1)..(253): part of T-DNA
  misc_feature (254)..(1339): part of T-DNA 3′ flanking region
gggaccactgtcggtagaggcatcttgaacgatagcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccagttatcacatcaatccatatttaaagttaaaacaaataattctaaaaaaaattgattggttttgatcgactcagttctgttttgattagatctataaatttaaactcatgatccaatctatatatgaatgattttgattggtttgattcgattttgatcattaaatacataacctaactcaaactgttcagatcgatttaagtcaattcatattcgttgtcctgtatcaaaaaatacccgtactcttgtttgttgttttcctgaagggaagggagcatcaaccccgttgatctctccaatctaaggaaaaaatgatgagaaaagaaacgatttgccttaccaagtattatttattttaattttatgttactatatcataaagataaaaaaaaacatttaatacaattatattttttattctcccgttcatccaattaaataagtagaggaataacttgacttttctttatcttcattctcctttttatttttttcaatttagataatttaaaatctacgtttatttttctttcttctaattatttctcctctatttattttcactacaaaacaaaaagttgaaagagacgaacaacaatgcacgtagctatagctgctagtaacaggtccccacagagacgaaaagttgatgcaaatccctccagcagcagattgtaattgtattgtgttgtcccttcctcgctccgtgagcctcgcttgctgccaatgccatttgtttttctcctttctatttctagcaagtagcaacactgtttatttggcagctcacgattttaacctaactcaaacctagctgatattataattcattcaatattttatcacacttaacccacccaactaataaatgagccacaaccggaccaatccttatttaaacaaaatacacaaatatttatgtcatatttttaaaataaattaaatcattaaaataacacgttttaatttaaatcaaaagatcttataaagcaggctaattacttaacttaatcatgaaatataacccaatcttataaagcacaggcttattattttcaagacatgccaatcttggggatcgtaatttctagggtagtgat

SEQ ID NO: 26 - DNA, 21 bp, Artificial Sequence
  primer GLPB173
cttcatctccccgttaaagtg

SEQ ID NO: 27 - DNA, 21 bp, Artificial Sequence
  primer GLPB175
gttgtcaacaatgaccagaag

SEQ ID NO: 28 - DNA, 20 bp, Artificial Sequence
  primer GLPB167
tacaacgtgctcgctattcc

SEQ ID NO: 29 - DNA, 20 bp, Artificial Sequence
  primer GLPB170
tctcggtatcagcgttcttg

SEQ ID NO: 30 - DNA, 2970 bp, Glycine max
  misc_feature (1)..(1000): 5′ flanking genomic sequence
  misc_feature (1001)..(1970): target site deletion
  misc_feature (1971)..(2970): 3′ flanking genomic sequence
cgcattaagactaaaaataaaattattttactatcggcatagttgggttcattggcattagttagaatgtgtcggatacttgacattaaccaaaataactaataaagacttatctaagtcagcaatgcaacctgttgctgtgctatggctctagtgcaaactcgtgattatgctatagccaccttttgaggattattcttaattgattaaaaagatatctatgtgttataaattttgttgatattattttcaattcctatttatgaaaaaccttcatcctaagggactttagcatgacatttgatatgtcaacaaacatgtaagtctatgactctgagataagcttatagggggaatctgagcaactttctagttccaatttgtttctgaggtcatctttttacccaataataattctgatatttttttttatcgcatatatatatagtctatctagtgccttttttctgtgttgcacattcagtatctagaagtcactttggagcttggaccttgactcatctaagatccactgggtggactcgctataaaatcctcctagcggctagcacccatttctttcttatacaaaattcaaatttaaaattttatttaagaaaaaacaaattccataatatttagactaatagacattggtatatctatccaatttcttcaaagctttgattgatcatttatagtctttaaataataaaattatttttaaaatttctccaaaagtagattgatcaatttaacttataataatgattaaattgaaactgttaaaggccgcttgagccgacttctagttatttattgttaattacatcattttgtttgttacttttcttcatatttctatgtcaatatttaatataatcttcatctgcaggggaaagctaggatactttattggttatataattcagtgtgcaaaaagatattgacaaatactactttgttaagtttagacccctttttagaaagttaggcccagacataggaaaataagccaatgtgttggtccaataattggaccttgcacacacaaaaagcttcaagaatacatgagaaatcactgtgcactgggcagatgcaaattggctctttgagacactcttttaagaaaagaaaagaaaaagtgtaaatttcacgtgttttcttgatatttaagaaaattatatatatatatatatatatatatatatatatatatatatataatgcggaaatcccattatccagattcacataaaatgcgttgttctgtaagtcacaaattaaaaaaaataatttgataaaaatatagtatttaatacattaaaaaaatatcttgtctaacataagaaggcattttacataacaacacacgtggactgatttggtcgattttgatcaaattcaggattcaattcaatcaaatttgattggttccattcgggtttcataattttatttttgaaacccaatacaacctcatccattgataaatgatttgatttgattcagattaacggattattcatttaaatttggttttttccttaaaactattattttatatcaagattcatccaaatatataatacaattaacacaatattatcacatgtctaaaagtaataaaattgattcattcaatatcattaatgtaatattctaaaatagataaatacgaattaaaacaaaatattcttcaacgagatttattatgatagtataaatcacaattatttcctacaaactaattcctaacaacaagctttgctgcaatcaaatttcctacaatcaattttgttacaaccagatttgctgaaacaaatgaacaaaatatgattcattaatattataaacatgtcagaacaaataaatatgaaaaaaggttaaacaaaaaaaattatacctaaaagtaatatattgagaagtttcaatactaatagtatcaatactcagaatccgagtattagccatatttaaagttaaaacaaataattctaaaaaaaattgattggttttgatcgactcagttctgttttgattagatctataaatttaaactcatgatccaatctatatatgaatgattttgattggtttgattcgattttgatcattaaatacataacctaactcaaactgttcagatcgatttaagtcaattcatattcgttgtcctgtatcaaaaaatacccgtactcttgtttgttgttttcctgaagggaagggagcatcaaccccgttgatctctccaatctaaggaaaaaatgatgagaaaagaaacgatttgccttaccaagtattatttattttaattttatgttactatatcataaagataaaaaaaaacatttaatacaattatattttttattctcccgttcatccaattaaataagtagaggaataacttgacttttctttatcttcattctcctttttatttttttcaatttagataatttaaaatctacgtttatttttctttcttctaattatttctcctctatttattttcactacaaaacaaaaagttgaaagagacgaacaacaatgcacgtagctatagctgctagtaacaggtccccacagagacgaaaagttgatgcaaatccctccagcagcagattgtaattgtattgtgttgtcccttcctcgctccgtgagcctcgcttgctgccaatgccatttgtttttctcctttctatttctagcaagtagcaacactgtttatttggcagctcacgattttaacctaactcaaacctagctgatattataattcattcaatattttatcacacttaacccacccaactaataaatgagccacaaccggaccaatccttatttaaacaaaatacacaaatatttatgtcatatttttaaaataaattaaatcattaaaataacacgttttaatttaaatcaaaagatcttataaagcaggctaattacttaacttaatca

(19) United States
(12) Patent Application Publication
(10) Pub. No.: US 2021/0198685 A1
(43) Pub. Date: Jul. 1, 2021

(54) PLANTS CONTAINING ELITE EVENT EE-GM4 AND METHODS AND KITS FOR IDENTIFYING SUCH EVENT IN BIOLOGICAL SAMPLES, AND TREATMENT THEREOF

(71) Applicant: BASF AGRICULTURAL SOLUTIONS SEED US LLC, Florham Park, NJ (US)

(72) Inventors: Hal MOSER, Oceanside, CA (US); Veerle HABEX, Wevelgem (BE); Julia DAUM, Apex, NC (US); Thomas KLEVEN, Lake Park, MN (US); Vadim BEILINSON, Cary, NC (US); Michael MCCARVILLE, Omaha, NE (US); Jeremiah MULLOCK, Youngsville, NC (US)

(21) Appl. No.: 17/058,457

(22) PCT Filed: May 24, 2019
(86) PCT No.: PCT/US2019/033982

§ 371 Date: Nov. 24, 2020

Related U.S. Application Data
(60) Provisional application No. 62/676,434, filed on May 25, 2018.
(60) Provisional application No. 62/686,662, filed on Jun. 18, 2018.

(51) Int. Cl. C12N 15/82

(52) U.S. Cl. CPC C12N 15/8274; C12N 15/8285

(57) ABSTRACT

The invention provides specific transgenic soybean plants, plant material and seeds, characterized in that these harbor a specific nematode resistance and herbicide tolerance elite transformation event at a specific location in the soybean genome, and also said plants, material and seeds treated with compounds and/or biological control agents or mixtures thereof. Tools are also provided which allow rapid and unequivocal identification of the event in biological sample.

### CROSS REFERENCE TO RELATED APPLICATIONS

This application claims the benefit of U.S. Provisional Application Ser. No. 62/676,434, filed May 25, 2018, and U.S. Provisional Application Ser. No. 62/686,662, filed Jun. 18, 2018, the contents of which are herein incorporated by reference in their entirety.

### FIELD OF THE INVENTION

This invention relates to transgenic soybean plants, plant material and seeds, characterized by harboring a specific transformation event conferring nematode resistance and herbicide tolerance, at a specific location in the soybean genome, treated with compounds and/or biological control agents or mixtures as described herein. The invention also relates to seeds treated with compounds and/or biological control agents or mixtures as described herein, and to methods to improve yield in soybean comprising at least the elite event as described, wherein the soybean plants or seeds, or the soil in which soybean plants or seeds are grown or are intended to be grown, are treated with the compounds and/or biological control agents or mixtures as described herein. The soybean plants of the invention combine the nematode resistance and herbicide tolerance phenotype with an agronomic performance, genetic stability and functionality in different genetic backgrounds equivalent to the corresponding non-transformed soybean genetic background in the absence of HPPD inhibitor herbicide(s) or nematode infestation.

### BACKGROUND OF THE INVENTION

The phenotypic expression of a transgene in a plant is determined both by the structure of the gene or genes itself and by its or their location in the plant genome. At the same time the presence of the transgenes or “inserted T-DNA” at different locations in the genome will influence the overall phenotype of the plant in different ways. The agronomically or industrially successful introduction of a commercially interesting trait in a plant by genetic manipulation can be a lengthy procedure dependent on different factors. The actual transformation and regeneration of genetically transformed plants are only the first in a series of selection steps, which include extensive genetic characterization, introgression, and evaluation in field trials, eventually leading to the selection of an elite event.

The unequivocal identification of an elite event is becoming increasingly important in view of discussions on Novel Food/Feed, segregation of GMO and non-GMO products and the identification of proprietary material. Ideally, such identification method is both quick and simple, without the need for an extensive laboratory set-up. Furthermore, the method should provide results that allow unequivocal determination of the elite event without expert interpretation, but which hold up under expert scrutiny if necessary. Specific tools for use in the identification of elite event EE-GM4 in biological samples are described herein.

In this invention, EE-GM4 has been identified as an elite event from a population of transgenic soybean plants in the development of nematode resistant soybean (Glycine max) comprising a gene coding for 4-hydroxy phenylpyruvate dioxygenase (HPPD) inhibitor tolerance combined with a gene conferring resistance to nematodes, each under control of a plant-expressible promoter.

Planting nematode resistant and herbicide tolerant soybean EE-GM4 varieties provides growers with new options for nematode and weed control, using HPPD inhibitor herbicides such as isoxaflutole (IFT), topramezone or mesotrione (MST) herbicide. HPPD inhibitor herbicides offer an alternative weed control option for the soybean grower to help manage problem weed species and as an alternative mode of action tool to help slow the spread of herbicide resistant weeds.

Soybean cyst nematode (SCN) Heterodera glycines (Ichinohe), a worldwide problem for soybean production, is a continuing threat to producers. Since its first detection in the US in 1954 from a single county in North Carolina, SCN has spread to nearly every soybean-producing state in the United States and is estimated to cause more than $1.2 billion in annual yield losses in the US, making it the most damaging soybean pathogen there. SCN was first detected in Brazil in the early 1990s and has since spread throughout South America, and is one of the most important pathogens in Brazil causing losses in practically all Brazilian growing regions. Similarly, SCN continues to spread across soybean producing regions of China with detection in 15 provinces and yield loss estimates of more than $120 million. A multi-year study in the state of Iowa, USA (2001 to 2015) where almost all SCN-resistant soybean varieties contain SCN resistance from PI 88788, found that the virulence of SCN populations increased over the years, resulting in increased end-of-season SCN population densities and reduced yields of SCN-resistant soybean varieties with the PI88788 source of resistance (Mitchum (2016), Phytopathology 106(12):1444-1450, Allen et al. (2017) Plant Health Progr. 18:19-27, Arias et al. (2017) on the world wide web at researchgate.net/publication/266907703_RESISTANCE_TO_SOYBEAN_CYST_NEMATODE_GENETIC S_AND_BREEDING_IN_BRAZIL; McCarville et al. (2017) Plant Health Progress 18:146-155).

The root lesion nematode Pratylenchus brachyurus has become an increasingly important pathogen of soybean. It has a broad host range and is widely distributed in tropical and subtropical regions, especially in Brazil, Africa, and the Southern United States. Pratylenchus brachyurus has become a concern among cotton and soybean growers in the Brazilian Cerrado region and is considered the main nematode pathogen of soybean in the region. In soybean, this nematode can reduce yields 30 to 50%, with greater damage being observed on sandy soils. The use of resistant soybean varieties would be the best way to control this nematode, however, P. brachyurus-resistant soybean varieties have not been identified to date. Although several soybean genotypes have been studied for Pratylenchus brachyurus resistance, and some cultivars identified with increased tolerance, breeding resistant cultivars against P. brachyurus is difficult due to the fact that this nematode is polyphagous and lacks a close interaction with its hosts (Machado (2014) Current Agricultural Science and Technology 20:26-35; Antonio et al. (2012) Soil productivity losses in area infested by the nematoid of the root lesions in Vera, Mont. In: Brazilian Congress of Soy, 6, 2012, Cuiabiá. Abstracts. Londrina: Embrapa Soja, 4 pp; Rios et al. (2016) Ciência Rural 46:580-584; Lima et al., 2017, Chapter 6 in the book: Soybean—The Basis of Yield, Biomass and Productivity; Edited by Minobu Kasai, ISBN 978-953-51-3118-2, Print ISBN 978-953-51-3117-5, InTech; Inomoto et al. (2011) Sucessao de culturas sob piv6 central para controle de fitonematoides: variação populacional, patogenicidade e estimativa de perdas. Tropical Plant Pathology 36:178-185).

It is known that protecting plants against nematodes such as SCN can help plants to better cope with other stresses such as soil composition/content, weather conditions, pathogen stress, herbicide applications, etc.. Particularly when such other stresses give a phenotype that is easily seen, such as chlorosis/yellowing of leaves, the effect of SCN control is more easy to see while otherwise SCN infestation is often not “visible” to the grower. E.g., when soybean plants have Sudden Death Syndrome (SDS) or Iron Deficiency Chlorosis (IDC), protection from SCN will result in plants that are greener or have less severe SDS/IDC symptoms. Despite extensive research and variety screening efforts, iron deficiency remains a challenge in large soybean production areas in the North Central U.S. The importance of this problem has increased due to expanded soybean production on soils susceptible to iron deficiency and to possible interactions with cropping system changes. Iron deficiency occurs in soils with high pH and carbonates, but the expression of iron deficiency is highly variable in space due to interactions with spatially variable soil properties such as moisture content, salinity, availability of iron, and other micronutrient and metal concentrations. Further, iron deficiency expression interacts with biotic factors such as nitrogen fixation, pests, diseases and with management induced stresses such as herbicide application. Variety selection is the most important means to manage iron deficiency, but selecting varieties is complicated by a large genotype by environment interaction related to chlorosis tolerance (Hansen et al. (2004) Soil Sci. Plant Nutr. 50(7):983-987).”

Sudden death syndrome (SDS) of soybean was first discovered in 1971 in Arkansas and since then has been confirmed throughout most soybean-growing areas of the USA. SDS is a fungal disease that also occurs in a disease complex with the soybean cyst nematode (SCN). SDS is among the most devastating soil-borne diseases of soybean in the USA. When this disease occurs in the presence of SCN, symptoms occur earlier and are more severe. SDS is caused by soil-borne fungi within a group of the Fusarium solani species complex. In North America, Fusarium virguliforme, formerly Fusarium solani f. sp. glycines, is the causal agent. In South America, F. brasiliense, F. cuneirostrum, F. tucumaniae, and F. virguliforme cause SDS symptoms. Although soybean cultivars that are less susceptible to SDS have been developed, no highly resistant cultivars are available. The fungus may infect roots of soybean seedlings soon after planting, but above ground symptoms of SDS rarely appear until soybean plants have reached reproductive stages. The fungus produces toxins in the roots that are translocated to the leaves. The first noticeable symptoms of SDS are yellowing and defoliation of upper leaves. If the disease develops early in the season, flowers and young pods will abort. When the disease develops later, the plant will produce fewer seeds per pod or smaller seeds. The earlier severe disease develops, the more the yield is reduced. Because the SDS fungus can persist in soil for long periods, larger areas of a field will show symptoms of the disease each growing season until most of the field is affected (Westphal et al. (2008). Sudden Death Syndrome of Soybean. The Plant Health Instructor. DOI:10.1094/PHI-I-2008-0102-01, on the world wide web at apsnet.org/edcenter/intropp/lessons/fungi/ascomycetes/Pages/SuddenDeath.aspx).

Currently, no soybean plants genetically engineered for nematode resistance are commercialized. Soybean plants comprising one or more herbicide tolerance genes have been disclosed in the art. WO2006/130436 describes a glyphosate tolerant soybean event comprising an epsps gene, and WO2011/034704 describes a dicamba-tolerant soybean event. WO2012/082548 describes soybean plants comprising both an hppd andpat gene. WO2011/063411 describes a soybean event with tolerance to HPPD inhibitors and glyphosate, while WO2011/063413 describes soybean plants with tolerance to HPPD inhibitors, glufosinate and glyphosate. WO2011/066384 describes a soybean event with tolerance to 2,4-D and glufosinate, while WO2012/075426 describes a soybean event with tolerance to 2,4-D, glufosinate and glyphosate and WO2017/059795 describes a soybean event with tolerance to glyphosate. WO2009/064652 describes a soybean event with resistance to lepidopteran insects, and WO2013/016527 describes a soybean event with resistance to lepidopteran insects and glufosinate tolerance.

HPPD genes and proteins that confer improved tolerance to HPPD inhibitor herbicides have been disclosed e.g., in WO2015138394, WO2015135881, WO2014043435, and nematicidal activity of Cry proteins has been described in, e.g., WO2010027805, WO2010027809, WO2010027804, WO2010027799, and in WO2007147029.

None of the prior art disclosures teach or suggest an elite event in soybean comprising a nematode-active Cry gene, treated with the compounds and/or biological control agents or mixtures as described herein, and certainly not an elite event in soybean comprising a nematode-active Cry gene combined with a gene conferring tolerance to HPPD inhibitors, treated with the compounds and/or biological control agents or mixtures as described herein.

It is known in the art that getting a commercial elite transformation event in soybean plants with acceptable agronomic performance is by no means straightforward.

### SUMMARY OF THE PREFERRED EMBODIMENTS OF THE INVENTION

The present invention relates to a treated transgenic soybean plant, plant part, seed, cell or tissue thereof, or treated soil wherein a plant or seed is grown or is intended to be grown (followed by planting or sowing of said plant or seed in said soil), comprising, stably integrated into its genome, an expression cassette which comprises a nematode resistance gene comprising the coding sequence of the cry14Ab-1.b gene and a herbicide tolerance gene comprising the coding sequence of the hppdPf-4 Pa gene (both as described in Example 1.1 herein and as represented in SEQ ID No. 7 and 9, respectively), which provide resistance to plant parasitic nematodes such as soybean cyst nematode and tolerance to an HPPD inhibitor herbicide such as isoxaflutole, topramezone or mesotrione. In the absence of HPPD inhibitor herbicide and nematode pressure, such soybean plant has an agronomic performance which is substantially equivalent to the non-transgenic isogenic line. When encountering soybean cyst nematode (SCN) pressure affecting plant performance in the field, the plants of the invention will have a superior agronomic phenotype compared to a non-transgenic plant. Also, in the presence of weeds, after application of an HPPD inhibitor herbicide to which tolerance is provided, the plants of the invention will have a superior agronomic phenotype compared to plants that were not treated with herbicides. In one embodiment, the invention also relates to such plants or seeds treated with one or more of the compounds and/or biological control agents or mixtures as described herein, and to methods to improve yield in soybean comprising at least the elite event as described, wherein the soybean plants or seeds, or the soil in which soybean plants or seeds are grown or are intended to be grown, are treated with the compounds and/or biological control agents or mixtures thereof as described herein. In one embodiment, such treated plants or seeds also comprise one or more native soybean SCN resistance loci or genes, such as one or more of the SCN resistance genes or loci from the resistance sources of Table 1, or one or more of the SCN resistance genes or loci from the resistance sources PI 88788, PI 548402, PI 209332, or PI 437654.

According to the present invention the soybean plant or seed, cells or tissues thereof comprise elite event EE-GM4 (also described herein as “the (elite) event of this invention”). In one embodiment, elite event EE-GM4 comprises the sequence of any one of SEQ ID No. 1, 3, 5, or 24, or the sequence of any one of SEQ ID No. 2, 4, 6, or 25. In one embodiment, EE-GM4 comprises the sequence of any one of SEQ ID No. 1, 3, 5, or 24 and the sequence of any one of SEQ ID No. 2, 4, 6, or 25, and the cry14Ab-1.b coding sequence of SEQ ID No. 7 and the hppdPf-4 Pa coding sequence of SEQ ID No. 9. In one embodiment, elite event EE-GM4 is a foreign DNA (or inserted T-DNA) inserted at a specific position in the soybean genome, as is contained in reference seed deposited at the ATCC under deposit number PTA-123624. In one embodiment, such inserted T-DNA in EE-GM4 comprises a chimeric Cry14Ab-1-encoding gene and an HPPD-4-encoding gene. In another embodiment, said event is characterized by the 5′ junction sequence of SEQ ID No. 1 or 3, or by the 3′ junction sequence of SEQ ID No. 2 or 4; or by the 5′ junction sequence of SEQ ID No. 1 or 3, and by the 3′ junction sequence of SEQ ID No. 2 or 4. In one embodiment, genomic DNA containing EE-GM4, when analyzed using a polymerase chain reaction (“PCR” herein) with two primers comprising the nucleotide sequence of SEQ ID No. 12 and SEQ ID No. 13 respectively, yields a DNA fragment of 126 bp. In one embodiment, genomic DNA containing EE-GM4, when analyzed using PCR with two primers comprising the nucleotide sequence of SEQ ID No. 20 and SEQ ID No. 21 respectively, yields a DNA fragment of 90 bp. In one embodiment, the invention also relates to such plants or seeds treated with one or more of the compounds and/or biological control agents or mixtures thereof as described herein, particularly such plant or seeds also comprising one or more native soybean SCN resistance loci or genes, such as one or more of the SCN resistance genes or loci from the resistance sources of Table 1, or one or more of the SCN resistance genes or loci from the resistance sources PI 88788, PI 548402, PI 209332, or PI 437654.

In one embodiment herein is provided a soybean plant, cell, plant part, seed or progeny thereof, each comprising elite event EE-GM4 in its genome, reference seed comprising said event having been deposited at the ATCC under deposit number PTA-123624. In one embodiment, a plant or seed comprising EE-GM4 is obtainable by propagation of and/or breeding with a soybean plant grown from the seed deposited at the ATCC under deposit number PTA-123624. In one embodiment, the invention relates to such plants or seeds treated with one or more of the compounds and/or biological control agents or mixtures as described herein, and in another embodiment such plants or seeds also comprise one or more native soybean SCN resistance loci or genes, such as one or more of the SCN resistance genes or loci from the resistance sources of Table 1, or one or more of the SCN resistance genes or loci from the resistance sources PI 88788, PI548402,PI209332, or PI437654.

More specifically, the present invention relates to a transgenic soybean plant, plant part, pollen, seed, cell or tissue thereof, the genomic DNA of which is characterized by the fact that, when analyzed in PCR as described herein, using at least two primers directed to the region formed by a part of the 5′ or 3′ T-DNA flanking region of EE-GM4 and part of the inserted T-DNA, a fragment is amplified that is specific for event EE-GM4. The primers may be directed against the 3′ T-DNA flanking region within SEQ ID NO: 6 or SEQ ID NO. 25 or soybean plant genomic DNA downstream thereof and contiguous therewith and the inserted T-DNA upstream thereof and contiguous therewith. The primers may also be directed against the 5′ T-DNA flanking region within SEQ ID NO: 5 or SEQ ID NO. 24 or soybean plant genomic DNA upstream thereof and contiguous therewith and the inserted T-DNA downstream of and contiguous therewith. In one embodiment, such primers comprise or consist (essentially) of the nucleotide sequence of SEQ ID NO: 12 and SEQ ID NO: 13, or of SEQ ID No. 20 and SEQ ID No. 21, or of SEQ ID NO. 26 and SEQ ID NO. 28, or of SEQ ID NO. 27 and SEQ ID NO. 29, respectively (e.g., a primer pair comprising a primer containing at its extreme 3′ end the nucleotide sequence of SEQ ID NO: 12 and a primer containing at its extreme 3′ end the nucleotide sequence of SEQ ID NO: 13, or a primer pair comprising a primer containing at its extreme 3′ end the nucleotide sequence of SEQ ID No. 20 and a primer containing at its extreme 3′ end the nucleotide sequence of SEQ ID No. 21, or a primer pair comprising a primer containing at its extreme 3′ end the nucleotide sequence of SEQ ID NO: 26 and a primer containing at its extreme 3′ end the nucleotide sequence of SEQ ID NO: 28, or a primer pair comprising a primer containing at its extreme 3′ end the nucleotide sequence of SEQ ID NO: 27 and a primer containing at its extreme 3′ end the nucleotide sequence of SEQ ID NO: 29), and yield a DNA fragment of between 50 and 1000 bp, such as a fragment of 126 bp or of 90 bp.

Reference seed comprising the elite event of the invention (also referred to herein as EE-GM4) has been deposited at the ATCC under accession number PTA-123624. One embodiment of the invention is the elite event EE-GM4 as contained in seed deposited under accession number PTA-123624, which when introduced in a soybean plant will provide resistance to nematodes and tolerance to herbicides, particularly resistance to soybean cyst nematode (Heterodera glycines, “SCN” herein) and/or lesion nematode (lesion nematode as used herein refers to Pratylenchus spp. soybean pest nematodes, including but not limited to Pratylenchus brachyurus) and tolerance to HPPD inhibitors such as isoxaflutole, topramezone or mesotrione. The plants with EE-GM4 of this invention also control root knot nematode (root-knot nematode as used herein refers to Meloidogyne spp. soybean pest nematodes, including but not limited to Meloidogyne incognita, Meloidogyne arenaria, Meloidogyne hapla, or Meloidogyne javanica, or any combination thereof), reniform nematode (Rotylenchulus renformis) and Lance nematode (Hoplolaimus spp. such as H. columbus, H. galeatus, and H. magnistylus). Included in this invention are minor variants of this event such as a soybean event with HPPD inhibitor tolerance and SCN nematode resistance that has a nucleotide sequence with at least 90%, at least 95%, at least 98%, at least 99%, at least 99.5%, or at least 99.9% sequence identity to the nucleotide sequence of EE-GM4 as contained in the seed deposited at the ATCC under deposit number PTA-123624, or a soybean event with HPPD inhibitor tolerance and SCN nematode resistance that has a nucleotide sequence differing in 1 to 200, 1 to 150, 1 to 100, 1 to 75, 1 to 50, 1 to 30, 1 to 20, 1 to 10, or 1 to 5 nucleotides from the nucleotide sequence of EE-GM4 as contained in the deposited seed of ATCC deposit PTA-123624, or that has a nucleotide sequence differing in 1 to 200, 1 to 150, 1 to 100, 1 to 75, 1 to 50, 1 to 30, 1 to 20, 1 to 10, or 1 to 5 nucleotides from the nucleotide sequence formed by the following consecutive nucleotide sequences (5′ to 3′): SEQ ID No. 5 or SEQ ID No. 24, SEQ ID No. 11 from nucleotide position 188 to nucleotide position 7368, and SEQ ID No. 6, or SEQ ID No. 25. In one embodiment, EE-GM4 comprises a nucleotide sequence with at least 95%, at least 96%, at least 97%, at least 98%, at least 99%, at least 99.5%, or at least 99.9% sequence identity to the sequence formed by the following consecutive nucleotide sequences (5′ to 3′): SEQ ID No. 5 or 24, SEQ ID No. 11 from nucleotide position 188 to nucleotide position 7368, and SEQ ID No. 6 or 25. Due to natural genetic variation, single DNA base differences and small insertions and deletions in homologous DNA sequences (e.g., single-nucleotide polymorphisms (SNPs)) are commonly found in plants of the same species (Zhu et al. (2003) Genetics 163:1123-1134).

The seed of ATCC deposit number PTA-123624, is a pure seed lot of transgenic seeds homozygous for elite event EE-GM4 of the invention, which will grow into nematode resistant plants, whereby the plants are also tolerant to an HPPD inhibitor such as isoxaflutole, topramezone or mesotrione. The seed or progeny seed obtainable from the deposited seed (e.g., following crossing with other soybean plants with a different genetic background) can be sown and the seed or the growing plants can be treated with an HPPD inhibitor such as isoxaflutole, topramezone or mesotrione as described herein or can be tested for the presence of EE-GM4 as described herein to obtain plants comprising the elite event of the invention. The invention further relates to cells, seeds, tissues, progeny, and descendants from a plant comprising the elite event of the invention grown from the seed deposited at the ATCC having accession number PTA-123624. The invention further relates to plants obtainable from (such as by propagation of and/or breeding with) a soybean plant comprising the elite event of the invention (such as a plant grown from the seed deposited at the ATCC having accession number PTA-123624, or a plant comprising the hppdPf-4 Pa coding sequence of SEQ ID No. 9 and the cry4Ab-1.b coding sequence of SEQ ID No. 7 located between the sequence of SEQ ID No. 1, 3 5, or 24 and the sequence of SEQ ID No. 2, 4 6, or 25, or a plant comprising the HPPD coding sequence of SEQ ID No. 9 and the cry14Ab-1.b coding sequence of SEQ ID No. 7 located between any one of the sequence of SEQ ID No. 1, 3, or 5 and the sequence of any one of SEQ ID No. 2, 4, or 6). The invention also relates to progeny plants and seeds obtained from the above plants or seed and that comprise the sequence of SEQ ID No. 1 and the sequence of SEQ ID No. 2, or the sequence of SEQ ID No. 3 and the sequence of SEQ ID No. 4, or the sequence of SEQ ID No. 5 and the sequence of SEQ ID No. 6, or the sequence of SEQ ID No. 24 and the sequence of SEQ ID No. 25. In one embodiment, the invention also relates to such plants or seeds treated with one or more of the compounds and/or biological control agents or mixtures thereof as described herein. In another embodiment, the invention relates to such plant or seeds also comprising one or more native soybean SCN resistance loci or genes, such as one or more of the SCN resistance genes or loci from the resistance sources of Table 1, or one or more of the SCN resistance genes or loci from the resistance sources PI 88788, PI 548402, PI 209332, or P1437654.

The transgenic plant, or cells or tissues thereof, comprising elite event EE-GM4 can be identified using methods described herein that are based on the presence of characterizing DNA sequences or amino acids encoded by such DNA sequences in the transgenic plant, cells or tissues. According to a preferred embodiment of the invention, such characterizing DNA sequences are sequences of 15 bp or at least 15 bp, preferably 20 bp or at least 20 bp, most preferably 30 bp or more which comprise the insertion site of the event, i.e., a sequence containing both a part of the inserted T-DNA containing an HPPD inhibitor and nematode resistance transgene and a part of the 5′ or 3′ T-DNA flanking region contiguous therewith that extends into the soybean plant genome, allowing specific identification of the elite event.

Also described herein are methods for identifying elite event EE-GM4 in biological samples, which methods are based on primers or probes which specifically recognize the 5′ and/or 3′ T-DNA flanking sequence and the inserted T-DNA contiguous therewith. Any other methods to identify EE-GM4, e.g., to identify its specific characterizing sequences, are also included herein, such as whole or partial (directed) genome sequencing.

Provided herein is a method for identifying elite event EE-GM4 in biological samples comprising amplifying a sequence of a nucleic acid present in said biological samples, using a polymerase chain reaction with at least two primers, or a polymerase chain reaction with at least two primers and a probe, wherein one of these primers recognizes the 5′ or 3′ T-DNA flanking region in EE-GM4, the other primer recognizes a sequence within the T-DNA comprising the herbicide tolerance and nematode resistance genes that is contiguous with said 5′ or 3′ T-DNA flanking region, preferably to obtain a DNA fragment of 50 to 1000 bp in size. In one embodiment, a first primer recognizes the 5′ T-DNA flanking region in EE-GM4, and a second primer recognizes a sequence within the T-DNA comprising the herbicide tolerance and nematode resistance genes that is contiguous with and downstream of said 5′ T-DNA flanking region, or a first primer recognizes the 3′ T-DNA flanking region in EE-GM4, and a second primer recognizes a sequence within the T-DNA comprising the herbicide tolerance and nematode resistance genes that is contiguous with and upstream of said 3′ T-DNA flanking region, to obtain a DNA fragment characteristic for elite event EE-GM4. In one embodiment, said polymerase chain reaction method further comprises the use of a probe that recognizes the DNA amplified by said primers, e.g., the junction DNA comprising part of the inserted T-DNA and part of the DNA flanking said T-DNA in EE-GM4 (at either the 5′ or 3′ side of the event, as applicable, such as a probe comprising the nucleotide sequence of SEQ ID No. 14 or 22 herein), so as to detect the amplification product produced by said primers. The primers may recognize a sequence within the 5′ T-DNA flanking region of EE-GM4 (SEQ ID No. 5, from nucleotide position 1 to nucleotide position 227, or SEQ ID No. 24 from nucleotide position 1 to nucleotide position 1058) or within the 3′ T-DNA flanking region of EE-GM4 (complement of SEQ ID No. 6 from nucleotide position 254 to nucleotide position 501, or SEQ ID No. 25 from nucleotide position 254 to nucleotide position 1339) and a sequence within the inserted T-DNA (SEQ ID No. 5 from nucleotide position 228 to 398, or SEQ ID No. 6 from nucleotide position 1 to nucleotide position 253, or SEQ ID No. 23 from nucleotide position 1059 to nucleotide position 8663, or the complement thereof), respectively. The primer recognizing the 5′ or 3′ T-DNA flanking region may comprise the nucleotide sequence of SEQ ID No. 13, SEQ ID No. 21, SEQ ID No. 26 or SEQ ID No. 27, and the primer recognizing a sequence within the inserted T-DNA may comprise the nucleotide sequence of SEQ ID No. 12, SEQ ID No. 20, SEQ ID No. 28 or SEQ ID No. 29 described herein. Also described herein is an event-specific primer pair and the specific DNA amplified using such primer pair, as can be obtained by a person of ordinary skill in the art or as can be obtained from commercial sources.

A method for identifying elite event EE-GM4 in biological samples, can comprise amplifying a sequence of a nucleic acid present in a biological sample, using a polymerase chain reaction with two primers comprising or consisting (essentially) of the nucleotide sequence of SEQ ID No. 12 and SEQ ID No. 13 respectively, to obtain a DNA fragment of 126 bp or with two primers comprising or consisting (essentially) of the nucleotide sequence of SEQ ID No. 20 and SEQ ID No. 21 respectively, to obtain a DNA fragment of 90 bp.

Also described herein are the specific T-DNA flanking sequences of EE-GM4, which can be used to develop specific identification methods for EE-GM4 in biological samples. Such specific T-DNA flanking sequences may also be used as reference control material in identification assays. More particularly, the invention relates to the 5′ and/or 3′ T-DNA flanking regions of EE-GM4 which can be used for the development of specific primers and probes as further described herein. Also suitable as reference material are nucleic acid molecules, preferably of about 150-850 bp, comprising the sequence which can be amplified by primers comprising or consisting (essentially) of the nucleotide sequence of SEQ ID No. 12 and SEQ ID No. 13 or of SEQ ID No. 20 and SEQ ID No. 21.

Identification methods for the presence of EE-GM4 in biological samples are described herein, based on the use of such specific primers or probes. Primers may comprise, consist or consist essentially of a nucleotide sequence of 17 to about 200 consecutive nucleotides selected from the nucleotide sequence of SEQ ID No. 5 from nucleotide 1 to nucleotide 227 or SEQ ID No. 24 from nucleotide 1 to nucleotide 1058, or the complement of the nucleotide sequence of SEQ ID 6 from nucleotide 254 to nucleotide 501 or the complement of the nucleotide sequence of SEQ ID No. 25 from nucleotide 254 to nucleotide 1339, combined with primers comprising, consisting, or consisting essentially of a nucleotide sequence of 17 to about 200 consecutive nucleotides selected from the nucleotide sequence of SEQ ID No. 11 from nucleotide 17 to nucleotide 7621 or SEQ ID No. 23 from nucleotide position 1059 to nucleotide position 8663, such as a nucleotide sequence of 17 to about 200 consecutive nucleotides selected from the nucleotide sequence of SEQ ID No. 5 from nucleotide 228 to nucleotide 398 or the nucleotide sequence of SEQ ID No. 6 from nucleotide 1 to nucleotide 253, or the complement thereof, or the nucleotide sequence of SEQ ID No. 11 from nucleotide position 17 to nucleotide position 7621, or the complement thereof. Primers may also comprise these nucleotide sequences located at their extreme 3′ end, and further comprise unrelated sequences or sequences derived from the mentioned nucleotide sequences, but comprising mismatches. In one embodiment, the primers as used herein, can also be identical to the target DNA or the complement thereof, wherein said target DNA is a hybrid containing nucleotide sequences from different origins, that do not occur in such combination in nature.

Kits for identifying elite event EE-GM4 in biological samples are also provided herein, said kits comprising at least one primer pair or probe which specifically recognizes the 5′ or 3′ T-DNA flanking region and the inserted T-DNA comprising a herbicide tolerance and a nematode resistance gene contiguous therewith in EE-GM4.

These kit may comprise, in addition to a primer which specifically recognizes the 5′ or 3′ T-DNA flanking region of EE-GM4, a second primer which specifically recognizes a sequence within the inserted T-DNA comprising an HPPD inhibitor herbicide tolerance and a nematode resistance gene of EE-GM4, for use in a PCR identification protocol. These kits may comprise at least two specific primers, one of which recognizes a sequence within the 5′ T-DNA flanking region of EE-GM4 or a sequence within the 3′ T-DNA flanking region of EE-GM4, and the other which recognizes a sequence within the inserted T-DNA comprising an HPPD inhibitor herbicide tolerance and a nematode resistance gene. The primer recognizing the 5′ T-DNA flanking region may comprise the nucleotide sequence of SEQ ID No. 21 and the primer recognizing the inserted T-DNA may comprise the nucleotide sequence of SEQ ID No. 20, or the primer recognizing the 3′ T-DNA flanking region may comprise the nucleotide sequence of SEQ ID No. 13 and the primer recognizing the inserted T-DNA may comprise the nucleotide sequence of SEQ ID No. 12, or any other primer or primer combination as described herein. The kit may further comprise a probe recognizing a sequence located between the primer recognizing the 5′ T-DNA flanking region and the primer recognizing the sequence within the inserted T-DNA, or recognizing a sequence located between the primer recognizing the 3′ T-DNA flanking region and the primer recognizing the sequence within the inserted T-DNA, such as a probe comprising the sequence of SEQ ID No. 14 or a probe comprising the sequence of SEQ ID No. 22.

A kit for identifying elite event EE-GM4 in biological samples, can also comprise the PCR primers comprising or consisting (essentially) of the nucleotide sequence of SEQ ID No. 12 and SEQ ID No. 13, or of the nucleotide sequence of SEQ ID No. 20 and SEQ ID No. 21 for use in the EE-GM4 PCR protocol described herein. Said kit comprising the primers comprising or consisting (essentially) of the nucleotide sequence of SEQ ID No. 12 and SEQ ID No. 13 may further comprise a probe comprising or consisting (essentially) of the nucleotide sequence of SEQ ID No. 14, and said kit comprising the primers comprising or consisting (essentially) of the nucleotide sequence of SEQ ID No. 20 and SEQ ID No. 21 may further comprise a probe comprising or consisting (essentially) of the nucleotide sequence of SEQ ID No. 22. Said kit can further comprise buffer and reagents such as anyone or each of the following compounds: dNTPs, (Taq) DNA polymerase, MgCl2, stabilizers, and optionally a dye.

A kit for identifying elite event EE-GM4 in biological samples, can also comprise a specific probe comprising or consisting (essentially) of a sequence which corresponds (or is complementary) to a sequence having 80% to 100% sequence identity with a specific region of EE-GM4, wherein such specific region comprises part of the 5′ or 3′ T-DNA flanking region of EE-GM4 and part of the inserted T-DNA contiguous therewith. In one embodiment, the sequence of the probe corresponds to a specific region comprising part of the 5′ or 3′ T-DNA flanking region of EE-GM4 and part of the inserted T-DNA contiguous therewith. Most preferably the specific probe comprises or consists (essentially) of (or is complementary to) a sequence having 80% to 100% sequence identity to the sequence of any one of SEQ ID No. 1, 3 or 5, or a sequence having 80% to 100% sequence identity to the sequence of any one of SEQ ID No. 2, 4 or 6. In one embodiment, the specific probe comprises or consists (essentially) of (or is complementary to) a sequence having 80% to 100% sequence identity to a part of at least 50 contiguous nucleotides of the sequence of SEQ ID No. 5, or a sequence having 80% to 100% sequence identity to a part of at least 50 contiguous nucleotides of the sequence of SEQ ID No. 6, wherein each of said part of SEQ ID No. 5 or 6 comprises sequences of inserted T-DNA and T-DNA flanking sequences of approximately equal length.

Also described herein are DNA molecules comprising sufficient length of polynucleotides of both the T-DNA flanking sequences and the inserted T-DNA of EE-GM4, so as to be useful as primer or probe for the detection of EE-GM4, or to characterize plants comprising event EE-GM4. Such sequences may comprise any one of at least 9, at least 10, at least 15, at least 20, or at least 30 nucleotides, or may comprise any one of 9, 10, 15, 20 or 30 nucleotides of the T-DNA flanking sequence and a similar number of nucleotides of the inserted T-DNA of EE-GM4, at each side of the junction site respectively, and this at either or both of the 5′ and 3′ junction site of the EE-GM4 event. Most preferably, such DNA molecules comprise the sequence of any one of SEQ ID No. 1, 3, or 5 or the sequence of any one of SEQ ID No. 2, 4, or 6. In one embodiment, such DNA molecules comprise the sequence of SEQ ID No. 23, 24 or 25. In one aspect of the invention, soybean plants and seeds are provided comprising such specific DNA molecules.

The methods and kits disclosed herein can be used for different purposes such as, but not limited to the following: to identify the presence or determine the (lower) threshold of EE-GM4 in plants, plant material or in products such as, but not limited to food or feed products (fresh or processed) comprising or derived from plant material; additionally or alternatively, the methods and kits of the present invention can be used to identify transgenic plant material for purposes of segregation between transgenic and non-transgenic material; additionally or alternatively, the methods and kits of the present invention can be used to determine the quality (i.e., percentage pure material) of plant material comprising EE-GM4.

Also provided herein is genomic DNA obtained from plants comprising elite event EE-GM4, particularly genomic DNA comprising EE-GM4 event-specific sequences, such as one or both of the EE-GM4 junction sequences (containing a part of T-DNA flanking DNA and inserted T-DNA contiguous therewith, characteristic for EE-GM4), e.g., any one of the sequences of SEQ ID No. 1, 3, 5, or 24 and/or any one of the sequences of SEQ ID No. 2, 4, 6, or 25. Such genomic DNA may be used as reference control material in the identification assays herein described.

Also provided herein is a transgenic nematode resistant and herbicide tolerant soybean plant, or cells, parts, seeds or progeny thereof, each comprising at least one elite event, said elite event comprises an inserted T-DNA comprising:
i) a first chimeric gene which comprises a cry14Ab-1.b gene derived from Bacillus thuringiensis encoding a Cry14Ab-1 protein under the control of a plant-expressible promoter, such as a chimeric gene comprising a plant-expressible promoter and the coding sequence of SEQ ID No. 7 and
ii) a second chimeric gene which comprises a modified hppdPf-4 Pa gene from Pseudomonas encoding a more tolerant HPPD enzyme under the control of a plant-expressible promoter, such as a chimeric comprising a plant-expressible promoter and the coding sequence of SEQ ID No. 9. In one embodiment, the invention also relates to such plants or seeds treated with one or more of the compounds and/or biological control agents or mixtures as described herein. In another embodiment such plants or seeds also comprise one or more native soybean SCN resistance loci or genes, such as one or more of the SCN resistance genes or loci from the resistance sources of Table 1, or one or more of the SCN resistance genes or loci from the resistance sources PI 88788, PI548402, PI209332, or PI437654.

In one embodiment, elite event EE-GM4 comprises nucleotides 1 to 227 of SEQ ID No. 5 or 1 to 1058 of SEQ ID No. 24 immediately upstream of and contiguous with said inserted T-DNA and nucleotides 254 to 501 of SEQ ID No. 6 or nucleotides 254 to 1339 of SEQ ID No. 25 immediately downstream of and contiguous with said inserted T-DNA.

In a further embodiment, said elite event is obtainable by breeding with a soybean plant grown from reference seed comprising said event having been deposited at the ATCC under deposit number PTA-123624.

In another embodiment, the genomic DNA of said soybean plant, or cells, parts, seeds or progeny thereof when analyzed using PCR with two primers comprising the nucleotide sequence of SEQ ID No. 12 and SEQ ID No. 13 respectively, yields a DNA fragment of 126 bp, or when analyzed using PCR with two primers comprising the nucleotide sequence of SEQ ID No. 20 and SEQ ID No. 21 respectively, yields a DNA fragment of 90 bp.

Also provided herein is a method for identifying a transgenic soybean plant, or cells, parts, seed or progeny thereof with nematode resistance, such as SCN and/or Pratylenchus and/or root-knot and/or reniform nematode resistance, and tolerance to an HPPD inhibitor herbicide, such as isoxaflutole, topramezone or mesotrione, in biological samples, said method comprising amplifying a DNA fragment of between 50 and 150 bp from a nucleic acid present in biological samples using a polymerase chain reaction with at least two primers, one of said primers recognizing the 5′ T-DNA flanking region of the elite event EE-GM4, said 5′ T-DNA flanking region comprising the nucleotide sequence of SEQ ID No. 5 from nucleotide 1 to nucleotide 227, or of SEQ ID No. 24 from nucleotide 1 to nucleotide 1058, or recognizing the 3′ T-DNA flanking region of said elite event, said 3′ T-DNA flanking region comprising the nucleotide sequence of the complement of SEQ ID No. 6 from nucleotide 254 to nucleotide 501, or the nucleotide sequence of the complement of SEQ ID No. 25 from nucleotide 254 to nucleotide 1339, the other primer of said primers recognizing a sequence within the inserted T-DNA comprising the nucleotide sequence of the complement of SEQ ID No. 5 from nucleotide 228 to nucleotide 398 or the nucleotide sequence of SEQ ID No. 6 from nucleotide 1 to nucleotide 253, or wherein said inserted T-DNA comprises the nucleotide sequence of SEQ ID No. 11 from nucleotide position 17 to nucleotide position 7621, or the complement thereof.

Also provided herein is a kit for identifying a transgenic soybean plant, or cells, parts, seed or progeny thereof with nematode resistance and tolerance to an HPPD inhibitor herbicide, in biological samples, said kit comprising one primer recognizing the 5′ T-DNA flanking region of elite event EE-GM4, said 5′ T-DNA flanking region comprising the nucleotide sequence of SEQ ID No. 5 from nucleotide 1 to nucleotide 227, or of SEQ ID No. 24 from nucleotide 1 to nucleotide 1058, or one primer recognizing the 3′ T-DNA flanking region of said elite event, said 3′ T-DNA flanking region comprising the nucleotide sequence of the complement of SEQ ID No. 6 from nucleotide 254 to nucleotide 501, or the nucleotide sequence of the complement of SEQ ID No. 25 from nucleotide 254 to nucleotide 1339, and one primer recognizing a sequence within the inserted T-DNA, said inserted T-DNA comprising the nucleotide sequence of the complement of SEQ ID No. 5 from nucleotide 228 to nucleotide 398 or the nucleotide sequence of SEQ ID No. 6 from nucleotide 1 to nucleotide 253, or said inserted T-DNA comprising the nucleotide sequence of SEQ ID No. 11 from nucleotide position 17 to nucleotide position 7621, or the complement thereof.

In one embodiment, the inserted T-DNA of elite event EE-GM4, as used herein, comprises the nucleotide sequence of SEQ ID No. 5 from nucleotide 228 to nucleotide 398 or its complement, and the nucleotide sequence of SEQ ID No. 6 from nucleotide 254 to nucleotide 501 or its complement, or comprises a sequence with at least 95, 98, 99, 99.5, or 99.9% sequence identity to the nucleotide sequence of SEQ ID No. 11 from nucleotide position 17 to nucleotide position 7621, or its complement.

Also provided herein is a soybean plant, plant cell, tissue, or seed, comprising in their genome a nucleic acid molecule comprising the nucleotide sequence of any one of SEQ ID No. 1, 3, 5 or 24 or a nucleotide sequence of 80 to 100% sequence identity thereto and/or SEQ ID No. 2, 4, 6 or 25 or a nucleotide sequence of 80 to 100% sequence identity thereto, and a nucleotide sequence with at least 80, 85, 90, 95, 97, 98, 99, 99.5 or at least 99.9% sequence identity to the nucleotide sequence of SEQ ID No. 11 from nucleotide position 188 to nucleotide position 7368 or the complement thereof, wherein such plant, plant cell, tissue or seed was treated with one or more of the compounds and/or biological control agents or mixtures as described herein. In one embodiment such plants or seeds also comprise one or more native soybean SCN resistance loci or genes, such as one or more of the SCN resistance genes or loci from the resistance sources of Table 1, or one or more of the SCN resistance genes or loci from the resistance sources PI 88788, PI548402,PI209332, or PI437654.

Also provided herein is the use of a nucleic acid molecule comprising a nucleotide sequence with at least 90%, at least 95%, at least 98%, or at least 99% sequence identity to the nucleotide sequence of SEQ ID No. 7 or the complement thereof, or an isolated nucleic acid molecule comprising a nucleotide sequence hybridizing under standard stringency conditions to the nucleotide sequence of SEQ ID No. 7 or the complement thereof, wherein such nucleic acid molecule encodes a nematicidal toxin active to cyst nematodes and/or lesion nematodes and/or root-knot nematodes and/or reniform nematode, such as Heterodera glycines and/or Pratylenchus brachyurus and/or Meloidogyne incognita and/or Rotylenchulus renformis, in combination with one or more of the compounds and/or biological control agents or mixtures as described herein, so as to ensure increased protection to such nematodes and/or delay or prevent the development of nematode resistance. In one embodiment, such nucleic acid molecule is operably-linked to a nucleic acid molecule comprising a (heterologous) plant-expressible promoter so as to form a chimeric gene. Also provided herein is the use of said nucleic acid molecule in transformed plants or seeds treated with said compounds and/or biological control agents or mixtures to control plant-pathogenic nematodes. Further provided herein is a method to control root-knot nematodes such as Meloidogyne incognita, Meloidogyne arenaria, Meloidogyne hapla, or Meloidogyne javanica, particularly Meloidogyne incognita, comprising using a Cry14Ab protein or a DNA encoding a Cry14Ab protein or a plant or seed containing said DNA under the control of a plant-expressible promoter, wherein said Cry14Ab protein is the protein comprising the amino acid sequence of SEQ ID No. 8 or a protein with at least 96% or at least 98 or at least 99% sequence identity thereto, or a protein comprising the amino acid sequence of SEQ ID No. 8 from amino acid position 1 to amino acid position 706, or a protein with at least 96% or at least 98 or at least 99% sequence identity thereto, wherein said use also includes the use of the compounds and/or biological control agents or mixtures described herein on plants or seeds comprising said (DNA encoding) said Cry protein. Further provided herein is a method to control reniform nematodes (Rotylenchulus renformis), comprising using a Cry14Ab protein or a DNA encoding a Cry14Ab protein, or a plant or seed containing said DNA, under the control of a plant-expressible promoter, wherein said Cry14Ab protein is the protein comprising the amino acid sequence of SEQ ID No. 8 or a protein with at least 96% or at least 98% or at least 99% sequence identity thereto, or a protein comprising the amino acid sequence of SEQ ID No. 8 from amino acid position 1 to amino acid position 706, or a protein with at least 96% or at least 98 or at least 99% sequence identity thereto, wherein said use also includes the use of the compounds and/or biological control agents or mixtures described herein on plants or seeds comprising said (DNA encoding) said Cry protein.

Also provided herein is a nucleic acid molecule comprising the nucleotide sequence of SEQ ID No. 11 from nucleotide position 131 to nucleotide position 7941, or a nucleotide sequence having at least 95%, at least 96%, at least 97%, at least 98%, or at least 99% sequence identity thereto, wherein said nucleic acid molecule encodes a nematicidal Cry14Ab protein and an HPPD protein tolerant to HPPD inhibitors, wherein said use also includes the use of the compounds and/or biological control agents or mixtures described herein on plants or seeds comprising said (DNA encoding) said Cry protein. In one embodiment, that nucleic acid molecule encodes the protein of SEQ ID No. 8 or a protein at least 99% identical thereto and the protein of SEQ ID No. 10, or a protein at least 99% identical thereto.

Also provided herein is a soybean plant, seed or cell comprising in its genome elite event EE-GM4 which is an inserted T-DNA at a defined locus, wherein the elite event EE-GM4 is as contained in reference seed deposited at the ATCC under deposit number PTA-123624, wherein said inserted T-DNA comprises a chimeric Cry14Ab-1-encoding gene and a chimeric HPPD-4-encoding gene, and wherein said elite event is characterized by the 5′ junction sequence of SEQ ID No. 1 or 3 and by the 3′ junction sequence of SEQ ID No. 2 or 4; or such cell which is a seed cell, or such cell, wherein the genomic DNA of said cell, when analyzed using PCR with two primers comprising the nucleotide sequence of SEQ ID 12 and SEQ ID 13 respectively, yields a DNA fragment of 126 bp, wherein said plant, seed or cell is treated with the compounds and/or biological control agents or mixtures described herein.

In one embodiment, elite event EE-GM4 is as contained in reference seed deposited at the ATCC under deposit number PTA-123624, and is characterized by comprising a chimeric Cry14Ab-1-encoding gene and an HPPD-4-encoding gene, and comprising the sequence of SEQ ID No. 1 or 3 and the sequence of SEQ ID No. 2 or 4.

In one embodiment, elite event EE-GM4 contains a nucleic acid molecule comprising in order the following nucleotide sequences: a) the nucleotide sequence of SEQ ID NO. 5 from nucleotide 1 to 227 or a sequence at least 99% identical thereto, b) the nucleotide sequence of SEQ ID No. 11 from nucleotide 188 to nucleotide 7368 or a sequence at least 99% identical thereto, and c) the nucleotide sequence of SEQ ID NO. 6 from nucleotide 254 to nucleotide 501 or a sequence at least 99% identical thereto, such as such nucleic acid molecule comprising a sequence b) that is at least 99.5% or at least 99.9% identical to the nucleotide sequence of SEQ ID No. 11 from nucleotide 188 to nucleotide 7368.

In one embodiment, elite event EE-GM4 contains a nucleic acid molecule comprising in order the following nucleotide sequences: a) the nucleotide sequence of SEQ ID NO. 24 from nucleotide 1 to 1058 or a sequence at least 99% identical thereto, b) the nucleotide sequence of SEQ ID No. 23 from nucleotide 1059 to nucleotide 8663 or a sequence at least 99% identical thereto, and c) the nucleotide sequence of SEQ ID NO. 25 from nucleotide 254 to nucleotide 1339 or a sequence at least 99% identical thereto, such as such nucleic acid molecule comprising a sequence b) that is at least 99.5% or at least 99.9% identical to the nucleotide sequence of SEQ ID No. 23.

Also provided herein is the use of soybean seed comprising elite event EE-GM4 to obtain a treated soybean seed, wherein said elite event comprises the sequence of any one of SEQ ID NO. 1, 3, 5 or 24 and/or the sequence of any one of SEQ ID No. 2, 4, 6 or 25, and wherein said treatment is with one or more of the compound(s) and/or biological control agent(s) or mixture(s) described herein.

Further, provided herein is a method for producing a soybean plant or seed comprising elite event EE-GM4 combined with another SCN resistance locus/gene, such as by combining elite event EE-GM4 with another SCN resistance locus/gene occurring in soybean, and planting seed comprising EE-GM4 and said other SCN resistance locus/gene. In one embodiment, the plants, cells or seeds of the invention contain one or more other SCN resistance loci/genes that occur in soybean, to get a combination of different SCN resistance sources in the soybean plants, cells or seeds of the invention. Several soybean SCN resistance loci or genes are known and one or more of those can be combined with EE-GM4 in the same plant, cell or seed, such as any one of the SCN resistance genes/loci from the resistance sources PI 88788, PI 548402 (Peking), PI 437654 (Hartwig or CystX®), or any combination thereof, or one or more of the native SCN resistance loci/genes rhg1, rhg1-b, rhg2, rhg3, Rhg4, Rhg5, qSCN11, cqSCN-003, cqSCN-005, cqSCN-006, cqSCN-007, or any of the SCN resistance loci identified on any one of soybean chromosomes 1, 3, 4, 5, 6, 7, 8, 9, 10, 11, 12, 13, 14, 15, 16, 17, 18, 19, or 20 or any combination thereof (Kim et al. 2016, Theor. Appl. Genet. 129(12):2295-2311; Kim and Diers 2013, Crop Science 53:775-785; Kazi et al. 2010, Theor. Appl. Gen. 120(3):633-644; Glover et al. 2004, Crop Science 44(3):936-941; on the world wide web at soybase.org; Concibido et al. 2004, Crop Science 44:1121-1131; Webb et al. 1995, Theor. Appl. Genet. 91:574-581). Also, in one embodiment the plants or seeds of the invention contain EE-GM4 when combined with one or more SCN resistance loci in soybean obtained from any one of SCN resistance sources PI 548316, PI 567305, PI 437654, PI 90763, PI 404198B, PI 88788, PI 468916, PI 567516C, PI 209332, PI 438489B, PI 89772, Peking, PI 548402, PI 404198A, PI 561389B, PI 629013, PI 507471, PI 633736, PI 507354, PI 404166, PI 437655, PI 467312, PI 567328, PI 22897, or PI 494182. Table 1 enclosed hereto provides a comprehensive list of soybean accessions reported as SCN resistant, of which the SCN resistance genes/loci (one or several) can be combined with EE-GM4 of the invention in the same soybean plant, cell or seed. In one embodiment, the invention also relates to such plants or seeds treated with one or more of the compounds and/or biological control agents or mixtures as described herein.

TABLE 1

| | | | | | | |
|---|---|---|---|---|---|---|
| FC 21340 | PI 404192C | PI 438498 | PI 507451 | PI 548974 | PI 567771C | PI 68465 |
| FC 31685 | PI 404198A | PI 438503A | PI 507470 | PI 548975 | PI 567773 | PI 68622 |
| PI 101404A | PI 404198B | PI 458506 | PI 507471 | PI 548981 | PI 603587A | PI 70027 |
| PI 153229 | PI 407022 | PI 458510 | PI 507475 | PI 548982 | PI 605743B | PI 70213 |
| PI 153297 | PI 407221 | PI 458519A | PI 507476 | PI 548988 | PI 606416A | PI 70229 |
| PI 153303 | PI 407729 | PI 458520 | PI 507686C | PI 549031 | PI 606420 | PI 70251 |
| PI 157430 | PI 416762 | PI 461509 | PI 509095 | PI 553040 | PI 606424 | PI 70519 |
| PI 157444 | PI 417091 | PI 464888A | PI 509100 | PI 553047 | PI 606430 | PI 71161 |
| PI 16790 | PI 423927 | PI 464910 | PI 511813 | PI 559370 | PI 606435 | PI 79620 |
| PI 17852-B | PI 424387 | PI 464912 | PI 518772 | PI 561389B | PI 606436 | PI 79712 |
| PI 181558 | PI 424595 | PI 464925B | PI 522186 | PI 56563 | PI 606437 | PI 80834-2 |
| PI 200495 | PI 437654 | PI 467312 | PI 522236 | PI 567305 | PI 606439 | PI 82308 |
| PI 209332 | PI 437655 | PI 467327 | PI 533605 | PI 567325B | PI 606441 | PI 84664 |
| PI 22897 | PI 437679 | PI 467332 | PI 540556 | PI 567328 | PI 606443 | PI 84751 |
| PI 232993 | PI 437690 | PI 468903 | PI 543855 | PI 567333A | PI 612610 | PI 84807 |
| PI 303652 | PI 437725 | PI 468915 | PI 54620-2 | PI 567354 | PI 612611 | PI 84896 |
| PI 339868B | PI 437770 | PI 468916 | PI 548316 | PI 567360 | PI 612612A | PI 87631-1 |
| PI 339871A | PI 437793 | PI 468916 | PI 548349 | PI 567387 | PI 612614 | PI 88788 |
| PI 346298 | PI 437844A | PI 494182 | PI 548376 | PI 567488B | PI 612615 | PI 89008 |
| PI 347544A | PI 437904 | PI 495017C | PI 548402 | PI 567491A | PI 612616 | PI 89772 |
| PI 371610 | PI 438342 | PI 506862 | PI 548402S | PI 567516C | PI 612617A | PI 89783 |
| PI 378690 | PI 438489B | PI 507354 | PI 548456 | PI 567676A | PI 62202 | PI 90763 |
| PI 398682 | PI438491 | PI 507422 | PI 548655 | PI 567726 | PI 629013 | PI 91102 |
| PI 399061 | PI 438496B | PI 507423 | PI 548665 | PI 567737 | PI 633736 | PI 92576 |
| PI 404166 | PI 438497 | PI 507443 | PI 548970 | PI 567741 | PI 63468 | PI 92595 |
| | | | | | | PI 96549 |

In one embodiment, the methods of the invention also comprise treating the soybean plants or seeds, or the soil in which the soybean plants or seeds are grown or are intended to be grown, with one or more of the compound(s) and/or biological control agent(s) or a mixture comprising them, as described herein, such as wherein said compound or biological control agent is nematicidal, insecticidal, or fungicidal. In another embodiment such plants or seeds also comprise one or more native soybean SCN resistance loci or genes, such as one or more of the SCN resistance genes or loci from the resistance sources of Table 1, or one or more of the SCN resistance genes or loci from the resistance sources PI 88788, PI 548402, PI 209332, or PI 437654.

Also provided herein is a method for protecting emerging soybean plants from competition by weeds, comprising treating a field in which seeds containing elite event EE-GM4 as described above were sown, with an HPPD inhibitor herbicide, wherein the plants are tolerant to the HPPD inhibitor herbicide. In one embodiment, in such method the HPPD inhibitor herbicide is isoxaflutole, topramezone or mesotrione. In one embodiment, such method also comprises treating the soybean plants or seeds, or the soil in which the soybean plants or seeds are grown or are intended to be grown, with one or more of the compound(s) and/or biological control agent(s) or a mixtures comprising them, as described herein, such as wherein said compound or biological control agent is nematicidal, insecticidal, or fungicidal, e.g. a compound or biological control agent or combination thereof selected from any one of group IAN1, IAN2, IAN3, IAN4, IAN5, IAN6, IAN7, IAN8, IAN9, IAN10, IAN11, IAN12, IAN13, IAN14, IAN15, IAN16, IAN17, IAN18, IAN19, IAN20, IAN21, IAN22, IAN23, IAN24, IAN25, IAN26, IAN27, IAN28, IAN29, IAN30, SIAN1, F1, F2, F3, F4, F5, F6, F7, F8, F9, F9, F10, F11, F12, F13, F14, F15, F16, SF1, P1, BCA1, BCA2, BCA3, BCA4, BCA5, BCA6, BCA7, BCA8, BCA9, BCA10, NC1, NC2, NC3, SBCA1, SAI1, SC1 or SC2, as described herein.

Also provided herein is method for protecting emerging soybean plants from competition by weeds and from damage caused by plant-pathogenic nematodes, comprising treating a field to be planted with soybean plants comprising elite event EE-GM4 as described above with an HPPD inhibitor herbicide and a nematicidal compound or biological control agent or combination thereof as described herein, before the soybean plants are planted or the seeds are sown, followed by planting or sowing of said soybean plants or seeds in said pre-treated field, wherein the plants are tolerant to the HPPD inhibitor herbicide. In one embodiment, provided herein is method for protecting emerging soybean plants from competition by weeds and from damage caused by plant-pathogenic nematodes, comprising treating a field to be planted with soybean plants comprising elite event EE-GM4 as described above with an HPPD inhibitor herbicide, before the soybean seeds are sown, followed by sowing of said soybean seeds in said pre-treated field, wherein the plants are tolerant to the HPPD inhibitor herbicide, and wherein said seeds are coated with a compound or biological control agent or combination thereof, as described herein, such as a compound or biological control agent or combination thereof selected from any one of group IAN1, IAN2, IAN3, IAN4, IAN5, IAN6, IAN7, IAN8, IAN9, IAN10, IAN11, IAN12, IAN13, IAN14, IAN15, IAN16, IAN17, IAN18, IAN19, IAN20, IAN21, IAN22, IAN23, IAN24, IAN25, IAN26, IAN27, IAN28, IAN29, IAN30, SIAN1, F1, F2, F3, F4, F5, F6, F7, F8, F9, F9, F10, F11, F12, F13, F14, F15, F16, SF1, P1, BCA1, BCA2, BCA3, BCA4, BCA5, BCA6, BCA7, BCA8, BCA9, BCA10, NC1, NC2, NC3, SBCA1, SAI1, SC1 or SC2, as described herein.

Also provided herein is a method for controlling weeds and for protection plants from damage caused by plant-pathogenic nematodes in a field of soybean plants comprising elite event EE-GM4 as described above, comprising treating said field with an effective amount of an HPPD inhibitor herbicide, and a nematicidal compound or biological control agent or combination thereof as described herein wherein the plants are tolerant to such herbicide, such as wherein said compound or biological control agent or combination is selected from any one of group IAN1, IAN2, IAN3, IAN4, IAN5, IAN6, IAN7, IAN8, IAN9, IAN10, IAN11, IAN12, IAN13, IAN14, IAN15, IAN16, IAN17, IAN18, IAN19, IAN20, IAN21, IAN22, IAN23, IAN24, IAN25, IAN26, IAN27, IAN28, IAN29, IAN30, SIAN1, F1, F2, F3, F4, F5, F6, F7, F8, F9, F9, F10, F11, F12, F13, F14, F15, F16, SF1, P1, BCA, BCA2, BCA3, BCA4, BCA5, BCA6, BCA7, BCA8, BCA9, BCA10, NC1, NC2, NC3, SBCA1, SA1, SC1 or SC2, as described herein.

In one embodiment, the methods of the invention, such as the methods for controlling weeds and for protecting plants from plant-pathogenic nematodes, comprise besides application of HPPD inhibitor herbicides, also comprise treating the soybean plants or seeds, or the soil in which the soybean plants or seeds are grown or are intended to be grown, with one or more of the compound(s) and/or biological control agent(s) or a mixture, as described herein, such as wherein said compound or biological control agent is nematicidal, insecticidal, or fungicidal, or a compound or biological control agent or combination thereof selected from any one of group IAN1, IAN2, IAN3, IAN4, IAN5, IAN6, IAN7, IAN8, IAN9, IAN10, IAN11, IAN12, IAN13, IAN14, IAN15, IAN16, IAN17, IAN18, IAN19, IAN20, IAN21, IAN22, IAN23, IAN24, IAN25, IAN26, IAN27, IAN28, IAN29, IAN30, SIAN1, F1, F2, F3, F4, F5, F6, F7, F8, F9, F9, F10, F11, F12, F13, F14, F15, F16, SF1, P1, BCA, BCA2, BCA3, BCA4, BCA5, BCA6, BCA7, BCA8, BCA9, BCA10, NC1, NC2, NC3, SBCA1, SAI1, SC1 or SC2, as described herein. In another embodiment such plants or seeds also comprise one or more native soybean SCN resistance loci or genes, such as one or more of the SCN resistance genes or loci from the resistance sources of Table 1, or one or more of the SCN resistance genes or loci from the resistance sources PI 88788, PI 548402, PI 209332, or PI 437654.

Even further provided herein is the use of a transgenic soybean plant, seed or progeny thereof, to control weeds in a soybean field, wherein each of said plant, seed or progeny comprises elite event EE-GM4 in its genome, wherein EE-GM4 which is an inserted T-DNA at a defined locus, as contained in reference seed deposited at ATCC under deposit number PTA-123624, wherein said inserted T-DNA comprises a chimeric Cry14Ab-1-encoding gene and a chimeric HPPD-4-encoding gene, and wherein said elite event is characterized by the 5′ junction sequence of SEQ ID No. 1 or 3 and by the 3′ junction sequence of SEQ ID No. 2 or 4. In one embodiment, in such use the transgenic soybean plant, seed or progeny thereof is resistant to nematodes and/or tolerant to an HPPD inhibitor herbicide. In one embodiment, said T-DNA comprises a chimeric Cry14Ab-1-encoding gene and a chimeric HPPD-4-encoding gene, and said elite event is characterized by the 5′ junction sequence of SEQ ID No. 5 and by the 3′ junction sequence of SEQ ID No. 6. In one embodiment, the invention also relates to such plants or seeds treated with one or more of the compounds and/or biological control agents or mixtures thereof as described herein. In another embodiment such plants or seeds also comprise one or more native soybean SCN resistance loci or genes, such as one or more of the SCN resistance genes or loci from the resistance sources of Table 1, or one or more of the SCN resistance genes or loci from the resistance sources PI 88788, PI 548402, PI 209332, or PI 437654.

Also provided herein is the use of a soybean plant or seed comprising elite event EE-GM4 in its genome to grow a nematode-resistant and/or herbicide-tolerant plant, wherein said elite event EE-GM4 is an inserted T-DNA at a defined locus, as contained in reference seed deposited at ATCC under deposit number PTA-123624, wherein said inserted T-DNA comprises a chimeric Cry14Ab-1-encoding gene and a chimeric HPPD-4-encoding gene, and wherein said elite event is characterized by the 5′ junction sequence of SEQ ID No. 1 or 3 and by the 3′ junction sequence of SEQ ID No. 2 or 4, and said use include the use of one or more of the compound(s) and/or biological control agent(s) or mixture, as described herein, such as wherein said compound or biological control agent is nematicidal, insecticidal, or fungicidal, or a compound or biological control agent or combination thereof selected from any one of group IAN1, IAN2, IAN3, IAN4, IAN5, IAN6, IAN7, IAN8, IAN9, IAN10, IAN11, IAN12, IAN13, IAN14, IAN15, IAN16, IAN17, IAN18, IAN19, IAN20, IAN21, IAN22, IAN23, IAN24, IAN25, IAN26, IAN27, IAN28, IAN29, IAN30, SIAN1, F1, F2, F3, F4, F5, F6, F7, F8, F9, F9, F10, F11, F12, F13, F14, F15, F16, SF1, P1, BCA, BCA2, BCA3, BCA4, BCA5, BCA6, BCA7, BCA8, BCA9, BCA10, NC1, NC2, NC3, SBCA1, SAI1, SC1 or SC2, as described herein. In one embodiment, in such use the soybean plant or seed is resistant to SCN nematodes and/or tolerant to an HPPD inhibitor herbicide. In one embodiment, said T-DNA comprises a chimeric Cry14Ab-1-encoding gene and a chimeric HPPD-4-encoding gene, and said elite event is characterized by the 5′ junction sequence of SEQ ID No. 5 or 24 and by the 3′ junction sequence of SEQ ID No. 6 or 25.

Also provided herein is the use of a soybean seed comprising elite event EE-GM4 as described herein, treated with at least one compound or biological control agent or combination as described herein, to produce a soybean crop. In one embodiment, said seed also comprises one or more native soybean SCN resistance loci or genes, such as one or more of the SCN resistance genes or loci from the resistance sources of Table 1, or one or more of the SCN resistance genes or loci from the resistance sources PI 88788, PI 548402, PI 209332, or PI 437654. In another embodiment, said seed comprises another soybean transformation event such as a soybean transformation event providing tolerance to additional herbicides, a soybean transformation event providing tolerance to nematodes via another mode of action compared to Cry14Ab-1, or a soybean transformation event providing insect control, or any one of the following soybean transformation events: Event MON87751, Event pDAB8264.42.32.1, Event DAS-81419-2, Event FG-072, Event SYHTOH2, Event DAS-68416-4, Event DAS-81615-9, Event DAS-44406-6, Event MON87708, Event MON89788, Event DAS-14536-7, Event GTS 40-3-2, Event A2704-12, Event BPS-CV127-9, Event A5547-127, Event MON87754, Event DP-3Ø5423-1, Event MON87701, Event MON87705, Event MON87712, Event pDAB4472-1606, Event DP-356043-5, Event MON87769, Event IND-ØØ41Ø-5, Event DP305423, or any of the following soybean event combinations: MON89788×MON87708, HOS×GTS 40-3-2, FG-072×A5547-127, MON87701×MON 89788, DAS-81419-2×DAS-44406-6, DAS-81419-2×DAS-68416-4, DAS-68416-4×MON 89788, MON 87705×MON 89788, MON 87769×MON 89788, DP305423×GTS 40-3-2, DP305423×MON87708, Event DP305423×MON87708×Event MON89788, DP305423×MON89788, MON87705×MON87708, MON87705×MON87708×MON89788, MON89788×MON87708×A5547-127, MON87751×MON87701×MON87708 x MON89788, SYHTOH2×MON89788, SYHTOH2×GTS 40-3-2, SYHTOH2×MON89788 x MON87708.

In one embodiment, the uses of a soybean plant or seed of the invention as described herein also includes the use of one or more of the compound(s) and/or biological control agent(s) or a mixture, as described herein, for treating the soybean plants or seeds, or for treating the soil in which the soybean plants or seeds are grown or are intended to be grown, such as wherein said compound or biological control agent is nematicidal, insecticidal, or fungicidal. In another embodiment such plants or seeds also comprise one or more native soybean SCN resistance loci or genes, such as one or more of the SCN resistance genes or loci from the resistance sources of Table 1, or one or more of the SCN resistance genes or loci from the resistance sources PI 88788, PI 548402, PI 209332, or PI 437654. In another embodiment, said plants or seeds (with or without a native soybean SCN resistance loci or genes) also comprise another soybean transformation event such as a soybean transformation event providing tolerance to additional herbicides, a soybean transformation event providing tolerance to nematodes (such as SCN, lesion nematodes, root-knot nematodes and/or reniform nematodes) via another mode of action compared to Cry14Ab-1, or a soybean transformation event providing insect control, or any one of the following soybean transformation events: Event MON87751, Event pDAB8264.42.32.1, Event DAS-81419-2, Event FG-072, Event SYHTOH2, Event DAS-68416-4, Event DAS-81615-9, Event DAS-44406-6, Event MON87708, Event MON89788, Event DAS-14536-7, Event GTS 40-3-2, Event A2704-12, Event BPS-CV127-9, Event A5547-127, Event MON87754, Event DP-305423-1, Event MON87701, Event MON87705, Event MON87712, Event pDAB4472-1606, Event DP-356043-5, Event MON87769, Event IND-ØØ41Ø-5, Event DP305423, or any of the following soybean event combinations: MON89788×MON87708, HOS×GTS 40-3-2, FG-072 x A5547-127, MON87701×MON 89788, DAS-81419-2×DAS-44406-6, DAS-81419-2 x DAS-68416-4, DAS-68416-4×MON 89788, MON 87705×MON 89788, MON 87769×MON 89788, DP305423×GTS 40-3-2, DP305423×MON87708, Event DP305423×MON87708 x Event MON89788, DP305423×MON89788, MON87705×MON87708, MON87705 x MON87708×MON89788, MON89788×MON87708×A5547-127, MON87751×MON87701 x MON87708×MON89788, SYHTOH2×MON89788, SYHTOH2×GTS 40-3-2, SYHTOH2 x MON89788×MON87708.

Also provided herein is a method for producing a soybean plant or seed comprising elite event EE-GM4, comprising crossing a plant comprising EE-GM4 with another soybean plant, and planting seed comprising EE-GM4 obtained from said cross. In one embodiment, such method includes a step of application of an HPPD inhibitor herbicide and a step of application of one or more of the compounds or biological control agents or combinations described herein on said seed or plant, or to the soil wherein said seed or plant is grown or is intended to be grown.

In accordance with this invention, also provided is the use of a soybean seed comprising elite event EE-GM4 as described above, and an HPPD inhibitor herbicide, to control weeds in a soybean field, and the use of a soybean seed comprising elite event EE-GM4 in a method of growing soybeans tolerant to HPPD inhibitor herbicides, wherein said seed is a seed treated with the compounds or biological control agents or combinations described herein, such as the compounds or biological control agents or combinations described in group SIAN1, SF1, BCA8, BCA9, BCA10, NC1, NC2, NC3, SBCA1, SAI1, SC1 or SC2 as described herein.

Further provided herein is the use of elite event EE-GM4 as described above to confer resistance to nematodes and/or tolerance to an HPPD inhibitor herbicide to a soybean plant or seed, or the use of a soybean plant or seed comprising elite event EE-GM4, in combination with an HPPD inhibitor herbicide, for growing soybeans.

Also provided herein is a primer pair specific for EE-GM4, as well as kits or methods using such primer pair, wherein at least one primer of said pair is labeled (such as with a (heterologous) detectable or screenable moiety that is added to the primer), or wherein the 5′ end of at least one of said primers comprises one or more mismatches or a nucleotide sequence unrelated to the 5′ or 3′ flanking sequences of EE-GM4 or unrelated to the T-DNA sequence of EE-GM4; or wherein at least one of said primers comprises a nucleotide sequence at their 3′ end spanning the joining region between the T-DNA flanking sequences and the T-DNA sequences, said joining region being at nucleotides 227-228 in SEQ ID No. 5, nucleotides 1058-1059 in SEQ ID No. 24, or at nucleotides 253-254 in SEQ ID No. 6 or 25, provided that the 17 consecutive nucleotides at the 3′ end are not derived exclusively from either the T-DNA or T-DNA flanking sequences in SEQ ID Nos. 5 or 24, or 6 or 25; or wherein at least one of said primers comprises a sequence which is between 80 and 100% identical to a sequence within the 5′ or 3′ flanking region of EE-GM4 or within the inserted T-DNA of EE-GM4, respectively, and said primer sequence comprises at least one mismatch with said 5′ or 3′ flanking region or said T-DNA, provided the at least one mismatch still allows specific identification of the elite event EE-GM4 with these primers under optimized detection conditions (e.g., optimized PCR conditions); or wherein the nucleotide sequence of at least one of said primers comprises the nucleotide sequence of a nucleic acid fused to a nucleic acid from another origin, or its complement.

The invention also relates to the above-described plants or seeds comprising elite event EE-GM4 treated with pesticidal (e.g., nematicidal, insecticidal and/or fungicidal) compounds and/or biological control agents or mixtures thereof, wherein said treatment can be achieved by treating the soil wherein said plants or seeds are to be grown, by treating a field sown with said seeds or planted with said plants, or by treatment of the seed to be planted. The treatment with compounds and/or biological control agents or mixtures as described herein, including the treatment with HPPD inhibitor herbicides can be sequentially or simultaneous. Application can be as a split application over time, or the application of the individual active agents or the mixtures comprising the active agents in a plurality of portions (sequential application), can be by pre-emergence application, by post-emergence application, by early post-emergence applications, or by medium or late post-emergence, or can be a combination thereof, particularly for different active ingredients. The skilled person knows the application timings and methods suited for each active ingredient/combination.

The invention further relates to methods to improve yield in soybean comprising elite event EE-GM4 as described above, wherein the soybean plants or seeds, or the soil in which soybean plants or seeds are grown or are intended to be grown, are treated with the compounds and/or biological control agents or mixtures thereof as described herein, as well as to use of the plants or seeds comprising EE-GM4 as described herein with compounds and/or biological control agents or mixtures thereof as described herein. One embodiment of the invention relates to seed comprising elite event EE-GM4 treated with pesticidal (e.g., nematicidal, insecticidal, acaricidal, or fungicidal) compounds and/or biological agents or mixtures comprising them, so as to ensure improved protection of the seed, the germinated plantlet and the plant grown from the seed from agricultural pests. In one embodiment of the invention, the seed comprising elite event EE-GM4 contains a coating of one or more nematicidal compounds and/or biological control agents, or mixtures comprising them, such as seed comprising elite event EE-GM4 coated with at least one nematicidal compound (such as tioxazafen, fluopyram, metam, oxamyl, or abamectin, or any of the nematicidal compounds described herein or known in the art), and at least one nematicidal biological control agent (such as Bacillus firmus, Pasteuria nishizawae, Bacillus subtilis, Bacillus lichenformis, Bacillus amyloliquefaciens, Burkholderia rinojensis, or any of the nematicidal biological control agents described herein or known in the art). In one embodiment of the invention, that seed coating also contains (besides the nematicidal agents) one or more insecticidal coumpounds or biological control agents as described herein (such as clothianidin, tetraniliprole, spirotetramat, flupyradifurone, thiamethoxam, chlorpyrifos, gamma-cyhalothrin, lambda-cyhalothrin, chlorantraniliprole, bifethrin, imidacloprid, zetacypermethrin, cyfluthrin, Bacillus thuringiensis, diflubenzuron), and/or one or more fungicidal coumpounds or biological control agents, as described herein (such as any one or more of Sedaxane, Fludioxonil, Mefenoxam, flutriafol, fluxapyroxad, pyraclostrobin, tetraconazole, azoxystrobin, propiconazole, benzovindiflupyr, tebuconazole, azoxystrobin).

In another embodiment such plants or seeds also comprise one or more native soybean SCN resistance loci or genes, such as one or more of the SCN resistance genes or loci from the resistance sources of Table 1, or one or more of the SCN resistance genes or loci from the resistance sources PI 88788, PI 548402, PI 209332, or PI 437654. In another embodiment, said plants or seeds (with or without said native soybean SCN resistance loci or genes) also comprise another soybean transformation event such as a soybean transformation event providing tolerance to additional herbicides, a soybean transformation event providing tolerance to nematodes (such as SCN, lesion nematodes, root-knot nematodes and/or reniform nematodes) via another mode of action compared to Cry14Ab-1, or a soybean transformation event providing insect control, or any one of the following soybean transformation events: Event MON87751, Event pDAB8264.42.32.1, Event DAS-81419-2, Event FG-072, Event SYHTOH2, Event DAS-68416-4, Event DAS-81615-9, Event DAS-44406-6, Event MON87708, Event MON89788, Event DAS-14536-7, Event GTS 40-3-2, Event A2704-12, Event BPS-CV127-9, Event A5547-127, Event MON87754, Event DP-3Ø5423-1, Event MON87701, Event MON87705, Event MON87712, Event pDAB4472-1606, Event DP-356043-5, Event MON87769, Event IND-ØØ41Ø-5, Event DP305423, or any of the following soybean event combinations: MON89788 x MON87708, HOS×GTS 40-3-2, FG-072×A5547-127, MON87701×MON 89788, DAS-81419-2×DAS-44406-6, DAS-81419-2×DAS-68416-4, DAS-68416-4×MON 89788, MON 87705×MON 89788, MON 87769×MON 89788, DP305423×GTS 40-3-2, DP305423 x MON87708, Event DP305423×MON87708×Event MON89788, DP305423×MON89788, MON87705×MON87708, MON87705×MON87708×MON89788, MON89788×MON87708 x A5547-127, MON87751×MON87701×MON87708×MON89788, SYHTOH2 x MON89788, SYHTOH2×GTS 40-3-2, SYHTOH2×MON89788×MON87708. In one embodiment, said compound or biological control agent or combination is selected from any one of group IAN1, IAN2, IAN3, IAN4, IAN5, IAN6, IAN7, IAN8, IAN9, IAN10, IAN11, IAN12, IAN13, IAN14, IAN15, IAN16, IAN17, IAN18, IAN19, IAN20, IAN21, IAN22, IAN23, IAN24, IAN25, IAN26, IAN27, IAN28, IAN29, IAN30, SIAN1, F1, F2, F3, F4, F5, F6, F7, F8, F9, F9, F10, F11, F12, F13, F14, F15, F16, SF1, P1, BCA1, BCA2, BCA3, BCA4, BCA5, BCA6, BCA7, BCA8, BCA9, BCA10, NC1, NC2, NC3, SBCA1, SAI1, SC1 or SC2, as described herein.

The invention also relates to methods for controlling soybean pests, such as soybean nematodes, on plants or seed of the invention by treating said plants or seeds with compounds and/or biological control agents or mixtures thereof that act on soybean pests, such as soybean nematodes, and/or their habitat.

Also provided herein is a method to prevent or delay nematode resistance development to the Cry14Ab-1 protein or to elite event of the invention, comprising treating the plants or seeds of the invention with one or more nematicidal compound(s) and/or nematicidal biological control agent(s), or mixtures containing them, or treating the soil wherein the plants or seeds of the invention will be grown (which can be followed by planting or sowing said plants or seeds in said soil). In one embodiment, said use is of one or more nematicidal compound(s) and one or more nematicidal biological control agent(s). In one embodiment, said nematode is soybean cyst nematode, a Pratylenchus species nematode, a root-knot nematode, and/or a reniform nematode, such as any of said nematodes feeding on soybean. In one embodiment more than one nematicidal compound or more than one nematicidal biological control agent is used or more than one nematicidal compound and more than one nematicidal biological control agent is used, particularly when they each have a different mode of action. In one embodiment, seeds comprising EE-GM4 are treated with said compounds or biological control agents.

In one embodiment of the above methods for controlling soybean pests or to prevent or delay nematode resistance development, such plants or seeds also comprise one or more native soybean SCN resistance loci or genes, such as one or more of the SCN resistance genes or loci from the resistance sources of Table 1, or one or more of the SCN resistance genes or loci from the resistance sources PI 88788, PI 548402, PI 209332, or PI 437654. In another embodiment, said plants or seeds (with or without said native soybean SCN resistance loci or genes) also comprise another soybean transformation event such as a soybean transformation event providing tolerance to additional herbicides, a soybean transformation event providing tolerance to nematodes (such as SCN, lesion nematodes, root-knot nematodes and/or reniform nematodes) via another mode of action compared to Cry14Ab-1, or a soybean transformation event providing insect control, or any one of the following soybean transformation events: Event MON87751, Event pDAB8264.42.32.1, Event DAS-81419-2, Event FG-072, Event SYHTOH2, Event DAS-68416-4, Event DAS-81615-9, Event DAS-44406-6, Event MON87708, Event MON89788, Event DAS-14536-7, Event GTS 40-3-2, Event A2704-12, Event BPS-CV127-9, Event A5547-127, Event MON87754, Event DP-3Ø5423-1, Event MON87701, Event MON87705, Event MON87712, Event pDAB4472-1606, Event DP-356043-5, Event MON87769, Event IND-ØØ41Ø-5, Event DP305423, or any of the following soybean event combinations. MON89788 x MON87708, HOS×GTS 40-3-2, FG-072×A5547-127, MON87701×MON 89788, DAS-81419-2×DAS-44406-6, DAS-81419-2×DAS-68416-4, DAS-68416-4×MON 89788, MON 87705×MON 89788, MON 87769×MON 89788, DP305423×GTS 40-3-2, DP305423 x MON87708, Event DP305423×MON87708×Event MON89788, DP305423×MON89788, MON87705×MON87708, MON87705×MON87708×MON89788, MON89788×MON87708 x A5547-127, MON87751×MON87701×MON87708×MON89788, SYHTOH2 x MON89788, SYHTOH2×GTS 40-3-2, SYHTOH2×MON89788×MON87708. In one embodiment, said compound or biological control agent or combination is selected from any one of group IAN1, IAN2, IAN3, IAN4, IAN5, IAN6, IAN7, IAN8, IAN9, IAN10, IAN11, IAN12, IAN13, IAN14, IAN15, IAN16, IAN17, IAN18, IAN19, IAN20, IAN21, IAN22, IAN23, IAN24, IAN25, IAN26, IAN27, IAN28, IAN29, IAN30, SIAN1, F1, F2, F3, F4, F5, F6, F7, F8, F9, F9, F10, F11, F12, F13, F14, F15, F16, SF1, P1, BCA1, BCA2, BCA3, BCA4, BCA5, BCA6, BCA7, BCA8, BCA9, BCA10, NC1, NC2, NC3, SBCA1, SAI1, SC1 or SC2, as described herein. In one embodiment, said compound or biological control agent or combination is selected from any one of group IAN1, IAN2, IAN3, IAN4, IAN5, IAN6, IAN7, IAN8, IAN9, IAN10, IAN11, IAN12, IAN13, IAN14, IAN15, IAN16, IAN17, IAN18, IAN19, IAN20, IAN21, IAN22, IAN23, IAN24, IAN25, IAN26, IAN27, IAN28, IAN29, IAN30, SIAN1, F1, F2, F3, F4, F5, F6, F7, F8, F9, F9, F10, F11, F12, F13, F14, F15, F16, SF1, P1, BCA1, BCA2, BCA3, BCA4, BCA5, BCA6, BCA7, BCA8, BCA9, BCA10, NC1, NC2, NC3, SBCA1, SAI1, SC1 or SC2, as described herein.

In one embodiment, the plants and seeds that were or are to be treated in accordance with the invention contain (different from event EE-GM4), SCN resistance loci or genes from one or more of SCN resistance sources PI 548316, PI 567305, PI 437654, PI 90763, PI 404198B, PI 88788, PI 468916, PI 567516C, PI 209332, PI 438489B, PI 89772, Peking, PI 548402, PI 404198A, PI 561389B, PI 629013, PI 507471, PI 633736, PI 507354, PI 404166, PI 437655, PI 467312,PI567328,PI22897, or PI494182 In one embodiment, the compound(s) or the biological control agent(s), or mixtures, of the invention as described herein are selected from any one of the groups as described herein, sudh as: H1, H2, H3, H4, H5, IAN1, IAN2, IAN3, IAN4, IAN5, IAN6, IAN7, IAN8, IAN9, IAN10, IAN11, IAN12, IAN13, IAN14, IAN15, IAN16, IAN17, IAN18, IAN19, IAN20, IAN21, IAN22, IAN23, IAN24, IAN25, IAN26, IAN27, IAN28, IAN29, IAN30, SIAN1, F1, F2, F3, F4, F5, F6, F7, F8, F9, F9, F10, F11, F12, F13, F14, F15, F16, SF1, P1, BCA1, BCA2, BCA3, BCA4, BCA5, BCA6, BCA7, BCA8, BCA9, BCA10, NC1, NC2, NC3, SBCA1, SAI1, SCI or SC2, as well as the preferred combinations or mixtures described herein.

In one embodiment, a seed of the invention is treated with compound(s) and/or the biological control agent(s), as described herein, or a combination described herein, wherein said compound or agent or combination is from group SIAN1, BCA8, BCA9, BCA10, NC1, NC2, NC3, SBCA1, SAI1, SCI or SC2.

Other embodiments referred to in this invention are summarized in the following paragraphs:
1. A method for identifying elite event EE-GM4 in biological samples, which method comprises detection of an EE-GM4 specific region with a specific primer pair or probe which specifically recognize(s) (at least a part of) the 5′ or 3′ T-DNA flanking region and (at least a part of) the inserted T-DNA contiguous therewith in EE-GM4.
2. The method of paragraph 1, said method comprising amplifying a DNA fragment of between 50 and 1000 bp from a nucleic acid present in said biological samples using a polymerase chain reaction with at least two primers, one of said primers recognizing the 5′ T-DNA flanking region of the inserted T-DNA in EE-GM4, said 5′ T-DNA flanking region comprising the nucleotide sequence of SEQ ID No. 5 from nucleotide 1 to nucleotide 227 or of SEQ ID No. 24 from nucleotide 1 to nucleotide 1058 or recognizing the 3′ T-DNA flanking region of the inserted T-DNA in EE-GM4, said 3′ T-DNA flanking region comprising the nucleotide sequence of the complement of SEQ ID No. 6 from nucleotide 254 to nucleotide 501 or the nucleotide sequence of the complement of SEQ ID No. 25 from nucleotide 254 to nucleotide 1339, the other primer of said primers recognizing a sequence within the inserted T-DNA comprising the nucleotide sequence of SEQ ID No. 5 from nucleotide 228 to nucleotide 398 or the complement thereof, or the nucleotide sequence of SEQ ID No. 6 from nucleotide 1 to nucleotide 253, or the nucleotide sequence of SEQ ID No. 11 from nucleotide 17 to nucleotide 7621 or of SEQ ID No. 23 from nucleotide position 1059 to nucleotide position 8663, or the complement thereof.
3. The method of paragraph 2, wherein said primer recognizing the 5′ T-DNA flanking region comprises a nucleotide sequence of 17 to 200 consecutive nucleotides selected from the nucleotide sequence of SEQ ID No. 5 from nucleotide 1 to nucleotide 227 or of SEQ ID No. 24 from nucleotide 1 to nucleotide 1058, or said primer recognizing the 3′ T-DNA flanking region of EE-GM4 comprises a nucleotide sequence of 17 to 200 consecutive nucleotides selected from the nucleotide sequence of the complement of SEQ ID No. 6 from nucleotide 254 to nucleotide 501 or the nucleotide sequence of the complement of SEQ ID No. 25 from nucleotide 254 to nucleotide 1339, and said primer recognizing a sequence within the inserted T-DNA comprises 17 to 200 consecutive nucleotides selected from the nucleotide sequence of SEQ ID No. 5 from nucleotide 228 to nucleotide 398 or the complement thereof, or the nucleotide sequence of SEQ ID No. 6 from nucleotide 1 to nucleotide 253 or the complement thereof, or the nucleotide sequence of SEQ ID No. 11 from nucleotide 17 to nucleotide 7621 or of SEQ ID No. 23 from nucleotide position 1059 to nucleotide position 8663, or the complement thereof.
4. The method of paragraph 2, wherein said primer recognizing the 5′ T-DNA flanking region comprises at its extreme 3′ end a nucleotide sequence of at least 17 consecutive nucleotides selected from the nucleotide sequence of SEQ ID No. 5 from nucleotide 1 to nucleotide 227 or of SEQ ID No. 24 from nucleotide 1 to nucleotide 1058, or said primer recognizing the 3′ T-DNA flanking region of EE-GM4 comprises at its extreme 3′ end a nucleotide sequence of at least 17 consecutive nucleotides selected from the nucleotide sequence of the complement of SEQ ID No. 6 from nucleotide 254 to nucleotide 501 or the nucleotide sequence of the complement of SEQ ID No. 25 from nucleotide 254 to nucleotide 1339, and said primer recognizing a sequence within the inserted T-DNA comprises at its 3′ end at least 17 consecutive nucleotides selected from the complement of the nucleotide sequence of SEQ ID No. 5 from nucleotide 228 to nucleotide 398, or the nucleotide sequence of SEQ ID No. 6 from nucleotide 1 to nucleotide 253, or the nucleotide sequence of SEQ ID No. 11 from nucleotide 17 to nucleotide 7621 or of SEQ ID No. 23 from nucleotide position 1059 to nucleotide position 8663, or the complement thereof.
5. The method of paragraph 4, wherein said primers comprise the sequence of SEQ ID No. 12 and SEQ ID No. 13, respectively, or the sequence of SEQ ID No. 20 and SEQ ID No. 21, respectively.
6. The method of paragraph 5, which method comprises amplifying an EE-GM4-specific fragment of 126 or 90 bp using PCR.
7. The method of any one of paragraphs 2 to 6, further comprising the step of hybridizing a probe specific for the DNA fragment amplified with said at least two primers.
8. The method of paragraph 7, wherein said probe recognizes part of said 5′ T-DNA flanking region and part of the inserted T-DNA contiguous therewith, or wherein said probe recognizes part of said 3′ T-DNA flanking region and part of the inserted T-DNA contiguous therewith, or recognizes part of said 5′ T-DNA flanking region and part of the inserted T-DNA contiguous therewith, such as wherein said probe comprises the nucleotide sequence of SEQ ID No. 1 or 3 or SEQ ID No 2 or 4.
9. The method of paragraph 8, wherein said primers comprise the sequence of SEQ ID No. 12 and SEQ ID No. 13, respectively, and wherein said probe comprises the sequence of SEQ ID No. 14, or wherein said primers comprise the sequence of SEQ ID No. 20 and SEQ ID No. 21, respectively, and wherein said probe comprises the sequence of SEQ ID No. 22.
10. A kit comprising one primer recognizing the 5′ T-DNA flanking region of the inserted T-DNA in EE-GM4, said 5′ T-DNA flanking region comprising the nucleotide sequence of SEQ ID No. 5 from nucleotide 1 to nucleotide 227 or of SEQ ID No. 24 from nucleotide 1 to nucleotide 1058, or one primer recognizing the 3′ T-DNA flanking region of the inserted T-DNA in EE-GM4, said 3′ T-DNA flanking region comprising the nucleotide sequence of the complement of SEQ ID No. 6 from nucleotide 254 to nucleotide 501 or the nucleotide sequence of the complement of SEQ ID No. 25 from nucleotide 254 to nucleotide 1339, and one primer recognizing a sequence within the inserted T-DNA, said inserted T-DNA comprising the complement of the nucleotide sequence of SEQ ID No. 5 from nucleotide 228 to nucleotide 398 or the nucleotide sequence of SEQ ID No. 6 from nucleotide 1 to nucleotide 253, or the nucleotide sequence of SEQ ID No. 11 from nucleotide 17 to nucleotide 7621 or of SEQ ID No. 23 from nucleotide position 1059 to nucleotide position 8663, or the complement thereof.
11. The kit of paragraph 10, wherein said primer recognizing the 5′ T-DNA flanking region comprises a nucleotide sequence of 17 to 200 consecutive nucleotides selected from the nucleotide sequence of SEQ ID No. 5 from nucleotide 1 to nucleotide 227 or of SEQ ID No. 24 from nucleotide 1 to nucleotide 1058, or said primer recognizing the 3′ T-DNA flanking region of EE-GM4 comprises a nucleotide sequence of 17 to 200 consecutive nucleotides selected from the nucleotide sequence of the complement of SEQ ID No. 6 from nucleotide 254 to nucleotide 501 or the nucleotide sequence of the complement of SEQ ID No. 25 from nucleotide 254 to nucleotide 1339, and said primer recognizing a sequence within the inserted T-DNA comprises 17 to 200 consecutive nucleotides selected from the complement of the nucleotide sequence of SEQ ID No. 5 from nucleotide 228 to nucleotide 398 or the nucleotide sequence of SEQ ID No. 6 from nucleotide 1 to nucleotide 253, or the nucleotide sequence of SEQ ID No. 11 from nucleotide 17 to nucleotide 7621 or of SEQ ID No. 23 from nucleotide position 1059 to nucleotide position 8663, or the complement thereof.
12. The kit of paragraph 10, wherein said primer recognizing the 5′ T-DNA flanking region comprises at its extreme 3′ end a nucleotide sequence of at least 17 consecutive nucleotides selected from the nucleotide sequence of SEQ ID No. 5 from nucleotide 1 to nucleotide 227 or of SEQ ID No. 24 from nucleotide 1 to nucleotide 1058, or said primer recognizing the 3′ T-DNA flanking region of EE-GM4 comprises at its extreme 3′ end a nucleotide sequence of at least 17 consecutive nucleotides selected from the nucleotide sequence of the complement of SEQ ID No. 6 from nucleotide 254 to nucleotide 501 or the nucleotide sequence of the complement of SEQ ID No. 25 from nucleotide 254 to nucleotide 1339, and said primer recognizing a sequence within the inserted T-DNA comprises at its 3′ end at least 17 consecutive nucleotides selected from the complement of the nucleotide sequence of SEQ ID No. 5 from nucleotide 228 to nucleotide 398 or the nucleotide sequence of SEQ ID No. 6 from nucleotide 1 to nucleotide 253, or the nucleotide sequence of SEQ ID No. 11 from nucleotide 17 to nucleotide 7621 or of SEQ ID No. 23 from nucleotide position 1059 to nucleotide position 8663, or the complement thereof.
13. The kit of paragraph 10, comprising a primer comprising the sequence of SEQ ID No. 12 and a primer comprising the sequence of SEQ ID No. 13 or comprising a primer comprising the sequence of SEQ ID No. 20 and a primer comprising the sequence of SEQ ID No. 21.
14. The kit of paragraph 10, further comprising a probe recognizing a sequence between the primer recognizing the 5′ T-DNA flanking region and the primer recognizing the sequence within the inserted T-DNA, or recognizing a sequence between the primer recognizing the 3′ T-DNA flanking region and the primer recognizing the sequence within the inserted T-DNA.
15. The kit of paragraph 14, wherein said probe recognizes part of said 5′ T-DNA flanking region and part of the inserted T-DNA contiguous therewith, or wherein said probe recognizes part of said 3′ T-DNA flanking region and part of the inserted T-DNA contiguous therewith.
16. The kit of paragraph 15, wherein said primers comprise the sequence of SEQ ID No. 12 and SEQ ID No. 13, and wherein said probe comprises the sequence of SEQ ID No. 14, or wherein said primers comprise the sequence of SEQ ID No. 20 and SEQ ID No. 21, and wherein said probe comprises the sequence of SEQ ID No. 22.
17. A primer pair suitable for use in an EE-GM4 specific detection, comprising a first primer comprising a sequence which, under optimized detection conditions specifically recognizes a sequence within the 5′ or 3′ T-DNA flanking region of the inserted T-DNA in EE-GM4, and a second primer comprising a sequence which, under optimized detection conditions specifically recognizes a sequence within the inserted T-DNA in EE-GM4 contiguous with said flanking 5′ or 3′ region, said 5′ T-DNA flanking region comprising the nucleotide sequence of SEQ ID No. 5 from nucleotide 1 to nucleotide 227 or of SEQ ID No. 24 from nucleotide 1 to nucleotide 1058, said 3′ T-DNA flanking region comprising the nucleotide sequence of the complement of SEQ ID No. 6 from nucleotide 254 to nucleotide 501 or the nucleotide sequence of the complement of SEQ ID No. 25 from nucleotide 254 to nucleotide 1339, said inserted T-DNA comprising the nucleotide sequence of SEQ ID No. 5 from nucleotide 228 to nucleotide 398, or the nucleotide sequence of SEQ ID No. 6 from nucleotide 1 to nucleotide 253, or the nucleotide sequence of SEQ ID No. 11 from nucleotide 17 to nucleotide 7621 or of SEQ ID No. 23 from nucleotide position 1059 to nucleotide position 8663, or the complement thereof.
18. A primer comprising at its extreme 3′ end the sequence of SEQ ID No. 12, or the sequence of SEQ ID No. 13, or the sequence of SEQ ID No. 20, or the sequence of SEQ ID No. 21.
19. A primer pair comprising a first primer comprising at its extreme 3′ end the sequence of SEQ ID No. 12 and a second primer comprising at its extreme 3′ end the sequence of SEQ ID No. 13, or comprising a first primer comprising at its extreme 3′ end the sequence of SEQ ID No. 20 and a second primer comprising at its extreme 3′ end the sequence of SEQ ID No. 21.
20. The method of paragraph 1, which method comprises hybridizing a nucleic acid of biological samples with a specific probe for EE-GM4.
21. The method of paragraph 20, wherein the sequence of said specific probe has at least 80% sequence identity with a sequence comprising part of the 5′ T-DNA flanking sequence or the 3′ T-DNA flanking sequence of EE-GM4 and the sequence of the inserted T-DNA contiguous therewith.
22. The method of paragraph 21, wherein the sequence of said specific probe comprises a sequence with at least 80% sequence identity to the sequence of any one of SEQ ID No. 1, 3, or 5 or the sequence of any one of SEQ ID No. 2, 4, or 6, or the complement of said sequences.
23. The method of paragraph 22, wherein said probe comprises the sequence of any one of SEQ ID No. 1 or 3 or the sequence of any one of SEQ ID No. 2 or 4.
24. A kit for identifying elite event EE-GM4 in biological samples, said kit comprising a specific probe, capable of hybridizing specifically to a specific region of EE-GM4.
25. The kit of paragraph 24, wherein the sequence of said specific probe has at least 80% sequence identity with a sequence comprising part of the 5′ T-DNA flanking sequence or part of the 3′ T-DNA flanking sequence and part of the inserted T-DNA contiguous therewith in EE-GM4.
26. The kit of paragraph 25, wherein the sequence of said specific probe comprises a nucleotide sequence having at least 80% sequence identity with any one of SEQ ID No. 1, 3 or 5 or any one of SEQ ID No. 2, 4 or 6, or the complement of said sequences.
27. A specific probe for the identification of elite event EE-GM4 in biological samples.
28. The probe of paragraph 27, which comprises a nucleotide sequence having at least 80% sequence identity with a sequence comprising part of the 5′ T-DNA flanking sequence or part of the 3′ T-DNA flanking sequence and part of the inserted T-DNA contiguous therewith in EE-GM4, or the complement thereof.
29. The probe of paragraph 28 which has at least 80% sequence identity with the sequence of any one of SEQ ID No. 1, 3 or 5 or the sequence of any one of SEQ ID No. 2, 4, or 6, or the complement of said sequences, or said probe comprising the sequence of any one of SEQ ID No. 1, 3 or 5 or the sequence of any one of SEQ ID No. 2, 4, or 6.
30. A specific probe comprising a nucleotide sequence being essentially similar to any one of SEQ ID No. 1, 3, or 5 or any one of SEQ ID No. 2, 4, or 6, or the complement of said sequences.
31. A specific probe comprising the sequence of SEQ ID No. 1 or 3 or the sequence of SEQ ID No. 2 or 4.
32. A method for confirming seed purity, which method comprises detection of an EE-GM4 specific region with a specific primer or probe which specifically recognizes the 5′ or 3′ T-DNA flanking region and the inserted T-DNA contiguous therewith in EE-GM4, in seed samples.
33. The method of paragraph 32, comprising amplifying a DNA fragment of between 50 and 1000 bp from a nucleic acid present in said biological samples using a polymerase chain reaction with at least two primers, one of said primers recognizing the 5′ T-DNA flanking region of the inserted T-DNA in EE-GM4, said 5′ T-DNA flanking region comprising the nucleotide sequence of SEQ ID No. 5 from nucleotide 1 to nucleotide 227 or of SEQ ID No. 24 from nucleotide 1 to nucleotide 1058, or the 3′ T-DNA flanking region of the inserted T-DNA in EE-GM4, said 3′ T-DNA flanking region comprising the nucleotide sequence of the complement of SEQ ID No. 6 from nucleotide 254 to nucleotide 501 or the nucleotide sequence of the complement of SEQ ID No. 25 from nucleotide 254 to nucleotide 1339, the other primer of said primers recognizing a sequence within the inserted T-DNA comprising the complement of the nucleotide sequence of SEQ ID No. 5 from nucleotide 228 to nucleotide 398 or the nucleotide sequence of SEQ ID No. 6 from nucleotide 1 to nucleotide 253, or the nucleotide sequence of SEQ ID No. 11 from nucleotide 17 to nucleotide 7621, or the nucleotide sequence of SEQ ID No. 23 from nucleotide position 1059 to nucleotide position 8663, or the complement thereof, and hybridizing a probe specific for the DNA fragment amplified with said at least two primers.
34. The method of paragraph 33, comprising amplifying a DNA fragment of 126 bp and wherein said primers comprise the sequence of SEQ ID No. 12 and SEQ ID No. 13, respectively, and wherein said probe comprises the sequence of SEQ ID No. 14, or amplifying a DNA fragment of 90 bp and wherein said primers comprise the sequence of SEQ ID No. 20 and SEQ ID No. 21, respectively, and wherein said probe comprises the sequence of SEQ ID No. 22.
35. A method for screening seeds for the presence of EE-GM4, which method comprises detection of an EE-GM4 specific region with a specific primer pair or probe which specifically recognize(s) the 5′ or 3′ T-DNA flanking region and the inserted T-DNA contiguous therewith in EE-GM4, in samples of seed lots.
36. The method of paragraph 35, comprising amplifying a DNA fragment of between 50 and 1000 bp from a nucleic acid present in said biological samples using a polymerase chain reaction with at least two primers, one of said primers recognizing the 5′ T-DNA flanking region of the inserted T-DNA in EE-GM4, said 5′ T-DNA flanking region comprising the nucleotide sequence of SEQ ID No. 5 from nucleotide 1 to nucleotide 227 or of SEQ ID No. 24 from nucleotide 1 to nucleotide 1058, or the 3′ T-DNA flanking region of the inserted T-DNA in EE-GM4, said 3′ T-DNA flanking region comprising the nucleotide sequence of the complement of SEQ ID No. 6 from nucleotide 254 to nucleotide 501 or the nucleotide sequence of the complement of SEQ ID No. 25 from nucleotide 254 to nucleotide 1339, the other primer of said primers recognizing a sequence within the inserted T-DNA comprising the complement of the nucleotide sequence of SEQ ID No. 5 from nucleotide 228 to nucleotide 398 or the nucleotide sequence of SEQ ID No. 6 from nucleotide 1 to nucleotide 253, or comprising the nucleotide sequence of SEQ ID No. 11 from nucleotide 17 to nucleotide 7621 or of SEQ ID No. 23 from nucleotide position 1059 to nucleotide position 8663, or the complement thereof, and hybridizing a probe specific for the DNA fragment amplified with said at least two primers, such as a probe comprising the sequence of SEQ ID No. 1 or 3, or SEQ ID No. 2 or 4, or the complement thereof.
37. The method of paragraph 36, comprising amplifying a DNA fragment of 126 bp and wherein said primers comprise the sequence of SEQ ID No. 12 and SEQ ID No. 13, respectively, and wherein said probe comprises the sequence of SEQ ID No. 14.
38. A method of detecting the presence of elite event EE-GM4 in biological samples through hybridization with a substantially complementary labeled nucleic acid probe in which the probe:target nucleic acid ratio is amplified through recycling of the target nucleic acid sequence, said method comprising:
   a) hybridizing said target nucleic acid sequence to a first nucleic acid oligonucleotide comprising the nucleotide sequence of SEQ ID No. 5 from nucleotide position 228 to nucleotide position 245 or its complement or said first nucleic acid oligonucleotide comprising the nucleotide sequence of SEQ ID No. 6 from nucleotide position 236 to nucleotide position 253 or its complement;
   b) hybridizing said target nucleic acid sequence to a second nucleic acid oligonucleotide comprising the nucleotide sequence of SEQ ID No. 5 from nucleotide 210 to nucleotide 227 or its complement or said nucleic acid oligonucleotide comprising the nucleotide sequence of SEQ ID No. 6 from nucleotide 254 to nucleotide 271 or its complement, wherein said first and second oligonucleotide overlap by at least one nucleotide and wherein either said first or said second oligonucleotide is labeled to be said labeled nucleic acid probe;
   c) cleaving only the labeled probe within the probe:target nucleic acid sequence duplex with an enzyme which causes selective probe cleavage resulting in duplex disassociation, leaving the target sequence intact;
   d) recycling of the target nucleic acid sequence by repeating steps (a) to (c); and
   e) detecting cleaved labeled probe, thereby determining the presence of said target nucleic acid sequence, and detecting the presence of elite event EE-GM4 in said biological samples.
39. A transgenic soybean plant, or cells, parts, seed or progeny thereof, each comprising elite event EE-GM4 in its genome, reference seed comprising said event having been deposited at the ATCC under deposit number PTA-123624, treated with the compound(s) or the biological control agent(s), or combinations, as described herein.
40. The transgenic soybean plant, seed, cells, parts or progeny of paragraph 39, the genomic DNA of which, when analyzed using PCR for EE-GM4 with two primers comprising the nucleotide sequence of SEQ ID 12 and SEQ ID 13 respectively, yields a DNA fragment of 126 bp.
41. Seed comprising elite event EE-GM4, which is an inserted T-DNA at a specific position in the soybean genome, as is contained in the seed deposited at the ATCC under deposit number PTA-123624 or in derivatives therefrom, wherein said seed is treated with one or more of the compound(s) or the biological control agent(s), or combinations, as described herein.
42. A soybean plant, plant part, cell or tissue, or seed comprising elite event EE-GM4 obtainable from the seed of paragraph 41, treated with the compound(s) or the biological control agent(s), or combinations, as described herein.
43. A soybean plant, or seed, cells or tissues thereof, each comprising elite event EE-GM4 in its genome, obtainable by propagation of and/or breeding with a soybean plant grown from the seed deposited at the ATCC under deposit number PTA-123624, treated with the compound(s) or the biological control agent(s), or combinations, as described herein.
44. The soybean plant cell according to any one of the above paragraphs, which is a non-propagating plant cell, or a plant cell that cannot regenerate into a plant 45. A method for producing a soybean plant or seed comprising elite event EE-GM4 comprising crossing a plant according to any one of the above paragraphs with another soybean plant, and planting the seed obtained from said cross.
46. Use of a soybean plant or seed comprising a nucleic acid molecule comprising a nucleotide sequence essentially similar to the sequence of any one of SEQ ID No. 1, 3 or 5 or the sequence of any one of SEQ ID No. 2, 4, or 6, or the complement of said sequences, such as a nucleic acid molecule comprising a nucleotide sequence with at least 99% or at least 99.5% sequence identity to the nucleotide sequence of SEQ ID No. 5 or 6, or the complement thereof, such as said nucleic acid molecule which confers tolerance to an HPPD inhibitor herbicide and/or SCN resistance in soybean, and the compound(s) or the biological control agent(s), or combinations, as described herein, to produce a soybean crop.
47. The use of paragraph 46 wherein the nucleic acid molecule comprises the nucleotide sequence of any one of SEQ ID No. 1 or 3 or SEQ ID No. 2 or 4, or the complement of said sequences, such as such nucleic acid molecule which also comprises the nucleotide sequence of SEQ ID No. 7 and 9 or a nucleotide sequence having at least 98% sequence identity thereto, or the complement thereof, or such nucleic acid molecule which comprises the nucleotide sequence of SEQ ID No. 11 from nucleotide position 188 to nucleotide position 7368, or a nucleotide sequence having at least 98% sequence identity thereto.
48. A soybean plant, cell, plant part, seed or progeny thereof comprising a nucleic acid molecule of any one of these paragraphs, such as a soybean plant, cell, plant part, seed or progeny thereof comprising a nucleic acid molecule comprising the nucleotide sequence of SEQ ID No. 1, 3 or 5 or the nucleotide sequence of SEQ ID No. 2, 4, or 6, or a soybean plant, cell, plant part, seed or progeny thereof, comprising in its genome the nucleotide sequence of SEQ ID No. 3 and the nucleotide sequence of SEQ ID No. 4, or a soybean plant, cell, plant part, seed or progeny thereof, comprising in its genome the nucleotide sequence of SEQ ID No. 5 and the nucleotide sequence of SEQ ID No. 6, or a soybean plant, cell, plant part, seed or progeny thereof, comprising in its genome the nucleotide sequence of SEQ ID No. 24 and the nucleotide sequence of SEQ ID No. 25, such as such a soybean plant also comprising a Cry14Ab-1-encoding chimeric and an HPPD-4-encoding chimeric gene, particularly such chimeric genes comprising the nucleotide sequence of SEQ ID No. 7 and 9, respectively, wherein said plant, cell, plant part, seed or progeny, or the soil in which they are grown or are intended to be grown, are treated with one or more of the compounds and/or biological control agents or mixtures as described herein.
49. Use of a soybean plant or seed comprising a nucleic acid molecule comprising the nucleotide sequence of any one of SEQ ID No. 1, 3, or 5 or SEQ ID No. 2, 4, or 6, such as a nucleic acid molecule, which comprises the nucleotide sequence of SEQ ID No. 5 and SEQ ID No. 6, or the complement thereof, or such as a nucleic acid molecule, which comprises the nucleotide sequence of SEQ ID No. 24 and SEQ ID No. 25, or the complement thereof, and the compound(s) or the biological control agent(s), or combinations, as described herein, to produce a soybean crop.
50. A transgenic soybean plant, plant cell, tissue, or seed, comprising in their genome event EE-GM4 characterized by a nucleic acid molecule comprising a nucleotide sequence essentially similar to the sequence of any one of SEQ ID No. 1, 3, 5 or 24 of the sequence of any one of SEQ ID No. 2, 4, 6 or 25, or the complement of said sequences, wherein said soybean plant also comprising a Cry14Ab-1-encoding chimeric and an HPPD-4-encoding chimeric gene, wherein said plant, cell, plant part, tissue or seed, or the soil or growth medium in which they are grown or are intended to be grown, are treated with one or more of the compounds and/or biological control agents or mixtures as described herein.
51. A soybean plant, cell, tissue or seed, comprising EE-GM4 and comprising in the genome of its cells a nucleic acid sequence with at least 80%, 90%, 95% or 100% sequence identity to a sequence of any one of SEQ ID No. 1, 3 or 5 or a sequence of any one of SEQ ID No. 2, 4, or 6, or the complement of said sequences, such as a soybean plant also comprising a Cry14Ab-1-encoding chimeric and an HPPD-4-encoding chimeric gene, or such soybean plant, cell, tissue or seed, comprising in the genome of its cells a nucleic acid sequence with at least 80%, 90%, 95% or 100% sequence identity to the sequence of SEQ ID No. 24 or SEQ ID No. 25, wherein said plant, cell, tissue or seed, or the soil or growth medium in which they are grown or are intended to be grown, are treated with one or more of the compounds and/or biological control agents or mixtures as described herein.
52. A soybean plant, plant cell, tissue, or seed, comprising in its genome a nucleic acid molecule comprising a nucleotide sequence with at least 99% sequence identity to the nucleotide sequence of SEQ ID No. 5 or 24 or 6 or 25, or the complement thereof, or such soybean plant, plant cell, tissue, or seed, comprising in its genome a nucleic acid molecule comprising a nucleotide sequence with at least 99% sequence identity to the nucleotide sequence of SEQ ID No. 5 or 24 and SEQ ID No. 6 or 25, or the complement thereof, wherein said plant, cell, tissue or seed, or the soil or growth medium in which they are grown or are intended to be grown, are treated with one or more of the compounds and/or biological control agents or mixtures as described herein.
53. A soybean plant, plant cell, tissue, or seed, comprising in its genome a nucleic acid molecule hybridizing under standard stringency conditions to the nucleotide sequence of SEQ ID No. 5 or 6, or the complement thereof, wherein said plant, cell, tissue or seed, or the soil or growth medium in which they are grown or are intended to be grown, are treated with one or more of the compounds and/or biological control agents or mixtures as described herein.
54. Use of a soybean plant or seed comprising a nucleic acid molecule comprising a nucleotide sequence with at least 99% sequence identity to the nucleotide sequence of SEQ ID No. 5 or 6, or the complement thereof, such as a nucleic acid molecule comprising a nucleotide sequence with at least 99% sequence identity to the nucleotide sequence of SEQ ID No. 5 or 24 and SEQ ID No. 6 or 25, or the complement thereof, and the compound(s) or the biological control agent(s), or combinations, as described herein, to produce a soybean crop.
55. Use of a soybean plant or seed comprising a nucleic acid molecule comprising a nucleotide sequence hybridizing under standard stringency conditions to the nucleotide sequence of SEQ ID No. 5 or 6, or the complement thereof, and the compound(s) or the biological control agent(s), or combinations, as described herein, to produce a soybean crop.
56. The use of the nucleic acid molecule of any one of the above paragraphs, which also comprises the nucleotide sequence of SEQ ID No. 7 and 9.
57. Use of a soybean plant or seed comprising a chimeric DNA comprising a T-DNA 5′ flanking region, an inserted T-DNA, and a T-DNA 3′ flanking region, wherein the sequence of said inserted T-DNA comprises the sequence of SEQ ID No. 11 from nucleotide 188 to nucleotide 7368 or a sequence at least 95, 96, 97, 98, 99, or at least 99.5% identical thereto, or wherein the sequence of said inserted T-DNA comprises the sequence of SEQ ID No. 7 and 9, and wherein said T-DNA 5′ flanking region is located immediately upstream of and contiguous with said inserted T-DNA and comprises the sequence of SEQ ID No. 5 from nucleotide 1 to nucleotide 227 or a sequence at least 95, 96, 97, 98, 99, or at least 99.5% identical thereto, or of SEQ ID No. 24 from nucleotide 1 to nucleotide 1058, or a sequence at least 95, 96, 97, 98, 99, or at least 99.5% identical thereto, and wherein said T-DNA 3′ flanking region is located immediately downstream of and contiguous with said inserted T-DNA and comprises the sequence of SEQ ID No. 6 from nucleotide 254 to nucleotide 501 or a sequence at least 95, 96, 97, 98, 99, or at least 99.5% identical thereto, or the nucleotide sequence of the complement of SEQ ID No. 25 from nucleotide 254 to nucleotide 1339, or a sequence at least 95, 96, 97, 98, 99, or at least 99.5% identical thereto, and the compound(s) or the biological control agent(s), or combinations, as described herein, to produce a soybean crop.
58. Use of a soybean plant or seed comprising a nucleic acid molecule comprising a nucleotide sequence with at least 98% sequence identity to the nucleotide sequence of SEQ ID No. 7 or the complement thereof, such as the nucleotide sequence of SEQ ID No. 7, such as a nucleic acid molecule comprising the nucleotide sequence of SEQ ID No. 11 from nucleotide 131 to 5276 or the complement thereof, or a sequence encoding a nematicidal Cry14Ab protein having at least 95, 96, 97, 98, or at least 99% sequence identity to SEQ ID No. 7 or to the sequence of SEQ ID No. 11 from nucleotide position 131 to nucleotide position 5276, or the complement thereof, and the compound(s) or the biological control agent(s), or combinations, as described herein, to produce a soybean crop.
59. Use of a soybean plant or seed comprising a nucleic acid molecule comprising a nucleotide sequence with at least 98% sequence identity to the nucleotide sequence of SEQ ID No. 9 or the complement thereof, such as the nucleotide sequence of SEQ ID No. 9, such as a DNA molecule comprising the nucleotide sequence of SEQ ID No. 11 from nucleotide 5382 to 7621, or the complement thereof, or a sequence having at least 95, 96, 97, 98, or at least 99% sequence identity to SEQ ID No. 9, or to the sequence of SEQ ID No. 11 from nucleotide position 5382 to nucleotide position 7621, or its complement, wherein said sequence encodes an HPPD protein providing tolerance to HPPD inhibitor herbicides when expressed in a plant, and the compound(s) or the biological control agent(s), or combinations, as described herein, to produce a soybean crop.
60. A method for producing a treated soybean seed, comprising treating the soybean seed comprising elite event EE-GM4 as described in any one of these numbered paragraphs with the compound(s) or the biological control agent(s), or combinations, as described herein.
61. A method for protecting emerging soybean plants from competition by weeds and from plant-pathogenic nematodes, comprising treating a field in which seeds containing elite event EE-GM4 as described in any of these paragraphs were sown, with an HPPD inhibitor herbicide, and treating the soil wherein said seeds are grown or are intended to be grown, or treating said seed, with a nematicidal compound or biological control agent or combination as described herein, wherein the plants are tolerant to the HPPD inhibitor herbicide.
62. A method for protecting emerging soybean plants from competition by weeds and from plant-pathogenic nematodes, comprising treating a field to be planted with soybean plants comprising elite event EE-GM4 as described above with an HPPD inhibitor herbicide, before the soybean plants are planted or the seeds are sown, followed by planting or sowing of said soybean plants or seeds in said pre-treated field, wherein the plants are tolerant to the HPPD inhibitor herbicide, wherein said plants or seeds, or the soil in which they are grown or are intended to be grown, are treated with one or more of the compounds and/or biological control agents or mixtures as described herein.
63. The method of any one of these numbered paragraphs, wherein the HPPD inhibitor herbicide is isoxaflutole, topramezone or mesotrione.
64. Use of a transgenic soybean plant, seed or progeny thereof, comprising elite event EE-GM4 as described in any one of these numbered paragraphs to produce soybean grain or seed.
65. Use of a soybean plant or seed comprising elite event EE-GM4 in its genome as described in any one of these numbered paragraphs to grow a nematode-resistant and/or HPPD inhibitor herbicide-tolerant plant.
66. Use of a soybean plant or seed comprising elite event EE-GM4 as described in any one of these numbered paragraphs, in combination with an HPPD inhibitor herbicide and one or more of the compounds and/or biological control agents or mixtures as described herein, for growing a field of soybean, or for growing a soybean crop.
67. Use of a soybean plant or seed comprising a nucleic acid molecule obtainable from the seed deposited at the ATCC under accession number PTA-123624, wherein said nucleic acid molecule comprises the nucleotide sequence of any one of SEQ ID No. 1, 3, or 5 and the nucleotide sequence of any one of SEQ ID No. 2, 4, or 6, and one or more of the compounds and/or biological control agents or mixtures as described herein, for growing a soybean crop.
68. A soybean plant, cell, part, or seed, each comprising in its genome elite event EE-GM4, wherein said elite event is the genetic locus comprising an inserted T-DNA containing a chimeric HPPD-4 protein-encoding gene and a chimeric Cry14Ab-1 protein-encoding gene, and 5′ and 3′ flanking sequences immediately surrounding said inserted T-DNA, as found in reference seed deposited at the ATCC under deposit number PTA-123624, wherein said plant, cell, part or seed, or the soil or growth medium in which they are grown or are intended to be grown, are treated with one or more of the compounds and/or biological control agents or mixtures as described herein.
69. A progeny plant, cell, plant part or seed of the plant, cell, plant part or seed of paragraph 68, wherein said progeny plant, cell, plant part or seed comprises the nucleotide sequence of SEQ ID No. 3 and the nucleotide sequence of SEQ ID No. 4, and wherein said plant, cell, part or seed, or the soil or growth medium in which they are grown or are intended to be grown, are treated with one or more of the compounds and/or biological control agents or mixtures as described herein.
70. The soybean plant, cell, part, seed or progeny of paragraph 69, the genomic DNA of which, when analyzed using PCR with two primers comprising the nucleotide sequence of SEQ ID No. 12 and SEQ ID No. 13 respectively, yields a DNA fragment of 126 bp.
71. The plant of any one of the above paragraphs which is tolerant to isoxaflutole and/or topramezone and/or mesotrione, such as such a plant tolerant to isoxaflutole, topramezoneand mesotrione.
72. A method for producing a soybean plant resistant to SCN and tolerant to HPPD inhibitor herbicides, comprising introducing resistance to SCN and tolerance to HPPD inhibitor herbicides into the genome of a soybean plant by crossing a first soybean plant lacking a Cry14Ab-1-encoding gene and lacking an HPPD-4-encoding gene with the soybean plant of any one of the above paragraphs, and selecting a progeny plant resistant to SCN and tolerant to HPPD inhibitor herbicides, wherein said plant, or the soil or growth medium in which it is grown or is intended to be grown, is treated with one or more of the compounds and/or biological control agents or mixtures as described herein.
73. Use of a soybean plant or seed comprising elite event EE-GM4 as described in any one of these numbered paragraphs to obtain a soybean crop, such as a soybean crop yielding better when infested by nematodes or Sudden Death Syndrome caused by a Fusarium species fungus.
74. A method of producing a soybean crop with improved resistance to nematodes or Sudden Death Syndrome caused by a Fusarium species fungus, comprising the steps (a) planting a field using the seed as described in any of the above paragraphs; and (b) harvesting the soybean seed produced on the plants grown from said seed, and optionally (c) applying to the field planted with said seeds before or after seed emergence, or on said soybean plants one or more doses of an HPPD inhibitor herbicide sufficient to kill weeds but which is tolerated by said soybean seeds or plants, such as wherein said nematodes are SCN or Pratylenchus species or root-knot nematode or reniform nematode species nematodes, and wherein said plant or seed, or the soil in which it is grown or is intended to be grown, is treated with one or more of the compounds and/or biological control agents or mixtures as described herein.
75. A soybean plant, seed or cell comprising in its genome elite event EE-GM4, wherein elite event EE-GM4 comprises a nucleotide sequence which is at least 90% identical to the sequence set forth in SEQ ID NO. 23, wherein said elite event comprises a chimeric HPPD-4-encoding gene and a chimeric Cry14Ab-1-encoding gene, wherein said plant, seed or cell is tolerant to an HPPD inhibitor herbicide and has SCN resistance, wherein said plant, seed or cell, or the soil or growth medium in which they are grown or are intended to be grown, is treated with one or more of the compounds and/or biological control agents or mixtures as described herein.
76. The plant of paragraph 75, wherein elite event EE-GM4 comprises a nucleotide sequence which is at least 95% identical to the sequence set forth in SEQ ID NO. 23.
77. The plant of paragraph 75, wherein elite event EE-GM4 comprises a nucleotide sequence which is at least 99% identical to the sequence set forth in SEQ ID NO. 23.
78. Use of a soybean plant or seed comprising a nucleic acid molecule comprising the nucleotide sequence of SEQ ID NO. 23 or a nucleotide sequence with at least 99% sequence identity to SEQ ID NO. 23, which confers tolerance to an HPPD inhibitor herbicide and/or nematode resistance, such as wherein said nematode is an SCN or Pratylenchus species or root-knot nematode or reniform nematode species nematode, to produce a soybean crop, wherein said plant or seed, or the soil or growth medium in which it is grown or is intended to be grown, is treated with one or more of the compounds and/or biological control agents or mixtures as described herein.
79. The use of the above paragraph, wherein said nucleic acid molecule comprises the nucleotide sequence of SEQ ID No. 11 from nucleotide position 131 to nucleotide position 7941, or a nucleotide sequence having at least 95%, at least 96%, at least 97%, at least 98%, or at least 99% sequence identity thereto.
80. The use of paragraph 79, wherein said nucleic acid molecule encodes an HPPD protein tolerant to an HPPD inhibitor and a protein negatively affecting plant pest nematodes, such as SCN, RKN or Pratylenchus spp. nematodes.
81. The use of paragraph 93, wherein said nucleic acid encodes the protein of SEQ ID No. 8 or a protein at least 99% identical thereto and the protein of SEQ ID No. 10, or a protein at least 99% identical thereto.
82. A method for controlling weeds and/or nematodes in a field to be planted with soybean plants, comprising the steps of: 1) treating said field with an HPPD inhibitor herbicide such as isoxaflutole, topramezone or mesotrione, and 2) planting or sowing of soybean plants or seeds comprising elite transformation event EE-GM4 as described above in said treated field, wherein reference seed comprising said elite event is deposited at the at the ATCC under deposit number PTA-123624, wherein said plants or seeds, or the soil or growth medium in which they are grown or are intended to be grown, are treated with one or more of the compounds and/or biological control agents or mixtures as described herein.
83. A method of weed and/or nematode control, characterized in that it comprises the steps of: 1) planting of soybean plants or seeds tolerant to an HPPD inhibitor herbicide such as isoxaflutole, topramezone or mesotrione, in a field, and 2) application of an HPPD inhibitor herbicide, such as isoxaflutole, topramezone or mesotrione, in said field before planting said plants or seeds, or on said soybean plants or seeds after planting (can be before or after seed germination), wherein said plants or seeds comprise soybean elite transformation event EE-GM4 in their genome, reference seed comprising said elite event being deposited at the ATCC under deposit number PTA-123624, wherein said plants or seeds, or the soil or growth medium in which they are grown or are intended to be grown, are treated with one or more of the compounds and/or biological control agents or mixtures as described herein.
84. A process for weed and/or nematode control, characterized in that it comprises the steps of: 1) treating a field to be planted with soybean plants or a field to be sown with soybean seeds with an HPPD inhibitor herbicide, such as isoxaflutole, topramezone or mesotrione, before the soybean plants are planted or the seeds are sown, and 2) planting soybean plants comprising soybean elite transformation event EE-GM4 or sowing soybean seeds comprising soybean elite transformation event EE-GM4 in said pre-treated field, wherein reference seed comprising said soybean elite transformation event EE-GM4 is deposited at the ATCC under deposit number PTA-123624, wherein said plants or seeds, or the soil or growth medium in which they are grown or are intended to be grown, are treated with one or more of the compounds and/or biological control agents or mixtures as described herein.
85. A method for reducing yield loss in a field to be planted with soybean plants, particularly a field that contains or is expected to contain nematodes such as SCN, RKN or Pratylenchus or reniform nematodes or a combination thereof, comprising the step of 1) obtaining plants or seeds comprising elite transformation event EE-GM4 as described above, and 2) planting or sowing of soybean plants or seeds, wherein reference seed comprising said elite event is deposited at the at the ATCC under deposit number PTA-123624, wherein said plants or seeds, or the soil or growth medium in which they are grown or are intended to be grown, are treated with one or more of the compounds and/or biological control agents or mixtures as described herein.
86. A method for increasing yield of soybean plants when planted in a field containing nematodes such as SCN, RKN or Pratylenchus or reniform nematodes or a combination thereof, comprising the step of 1) obtaining plants or seed comprising elite transformation event EE-GM4 as described above, and 2) planting or sowing of soybean plants or seeds, wherein reference seed comprising said elite event is deposited at the at the ATCC under deposit number PTA-123624, wherein said plants or seeds, or the soil or growth medium in which they are grown or are intended to be grown, are treated with one or more of the compounds and/or biological control agents or mixtures as described herein.
87. A method for producing a soybean plant or seed tolerant to an HPPD inhibitor herbicide, such as isoxaflutole, topramezone or mesotrione, or for producing a soybean plant or seed tolerant to nematodes, such as SCN, RKN or Pratylenchus or reniform nematodes, or for producing a soybean plant or seed tolerant to an HPPD inhibitor herbicide, such as isoxaflutole, topramezone or mesotrione, and tolerant to nematodes, such as SCN, RKN or Pratylenchus or reniform nematodes, characterized by the step of introducing into the genome of a soybean plant or seed elite soybean transformation event EE-GM4 as described above, and optionally treating said plant or seed with an HPPD inhibitor herbicide, such as isoxaflutole, topramezone or mesotrione, or optionally treating the field in which said plant or seed will be planted with an HPPD inhibitor herbicide, such as isoxaflutole, topramezone or mesotrione, and planting said plant or seed in said pre-treated field, wherein said plant or seed, or the soil or growth medium in which it is grown or is intended to be grown, is treated with one or more of the compounds and/or biological control agents or mixtures as described herein.
88. The use of a nucleic acid molecule as described in any of these numbered paragrpahs, wherein said nucleic acid molecule specifically characterizes soybean elite transformation event EE-GM4, characterized in that it comprises the nucleotide sequence of any one of SEQ ID No. 1, 3 or 5, which contains a part of soybean plant genomic DNA and a part of inserted foreign DNA of EE-GM4 downstream thereof and contiguous therewith, and/or characterized in that it comprises the nucleotide sequence of SEQ ID No. 2, 4, or 6, which contains a part of inserted foreign DNA of EE-GM4 and a part of soybean plant genomic DNA downstream thereof and contiguous therewith.
89. The plant or seed comprising EE-GM4 as described in any of these numbered paragraphs, also comprising tolerance or resistance to SCN, RKN or Pratylenchus or reniform nematodes, or a combination thereof, as provided by native soybean resistance loci/genes or by one or more other soybean transformation events.
90. The plant or seed of any one of these numbered paragraphs, wherein said plant or seed comprises EE-GM4 and any one or a combination of the SCN resistance alleles/loci from the resistance sources in table 1 or from PI 548316, PI 567305, PI 437654, PI 90763, PI 404198B, PI 88788, PI 468916, PI 567516C, PI 209332, PI 438489B, PI 89772, Peking, PI 548402, PI 404198A, PI 561389B, PI 629013, PI 507471, PI 633736, PI 507354, PI 404166, PI 437655, PI 467312, PI 567328, PI 22897, or PI 494182, such as one or more of the SCN resistance genes or loci from the resistance sources PI 88788, PI 548402, PI 209332, or PI 437654.
91. A plant or seed comprising EE-GM4 as described in any one of these numbered paragraphs, also comprising tolerance to other herbicides, as provided by herbicide tolerance genes (either native or mutated soybean genes or transgenes), such as tolerance to glyphosate-. glufosinate-, sulfonylurea-, imidazolinone-, HPPD inhibitor-, dicamba-, 2,4-D-, or PPO inhibitor-based herbicides, or any combination thereof.
92. The plant or seed of paragraph 91 wherein said plant or seed comprises EE-GM4 as described above and one or more of the following soybean transformation events conferring herbicide tolerance: MST-FGØ72-3, SYHTOH2, DAS-68416-4, DAS-44406-6, MON87708, MON89788, GTS 40-3-2, A2704-12, BPS-CV127-9, A5547-127, MON87705, IND-ØØ41Ø-5, DP305423, DAS-81419-2, DP-356043-5, MON87712, MON87769, or said plant or seed comprises EE-GM4 and a combination of the following events: MON89788×MON87708, HOS×GTS 40-3-2, MST-FGØ72-3×A5547-127, MON87701×MON 89788, DAS-81419-2 x DAS-44406-6, DAS-68416-4×MON 89788, MON87705×Event MON 89788, DP305423 x GTS 40-3-2, DP305423×MON87708, DP305423×MON87708×MON89788, DP305423 x MON89788, MON87705×MON87708, MON87705×MON87708×MON89788, MON89788×MON87708×A5547-127, MON87751×MON87701×MON87708 x MON89788, or MON87769×Event MON89788.
93. A method to reduce severity of effects of Sudden Death Syndrome or Iron Deficiency Chlorosis on soybean plants in the presence of SCN infestation, or to increase yield of soybean plants in SCN-containing fields infested with Sudden Death Syndrome or in SCN-containing fields causing Iron Deficiency Chlorosis in soybean, which method comprises planting soybean plants or sowing soybean seeds comprising elite event EE-GM4, wherein reference seed comprising said elite event is deposited at the at the ATCC under deposit number PTA-123624, wherein said plants or seeds, or the soil or growth medium in which they are grown or are intended to be grown, are treated with one or more of the compounds and/or biological control agents or mixtures as described herein.
94. A soybean plant, cell, plant part, or seed, of any one of these numbered paragraphs, comprising any of the nucleic acid molecules: a) nucleic acid molecule comprising a nucleotide sequence essentially similar to the sequence of any one of SEQ ID No. 1, 3 or 5 or to the sequence of any one of SEQ ID No. 2, 4, or 6, or the complement of said sequences, b) a nucleic acid molecule comprising a nucleotide sequence with at least 99% sequence identity to the nucleotide sequence of SEQ ID No. 3, 4, 5, 6, 24 or 25, or the complement thereof, c) a nucleic acid molecule comprising the nucleotide sequence of any one of SEQ ID No. 1 or 3 or SEQ ID No. 2 or 4, or the complement of said sequences, or comprising the nucleotide sequence of SEQ ID No. 1 or 3 and SEQ ID No. 2 or 4, d) a nucleic acid molecule comprising the nucleotide sequence of any one of SEQ ID No. 1 or 3 or SEQ ID No. 2 or 4, or the complement of said sequences, and the nucleotide sequence of SEQ ID No. 7 and 9, or the complement thereof, or comprising the nucleotide sequence of SEQ ID No. 1 or 3 and SEQ ID No. 2 or 4, or the complement of said sequences, and the nucleotide sequence of SEQ ID No. 7 and 9, or the complement thereof, e) a nucleic acid molecule comprising the nucleotide sequence of SEQ ID No. 11 from nucleotide position 188 to nucleotide position 7101, or a nucleotide sequence having at least 98%, or least 99%, or at least 99.5% or at least 99.9% sequence identity thereto, f) a nucleic acid molecule comprising the nucleotide sequence of SEQ ID No. 11 from nucleotide position 188 to nucleotide position 7101, which comprises the nucleotide sequence of SEQ ID No. 5 or 24 and SEQ ID No. 6 or 25, or the complement thereof, or g) a nucleic acid molecule obtainable from the seed deposited at the ATCC under accession number PTA-123624, wherein said nucleic acid molecule comprises the nucleotide sequence of any one of SEQ ID No. 1, 3, or 5 and the nucleotide sequence of any one of SEQ ID No. 2, 4, or 6, wherein said soybean plant, cell, plant part, seed or progeny thereof, or the soil in which they are grown or are intended to be grown, are treated with one or more of the compounds and/or biological control agents or mixtures thereof as described herein, such as a compound and/or biological control agent or mixture from any one of H1, H2, H3, H4, H5, IAN1, IAN2, IAN3, IAN4, IAN5, IAN6, IAN7, IAN8, IAN9, IAN10, IAN11, IAN12, IAN13, IAN14, IAN15, IAN16, IAN17, IAN18, IAN19, IAN20, IAN21, IAN22, IAN23, IAN24, IAN25, IAN26, IAN27, IAN28, IAN29, IAN30, SIAN1, F1, F2, F3, F4, F5, F6, F7, F8, F9, F9, F10, F11, F12, F13, F14, F15, F16, SF1, P1, BCA, BCA2, BCA3, BCA4, BCA5, BCA6, BCA7, BCA8; BCA9, BCA10, NC1, NC2, NC3, SBCA1, SAI1, SCI and SC2.
95. A soybean plant, cell, plant part, or seed, each comprising in its genome elite event EE-GM4, wherein said elite event is the genetic locus comprising an inserted T-DNA containing a chimeric HPPD-4 protein-encoding gene and a chimeric Cry14Ab-1 protein-encoding gene, and 5′ and 3′ flanking sequences immediately surrounding said inserted T-DNA, as found in reference seed deposited at the ATCC under deposit number PTA-123624, such as said plant, cell, part or seed which also comprises the nucleotide sequence of SEQ ID No. 3 and the nucleotide sequence of SEQ ID No. 4, wherein said soybean plant, cell, plant part, seed or progeny thereof, or the soil in which they are grown or are intended to be grown, are treated with one or more of the compounds and/or biological control agents or mixtures as described herein.
96. The plant, plant part, or seed of paragraph 94 or 95, wherein the soil in which they are grown or are intended to be grown, are treated with one or more of said compounds and/or biological control agents or mixtures thereof, and wherein said plant, plant part, seed or progeny are planted or sown in the soil treated with one or more of the compounds and/or biological control agents or mixtures as described herein.
97. The plants or seeds of the above paragraphs, comprising native soybean SCN resistance loci or genes, such as any one of the SCN resistance genes or loci from the resistance sources PI 88788, PI 548402, PI 437654, or a combination thereof.
98. A method for weed control, comprising treating a field in which the soybean seeds of any of the above paragraphs were sown with an HPPD inhibitor herbicide and with a nematicidal compound and/or a nematicidal biological control agent, before the soybean plants emerge but after the seeds are sown.
99. A method for increasing yield on the soybean plants of any one of the above paragraphs, comprising treating a field to be sown or planted with said soybean seeds or plants, with an HPPD inhibitor herbicide and a nematicide, before the soybean plants are planted or the seeds are sown, followed by planting or sowing of said soybean plants or seeds in said pre-treated field.
100. The method of paragraphs 98 or 99, wherein said nematicide contains a nematicidal compound and/or a nematicidal biological control agent as described herein.
101. A method for producing a soybean plant resistant to SCN and/or tolerant to HPPD inhibitor herbicides, comprising introducing resistance to SCN and/or tolerance to HPPD inhibitor herbicides into the genome of a soybean plant by crossing a first soybean plant, such as a soybean plant lacking a Cry14Ab-1-encoding gene and lacking an HPPD-4-encoding gene, with a second soybean plant comprising elite event EE-GM4, and selecting seed of a progeny plant comprising elite event EE-GM4, which method includes the step of treating said seed with one or more of the compound(s) and/or biological control agent(s) or mixtures comprising them, as described herein, wherein said elite event comprises a nucleic acid molecule as described in paragraph 107, or wherein said elite event is the genetic locus comprising an inserted T-DNA containing a chimeric HPPD-4 protein-encoding gene and a chimeric Cry14Ab-1 protein-encoding gene, and 5′ and 3′ flanking sequences immediately surrounding said inserted T-DNA, as found in reference seed deposited at the ATCC under deposit number PTA-123624, such as said plant which also comprises the nucleotide sequence of SEQ ID No. 3 and the nucleotide sequence of SEQ ID No. 4.
102. Use of the plant, seed, part, or cell of any one of the above paragraphs, and said compound and/or biological control agent or a mixture containing it, to produce soybean seed.
103. Use of the treated soybean plant or seed of any one of the above paragraphs to grow a nematode-resistant and/or HPPD inhibitor herbicide-tolerant plant.
104. A method for increasing yield of soybean plants when planted in a field that contains, contained or is expected to contain nematodes or nematode eggs such as SCN, RKN, Pratylenchus or reniform nematodes or their eggs, comprising the step of 1) obtaining soybean plants or seed comprising elite event EE-GM4, and 2) planting or sowing of said plants or seeds, wherein reference seed comprising said elite event is deposited at the at the ATCC under deposit number PTA-123624, which method comprises the step of treating said plant or seed with one or more of the compound(s) and/or biological control agent(s) or mixtures, as described herein.
105. A method to increase yield of soybean plants in SCN-containing fields infested with Sudden Death Syndrome or in SCN-containing fields causing Iron Deficiency Chlorosis in soybean, which method comprises sowing treated seeds comprising elite event EE-GM4, wherein reference seed comprising said elite event is deposited at the at the ATCC under deposit number PTA-123624, wherein said seeds are treated with one or more of said other events, treated with one or more of the compound(s) and/or biological control agent(s) or mixtures comprising them, as described herein, such as a nematicidal, insecticidal or fungicidal compound(s) and/or biological control agent, or a combination of a nematicidal, insecticidal and fungicidal compound(s) and/or biological control agent.
106. A plant, seed, part or cell comprising elite event EE-GM4 as described above, which also comprises one or more of the following other events. Event MON87751, Event pDAB8264.42.32.1, Event DAS-81419-2, Event FG-072, Event SYHTOH2, Event DAS-68416-4, Event DAS-81615-9, Event DAS-44406-6, Event MON87708, Event MON89788, Event DAS-14536-7, Event GTS 40-3-2, Event A2704-12, Event BPS-CV127-9, Event A5547-127, Event MON87754, Event DP-3Ø5423-1, Event MON87701, Event MON87705, Event MON87712, Event pDAB4472-1606, Event DP-356043-5, Event MON87769, Event IND-ØØ41Ø-5, Event DP305423, such as a seed of any of the above paragraphs also comprising one or more of said other events, treated with one or more of the compound(s) and/or biological control agent(s) or mixtures comprising them, as described herein.
107. A plant, seed, part or cell comprising elite event EE-GM4 as described above, which also comprises one or more of the following combinations of other soybean events: MON89788 x MON87708, HOS×GTS 40-3-2, FG-072×A5547-127, MON87701×MON 89788, DAS-81419-2×DAS-44406-6, DAS-81419-2×DAS-68416-4, DAS-68416-4×MON 89788, MON 87705×MON 89788, MON 87769×MON 89788, DP305423×GTS 40-3-2, DP305423 x MON87708, Event DP305423×MON87708×Event MON89788, DP305423×MON89788, MON87705×MON87708, MON87705×MON87708×MON89788, MON89788 x MON87708×A5547-127, MON87751×MON87701×MON87708×MON89788, SYHTOH2×MON89788, SYHTOH2×GTS 40-3-2, SYHTOH2×MON89788×MON87708, such as a plant or seed of any of the above paragraphs also comprising one or more of said combination of other events, treated with one or more of the compound(s) and/or biological control agent(s) or mixtures comprising them, as described herein.
108. The plant, cell, plant part or seed of any one of these numbered paragraphs, or the soil in which they are grown or are intended to be grown, treated with any of the following nematicidal agents: alanycarb, aldicarb, carbofuran, carbosulfan, fosthiazate, cadusafos, oxamyl, thiodicarb, dimethoate, ethoprophos, terbufos, abamectin, methyl bromide and other alkyl halides, methyl isocyanate generators selected from diazomet and metam, fluazaindolizine, fluensulfone, fluopyram, tioxazafen, N-[1-(2,6-difluorophenyl)-1H-pyrazol-3-yl]-2-trifluoromethylbenzamide, cis-Jasmone, harpin, Azadirachta indica oil, or Azadirachtin.
109. The plant, cell, plant part or seed of any one of these numbered paragraphs, or the soil in which they are grown or are intended to be grown, treated with any of the following nematicidal agents: fosthiazate, cadusafos, thiodicarb, abamectin, fluazaindolizine, fluopyram, tioxazafen, N-[1-(2,6-difluorophenyl)-1H-pyrazol-3-yl]-2-trifluoromethylbenzamide, cis-Jasmone, harpin, Azadirachta indica oil, or Azadirachtin.
110. The plant, cell, plant part or seed of paragraph claim 109, treated with a combination selected from the group consisting of: fosthiazate and cadusafos, fosthiazate and thiodicarb, fosthiazate and abamectin, fosthiazate and fluazaindolizine, fosthiazate and fluopyram, fosthiazate and tioxazafen, fosthiazate and N-[1-(2,6-difluorophenyl)-1H-pyrazol-3-yl]-2-trifluoromethylbenzamide, fosthiazate and cis-Jasmone, fosthiazate and harpin, fosthiazate and Azadirachta indica oil, fosthiazate and Azadirachtin, cadusafos and fosthiazate, cadusafos and thiodicarb, cadusafos and abamectin, cadusafos and fluazaindolizine, cadusafos and fluopyram, cadusafos and tioxazafen, cadusafos and N-[1-(2,6-difluorophenyl)-1H-pyrazol-3-yl]-2-trifluoromethylbenzamide, cadusafos and cis-Jasmone, cadusafos and harpin, cadusafos and Azadirachta indica oil, cadusafos and Azadirachtin, thiodicarb and fosthiazate, thiodicarb and cadusafos, thiodicarb and abamectin, thiodicarb and fluazaindolizine, thiodicarb and fluopyram, thiodicarb and tioxazafen, thiodicarb and N-[1-(2,6-difluorophenyl)-1H-pyrazol-3-yl]-2-trifluoromethylbenzamide, thiodicarb and cis-Jasmone, thiodicarb and harpin, thiodicarb and Azadirachta indica oil, abamectin and cadusafos, abamectin and thiodicarb, abamectin and fluazaindolizine, abamectin and fluopyram, abamectin and tioxazafen, abamectin and N-[1-(2,6-difluorophenyl)-1H-pyrazol-3-yl]-2-trifluoromethylbenzamide, abamectin and cis-Jasmone, abamectin and harpin, abamectin and Azadirachta indica oil, abamectin and Azadirachtin, fluazaindolizine and abamectin, fluazaindolizine and fluopyram, fluazaindolizine and tioxazafen, fluazaindolizine and N-[1-(2,6-difluorophenyl)-1H-pyrazol-3-yl]-2-trifluoromethylbenzamide, fluazaindolizine and cis-Jasmone, fluazaindolizine and harpin, fluazaindolizine and Azadirachta indica oil, fluazaindolizine and Azadirachtin, fluopyram and fluazaindolizine, fluopyram and tioxazafen, fluopyram and N-[1-(2,6-difluorophenyl)-1H-pyrazol-3-yl]-2-trifluoromethylbenzamide, fluopyram and cis-Jasmone, fluopyram and harpin, fluopyram and Azadirachta indica oil, fluopyram and Azadirachtin, tioxazafen and N-[1-(2,6-difluorophenyl)-1H-pyrazol-3-yl]-2-trifluoromethylbenzamide, tioxazafen and cis-Jasmone, tioxazafen and harpin, tioxazafen and Azadirachta indica oil, tioxazafen and Azadirachtin, N-[1-(2,6-difluorophenyl)-1H-pyrazol-3-yl]-2-trifluoromethylbenzamide and cis-Jasmone, N-[1-(2,6-difluorophenyl)-1H-pyrazol-3-yl]-2-trifluoromethylbenzamide and harpin, N-[1-(2,6-difluorophenyl)-1H-pyrazol-3-yl]-2-trifluoromethylbenzamide and Azadirachta indica oil, N-[1-(2,6-difluorophenyl)-1H-pyrazol-3-yl]-2-trifluoromethylbenzamide and Azadirachtin, cis-Jasmone and harpin, cis-Jasmone and Azadirachta indica oil, cis-Jasmone and Azadirachtin, harpin and Azadirachta indica oil, and harpin and Azadirachtin.
111. The plant, cell, plant part or seed of any one of these numbered paragraphs, or the soil in which they are grown or are intended to be grown, treated with a biological control agent selected from the group consisting of: a Bacillus species strain, a Brevibacillus species strain, a Burkholderia species strain, a Lysobacter species strain, a Pasteuria species strain, an Arthrobotrys species strain, a Nematoctonus species strain, a Myrothecium species strain, a Paecilomyces species strain, a Trichoderma species strain, a Tsukamurella species strain.
112. The plant, cell, plant part or seed of paragraph 111, or the soil in which they are grown or are intended to be grown, treated with a biological control agent selected from the group consisting of: Bacillus amyloliquefaciens, Bacillus firmus, Bacillus laterosporus, Bacillus lentus, Bacillus licheniformis, Bacillus nematocida, Bacillus pumilus, Bacillus subtilis, Bacillus penetrans, Bacillus thuringiensis, Brevibacillus laterosporus, Burkholderia rinojensis, Lysobacter antibioticus, Lysobacter enzymogenes, Pasteuria nishizawae, Pasteuria penetrans, Pasteuria ramosa, Pasteuria reniformis, Pasteuria thornei, Pasteuria usage, Arthrobotrys dactyloides, Arthrobotrys oligospora, Arthrobotrys superba, Nematoctonus geogenius, Nematoctonus leiosporus, Myrothecium verrucaria, Paecilomyces lilacinus, Paecilomyces variotii, Trichoderma asperellum, Trichoderma harzianum, Trichoderma viride, Trichoderma harzianum rifai, and Tsukamurella paurometabola.
113. The plant, cell, plant part or seed of paragraph 112, or the soil in which they are grown or are intended to be grown, treated with a biological control agent selected from the group consisting of: Bacillus amyloliquefaciens strain IN937a, Bacillus amyloliquefaciens strain FZB42, Bacillus amyloliquefaciens strain FZB24, Bacillus amyloliquefaciens strain NRRL B-50349, Bacillus amyloliquefaciens strain ABI01, Bacillus amyloliquefaciens strain B3, Bacillus amyloliquefaciens strain D747, Bacillus amyloliquefaciens strain APM-1, Bacillus amyloliquefaciens strain TJ1000, Bacillus amyloliquefaciens strain AP-136, Bacillus amyloliquefaciens strain AP-188, Bacillus amyloliquefaciens strain AP-218, Bacillus amyloliquefaciens strain AP-219, Bacillus amyloliquefaciens strain AP-295, Bacillus amyloliquefaciens strain MBI 600, Bacillus amyloliquefaciens strain PTA-4838, Bacillus amyloliquefaciens strain F727, Bacillus firmus strain I-1582, Bacillus firmus strain NRRL B-67003, Bacillus firmus strain NRRL B-67518, Bacillus firmus strain GB126, Bacillus laterosporus strain ATCC PTA-3952, Bacillus laterosporus strain ATCC PTA-3593, Bacillus licheniformis strain ATCC PTA-6175, Bacillus licheniformis SB3086, licheniformis CH200, licheniformis RTI 184, a combination of licheniformis CH200 and licheniformis RTI 184, Bacillus subtilis var. amyloliquefaciens strain FZB24, Bacillus thuringiensis strain EX297512, Bacillus thuringiensis strain CR-371, or Bacillus thuringiensis strain AQ52, Brevibacillus laterosporus strain ATCC 64, Brevibacillus laterosporus strain NRS 1111, Brevibacillus laterosporus strain NRS 1645, Brevibacillus laterosporus strain NRS 1647, Brevibacillus laterosporus strain BPM3, Brevibacillus laterosporus strain G4, Brevibacillus laterosporus strain NCIMB 41419, Burkholderia rinojensis, Burkholderia rinojensis strain A396, Lysobacter antibioticus strain 13-1, Lysobacter enzymogenes strain C3, Myrothecium verrucaria strain AARC-0255, Paecilomyces lilacinus strain 251, Paecilomyces variotii strain Q-09, Pasteuria nishizawae strain Pn1, Trichoderma asperellum strain ICC 012, Trichoderma asperellum strain SKT-1, Trichoderma asperellum strain T34, Trichoderma asperellum strain T25, Trichoderma asperellum strain SF04, Trichoderma asperellum strain TV1, Trichoderma asperellum strain T11, Trichoderma harzianum strain ICC012, Trichoderma harzianum rifai T39, Trichoderma harzianum rifai strain KRL-AG2, Trichoderma viride strain TV1, Trichoderma viride strain TV25, Trichoderma atroviride strain CNCM I-1237, Trichoderma atroviride strain CNCM I-1237, Trichoderma atroviride strain NMI No. V08/002387, Trichoderma atroviride strain NMI No. V08/002388, Trichoderma atroviride strain NMI No. V08/002389, Trichoderma atroviride strain NMI No. V08/002390, Trichoderma atroviride strain ATCC 20476, Trichoderma atroviride strain T11, Trichoderma atroviride strain LC52, Trichoderma atroviride strain SC1, Trichoderma atroviride strain SKT-1, Trichoderma atroviride strain SKT-2, Trichoderma atroviride strain SKT-3, and Tsukamurella paurometabola strain C-924.
114. The plant, cell, plant part or seed of any one of these numbered paragraphs, wherein said treatment is a seed treatment.
115. The plant, cell, plant part or seed of any one of these numbered paragraphs, or the soil in which they are grown or are intended to be grown, treated with a combination of a nematicidal agent and a biological control agent selected from: a Bacillus amyloliquefaciens strain and alanycarb, a Bacillus amyloliquefaciens strain and aldicarb, a Bacillus amyloliquefaciens strain and carbofuran, a Bacillus amyloliquefaciens strain and carbosulfan, a Bacillus amyloliquefaciens strain and fosthiazate, a Bacillus amyloliquefaciens strain and cadusafos, a Bacillus amyloliquefaciens strain and oxamyl, a Bacillus amyloliquefaciens strain and thiodicarb, a Bacillus amyloliquefaciens strain and dimethoate, a Bacillus amyloliquefaciens strain and ethoprophos, a Bacillus amyloliquefaciens strain and terbufos, a Bacillus amyloliquefaciens strain and abamectin, a Bacillus amyloliquefaciens strain and methyl bromide and other alkyl halides, a Bacillus amyloliquefaciens strain and methyl isocyanate generators selected from diazomet and metam, a Bacillus amyloliquefaciens strain and fluazaindolizine, a Bacillus amyloliquefaciens strain and fluensulfone, a Bacillus amyloliquefaciens strain and fluopyram, a Bacillus amyloliquefaciens strain and tioxazafen, a Bacillus amyloliquefaciens strain and N-[1-(2,6-difluorophenyl)-1H-pyrazol-3-yl]-2-trifluoromethylbenzamide, a Bacillus amyloliquefaciens strain and cis-Jasmone, a Bacillus amyloliquefaciens strain and harpin, a Bacillus amyloliquefaciens strain and Azadirachta indica oil, a Bacillus amyloliquefaciens strain and Azadirachtin, a Bacillus firmus strain and alanycarb, a Bacillus firmus strain and aldicarb, a Bacillus firmus strain and carbofuran, a Bacillus firmus strain and carbosulfan, a Bacillus firmus strain and fosthiazate, a Bacillus firmus strain and cadusafos, a Bacillus firmus strain and oxamyl, a Bacillus firmus strain and thiodicarb, a Bacillus firmus strain and dimethoate, a Bacillus firmus strain and ethoprophos, a Bacillus firmus strain and terbufos, a Bacillus firmus strain and abamectin, a Bacillus firmus strain and methyl bromide and other alkyl halides, a Bacillus firmus strain and methyl isocyanate generators selected from diazomet and metam, a Bacillus firmus strain and fluazaindolizine, a Bacillus firmus strain and fluensulfone, a Bacillus firmus strain and fluopyram, a Bacillus firmus strain and tioxazafen, a Bacillus firmus strain and N-[1-(2,6-difluorophenyl)-1H-pyrazol-3-yl]-2-trifluoromethylbenzamide, a Bacillus firmus strain and cis-Jasmone, a Bacillus firmus strain and harpin, a Bacillus firmus strain and Azadirachta indica oil, a Bacillus firmus strain and Azadirachtin, a Bacillus laterosporus strain and alanycarb, a Bacillus laterosporus strain and aldicarb, a Bacillus laterosporus strain and carbofuran, a Bacillus laterosporus strain and carbosulfan, a Bacillus laterosporus strain and fosthiazate, a Bacillus laterosporus strain and cadusafos, a Bacillus laterosporus strain and oxamyl, a Bacillus laterosporus strain and thiodicarb, a Bacillus laterosporus strain and dimethoate, a Bacillus laterosporus strain and ethoprophos, a Bacillus laterosporus strain and terbufos, a Bacillus laterosporus strain and abamectin, a Bacillus laterosporus strain and methyl bromide and other alkyl halides, a Bacillus laterosporus strain and methyl isocyanate generators selected from diazomet and metam, a Bacillus laterosporus strain and fluazaindolizine, a Bacillus laterosporus strain and fluensulfone, a Bacillus laterosporus strain and fluopyram, a Bacillus laterosporus strain and tioxazafen, a Bacillus laterosporus strain and N-[1-(2,6-difluorophenyl)-1H-pyrazol-3-yl]-2-trifluoromethylbenzamide, a Bacillus laterosporus strain and cis-Jasmone, a Bacillus laterosporus strain and harpin, a Bacillus laterosporus strain and Azadirachta indica oil, a Bacillus laterosporus strain and Azadirachtin, a Bacillus lentus strain and alanycarb, a Bacillus lentus strain and aldicarb, a Bacillus lentus strain and carbofuran, a Bacillus lentus strain and carbosulfan, a Bacillus lentus strain and fosthiazate, a Bacillus lentus strain and cadusafos, a Bacillus lentus strain and oxamyl, a Bacillus lentus strain and thiodicarb, a Bacillus lentus strain and dimethoate, a Bacillus lentus strain and ethoprophos, a Bacillus lentus strain and terbufos, a Bacillus lentus strain and abamectin, a Bacillus lentus strain and methyl bromide and other alkyl halides, a Bacillus lentus strain and methyl isocyanate generators selected from diazomet and metam, a Bacillus lentus strain and fluazaindolizine, a Bacillus lentus strain and fluensulfone, a Bacillus lentus strain and fluopyram, a Bacillus lentus strain and tioxazafen, a Bacillus lentus strain and N-[1-(2,6-difluorophenyl)-1H-pyrazol-3-yl]-2-trifluoromethylbenzamide, a Bacillus lentus strain and cis-Jasmone, a Bacillus lentus strain and harpin, a Bacillus lentus strain and Azadirachta indica oil, a Bacillus lentus strain and Azadirachtin, a Bacillus licheniformis strain and alanycarb, a Bacillus licheniformis strain and aldicarb, a Bacillus licheniformis strain and carbofuran, a Bacillus licheniformis strain and carbosulfan, a Bacillus licheniformis strain and fosthiazate, a Bacillus licheniformis strain and cadusafos, a Bacillus licheniformis strain and oxamyl, a Bacillus licheniformis strain and thiodicarb, a Bacillus licheniformis strain and dimethoate, a Bacillus licheniformis strain and ethoprophos, a Bacillus licheniformis strain and terbufos, a Bacillus licheniformis strain and abamectin, a Bacillus licheniformis strain and methyl bromide and other alkyl halides, a Bacillus licheniformis strain and methyl isocyanate generators selected from diazomet and metam, a Bacillus licheniformis strain and fluazaindolizine, a Bacillus licheniformis strain and fluensulfone, a Bacillus licheniformis strain and fluopyram, a Bacillus licheniformis strain and tioxazafen, a Bacillus licheniformis strain and N-[1-(2,6-difluorophenyl)-1H-pyrazol-3-yl]-2-trifluoromethylbenzamide, a Bacillus licheniformis strain and cis-Jasmone, a Bacillus licheniformis strain and harpin, a Bacillus licheniformis strain and Azadirachta indica oil, a Bacillus licheniformis strain and Azadirachtin, a Bacillus nematocida strain and alanycarb, a Bacillus nematocida strain and aldicarb, a Bacillus nematocida strain and carbofuran, a Bacillus nematocida strain and carbosulfan, a Bacillus nematocida strain and fosthiazate, a Bacillus nematocida strain and cadusafos, a Bacillus nematocida strain and oxamyl, a Bacillus nematocida strain and thiodicarb, a Bacillus nematocida strain and dimethoate, a Bacillus nematocida strain and ethoprophos, a Bacillus nematocida strain and terbufos, a Bacillus nematocida strain and abamectin, a Bacillus nematocida strain and methyl bromide and other alkyl halides, a Bacillus nematocida strain and methyl isocyanate generators selected from diazomet and metam, a Bacillus nematocida strain and fluazaindolizine, a Bacillus nematocida strain and fluensulfone, a Bacillus nematocida strain and fluopyram, a Bacillus nematocida strain and tioxazafen, a Bacillus nematocida strain and N-[1-(2,6-difluorophenyl)-1H-pyrazol-3-yl]-2-trifluoromethylbenzamide, a Bacillus nematocida strain and cis-Jasmone, a Bacillus nematocida strain and harpin, a Bacillus nematocida strain and Azadirachta indica oil, a Bacillus nematocida strain and Azadirachtin, a Bacillus pumilus strain and alanycarb, a Bacillus pumilus strain and aldicarb, a Bacillus pumilus strain and carbofuran, a Bacillus pumilus strain and carbosulfan, a Bacillus pumilus strain and fosthiazate, a Bacillus pumilus strain and cadusafos, a Bacillus pumilus strain and oxamyl, a Bacillus pumilus strain and thiodicarb, a Bacillus pumilus strain and dimethoate, a Bacillus pumilus strain and ethoprophos, a Bacillus pumilus strain and terbufos, a Bacillus pumilus strain and abamectin, a Bacillus pumilus strain and methyl bromide and other alkyl halides, a Bacillus pumilus strain and methyl isocyanate generators selected from diazomet and metam, a Bacillus pumilus strain and fluazaindolizine, a Bacillus pumilus strain and fluensulfone, a Bacillus pumilus strain and fluopyram, a Bacillus pumilus strain and tioxazafen, a Bacillus pumilus strain and N-[1-(2,6-difluorophenyl)-1H-pyrazol-3-yl]-2-trifluoromethylbenzamide, a Bacillus pumilus strain and cis-Jasmone, a Bacillus pumilus strain and harpin, a Bacillus pumilus strain and Azadirachta indica oil, a Bacillus pumilus strain and Azadirachtin, a Bacillus subtilis strain and alanycarb, a Bacillus subtilis strain and aldicarb, a Bacillus subtilis strain and carbofuran, a Bacillus subtilis strain and carbosulfan, a Bacillus subtilis strain and fosthiazate, a Bacillus subtilis strain and cadusafos, a Bacillus subtilis strain and oxamyl, a Bacillus subtilis strain and thiodicarb, a Bacillus subtilis strain and dimethoate, a Bacillus subtilis strain and ethoprophos, a Bacillus subtilis strain and terbufos, a Bacillus subtilis strain and abamectin, a Bacillus subtilis strain and methyl bromide and other alkyl halides, a Bacillus subtilis strain and methyl isocyanate generators selected from diazomet and metam, a Bacillus subtilis strain and fluazaindolizine, a Bacillus subtilis strain and fluensulfone, a Bacillus subtilis strain and fluopyram, a Bacillus subtilis strain and tioxazafen, a Bacillus subtilis strain and N-[1-(2,6-difluorophenyl)-1H-pyrazol-3-yl]-2-trifluoromethylbenzamide, a Bacillus subtilis strain and cis-Jasmone, a Bacillus subtilis strain and harpin, a Bacillus subtilis strain and Azadirachta indica oil, a Bacillus subtilis strain and Azadirachtin, a Bacillus penetrans strain and alanycarb, a Bacillus penetrans strain and aldicarb, a Bacillus penetrans strain and carbofuran, a Bacillus penetrans strain and carbosulfan, a Bacillus penetrans strain and fosthiazate, a Bacillus penetrans strain and cadusafos, a Bacillus penetrans strain and oxamyl, a Bacillus penetrans strain and thiodicarb, a Bacillus penetrans strain and dimethoate, a Bacillus penetrans strain and ethoprophos, a Bacillus penetrans strain and terbufos, a Bacillus penetrans strain and abamectin, a Bacillus penetrans strain and methyl bromide and other alkyl halides, a Bacillus penetrans strain and methyl isocyanate generators selected from diazomet and metam, a Bacillus penetrans strain and fluazaindolizine, a Bacillus penetrans strain and fluensulfone, a Bacillus penetrans strain and fluopyram, a Bacillus penetrans strain and tioxazafen, a Bacillus penetrans strain and N-[1-(2,6-difluorophenyl)-1H-pyrazol-3-yl]-2-trifluoromethylbenzamide, a Bacillus penetrans strain and cis-Jasmone, a Bacillus penetrans strain and harpin, a Bacillus penetrans strain and Azadirachta indica oil, a Bacillus penetrans strain and Azadirachtin, a Bacillus thuringiensis strain and alanycarb, a Bacillus thuringiensis strain and aldicarb, a Bacillus thuringiensis strain and carbofuran, a Bacillus thuringiensis strain and carbosulfan, a Bacillus thuringiensis strain and fosthiazate, a Bacillus thuringiensis strain and cadusafos, a Bacillus thuringiensis strain and oxamyl, a Bacillus thuringiensis strain and thiodicarb, a Bacillus thuringiensis strain and dimethoate, a Bacillus thuringiensis strain and ethoprophos, a Bacillus thuringiensis strain and terbufos, a Bacillus thuringiensis strain and abamectin, a Bacillus thuringiensis strain and methyl bromide and other alkyl halides, a Bacillus thuringiensis strain and methyl isocyanate generators selected from diazomet and metam, a Bacillus thuringiensis strain and fluazaindolizine, a Bacillus thuringiensis strain and fluensulfone, a Bacillus thuringiensis strain and fluopyram, a Bacillus thuringiensis strain and tioxazafen, a Bacillus thuringiensis strain and N-[1-(2,6-difluorophenyl)-1H-pyrazol-3-yl]-2-trifluoromethylbenzamide, a Bacillus thuringiensis strain and cis-Jasmone, a Bacillus thuringiensis strain and harpin, a Bacillus thuringiensis strain and Azadirachta indica oil, a Bacillus thuringiensis strain and Azadirachtin, a Brevibacillus laterosporus strain and alanycarb, a Brevibacillus laterosporus strain and aldicarb, a Brevibacillus laterosporus strain and carbofuran, a Brevibacillus laterosporus strain and carbosulfan, a Brevibacillus laterosporus strain and fosthiazate, a Brevibacillus laterosporus strain and cadusafos, a Brevibacillus laterosporus strain and oxamyl, a Brevibacillus laterosporus strain and thiodicarb, a Brevibacillus laterosporus strain and dimethoate, a Brevibacillus laterosporus strain and ethoprophos, a Brevibacillus laterosporus strain and terbufos, a Brevibacillus laterosporus strain and abamectin, a Brevibacillus laterosporus strain and methyl bromide and other alkyl halides, a Brevibacillus laterosporus strain and methyl isocyanate generators selected from diazomet and metam, a Brevibacillus laterosporus strain and fluazaindolizine, a Brevibacillus laterosporus strain and fluensulfone, a Brevibacillus laterosporus strain and fluopyram, a Brevibacillus laterosporus strain and tioxazafen, a Brevibacillus laterosporus strain and N-[1-(2,6-difluorophenyl)-1H-pyrazol-3-yl]-2-trifluoromethylbenzamide, a Brevibacillus laterosporus strain and cis-Jasmone, a Brevibacillus laterosporus strain and harpin, a Brevibacillus laterosporus strain and Azadirachta indica oil, a Brevibacillus laterosporus strain and Azadirachtin, a Burkholderia rinojensis strain and alanycarb, a Burkholderia rinojensis strain and aldicarb, a Burkholderia rinojensis strain and carbofuran, a Burkholderia rinojensis strain and carbosulfan, a Burkholderia rinojensis strain and fosthiazate, a Burkholderia rinojensis strain and cadusafos, a Burkholderia rinojensis strain and oxamyl, a Burkholderia rinojensis strain and thiodicarb, a Burkholderia rinojensis strain and dimethoate, a Burkholderia rinojensis strain and ethoprophos, a Burkholderia rinojensis strain and terbufos, a Burkholderia rinojensis strain and abamectin, a Burkholderia rinojensis strain and methyl bromide and other alkyl halides, a Burkholderia rinojensis strain and methyl isocyanate generators selected from diazomet and metam, a Burkholderia rinojensis strain and fluazaindolizine, a Burkholderia rinojensis strain and fluensulfone, a Burkholderia rinojensis strain and fluopyram, a Burkholderia rinojensis strain and tioxazafen, a Burkholderia rinojensis strain and N-[1-(2,6-difluorophenyl)-1H-pyrazol-3-yl]-2-trifluoromethylbenzamide, a Burkholderia rinojensis strain and cis-Jasmone, a Burkholderia rinojensis strain and harpin, a Burkholderia rinojensis strain and Azadirachta indica oil, a Burkholderia rinojensis strain and Azadirachtin, a Lysobacter antibioticus strain and alanycarb, a Lysobacter antibioticus strain and aldicarb, a Lysobacter antibioticus strain and carbofuran, a Lysobacter antibioticus strain and carbosulfan, a Lysobacter antibioticus strain and fosthiazate, a Lysobacter antibioticus strain and cadusafos, a Lysobacter antibioticus strain and oxamyl, a Lysobacter antibioticus strain and thiodicarb, a Lysobacter antibioticus strain and dimethoate, a Lysobacter antibioticus strain and ethoprophos, a Lysobacter antibioticus strain and terbufos, a Lysobacter antibioticus strain and abamectin, a Lysobacter antibioticus strain and methyl bromide and other alkyl halides, a Lysobacter antibioticus strain and methyl isocyanate generators selected from diazomet and metam, a Lysobacter antibioticus strain and fluazaindolizine, a Lysobacter antibioticus strain and fluensulfone, a Lysobacter antibioticus strain and fluopyram, a Lysobacter antibioticus strain and tioxazafen, a Lysobacter antibioticus strain and N-[1-(2,6-difluorophenyl)-1H-pyrazol-3-yl]-2-trifluoromethylbenzamide, a Lysobacter antibioticus strain and cis-Jasmone, a Lysobacter antibioticus strain and harpin, a Lysobacter antibioticus strain and Azadirachta indica oil, a Lysobacter antibioticus strain and Azadirachtin, a Lysobacter enzymogenes strain and alanycarb, a Lysobacter enzymogenes strain and aldicarb, a Lysobacter enzymogenes strain and carbofuran, a Lysobacter enzymogenes strain and carbosulfan, a Lysobacter enzymogenes strain and fosthiazate, a Lysobacter enzymogenes strain and cadusafos, a Lysobacter enzymogenes strain and oxamyl, a Lysobacter enzymogenes strain and thiodicarb, a Lysobacter enzymogenes strain and dimethoate, a Lysobacter enzymogenes strain and ethoprophos, a Lysobacter enzymogenes strain and terbufos, a Lysobacter enzymogenes strain and abamectin, a Lysobacter enzymogenes strain and methyl bromide and other alkyl halides, a Lysobacter enzymogenes strain and methyl isocyanate generators selected from diazomet and metam, a Lysobacter enzymogenes strain and fluazaindolizine, a Lysobacter enzymogenes strain and fluensulfone, a Lysobacter enzymogenes strain and fluopyram, a Lysobacter enzymogenes strain and tioxazafen, a Lysobacter enzymogenes strain and N-[1-(2,6-difluorophenyl)-1H-pyrazol-3-yl]-2-trifluoromethylbenzamide, a Lysobacter enzymogenes strain and cis-Jasmone, a Lysobacter enzymogenes strain and harpin, a Lysobacter enzymogenes strain and Azadirachta indica oil, a Lysobacter enzymogenes strain and Azadirachtin, a Pasteuria nishizawae strain and alanycarb, a Pasteuria nishizawae strain and aldicarb, a Pasteuria nishizawae strain and carbofuran, a Pasteuria nishizawae strain and carbosulfan, a Pasteuria nishizawae strain and fosthiazate, a Pasteuria nishizawae strain and cadusafos, a Pasteuria nishizawae strain and oxamyl, a Pasteuria nishizawae strain and thiodicarb, a Pasteuria nishizawae strain and dimethoate, a Pasteuria nishizawae strain and ethoprophos, a Pasteuria nishizawae strain and terbufos, a Pasteuria nishizawae strain and abamectin, a Pasteuria nishizawae strain and methyl bromide and other alkyl halides, a Pasteuria nishizawae strain and methyl isocyanate generators selected from diazomet and metam, a Pasteuria nishizawae strain and fluazaindolizine, a Pasteuria nishizawae strain and fluensulfone, a Pasteuria nishizawae strain and fluopyram, a Pasteuria nishizawae strain and tioxazafen, a Pasteuria nishizawae strain and N-[1-(2,6-difluorophenyl)-1H-pyrazol-3-yl]-2-trifluoromethylbenzamide, a Pasteuria nishizawae strain and cis-Jasmone, a Pasteuria nishizawae strain and harpin, a Pasteuria nishizawae strain and Azadirachta indica oil, a Pasteuria nishizawae strain and Azadirachtin, a Pasteuria penetrans strain and alanycarb, a Pasteuria penetrans strain and aldicarb, a Pasteuria penetrans strain and carbofuran, a Pasteuria penetrans strain and carbosulfan, a Pasteuria penetrans strain and fosthiazate, a Pasteuria penetrans strain and cadusafos, a Pasteuria penetrans strain and oxamyl, a Pasteuria penetrans strain and thiodicarb, a Pasteuria penetrans strain and dimethoate, a Pasteuria penetrans strain and ethoprophos, a Pasteuria penetrans strain and terbufos, a Pasteuria penetrans strain and abamectin, a Pasteuria penetrans strain and methyl bromide and other alkyl halides, a Pasteuria penetrans strain and methyl isocyanate generators selected from diazomet and metam, a Pasteuria penetrans strain and fluazaindolizine, a Pasteuria penetrans strain and fluensulfone, a Pasteuria penetrans strain and fluopyram, a Pasteuria penetrans strain and tioxazafen, a Pasteuria penetrans strain and N-[1-(2,6-difluorophenyl)-1H-pyrazol-3-yl]-2-trifluoromethylbenzamide, a Pasteuria penetrans strain and cis-Jasmone, a Pasteuria penetrans strain and harpin, a Pasteuria penetrans strain and Azadirachta indica oil, a Pasteuria penetrans strain and Azadirachtin, a Pasteuria ramosa strain and alanycarb, a Pasteuria ramosa strain and aldicarb, a Pasteuria ramosa strain and carbofuran, a Pasteuria ramosa strain and carbosulfan, a Pasteuria ramosa strain and fosthiazate, a Pasteuria ramosa strain and cadusafos, a Pasteuria ramosa strain and oxamyl, a Pasteuria ramosa strain and thiodicarb, a Pasteuria ramosa strain and dimethoate, a Pasteuria ramosa strain and ethoprophos, a Pasteuria ramosa strain and terbufos, a Pasteuria ramosa strain and abamectin, a Pasteuria ramosa strain and methyl bromide and other alkyl halides, a Pasteuria ramosa strain and methyl isocyanate generators selected from diazomet and metam, a Pasteuria ramosa strain and fluazaindolizine, a Pasteuria ramosa strain and fluensulfone, a Pasteuria ramosa strain and fluopyram, a Pasteuria ramosa strain and tioxazafen, a Pasteuria ramosa strain and N-[1-(2,6-difluorophenyl)-1H-pyrazol-3-yl]-2-trifluoromethylbenzamide, a Pasteuria ramosa strain and cis-Jasmone, a Pasteuria ramosa strain and harpin, a Pasteuria ramosa strain and Azadirachta indica oil, a Pasteuria ramosa strain and Azadirachtin, a Pasteuria reniformis strain and alanycarb, a Pasteuria reniformis strain and aldicarb, a Pasteuria reniformis strain and carbofuran, a Pasteuria reniformis strain and carbosulfan, a Pasteuria reniformis strain and fosthiazate, a Pasteuria reniformis strain and cadusafos, a Pasteuria reniformis strain and oxamyl, a Pasteuria reniformis strain and thiodicarb, a Pasteuria reniformis strain and dimethoate, a Pasteuria reniformis strain and ethoprophos, a Pasteuria reniformis strain and terbufos, a Pasteuria reniformis strain and abamectin, a Pasteuria reniformis strain and methyl bromide and other alkyl halides, a Pasteuria reniformis strain and methyl isocyanate generators selected from diazomet and metam, a Pasteuria reniformis strain and fluazaindolizine, a Pasteuria reniformis strain and fluensulfone, a Pasteuria reniformis strain and fluopyram, a Pasteuria reniformis strain and tioxazafen, a Pasteuria reniformis strain and N-[1-(2,6-difluorophenyl)-1H-pyrazol-3-yl]-2-trifluoromethylbenzamide, a Pasteuria reniformis strain and cis-Jasmone, a Pasteuria reniformis strain and harpin, a Pasteuria reniformis strain and Azadirachta indica oil, a Pasteuria reniformis strain and Azadirachtin, a Pasteuria thornei strain and alanycarb, a Pasteuria thornei strain and aldicarb, a Pasteuria thornei strain and carbofuran, a Pasteuria thornei strain and carbosulfan, a Pasteuria thornei strain and fosthiazate, a Pasteuria thornei strain and cadusafos, a Pasteuria thornei strain and oxamyl, a Pasteuria thornei strain and thiodicarb, a Pasteuria thornei strain and dimethoate, a Pasteuria thornei strain and ethoprophos, a Pasteuria thornei strain and terbufos, a Pasteuria thornei strain and abamectin, a Pasteuria thornei strain and methyl bromide and other alkyl halides, a Pasteuria thornei strain and methyl isocyanate generators selected from diazomet and metam, a Pasteuria thornei strain and fluazaindolizine, a Pasteuria thornei strain and fluensulfone, a Pasteuria thornei strain and fluopyram, a Pasteuria thornei strain and tioxazafen, a Pasteuria thornei strain and N-[1-(2,6-difluorophenyl)-1H-pyrazol-3-yl]-2-trifluoromethylbenzamide, a Pasteuria thornei strain and cis-Jasmone, a Pasteuria thornei strain and harpin, a Pasteuria thornei strain and Azadirachta indica oil, a Pasteuria thornei strain and Azadirachtin, a Pasteuria usage strain and alanycarb, a Pasteuria usage strain and aldicarb, a Pasteuria usage strain and carbofuran, a Pasteuria usage strain and carbosulfan, a Pasteuria usage strain and fosthiazate, a Pasteuria usage strain and cadusafos, a Pasteuria usage strain and oxamyl, a Pasteuria usage strain and thiodicarb, a Pasteuria usage strain and dimethoate, a Pasteuria usage strain and ethoprophos, a Pasteuria usage strain and terbufos, a Pasteuria usage strain and abamectin, a Pasteuria usage strain and methyl bromide and other alkyl halides, a Pasteuria usage strain and methyl isocyanate generators selected from diazomet and metam, a Pasteuria usage strain and fluazaindolizine, a Pasteuria usage strain and fluensulfone, a Pasteuria usage strain and fluopyram, a Pasteuria usage strain and tioxazafen, a Pasteuria usage strain and N-[1-(2,6-difluorophenyl)-1H-pyrazol-3-yl]-2-trifluoromethylbenzamide, a Pasteuria usage strain and cis-Jasmone, a Pasteuria usage strain and harpin, a Pasteuria usage strain and Azadirachta indica oil, a Pasteuria usage strain and Azadirachtin, an Arthrobotrys dactyloides strain and alanycarb, an Arthrobotrys dactyloides strain and aldicarb, an Arthrobotrys dactyloides strain and carbofuran, an Arthrobotrys dactyloides strain and carbosulfan, an Arthrobotrys dactyloides strain and fosthiazate, an Arthrobotrys dactyloides strain and cadusafos, an Arthrobotrys dactyloides strain and oxamyl, an Arthrobotrys dactyloides strain and thiodicarb, an Arthrobotrys dactyloides strain and dimethoate, an Arthrobotrys dactyloides strain and ethoprophos, an Arthrobotrys dactyloides strain and terbufos, an Arthrobotrys dactyloides strain and abamectin, an Arthrobotrys dactyloides strain and methyl bromide and other alkyl halides, an Arthrobotrys dactyloides strain and methyl isocyanate generators selected from diazomet and metam, an Arthrobotrys dactyloides strain and fluazaindolizine, an Arthrobotrys dactyloides strain and fluensulfone, an Arthrobotrys dactyloides strain and fluopyram, an Arthrobotrys dactyloides strain and tioxazafen, an Arthrobotrys dactyloides strain and N-[1-(2,6-difluorophenyl)-1H-pyrazol-3-yl]-2-trifluoromethylbenzamide, an Arthrobotrys dactyloides strain and cis-Jasmone, an Arthrobotrys dactyloides strain and harpin, an Arthrobotrys dactyloides strain and Azadirachta indica oil, an Arthrobotrys dactyloides strain and Azadirachtin, an Arthrobotrys oligospora strain and alanycarb, an Arthrobotrys oligospora strain and aldicarb, an Arthrobotrys oligospora strain and carbofuran, an Arthrobotrys oligospora strain and carbosulfan, an Arthrobotrys oligospora strain and fosthiazate, an Arthrobotrys oligospora strain and cadusafos, an Arthrobotrys oligospora strain and oxamyl, an Arthrobotrys oligospora strain and thiodicarb, an Arthrobotrys oligospora strain and dimethoate, an Arthrobotrys oligospora strain and ethoprophos, an Arthrobotrys oligospora strain and terbufos, an Arthrobotrys oligospora strain and abamectin, an Arthrobotrys oligospora strain and methyl bromide and other alkyl halides, an Arthrobotrys oligospora strain and methyl isocyanate generators selected from diazomet and metam, an Arthrobotrys oligospora strain and fluazaindolizine, an Arthrobotrys oligospora strain and fluensulfone, an Arthrobotrys oligospora strain and fluopyram, an Arthrobotrys oligospora strain and tioxazafen, an Arthrobotrys oligospora strain and N-[1-(2,6-difluorophenyl)-1H-pyrazol-3-yl]-2-trifluoromethylbenzamide, an Arthrobotrys oligospora strain and cis-Jasmone, an Arthrobotrys oligospora strain and harpin, an Arthrobotrys oligospora strain and Azadirachta indica oil, an Arthrobotrys oligospora strain and Azadirachtin, an Arthrobotrys superba strain and alanycarb, an Arthrobotrys superba strain and aldicarb, an Arthrobotrys superba strain and carbofuran, an Arthrobotrys superba strain and carbosulfan, an Arthrobotrys superba strain and fosthiazate, an Arthrobotrys superba strain and cadusafos, an Arthrobotrys superba strain and oxamyl, an Arthrobotrys superba strain and thiodicarb, an Arthrobotrys superba strain and dimethoate, an Arthrobotrys superba strain and ethoprophos, an Arthrobotrys superba strain and terbufos, an Arthrobotrys superba strain and abamectin, an Arthrobotrys superba strain and methyl bromide and other alkyl halides, an Arthrobotrys superba strain and methyl isocyanate generators selected from diazomet and metam, an Arthrobotrys superba strain and fluazaindolizine, an Arthrobotrys superba strain and fluensulfone, an Arthrobotrys superba strain and fluopyram, an Arthrobotrys superba strain and tioxazafen, an Arthrobotrys superba strain and N-[1-(2,6-difluorophenyl)-1H-pyrazol-3-yl]-2-trifluoromethylbenzamide, an Arthrobotrys superba strain and cis-Jasmone, an Arthrobotrys superba strain and harpin, an Arthrobotrys superba strain and Azadirachta indica oil, an Arthrobotrys superba strain and Azadirachtin, a Nematoctonus geogenius strain and alanycarb, a Nematoctonus geogenius strain and aldicarb, a Nematoctonus geogenius strain and carbofuran, a Nematoctonus geogenius strain and carbosulfan, a Nematoctonus geogenius strain and fosthiazate, a Nematoctonus geogenius strain and cadusafos, a Nematoctonus geogenius strain and oxamyl, a Nematoctonus geogenius strain and thiodicarb, a Nematoctonus geogenius strain and dimethoate, a Nematoctonus geogenius strain and ethoprophos, a Nematoctonus geogenius strain and terbufos, a Nematoctonus geogenius strain and abamectin, a Nematoctonus geogenius strain and methyl bromide and other alkyl halides, a Nematoctonus geogenius strain and methyl isocyanate generators selected from diazomet and metam, a Nematoctonus geogenius strain and fluazaindolizine, a Nematoctonus geogenius strain and fluensulfone, a Nematoctonus geogenius strain and fluopyram, a Nematoctonus geogenius strain and tioxazafen, a Nematoctonus geogenius strain and N-[1-(2,6-difluorophenyl)-1H-pyrazol-3-yl]-2-trifluoromethylbenzamide, a Nematoctonus geogenius strain and cis-Jasmone, a Nematoctonus geogenius strain and harpin, a Nematoctonus geogenius strain and Azadirachta indica oil, a Nematoctonus geogenius strain and Azadirachtin, a Nematoctonus leiosporus strain and alanycarb, a Nematoctonus leiosporus strain and aldicarb, a Nematoctonus leiosporus strain and carbofuran, a Nematoctonus leiosporus strain and carbosulfan, a Nematoctonus leiosporus strain and fosthiazate, a Nematoctonus leiosporus strain and cadusafos, a Nematoctonus leiosporus strain and oxamyl, a Nematoctonus leiosporus strain and thiodicarb, a Nematoctonus leiosporus strain and dimethoate, a Nematoctonus leiosporus strain and ethoprophos, a Nematoctonus leiosporus strain and terbufos, a Nematoctonus leiosporus strain and abamectin, a Nematoctonus leiosporus strain and methyl bromide and other alkyl halides, a Nematoctonus leiosporus strain and methyl isocyanate generators selected from diazomet and metam, a Nematoctonus leiosporus strain and fluazaindolizine, a Nematoctonus leiosporus strain and fluensulfone, a Nematoctonus leiosporus strain and fluopyram, a Nematoctonus leiosporus strain and tioxazafen, a Nematoctonus leiosporus strain and N-[1-(2,6-difluorophenyl)-1H-pyrazol-3-yl]-2-trifluoromethylbenzamide, a Nematoctonus leiosporus strain and cis-Jasmone, a Nematoctonus leiosporus strain and harpin, a Nematoctonus leiosporus strain and Azadirachta indica oil, a Nematoctonus leiosporus strain and Azadirachtin, a Myrothecium verrucaria strain and alanycarb, a Myrothecium verrucaria strain and aldicarb, a Myrothecium verrucaria strain and carbofuran, a Myrothecium verrucaria strain and carbosulfan, a Myrothecium verrucaria strain and fosthiazate, a Myrothecium verrucaria strain and cadusafos, a Myrothecium verrucaria strain and oxamyl, a Myrothecium verrucaria strain and thiodicarb, a Myrothecium verrucaria strain and dimethoate, a Myrothecium verrucaria strain and ethoprophos, a Myrothecium verrucaria strain and terbufos, a Myrothecium verrucaria strain and abamectin, a Myrothecium verrucaria strain and methyl bromide and other alkyl halides, a Myrothecium verrucaria strain and methyl isocyanate generators selected from diazomet and metam, a Myrothecium verrucaria strain and fluazaindolizine, a Myrothecium verrucaria strain and fluensulfone, a Myrothecium verrucaria strain and fluopyram, a Myrothecium verrucaria strain and tioxazafen, a Myrothecium verrucaria strain and N-[1-(2,6-difluorophenyl)-1H-pyrazol-3-yl]-2-trifluoromethylbenzamide, a Myrothecium verrucaria strain and cis-Jasmone, a Myrothecium verrucaria strain and harpin, a Myrothecium verrucaria strain and Azadirachta indica oil, a Myrothecium verrucaria strain and Azadirachtin, a Paecilomyces lilacinus strain and alanycarb, a Paecilomyces lilacinus strain and aldicarb, a Paecilomyces lilacinus strain and carbofuran, a Paecilomyces lilacinus strain and carbosulfan, a Paecilomyces lilacinus strain and fosthiazate, a Paecilomyces lilacinus strain and cadusafos, a Paecilomyces lilacinus strain and oxamyl, a Paecilomyces lilacinus strain and thiodicarb, a Paecilomyces lilacinus strain and dimethoate, a Paecilomyces lilacinus strain and ethoprophos, a Paecilomyces lilacinus strain and terbufos, a Paecilomyces lilacinus strain and abamectin, a Paecilomyces lilacinus strain and methyl bromide and other alkyl halides, a Paecilomyces lilacinus strain and methyl isocyanate generators selected from diazomet and metam, a Paecilomyces lilacinus strain and fluazaindolizine, a Paecilomyces lilacinus strain and fluensulfone, a Paecilomyces lilacinus strain and fluopyram, a Paecilomyces lilacinus strain and tioxazafen, a Paecilomyces lilacinus strain and N-[1-(2,6-difluorophenyl)-1H-pyrazol-3-yl]-2-trifluoromethylbenzamide, a Paecilomyces lilacinus strain and cis-Jasmone, a Paecilomyces lilacinus strain and harpin, a Paecilomyces lilacinus strain and Azadirachta indica oil, a Paecilomyces lilacinus strain and Azadirachtin, a Paecilomyces variotii strain and alanycarb, a Paecilomyces variotii strain and aldicarb, a Paecilomyces variotii strain and carbofuran, a Paecilomyces variotii strain and carbosulfan, a Paecilomyces variotii strain and fosthiazate, a Paecilomyces variotii strain and cadusafos, a Paecilomyces variotii strain and oxamyl, a Paecilomyces variotii strain and thiodicarb, a Paecilomyces variotii strain and dimethoate, a Paecilomyces variotii strain and ethoprophos, a Paecilomyces variotii strain and terbufos, a Paecilomyces variotii strain and abamectin, a Paecilomyces variotii strain and methyl bromide and other alkyl halides, a Paecilomyces variotii strain and methyl isocyanate generators selected from diazomet and metam, a Paecilomyces variotii strain and fluazaindolizine, a Paecilomyces variotii strain and fluensulfone, a Paecilomyces variotii strain and fluopyram, a Paecilomyces variotii strain and tioxazafen, a Paecilomyces variotii strain and N-[1-(2,6-difluorophenyl)-1H-pyrazol-3-yl]-2-trifluoromethylbenzamide, a Paecilomyces variotii strain and cis-Jasmone, a Paecilomyces variotii strain and harpin, a Paecilomyces variotii strain and Azadirachta indica oil, a Paecilomyces variotii strain and Azadirachtin, a Trichoderma asperellum strain and alanycarb, a Trichoderma asperellum strain and aldicarb, a Trichoderma asperellum strain and carbofuran, a Trichoderma asperellum strain and carbosulfan, a Trichoderma asperellum strain and fosthiazate, a Trichoderma asperellum strain and cadusafos, a Trichoderma asperellum strain and oxamyl, a Trichoderma asperellum strain and thiodicarb, a Trichoderma asperellum strain and dimethoate, a Trichoderma asperellum strain and ethoprophos, a Trichoderma asperellum strain and terbufos, a Trichoderma asperellum strain and abamectin, a Trichoderma asperellum strain and methyl bromide and other alkyl halides, a Trichoderma asperellum strain and methyl isocyanate generators selected from diazomet and metam, a Trichoderma asperellum strain and fluazaindolizine, a Trichoderma asperellum strain and fluensulfone, a Trichoderma asperellum strain and fluopyram, a Trichoderma asperellum strain and tioxazafen, a Trichoderma asperellum strain and N-[1-(2,6-difluorophenyl)-1H-pyrazol-3-yl]-2-trifluoromethylbenzamide, a Trichoderma asperellum strain and cis-Jasmone, a Trichoderma asperellum strain and harpin, a Trichoderma asperellum strain and Azadirachta indica oil, a Trichoderma asperellum strain and Azadirachtin, a Trichoderma atroviride strain and alanycarb, a Trichoderma atroviride strain and aldicarb, a Trichoderma atroviride strain and carbofuran, a Trichoderma atroviride strain and carbosulfan, a Trichoderma atroviride strain and fosthiazate, a Trichoderma atroviride strain and cadusafos, a Trichoderma atroviride strain and oxamyl, a Trichoderma atroviride strain and thiodicarb, a Trichoderma atroviride strain and dimethoate, a Trichoderma atroviride strain and ethoprophos, a Trichoderma atroviride strain and terbufos, a Trichoderma atroviride strain and abamectin, a Trichoderma atroviride strain and methyl bromide and other alkyl halides, a Trichoderma atroviride strain and methyl isocyanate generators selected from diazomet and metam, a Trichoderma atroviride strain and fluazaindolizine, a Trichoderma atroviride strain and fluensulfone, a Trichoderma atroviride strain and fluopyram, a Trichoderma atroviride strain and tioxazafen, a Trichoderma atroviride strain and N-[1-(2,6-difluorophenyl)-1H-pyrazol-3-yl]-2-trifluoromethylbenzamide, a Trichoderma atroviride strain and cis-Jasmone, a Trichoderma atroviride strain and harpin, a Trichoderma atroviride strain and Azadirachta indica oil, a Trichoderma atroviride strain and Azadirachtin, a Trichoderma harzianum strain and alanycarb, a Trichoderma harzianum strain and aldicarb, a Trichoderma harzianum strain and carbofuran, a Trichoderma harzianum strain and carbosulfan, a Trichoderma harzianum strain and fosthiazate, a Trichoderma harzianum strain and cadusafos, a Trichoderma harzianum strain and oxamyl, a Trichoderma harzianum strain and thiodicarb, a Trichoderma harzianum strain and dimethoate, a Trichoderma harzianum strain and ethoprophos, a Trichoderma harzianum strain and terbufos, a Trichoderma harzianum strain and abamectin, a Trichoderma harzianum strain and methyl bromide and other alkyl halides, a Trichoderma harzianum strain and methyl isocyanate generators selected from diazomet and metam, a Trichoderma harzianum strain and fluazaindolizine, a Trichoderma harzianum strain and fluensulfone, a Trichoderma harzianum strain and fluopyram, a Trichoderma harzianum strain and tioxazafen, a Trichoderma harzianum strain and N-[1-(2,6-difluorophenyl)-1H-pyrazol-3-yl]-2-trifluoromethylbenzamide, a Trichoderma harzianum strain and cis-Jasmone, a Trichoderma harzianum strain and harpin, a Trichoderma harzianum strain and Azadirachta indica oil, a Trichoderma harzianum strain and Azadirachtin, a Trichoderma viride strain and alanycarb, a Trichoderma viride strain and aldicarb, a Trichoderma viride strain and carbofuran, a Trichoderma viride strain and carbosulfan, a Trichoderma viride strain and fosthiazate, a Trichoderma viride strain and cadusafos, a Trichoderma viride strain and oxamyl, a Trichoderma viride strain and thiodicarb, a Trichoderma viride strain and dimethoate, a Trichoderma viride strain and ethoprophos, a Trichoderma viride strain and terbufos, a Trichoderma viride strain and abamectin, a Trichoderma viride strain and methyl bromide and other alkyl halides, a Trichoderma viride strain and methyl isocyanate generators selected from diazomet and metam, a Trichoderma viride strain and fluazaindolizine, a Trichoderma viride strain and fluensulfone, a Trichoderma viride strain and fluopyram, a Trichoderma viride strain and tioxazafen, a Trichoderma viride strain and N-[1-(2,6-difluorophenyl)-1H-pyrazol-3-yl]-2-trifluoromethylbenzamide, a Trichoderma viride strain and cis-Jasmone, a Trichoderma viride strain and harpin, a Trichoderma viride strain and Azadirachta indica oil, a Trichoderma viride strain and Azadirachtin, a Trichoderma harzianum rifai strain and alanycarb, a Trichoderma harzianum rifai strain and aldicarb, a Trichoderma harzianum rifai strain and carbofuran, a Trichoderma harzianum rifai strain and carbosulfan, a Trichoderma harzianum rifai strain and fosthiazate, a Trichoderma harzianum rifai strain and cadusafos, a Trichoderma harzianum rifai strain and oxamyl, a Trichoderma harzianum rifai strain and thiodicarb, a Trichoderma harzianum rifai strain and dimethoate, a Trichoderma harzianum rifai strain and ethoprophos, a Trichoderma harzianum rifai strain and terbufos, a Trichoderma harzianum rifai strain and abamectin, a Trichoderma harzianum rifai strain and methyl bromide and other alkyl halides, a Trichoderma harzianum rifai strain and methyl isocyanate generators selected from diazomet and metam, a Trichoderma harzianum rifai strain and fluazaindolizine, a Trichoderma harzianum rifai strain and fluensulfone, a Trichoderma harzianum rifai strain and fluopyram, a Trichoderma harzianum rifai strain and tioxazafen, a Trichoderma harzianum rifai strain and N-[1-(2,6-difluorophenyl)-1H-pyrazol-3-yl]-2-trifluoromethylbenzamide, a Trichoderma harzianum rifai strain and cis-Jasmone, a Trichoderma harzianum rifai strain and harpin, a Trichoderma harzianum rifai strain and Azadirachta indica oil, a Trichoderma harzianum rifai strain and Azadirachtin, a Tsukamurella paurometabola strain and alanycarb, a Tsukamurella paurometabola strain and aldicarb, a Tsukamurella paurometabola strain and carbofuran, a Tsukamurella paurometabola strain and carbosulfan, a Tsukamurella paurometabola strain and fosthiazate, a Tsukamurella paurometabola strain and cadusafos, a Tsukamurella paurometabola strain and oxamyl, a Tsukamurella paurometabola strain and thiodicarb, a Tsukamurella paurometabola strain and dimethoate, a Tsukamurella paurometabola strain and ethoprophos, a Tsukamurella paurometabola strain and terbufos, a Tsukamurella paurometabola strain and abamectin, a Tsukamurella paurometabola strain and methyl bromide and other alkyl halides, a Tsukamurella paurometabola strain and methyl isocyanate generators selected from diazomet and metam, a Tsukamurella paurometabola strain and fluazaindolizine, a Tsukamurella paurometabola strain and fluensulfone, a Tsukamurella paurometabola strain and fluopyram, a Tsukamurella paurometabola strain and tioxazafen, a Tsukamurella paurometabola strain and N-[1-(2,6-difluorophenyl)-1H-pyrazol-3-yl]-2-trifluoromethylbenzamide, a Tsukamurella paurometabola strain and cis-Jasmone, a Tsukamurella paurometabola strain and harpin, a Tsukamurella paurometabola strain and Azadirachta indica oil, and a Tsukamurella paurometabola strain and Azadirachtin.
116. The plant, cell, plant part or seed of any one of these numbered paragraphs, or the soil in which they are grown or are intended to be grown, treated with a combination of a nematicidal agent and a biological control agent selected from: Bacillus amyloliquefaciens and alanycarb; Bacillus amyloliquefaciens and aldicarb; Bacillus amyloliquefaciens and carbofuran; Bacillus amyloliquefaciens and carbosulfan; Bacillus amyloliquefaciens and fosthiazate; Bacillus amyloliquefaciens and cadusafos; Bacillus amyloliquefaciens and oxamyl; Bacillus amyloliquefaciens and thiodicarb; Bacillus amyloliquefaciens and dimethoate; Bacillus amyloliquefaciens and ethoprophos; Bacillus amyloliquefaciens and terbufos; Bacillus amyloliquefaciens and abamectin; Bacillus amyloliquefaciens and methyl bromide and other alkyl halides; Bacillus amyloliquefaciens and methyl isocyanate generators selected from diazomet and metam; Bacillus amyloliquefaciens and fluazaindolizine; Bacillus amyloliquefaciens and fluensulfone; Bacillus amyloliquefaciens and fluopyram; Bacillus amyloliquefaciens and tioxazafen; Bacillus amyloliquefaciens and N-[1-(2,6-difluorophenyl)-1H-pyrazol-3-yl]-2-trifluoromethylbenzamide; Bacillus amyloliquefaciens and cis-Jasmone; Bacillus amyloliquefaciens and harpin; Bacillus amyloliquefaciens and Azadirachta indica oil; Bacillus amyloliquefaciens and Azadirachtin; Bacillus firmus and alanycarb; Bacillus firmus and aldicarb; Bacillus firmus and carbofuran; Bacillus firmus and carbosulfan; Bacillus firmus and fosthiazate; Bacillus firmus and cadusafos; Bacillus firmus and oxamyl; Bacillus firmus and thiodicarb; Bacillus firmus and dimethoate; Bacillus firmus and ethoprophos; Bacillus firmus and terbufos; Bacillus firmus and abamectin; Bacillus firmus and methyl bromide and other alkyl halides; Bacillus firmus and methyl isocyanate generators selected from diazomet and metam; Bacillus firmus and fluazaindolizine; Bacillus firmus and fluensulfone; Bacillus firmus and fluopyram; Bacillus firmus and tioxazafen; Bacillus firmus and N-[1-(2,6-difluorophenyl)-1H-pyrazol-3-yl]-2-trifluoromethylbenzamide; Bacillus firmus and cis-Jasmone; Bacillus firmus and harpin; Bacillus firmus and Azadirachta indica oil; Bacillus firmus and Azadirachtin; Bacillus laterosporus and alanycarb; Bacillus laterosporus and aldicarb; Bacillus laterosporus and carbofuran; Bacillus laterosporus and carbosulfan; Bacillus laterosporus and fosthiazate; Bacillus laterosporus and cadusafos; Bacillus laterosporus and oxamyl; Bacillus laterosporus and thiodicarb; Bacillus laterosporus and dimethoate; Bacillus laterosporus and ethoprophos; Bacillus laterosporus and terbufos; Bacillus laterosporus and abamectin; Bacillus laterosporus and methyl bromide and other alkyl halides; Bacillus laterosporus and methyl isocyanate generators selected from diazomet and metam; Bacillus laterosporus and fluazaindolizine; Bacillus laterosporus and fluensulfone; Bacillus laterosporus and fluopyram; Bacillus laterosporus and tioxazafen; Bacillus laterosporus and N-[1-(2,6-difluorophenyl)-1H-pyrazol-3-yl]-2-trifluoromethylbenzamide; Bacillus laterosporus and cis-Jasmone; Bacillus laterosporus and harpin; Bacillus laterosporus and Azadirachta indica oil; Bacillus laterosporus and Azadirachtin; Bacillus lentus and alanycarb; Bacillus lentus and aldicarb; Bacillus lentus and carbofuran; Bacillus lentus and carbosulfan; Bacillus lentus and fosthiazate; Bacillus lentus and cadusafos; Bacillus lentus and oxamyl; Bacillus lentus and thiodicarb; Bacillus lentus and dimethoate; Bacillus lentus and ethoprophos; Bacillus lentus and terbufos; Bacillus lentus and abamectin; Bacillus lentus and methyl bromide and other alkyl halides; Bacillus lentus and methyl isocyanate generators selected from diazomet and metam; Bacillus lentus and fluazaindolizine; Bacillus lentus and fluensulfone; Bacillus lentus and fluopyram; Bacillus lentus and tioxazafen; Bacillus lentus and N-[1-(2,6-difluorophenyl)-1H-pyrazol-3-yl]-2-trifluoromethylbenzamide; Bacillus lentus and cis-Jasmone; Bacillus lentus and harpin; Bacillus lentus and Azadirachta indica oil; Bacillus lentus and Azadirachtin; Bacillus licheniformis and alanycarb; Bacillus licheniformis and aldicarb; Bacillus licheniformis and carbofuran; Bacillus licheniformis and carbosulfan; Bacillus licheniformis and fosthiazate; Bacillus licheniformis and cadusafos; Bacillus licheniformis and oxamyl; Bacillus licheniformis and thiodicarb; Bacillus licheniformis and dimethoate; Bacillus licheniformis and ethoprophos; Bacillus licheniformis and terbufos; Bacillus licheniformis and abamectin; Bacillus licheniformis and methyl bromide and other alkyl halides; Bacillus licheniformis and methyl isocyanate generators selected from diazomet and metam; Bacillus licheniformis and fluazaindolizine; Bacillus licheniformis and fluensulfone; Bacillus licheniformis and fluopyram; Bacillus licheniformis and tioxazafen; Bacillus licheniformis and N-[1-(2,6-difluorophenyl)-1H-pyrazol-3-yl]-2-trifluoromethylbenzamide; Bacillus licheniformis and cis-Jasmone; Bacillus licheniformis and harpin; Bacillus licheniformis and Azadirachta indica oil; Bacillus licheniformis and Azadirachtin; Bacillus nematocida and alanycarb; Bacillus nematocida and aldicarb; Bacillus nematocida and carbofuran; Bacillus nematocida and carbosulfan; Bacillus nematocida and fosthiazate; Bacillus nematocida and cadusafos; Bacillus nematocida and oxamyl; Bacillus nematocida and thiodicarb; Bacillus nematocida and dimethoate; Bacillus nematocida and ethoprophos; Bacillus nematocida and terbufos; Bacillus nematocida and abamectin; Bacillus nematocida and methyl bromide and other alkyl halides; Bacillus nematocida and methyl isocyanate generators selected from diazomet and metam; Bacillus nematocida and fluazaindolizine; Bacillus nematocida and fluensulfone; Bacillus nematocida and fluopyram; Bacillus nematocida and tioxazafen; Bacillus nematocida and N-[1-(2,6-difluorophenyl)-1H-pyrazol-3-yl]-2-trifluoromethylbenzamide; Bacillus nematocida and cis-Jasmone; Bacillus nematocida and harpin; Bacillus nematocida and Azadirachta indica oil; Bacillus nematocida and Azadirachtin; Bacillus pumilus and alanycarb; Bacillus pumilus and aldicarb; Bacillus pumilus and carbofuran; Bacillus pumilus and carbosulfan; Bacillus pumilus and fosthiazate; Bacillus pumilus and cadusafos; Bacillus pumilus and oxamyl; Bacillus pumilus and thiodicarb; Bacillus pumilus and dimethoate; Bacillus pumilus and ethoprophos; Bacillus pumilus and terbufos; Bacillus pumilus and abamectin; Bacillus pumilus and methyl bromide and other alkyl halides; Bacillus pumilus and methyl isocyanate generators selected from diazomet and metam; Bacillus pumilus and fluazaindolizine; Bacillus pumilus and fluensulfone; Bacillus pumilus and fluopyram; Bacillus pumilus and tioxazafen; Bacillus pumilus and N-[1-(2,6-difluorophenyl)-1H-pyrazol-3-yl]-2-trifluoromethylbenzamide; Bacillus pumilus and cis-Jasmone; Bacillus pumilus and harpin; Bacillus pumilus and Azadirachta indica oil; Bacillus pumilus and Azadirachtin; Bacillus subtilis and alanycarb; Bacillus subtilis and aldicarb; Bacillus subtilis and carbofuran; Bacillus subtilis and carbosulfan; Bacillus subtilis and fosthiazate; Bacillus subtilis and cadusafos; Bacillus subtilis and oxamyl; Bacillus subtilis and thiodicarb; Bacillus subtilis and dimethoate; Bacillus subtilis and ethoprophos; Bacillus subtilis and terbufos; Bacillus subtilis and abamectin; Bacillus subtilis and methyl bromide and other alkyl halides; Bacillus subtilis and methyl isocyanate generators selected from diazomet and metam; Bacillus subtilis and fluazaindolizine; Bacillus subtilis and fluensulfone; Bacillus subtilis and fluopyram; Bacillus subtilis and tioxazafen; Bacillus subtilis and N-[1-(2,6-difluorophenyl)-1H-pyrazol-3-yl]-2-trifluoromethylbenzamide; Bacillus subtilis and cis-Jasmone; Bacillus subtilis and harpin; Bacillus subtilis and Azadirachta indica oil; Bacillus subtilis and Azadirachtin; Bacillus penetrans and alanycarb; Bacillus penetrans and aldicarb; Bacillus penetrans and carbofuran; Bacillus penetrans and carbosulfan; Bacillus penetrans and fosthiazate; Bacillus penetrans and cadusafos; Bacillus penetrans and oxamyl; Bacillus penetrans and thiodicarb; Bacillus penetrans and dimethoate; Bacillus penetrans and ethoprophos; Bacillus penetrans and terbufos; Bacillus penetrans and abamectin; Bacillus penetrans and methyl bromide and other alkyl halides; Bacillus penetrans and methyl isocyanate generators selected from diazomet and metam; Bacillus penetrans and fluazaindolizine; Bacillus penetrans and fluensulfone; Bacillus penetrans and fluopyram; Bacillus penetrans and tioxazafen; Bacillus penetrans and N-[1-(2,6-difluorophenyl)-1H-pyrazol-3-yl]-2-trifluoromethylbenzamide; Bacillus penetrans and cis-Jasmone; Bacillus penetrans and harpin; Bacillus penetrans and Azadirachta indica oil; Bacillus penetrans and Azadirachtin; Bacillus thuringiensis and alanycarb; Bacillus thuringiensis and aldicarb; Bacillus thuringiensis and carbofuran; Bacillus thuringiensis and carbosulfan; Bacillus thuringiensis and fosthiazate; Bacillus thuringiensis and cadusafos; Bacillus thuringiensis and oxamyl; Bacillus thuringiensis and thiodicarb; Bacillus thuringiensis and dimethoate; Bacillus thuringiensis and ethoprophos; Bacillus thuringiensis and terbufos; Bacillus thuringiensis and abamectin; Bacillus thuringiensis and methyl bromide and other alkyl halides; Bacillus thuringiensis and methyl isocyanate generators selected from diazomet and metam; Bacillus thuringiensis and fluazaindolizine; Bacillus thuringiensis and fluensulfone; Bacillus thuringiensis and fluopyram; Bacillus thuringiensis and tioxazafen; Bacillus thuringiensis and N-[1-(2,6-difluorophenyl)-1H-pyrazol-3-yl]-2-trifluoromethylbenzamide; Bacillus thuringiensis and cis-Jasmone; Bacillus thuringiensis and harpin; Bacillus thuringiensis and Azadirachta indica oil; Bacillus thuringiensis and Azadirachtin; Brevibacillus laterosporus and alanycarb; Brevibacillus laterosporus and aldicarb; Brevibacillus laterosporus and carbofuran; Brevibacillus laterosporus and carbosulfan; Brevibacillus laterosporus and fosthiazate; Brevibacillus laterosporus and cadusafos; Brevibacillus laterosporus and oxamyl; Brevibacillus laterosporus and thiodicarb; Brevibacillus laterosporus and dimethoate; Brevibacillus laterosporus and ethoprophos; Brevibacillus laterosporus and terbufos; Brevibacillus laterosporus and abamectin; Brevibacillus laterosporus and methyl bromide and other alkyl halides; Brevibacillus laterosporus and methyl isocyanate generators selected from diazomet and metam; Brevibacillus laterosporus and fluazaindolizine; Brevibacillus laterosporus and fluensulfone; Brevibacillus laterosporus and fluopyram; Brevibacillus laterosporus and tioxazafen; Brevibacillus laterosporus and N-[1-(2,6-difluorophenyl)-1H-pyrazol-3-yl]-2-trifluoromethylbenzamide; Brevibacillus laterosporus and cis-Jasmone; Brevibacillus laterosporus and harpin; Brevibacillus laterosporus and Azadirachta indica oil; Brevibacillus laterosporus and Azadirachtin; Burkholderia rinojensis and alanycarb; Burkholderia rinojensis and aldicarb; Burkholderia rinojensis and carbofuran; Burkholderia rinojensis and carbosulfan; Burkholderia rinojensis and fosthiazate; Burkholderia rinojensis and cadusafos; Burkholderia rinojensis and oxamyl; Burkholderia rinojensis and thiodicarb; Burkholderia rinojensis and dimethoate; Burkholderia rinojensis and ethoprophos; Burkholderia rinojensis and terbufos; Burkholderia rinojensis and abamectin; Burkholderia rinojensis and methyl bromide and other alkyl halides; Burkholderia rinojensis and methyl isocyanate generators selected from diazomet and metam; Burkholderia rinojensis and fluazaindolizine; Burkholderia rinojensis and fluensulfone; Burkholderia rinojensis and fluopyram; Burkholderia rinojensis and tioxazafen; Burkholderia rinojensis and N-[1-(2,6-difluorophenyl)-1H-pyrazol-3-yl]-2-trifluoromethylbenzamide; Burkholderia rinojensis and cis-Jasmone; Burkholderia rinojensis and harpin; Burkholderia rinojensis and Azadirachta indica oil; Burkholderia rinojensis and Azadirachtin; Lysobacter antibioticus and alanycarb; Lysobacter antibioticus and aldicarb; Lysobacter antibioticus and carbofuran; Lysobacter antibioticus and carbosulfan; Lysobacter antibioticus and fosthiazate; Lysobacter antibioticus and cadusafos; Lysobacter antibioticus and oxamyl; Lysobacter antibioticus and thiodicarb; Lysobacter antibioticus and dimethoate; Lysobacter antibioticus and ethoprophos; Lysobacter antibioticus and terbufos; Lysobacter antibioticus and abamectin; Lysobacter antibioticus and methyl bromide and other alkyl halides; Lysobacter antibioticus and methyl isocyanate generators selected from diazomet and metam; Lysobacter antibioticus and fluazaindolizine; Lysobacter antibioticus and fluensulfone; Lysobacter antibioticus and fluopyram; Lysobacter antibioticus and tioxazafen; Lysobacter antibioticus and N-[1-(2,6-difluorophenyl)-1H-pyrazol-3-yl]-2-trifluoromethylbenzamide; Lysobacter antibioticus and cis-Jasmone; Lysobacter antibioticus and harpin; Lysobacter antibioticus and Azadirachta indica oil; Lysobacter antibioticus and Azadirachtin; Lysobacter enzymogenes and alanycarb; Lysobacter enzymogenes and aldicarb; Lysobacter enzymogenes and carbofuran; Lysobacter enzymogenes and carbosulfan; Lysobacter enzymogenes and fosthiazate; Lysobacter enzymogenes and cadusafos; Lysobacter enzymogenes and oxamyl; Lysobacter enzymogenes and thiodicarb; Lysobacter enzymogenes and dimethoate; Lysobacter enzymogenes and ethoprophos; Lysobacter enzymogenes and terbufos; Lysobacter enzymogenes and abamectin; Lysobacter enzymogenes and methyl bromide and other alkyl halides; Lysobacter enzymogenes and methyl isocyanate generators selected from diazomet and metam; Lysobacter enzymogenes and fluazaindolizine; Lysobacter enzymogenes and fluensulfone; Lysobacter enzymogenes and fluopyram; Lysobacter enzymogenes and tioxazafen; Lysobacter enzymogenes and N-[1-(2,6-difluorophenyl)-1H-pyrazol-3-yl]-2-trifluoromethylbenzamide; Lysobacter enzymogenes and cis-Jasmone; Lysobacter enzymogenes and harpin; Lysobacter enzymogenes and Azadirachta indica oil; Lysobacter enzymogenes and Azadirachtin; Pasteuria nishizawae and alanycarb; Pasteuria nishizawae and aldicarb; Pasteuria nishizawae and carbofuran; Pasteuria nishizawae and carbosulfan; Pasteuria nishizawae and fosthiazate; Pasteuria nishizawae and cadusafos; Pasteuria nishizawae and oxamyl; Pasteuria nishizawae and thiodicarb; Pasteuria nishizawae and dimethoate; Pasteuria nishizawae and ethoprophos; Pasteuria nishizawae and terbufos; Pasteuria nishizawae and abamectin; Pasteuria nishizawae and methyl bromide and other alkyl halides; Pasteuria nishizawae and methyl isocyanate generators selected from diazomet and metam; Pasteuria nishizawae and fluazaindolizine; Pasteuria nishizawae and fluensulfone; Pasteuria nishizawae and fluopyram; Pasteuria nishizawae and tioxazafen; Pasteuria nishizawae and N-[1-(2,6-difluorophenyl)-1H-pyrazol-3-yl]-2-trifluoromethylbenzamide; Pasteuria nishizawae and cis-Jasmone; Pasteuria nishizawae and harpin; Pasteuria nishizawae and Azadirachta indica oil; Pasteuria nishizawae and Azadirachtin; Pasteuria penetrans and alanycarb; Pasteuria penetrans and aldicarb; Pasteuria penetrans and carbofuran; Pasteuria penetrans and carbosulfan; Pasteuria penetrans and fosthiazate; Pasteuria penetrans and cadusafos; Pasteuria penetrans and oxamyl; Pasteuria penetrans and thiodicarb; Pasteuria penetrans and dimethoate; Pasteuria penetrans and ethoprophos; Pasteuria penetrans and terbufos; Pasteuria penetrans and abamectin; Pasteuria penetrans and methyl bromide and other alkyl halides; Pasteuria penetrans and methyl isocyanate generators selected from diazomet and metam; Pasteuria penetrans and fluazaindolizine; Pasteuria penetrans and fluensulfone; Pasteuria penetrans and fluopyram; Pasteuria penetrans and tioxazafen; Pasteuria penetrans and N-[1-(2,6-difluorophenyl)-1H-pyrazol-3-yl]-2-trifluoromethylbenzamide; Pasteuria penetrans and cis-Jasmone; Pasteuria penetrans and harpin; Pasteuria penetrans and Azadirachta indica oil; Pasteuria penetrans and Azadirachtin; Pasteuria ramosa and alanycarb; Pasteuria ramosa and aldicarb; Pasteuria ramosa and carbofuran; Pasteuria ramosa and carbosulfan; Pasteuria ramosa and fosthiazate; Pasteuria ramosa and cadusafos; Pasteuria ramosa and oxamyl; Pasteuria ramosa and thiodicarb; Pasteuria ramosa and dimethoate; Pasteuria ramosa and ethoprophos; Pasteuria ramosa and terbufos; Pasteuria ramosa and abamectin; Pasteuria ramosa and methyl bromide and other alkyl halides; Pasteuria ramosa and methyl isocyanate generators selected from diazomet and metam; Pasteuria ramosa and fluazaindolizine; Pasteuria ramosa and fluensulfone; Pasteuria ramosa and fluopyram; Pasteuria ramosa and tioxazafen; Pasteuria ramosa and N-[1-(2,6-difluorophenyl)-1H-pyrazol-3-yl]-2-trifluoromethylbenzamide; Pasteuria ramosa and cis-Jasmone; Pasteuria ramosa and harpin; Pasteuria ramosa and Azadirachta indica oil; Pasteuria ramosa and Azadirachtin; Pasteuria reniformis and alanycarb; Pasteuria reniformis and aldicarb; Pasteuria reniformis and carbofuran; Pasteuria reniformis and carbosulfan; Pasteuria reniformis and fosthiazate; Pasteuria reniformis and cadusafos; Pasteuria reniformis and oxamyl; Pasteuria reniformis and thiodicarb; Pasteuria reniformis and dimethoate; Pasteuria reniformis and ethoprophos; Pasteuria reniformis and terbufos; Pasteuria reniformis and abamectin; Pasteuria reniformis and methyl bromide and other alkyl halides; Pasteuria reniformis and methyl isocyanate generators selected from diazomet and metam; Pasteuria reniformis and fluazaindolizine; Pasteuria reniformis and fluensulfone; Pasteuria reniformis and fluopyram; Pasteuria reniformis and tioxazafen; Pasteuria reniformis and N-[1-(2,6-difluorophenyl)-1H-pyrazol-3-yl]-2-trifluoromethylbenzamide; Pasteuria reniformis and cis-Jasmone; Pasteuria reniformis and harpin; Pasteuria reniformis and Azadirachta indica oil; Pasteuria reniformis and Azadirachtin; Pasteuria thornei and alanycarb; Pasteuria thornei and aldicarb; Pasteuria thornei and carbofuran; Pasteuria thornei and carbosulfan; Pasteuria thornei and fosthiazate; Pasteuria thornei and cadusafos; Pasteuria thornei and oxamyl; Pasteuria thornei and thiodicarb; Pasteuria thornei and dimethoate; Pasteuria thornei and ethoprophos; Pasteuria thornei and terbufos; Pasteuria thornei and abamectin; Pasteuria thornei and methyl bromide and other alkyl halides; Pasteuria thornei and methyl isocyanate generators selected from diazomet and metam; Pasteuria thornei and fluazaindolizine; Pasteuria thornei and fluensulfone; Pasteuria thornei and fluopyram; Pasteuria thornei and tioxazafen; Pasteuria thornei and N-[1-(2,6-difluorophenyl)-1H-pyrazol-3-yl]-2-trifluoromethylbenzamide; Pasteuria thornei and cis-Jasmone; Pasteuria thornei and harpin; Pasteuria thornei and Azadirachta indica oil; Pasteuria thornei and Azadirachtin; Pasteuria usage and alanycarb; Pasteuria usage and aldicarb; Pasteuria usage and carbofuran; Pasteuria usage and carbosulfan; Pasteuria usage and fosthiazate; Pasteuria usage and cadusafos; Pasteuria usage and oxamyl; Pasteuria usage and thiodicarb; Pasteuria usage and dimethoate; Pasteuria usage and ethoprophos; Pasteuria usage and terbufos; Pasteuria usage and abamectin; Pasteuria usage and methyl bromide and other alkyl halides; Pasteuria usage and methyl isocyanate generators selected from diazomet and metam; Pasteuria usage and fluazaindolizine; Pasteuria usage and fluensulfone; Pasteuria usage and fluopyram; Pasteuria usage and tioxazafen; Pasteuria usage and N-[1-(2,6-difluorophenyl)-1H-pyrazol-3-yl]-2-trifluoromethylbenzamide; Pasteuria usage and cis-Jasmone; Pasteuria usage and harpin; Pasteuria usage and Azadirachta indica oil; Pasteuria usage and Azadirachtin; Arthrobotrys dactyloides and alanycarb; Arthrobotrys dactyloides and aldicarb; Arthrobotrys dactyloides and carbofuran; Arthrobotrys dactyloides and carbosulfan; Arthrobotrys dactyloides and fosthiazate; Arthrobotrys dactyloides and cadusafos; Arthrobotrys dactyloides and oxamyl; Arthrobotrys dactyloides and thiodicarb; Arthrobotrys dactyloides and dimethoate; Arthrobotrys dactyloides and ethoprophos; Arthrobotrys dactyloides and terbufos; Arthrobotrys dactyloides and abamectin; Arthrobotrys dactyloides and methyl bromide and other alkyl halides; Arthrobotrys dactyloides and methyl isocyanate generators selected from diazomet and metam; Arthrobotrys dactyloides and fluazaindolizine; Arthrobotrys dactyloides and fluensulfone; Arthrobotrys dactyloides and fluopyram; Arthrobotrys dactyloides and tioxazafen; Arthrobotrys dactyloides and N-[1-(2,6-difluorophenyl)-1H-pyrazol-3-yl]-2-trifluoromethylbenzamide; Arthrobotrys dactyloides and cis-Jasmone; Arthrobotrys dactyloides and harpin; Arthrobotrys dactyloides and Azadirachta indica oil; Arthrobotrys dactyloides and Azadirachtin; Arthrobotrys oligospora and alanycarb; Arthrobotrys oligospora and aldicarb; Arthrobotrys oligospora and carbofuran; Arthrobotrys oligospora and carbosulfan; Arthrobotrys oligospora and fosthiazate; Arthrobotrys oligospora and cadusafos; Arthrobotrys oligospora and oxamyl; Arthrobotrys oligospora and thiodicarb; Arthrobotrys oligospora and dimethoate; Arthrobotrys oligospora and ethoprophos; Arthrobotrys oligospora and terbufos; Arthrobotrys oligospora and abamectin; Arthrobotrys oligospora and methyl bromide and other alkyl halides; Arthrobotrys oligospora and methyl isocyanate generators selected from diazomet and metam; Arthrobotrys oligospora and fluazaindolizine; Arthrobotrys oligospora and fluensulfone; Arthrobotrys oligospora and fluopyram; Arthrobotrys oligospora and tioxazafen; Arthrobotrys oligospora and N-[1-(2,6-difluorophenyl)-1H-pyrazol-3-yl]-2-trifluoromethylbenzamide; Arthrobotrys oligospora and cis-Jasmone; Arthrobotrys oligospora and harpin; Arthrobotrys oligospora and Azadirachta indica oil; Arthrobotrys oligospora and Azadirachtin; Arthrobotrys superba and alanycarb; Arthrobotrys superba and aldicarb; Arthrobotrys superba and carbofuran; Arthrobotrys superba and carbosulfan; Arthrobotrys superba and fosthiazate; Arthrobotrys superba and cadusafos; Arthrobotrys superba and oxamyl; Arthrobotrys superba and thiodicarb; Arthrobotrys superba and dimethoate; Arthrobotrys superba and ethoprophos; Arthrobotrys superba and terbufos; Arthrobotrys superba and abamectin; Arthrobotrys superba and methyl bromide and other alkyl halides; Arthrobotrys superba and methyl isocyanate generators selected from diazomet and metam; Arthrobotrys superba and fluazaindolizine; Arthrobotrys superba and fluensulfone; Arthrobotrys superba and fluopyram; Arthrobotrys superba and tioxazafen; Arthrobotrys superba and N-[1-(2,6-difluorophenyl)-1H-pyrazol-3-yl]-2-trifluoromethylbenzamide; Arthrobotrys superba and cis-Jasmone; Arthrobotrys superba and harpin; Arthrobotrys superba and Azadirachta indica oil; Arthrobotrys superba and Azadirachtin; Nematoctonus geogenius and alanycarb; Nematoctonus geogenius and aldicarb; Nematoctonus geogenius and carbofuran; Nematoctonus geogenius and carbosulfan; Nematoctonus geogenius and fosthiazate; Nematoctonus geogenius and cadusafos; Nematoctonus geogenius and oxamyl; Nematoctonus geogenius and thiodicarb; Nematoctonus geogenius and dimethoate; Nematoctonus geogenius and ethoprophos; Nematoctonus geogenius and terbufos; Nematoctonus geogenius and abamectin; Nematoctonus geogenius and methyl bromide and other alkyl halides; Nematoctonus geogenius and methyl isocyanate generators selected from diazomet and metam; Nematoctonus geogenius and fluazaindolizine; Nematoctonus geogenius and fluensulfone; Nematoctonus geogenius and fluopyram; Nematoctonus geogenius and tioxazafen; Nematoctonus geogenius and N-[1-(2,6-difluorophenyl)-1H-pyrazol-3-yl]-2-trifluoromethylbenzamide; Nematoctonus geogenius and cis-Jasmone; Nematoctonus geogenius and harpin; Nematoctonus geogenius and Azadirachta indica oil; Nematoctonus geogenius and Azadirachtin; Nematoctonus leiosporus and alanycarb; Nematoctonus leiosporus and aldicarb; Nematoctonus leiosporus and carbofuran; Nematoctonus leiosporus and carbosulfan; Nematoctonus leiosporus and fosthiazate; Nematoctonus leiosporus and cadusafos; Nematoctonus leiosporus and oxamyl; Nematoctonus leiosporus and thiodicarb; Nematoctonus leiosporus and dimethoate; Nematoctonus leiosporus and ethoprophos; Nematoctonus leiosporus and terbufos; Nematoctonus leiosporus and abamectin; Nematoctonus leiosporus and methyl bromide and other alkyl halides; Nematoctonus leiosporus and methyl isocyanate generators selected from diazomet and metam; Nematoctonus leiosporus and fluazaindolizine; Nematoctonus leiosporus and fluensulfone; Nematoctonus leiosporus and fluopyram; Nematoctonus leiosporus and tioxazafen; Nematoctonus leiosporus and N-[1-(2,6-difluorophenyl)-1H-pyrazol-3-yl]-2-trifluoromethylbenzamide; Nematoctonus leiosporus and cis-Jasmone; Nematoctonus leiosporus and harpin; Nematoctonus leiosporus and Azadirachta indica oil; Nematoctonus leiosporus and Azadirachtin; Myrothecium verrucaria and alanycarb; Myrothecium verrucaria and aldicarb; Myrothecium verrucaria and carbofuran; Myrothecium verrucaria and carbosulfan; Myrothecium verrucaria and fosthiazate; Myrothecium verrucaria and cadusafos; Myrothecium verrucaria and oxamyl; Myrothecium verrucaria and thiodicarb; Myrothecium verrucaria and dimethoate; Myrothecium verrucaria and ethoprophos; Myrothecium verrucaria and terbufos; Myrothecium verrucaria and abamectin; Myrothecium verrucaria and methyl bromide and other alkyl halides; Myrothecium verrucaria and methyl isocyanate generators selected from diazomet and metam; Myrothecium verrucaria and fluazaindolizine; Myrothecium verrucaria and fluensulfone; Myrothecium verrucaria and fluopyram; Myrothecium verrucaria and tioxazafen; Myrothecium verrucaria and N-[1-(2,6-difluorophenyl)-1H-pyrazol-3-yl]-2-trifluoromethylbenzamide; Myrothecium verrucaria and cis-Jasmone; Myrothecium verrucaria and harpin; Myrothecium verrucaria and Azadirachta indica oil; Myrothecium verrucaria and Azadirachtin; Paecilomyces lilacinus and alanycarb; Paecilomyces lilacinus and aldicarb; Paecilomyces lilacinus and carbofuran; Paecilomyces lilacinus and carbosulfan; Paecilomyces lilacinus and fosthiazate; Paecilomyces lilacinus and cadusafos; Paecilomyces lilacinus and oxamyl; Paecilomyces lilacinus and thiodicarb; Paecilomyces lilacinus and dimethoate; Paecilomyces lilacinus and ethoprophos; Paecilomyces lilacinus and terbufos; Paecilomyces lilacinus and abamectin; Paecilomyces lilacinus and methyl bromide and other alkyl halides; Paecilomyces lilacinus and methyl isocyanate generators selected from diazomet and metam; Paecilomyces lilacinus and fluazaindolizine; Paecilomyces lilacinus and fluensulfone; Paecilomyces lilacinus and fluopyram; Paecilomyces lilacinus and tioxazafen; Paecilomyces lilacinus and N-[1-(2,6-difluorophenyl)-1H-pyrazol-3-yl]-2-trifluoromethylbenzamide; Paecilomyces lilacinus and cis-Jasmone; Paecilomyces lilacinus and harpin; Paecilomyces lilacinus and Azadirachta indica oil; Paecilomyces lilacinus and Azadirachtin; Paecilomyces variotii and alanycarb; Paecilomyces variotii and aldicarb; Paecilomyces variotii and carbofuran; Paecilomyces variotii and carbosulfan; Paecilomyces variotii and fosthiazate; Paecilomyces variotii and cadusafos; Paecilomyces variotii and oxamyl; Paecilomyces variotii and thiodicarb; Paecilomyces variotii and dimethoate; Paecilomyces variotii and ethoprophos; Paecilomyces variotii and terbufos; Paecilomyces variotii and abamectin; Paecilomyces variotii and methyl bromide and other alkyl halides; Paecilomyces variotii and methyl isocyanate generators selected from diazomet and metam; Paecilomyces variotii and fluazaindolizine; Paecilomyces variotii and fluensulfone; Paecilomyces variotii and fluopyram; Paecilomyces variotii and tioxazafen; Paecilomyces variotii and N-[1-(2,6-difluorophenyl)-1H-pyrazol-3-yl]-2-trifluoromethylbenzamide; Paecilomyces variotii and cis-Jasmone; Paecilomyces variotii and harpin; Paecilomyces variotii and Azadirachta indica oil; Paecilomyces variotii and Azadirachtin; Trichoderma asperellum and alanycarb; Trichoderma asperellum and aldicarb; Trichoderma asperellum and carbofuran; Trichoderma asperellum and carbosulfan; Trichoderma asperellum and fosthiazate; Trichoderma asperellum and cadusafos; Trichoderma asperellum and oxamyl; Trichoderma asperellum and thiodicarb; Trichoderma asperellum and dimethoate; Trichoderma asperellum and ethoprophos; Trichoderma asperellum and terbufos; Trichoderma asperellum and abamectin; Trichoderma asperellum and methyl bromide and other alkyl halides; Trichoderma asperellum and methyl isocyanate generators selected from diazomet and metam; Trichoderma asperellum and fluazaindolizine; Trichoderma asperellum and fluensulfone; Trichoderma asperellum and fluopyram; Trichoderma asperellum and tioxazafen; Trichoderma asperellum and N-[1-(2,6-difluorophenyl)-1H-pyrazol-3-yl]-2-trifluoromethylbenzamide; Trichoderma asperellum and cis-Jasmone; Trichoderma asperellum and harpin; Trichoderma asperellum and Azadirachta indica oil; Trichoderma asperellum and Azadirachtin; Trichoderma harzianum and alanycarb; Trichoderma harzianum and aldicarb; Trichoderma harzianum and carbofuran; Trichoderma harzianum and carbosulfan; Trichoderma harzianum and fosthiazate; Trichoderma harzianum and cadusafos; Trichoderma harzianum and oxamyl; Trichoderma harzianum and thiodicarb; Trichoderma harzianum and dimethoate; Trichoderma harzianum and ethoprophos; Trichoderma harzianum and terbufos; Trichoderma harzianum and abamectin; Trichoderma harzianum and methyl bromide and other alkyl halides; Trichoderma harzianum and methyl isocyanate generators selected from diazomet and metam; Trichoderma harzianum and fluazaindolizine; Trichoderma harzianum and fluensulfone; Trichoderma harzianum and fluopyram; Trichoderma harzianum and tioxazafen; Trichoderma harzianum and N-[1-(2,6-difluorophenyl)-1H-pyrazol-3-yl]-2-trifluoromethylbenzamide; Trichoderma harzianum and cis-Jasmone; Trichoderma harzianum and harpin; Trichoderma harzianum and Azadirachta indica oil; Trichoderma harzianum and Azadirachtin; Trichoderma viride and alanycarb; Trichoderma viride and aldicarb; Trichoderma viride and carbofuran; Trichoderma viride and carbosulfan; Trichoderma viride and fosthiazate; Trichoderma viride and cadusafos; Trichoderma viride and oxamyl; Trichoderma viride and thiodicarb; Trichoderma viride and dimethoate; Trichoderma viride and ethoprophos; Trichoderma viride and terbufos; Trichoderma viride and abamectin; Trichoderma viride and methyl bromide and other alkyl halides; Trichoderma viride and methyl isocyanate generators selected from diazomet and metam; Trichoderma viride and fluazaindolizine; Trichoderma viride and fluensulfone; Trichoderma viride and fluopyram; Trichoderma viride and tioxazafen; Trichoderma viride and N-[1-(2,6-difluorophenyl)-1H-pyrazol-3-yl]-2-trifluoromethylbenzamide; Trichoderma viride and cis-Jasmone; Trichoderma viride and harpin; Trichoderma viride and Azadirachta indica oil; Trichoderma viride and Azadirachtin; Trichoderma harzianum rifai and alanycarb; Trichoderma harzianum rifai and aldicarb; Trichoderma harzianum rifai and carbofuran; Trichoderma harzianum rifai and carbosulfan; Trichoderma harzianum rifai and fosthiazate; Trichoderma harzianum rifai and cadusafos; Trichoderma harzianum rifai and oxamyl; Trichoderma harzianum rifai and thiodicarb; Trichoderma harzianum rifai and dimethoate; Trichoderma harzianum rifai and ethoprophos; Trichoderma harzianum rifai and terbufos; Trichoderma harzianum rifai and abamectin; Trichoderma harzianum rifai and methyl bromide and other alkyl halides; Trichoderma harzianum rifai and methyl isocyanate generators selected from diazomet and metam; Trichoderma harzianum rifai and fluazaindolizine; Trichoderma harzianum rifai and fluensulfone; Trichoderma harzianum rifai and fluopyram; Trichoderma harzianum rifai and tioxazafen; Trichoderma harzianum rifai and N-[1-(2,6-difluorophenyl)-1H-pyrazol-3-yl]-2-trifluoromethylbenzamide; Trichoderma harzianum rifai and cis-Jasmone; Trichoderma harzianum rifai and harpin; Trichoderma harzianum rifai and Azadirachta indica oil; Trichoderma harzianum rifai and Azadirachtin; Tsukamurella paurometabola and alanycarb; Tsukamurella paurometabola and aldicarb; Tsukamurella paurometabola and carbofuran; Tsukamurella paurometabola and carbosulfan; Tsukamurella paurometabola and fosthiazate; Tsukamurella paurometabola and cadusafos; Tsukamurella paurometabola and oxamyl; Tsukamurella paurometabola and thiodicarb; Tsukamurella paurometabola and dimethoate; Tsukamurella paurometabola and ethoprophos; Tsukamurella paurometabola and terbufos; Tsukamurella paurometabola and abamectin; Tsukamurella paurometabola and methyl bromide and other alkyl halides; Tsukamurella paurometabola and methyl isocyanate generators selected from diazomet and metam; Tsukamurella paurometabola and fluazaindolizine; Tsukamurella paurometabola and fluensulfone; Tsukamurella paurometabola and fluopyram; Tsukamurella paurometabola and tioxazafen; Tsukamurella paurometabola and N-[1-(2,6-difluorophenyl)-1H-pyrazol-3-yl]-2-trifluoromethylbenzamide; Tsukamurella paurometabola and cis-Jasmone; Tsukamurella paurometabola and harpin; Tsukamurella paurometabola and Azadirachta indica oil; and Tsukamurella paurometabola and Azadirachtin.
117. The plant, cell, plant part or seed of any one of these numbered paragraphs, or the soil in which they are grown or are intended to be grown, treated with a combination of a nematicidal agent and a biological control agent selected from: Bacillus amyloliquefaciens strain IN937a and alanycarb, Bacillus amyloliquefaciens strain IN937a and aldicarb, Bacillus amyloliquefaciens strain IN937a and carbofuran, Bacillus amyloliquefaciens strain IN937a and carbosulfan, Bacillus amyloliquefaciens strain IN937a and fosthiazate, Bacillus amyloliquefaciens strain IN937a and cadusafos, Bacillus amyloliquefaciens strain IN937a and oxamyl, Bacillus amyloliquefaciens strain IN937a and thiodicarb, Bacillus amyloliquefaciens strain IN937a and dimethoate, Bacillus amyloliquefaciens strain IN937a and ethoprophos, Bacillus amyloliquefaciens strain IN937a and terbufos, Bacillus amyloliquefaciens strain IN937a and abamectin, Bacillus amyloliquefaciens strain IN937a and methyl bromide and other alkyl halides, Bacillus amyloliquefaciens strain IN937a and methyl isocyanate generators selected from diazomet and metam, Bacillus amyloliquefaciens strain IN937a and fluazaindolizine, Bacillus amyloliquefaciens strain IN937a and fluensulfone, Bacillus amyloliquefaciens strain IN937a and fluopyram, Bacillus amyloliquefaciens strain IN937a and tioxazafen, Bacillus amyloliquefaciens strain IN937a and N-[1-(2,6-difluorophenyl)-1H-pyrazol-3-yl]-2-trifluoromethylbenzamide, Bacillus amyloliquefaciens strain IN937a and cis-Jasmone, Bacillus amyloliquefaciens strain IN937a and harpin, Bacillus amyloliquefaciens strain IN937a and Azadirachta indica oil, Bacillus amyloliquefaciens strain IN937a and Azadirachtin, Bacillus amyloliquefaciens strain FZB42 and alanycarb, Bacillus amyloliquefaciens strain FZB42 and aldicarb, Bacillus amyloliquefaciens strain FZB42 and carbofuran, Bacillus amyloliquefaciens strain FZB42 and carbosulfan, Bacillus amyloliquefaciens strain FZB42 and fosthiazate, Bacillus amyloliquefaciens strain FZB42 and cadusafos, Bacillus amyloliquefaciens strain FZB42 and oxamyl, Bacillus amyloliquefaciens strain FZB42 and thiodicarb, Bacillus amyloliquefaciens strain FZB42 and dimethoate, Bacillus amyloliquefaciens strain FZB42 and ethoprophos, Bacillus amyloliquefaciens strain FZB42 and terbufos, Bacillus amyloliquefaciens strain FZB42 and abamectin, Bacillus amyloliquefaciens strain FZB42 and methyl bromide and other alkyl halides, Bacillus amyloliquefaciens strain FZB42 and methyl isocyanate generators selected from diazomet and metam, Bacillus amyloliquefaciens strain FZB42 and fluazaindolizine, Bacillus amyloliquefaciens strain FZB42 and fluensulfone, Bacillus amyloliquefaciens strain FZB42 and fluopyram, Bacillus amyloliquefaciens strain FZB42 and tioxazafen, Bacillus amyloliquefaciens strain FZB42 and N-[1-(2,6-difluorophenyl)-1H-pyrazol-3-yl]-2-trifluoromethylbenzamide, Bacillus amyloliquefaciens strain FZB42 and cis-Jasmone, Bacillus amyloliquefaciens strain FZB42 and harpin, Bacillus amyloliquefaciens strain FZB42 and Azadirachta indica oil, Bacillus amyloliquefaciens strain FZB42 and Azadirachtin, Bacillus amyloliquefaciens strain FZB24 and alanycarb, Bacillus amyloliquefaciens strain FZB24 and aldicarb, Bacillus amyloliquefaciens strain FZB24 and carbofuran, Bacillus amyloliquefaciens strain FZB24 and carbosulfan, Bacillus amyloliquefaciens strain FZB24 and fosthiazate, Bacillus amyloliquefaciens strain FZB24 and cadusafos, Bacillus amyloliquefaciens strain FZB24 and oxamyl, Bacillus amyloliquefaciens strain FZB24 and thiodicarb, Bacillus amyloliquefaciens strain FZB24 and dimethoate, Bacillus amyloliquefaciens strain FZB24 and ethoprophos, Bacillus amyloliquefaciens strain FZB24 and terbufos, Bacillus amyloliquefaciens strain FZB24 and abamectin, Bacillus amyloliquefaciens strain FZB24 and methyl bromide and other alkyl halides, Bacillus amyloliquefaciens strain FZB24 and methyl isocyanate generators selected from diazomet and metam, Bacillus amyloliquefaciens strain FZB24 and fluazaindolizine, Bacillus amyloliquefaciens strain FZB24 and fluensulfone, Bacillus amyloliquefaciens strain FZB24 and fluopyram, Bacillus amyloliquefaciens strain FZB24 and tioxazafen, Bacillus amyloliquefaciens strain FZB24 and N-[1-(2,6-difluorophenyl)-1H-pyrazol-3-yl]-2-trifluoromethylbenzamide, Bacillus amyloliquefaciens strain FZB24 and cis-Jasmone, Bacillus amyloliquefaciens strain FZB24 and harpin, Bacillus amyloliquefaciens strain FZB24 and Azadirachta indica oil, Bacillus amyloliquefaciens strain FZB24 and Azadirachtin, Bacillus amyloliquefaciens strain NRRL B-50349 and alanycarb, Bacillus amyloliquefaciens strain NRRL B-50349 and aldicarb, Bacillus amyloliquefaciens strain NRRL B-50349 and carbofuran, Bacillus amyloliquefaciens strain NRRL B-50349 and carbosulfan, Bacillus amyloliquefaciens strain NRRL B-50349 and fosthiazate, Bacillus amyloliquefaciens strain NRRL B-50349 and cadusafos, Bacillus amyloliquefaciens strain NRRL B-50349 and oxamyl, Bacillus amyloliquefaciens strain NRRL B-50349 and thiodicarb, Bacillus amyloliquefaciens strain NRRL B-50349 and dimethoate, Bacillus amyloliquefaciens strain NRRL B-50349 and ethoprophos, Bacillus amyloliquefaciens strain NRRL B-50349 and terbufos, Bacillus amyloliquefaciens strain NRRL B-50349 and abamectin, Bacillus amyloliquefaciens strain NRRL B-50349 and methyl bromide and other alkyl halides, Bacillus amyloliquefaciens strain NRRL B-50349 and methyl isocyanate generators selected from diazomet and metam, Bacillus amyloliquefaciens strain NRRL B-50349 and fluazaindolizine, Bacillus amyloliquefaciens strain NRRL B-50349 and fluensulfone, Bacillus amyloliquefaciens strain NRRL B-50349 and fluopyram, Bacillus amyloliquefaciens strain NRRL B-50349 and tioxazafen, Bacillus amyloliquefaciens strain NRRL B-50349 and N-[1-(2,6-difluorophenyl)-1H-pyrazol-3-yl]-2-trifluoromethylbenzamide, Bacillus amyloliquefaciens strain NRRL B-50349 and cis-Jasmone, Bacillus amyloliquefaciens strain NRRL B-50349 and harpin, Bacillus amyloliquefaciens strain NRRL B-50349 and Azadirachta indica oil, Bacillus amyloliquefaciens strain NRRL B-50349 and Azadirachtin, Bacillus amyloliquefaciens strain ABI01 and alanycarb, Bacillus amyloliquefaciens strain ABI01 and aldicarb, Bacillus amyloliquefaciens strain ABI01 and carbofuran, Bacillus amyloliquefaciens strain ABI01 and carbosulfan, Bacillus amyloliquefaciens strain ABI01 and fosthiazate, Bacillus amyloliquefaciens strain ABI01 and cadusafos, Bacillus amyloliquefaciens strain ABI01 and oxamyl, Bacillus amyloliquefaciens strain ABI01 and thiodicarb, Bacillus amyloliquefaciens strain ABI01 and dimethoate, Bacillus amyloliquefaciens strain ABI01 and ethoprophos, Bacillus amyloliquefaciens strain ABI01 and terbufos, Bacillus amyloliquefaciens strain ABI01 and abamectin, Bacillus amyloliquefaciens strain ABI01 and methyl bromide and other alkyl halides, Bacillus amyloliquefaciens strain ABI01 and methyl isocyanate generators selected from diazomet and metam, Bacillus amyloliquefaciens strain ABI01 and fluazaindolizine, Bacillus amyloliquefaciens strain ABI01 and fluensulfone, Bacillus amyloliquefaciens strain ABI01 and fluopyram, Bacillus amyloliquefaciens strain ABI01 and tioxazafen, Bacillus amyloliquefaciens strain ABI01 and N-[1-(2,6-difluorophenyl)-1H-pyrazol-3-yl]-2-trifluoromethylbenzamide, Bacillus amyloliquefaciens strain ABI01 and cis-Jasmone, Bacillus amyloliquefaciens strain ABI01 and harpin, Bacillus amyloliquefaciens strain ABI01 and Azadirachta indica oil, Bacillus amyloliquefaciens strain ABI01 and Azadirachtin, Bacillus amyloliquefaciens strain B3 and alanycarb, Bacillus amyloliquefaciens strain B3 and aldicarb, Bacillus amyloliquefaciens strain B3 and carbofuran, Bacillus amyloliquefaciens strain B3 and carbosulfan, Bacillus amyloliquefaciens strain B3 and fosthiazate, Bacillus amyloliquefaciens strain B3 and cadusafos, Bacillus amyloliquefaciens strain B3 and oxamyl, Bacillus amyloliquefaciens strain B3 and thiodicarb, Bacillus amyloliquefaciens strain B3 and dimethoate, Bacillus amyloliquefaciens strain B3 and ethoprophos, Bacillus amyloliquefaciens strain B3 and terbufos, Bacillus amyloliquefaciens strain B3 and abamectin, Bacillus amyloliquefaciens strain B3 and methyl bromide and other alkyl halides, Bacillus amyloliquefaciens strain B3 and methyl isocyanate generators selected from diazomet and metam, Bacillus amyloliquefaciens strain B3 and fluazaindolizine, Bacillus amyloliquefaciens strain B3 and fluensulfone, Bacillus amyloliquefaciens strain B3 and fluopyram, Bacillus amyloliquefaciens strain B3 and tioxazafen, Bacillus amyloliquefaciens strain B3 and N-[1-(2,6-difluorophenyl)-1H-pyrazol-3-yl]-2-trifluoromethylbenzamide, Bacillus amyloliquefaciens strain B3 and cis-Jasmone, Bacillus amyloliquefaciens strain B3 and harpin, Bacillus amyloliquefaciens strain B3 and Azadirachta indica oil, Bacillus amyloliquefaciens strain B3 and Azadirachtin, Bacillus amyloliquefaciens strain D747 and alanycarb, Bacillus amyloliquefaciens strain D747 and aldicarb, Bacillus amyloliquefaciens strain D747 and carbofuran, Bacillus amyloliquefaciens strain D747 and carbosulfan, Bacillus amyloliquefaciens strain D747 and fosthiazate, Bacillus amyloliquefaciens strain D747 and cadusafos, Bacillus amyloliquefaciens strain D747 and oxamyl, Bacillus amyloliquefaciens strain D747 and thiodicarb, Bacillus amyloliquefaciens strain D747 and dimethoate, Bacillus amyloliquefaciens strain D747 and ethoprophos, Bacillus amyloliquefaciens strain D747 and terbufos, Bacillus amyloliquefaciens strain D747 and abamectin, Bacillus amyloliquefaciens strain D747 and methyl bromide and other alkyl halides, Bacillus amyloliquefaciens strain D747 and methyl isocyanate generators selected from diazomet and metam, Bacillus amyloliquefaciens strain D747 and fluazaindolizine, Bacillus amyloliquefaciens strain D747 and fluensulfone, Bacillus amyloliquefaciens strain D747 and fluopyram, Bacillus amyloliquefaciens strain D747 and tioxazafen, Bacillus amyloliquefaciens strain D747 and N-[1-(2,6-difluorophenyl)-1H-pyrazol-3-yl]-2-trifluoromethylbenzamide, Bacillus amyloliquefaciens strain D747 and cis-Jasmone, Bacillus amyloliquefaciens strain D747 and harpin, Bacillus amyloliquefaciens strain D747 and Azadirachta indica oil, Bacillus amyloliquefaciens strain D747 and Azadirachtin, Bacillus amyloliquefaciens strain APM-1 and alanycarb, Bacillus amyloliquefaciens strain APM-1 and aldicarb, Bacillus amyloliquefaciens strain APM-1 and carbofuran, Bacillus amyloliquefaciens strain APM-1 and carbosulfan, Bacillus amyloliquefaciens strain APM-1 and fosthiazate, Bacillus amyloliquefaciens strain APM-1 and cadusafos, Bacillus amyloliquefaciens strain APM-1 and oxamyl, Bacillus amyloliquefaciens strain APM-1 and thiodicarb, Bacillus amyloliquefaciens strain APM-1 and dimethoate, Bacillus amyloliquefaciens strain APM-1 and ethoprophos, Bacillus amyloliquefaciens strain APM-1 and terbufos, Bacillus amyloliquefaciens strain APM-1 and abamectin, Bacillus amyloliquefaciens strain APM-1 and methyl bromide and other alkyl halides, Bacillus amyloliquefaciens strain APM-1 and methyl isocyanate generators selected from diazomet and metam, Bacillus amyloliquefaciens strain APM-1 and fluazaindolizine, Bacillus amyloliquefaciens strain APM-1 and fluensulfone, Bacillus amyloliquefaciens strain APM-1 and fluopyram, Bacillus amyloliquefaciens strain APM-1 and tioxazafen, Bacillus amyloliquefaciens strain APM-1 and N-[1-(2,6-difluorophenyl)-1H-pyrazol-3-yl]-2-trifluoromethylbenzamide, Bacillus amyloliquefaciens strain APM-1 and cis-Jasmone, Bacillus amyloliquefaciens strain APM-1 and harpin, Bacillus amyloliquefaciens strain APM-1 and Azadirachta indica oil, Bacillus amyloliquefaciens strain APM-1 and Azadirachtin, Bacillus amyloliquefaciens strain TJ1000 and alanycarb, Bacillus amyloliquefaciens strain TJ1000 and aldicarb, Bacillus amyloliquefaciens strain TJ1000 and carbofuran, Bacillus amyloliquefaciens strain TJ1000 and carbosulfan, Bacillus amyloliquefaciens strain TJ1000 and fosthiazate, Bacillus amyloliquefaciens strain TJ1000 and cadusafos, Bacillus amyloliquefaciens strain TJ1000 and oxamyl, Bacillus amyloliquefaciens strain TJ1000 and thiodicarb, Bacillus amyloliquefaciens strain TJ1000 and dimethoate, Bacillus amyloliquefaciens strain TJ1000 and ethoprophos, Bacillus amyloliquefaciens strain TJ1000 and terbufos, Bacillus amyloliquefaciens strain TJ1000 and abamectin, Bacillus amyloliquefaciens strain TJ1000 and methyl bromide and other alkyl halides, Bacillus amyloliquefaciens strain TJ1000 and methyl isocyanate generators selected from diazomet and metam, Bacillus amyloliquefaciens strain TJ1000 and fluazaindolizine, Bacillus amyloliquefaciens strain TJ1000 and fluensulfone, Bacillus amyloliquefaciens strain TJ1000 and fluopyram, Bacillus amyloliquefaciens strain TJ1000 and tioxazafen, Bacillus amyloliquefaciens strain TJ1000 and N-[1-(2,6-difluorophenyl)-1H-pyrazol-3-yl]-2-trifluoromethylbenzamide, Bacillus amyloliquefaciens strain TJ1000 and cis-Jasmone, Bacillus amyloliquefaciens strain TJ1000 and harpin, Bacillus amyloliquefaciens strain TJ1000 and Azadirachta indica oil, Bacillus amyloliquefaciens strain TJ1000 and Azadirachtin, Bacillus amyloliquefaciens strain AP-136 and alanycarb, Bacillus amyloliquefaciens strain AP-136 and aldicarb, Bacillus amyloliquefaciens strain AP-136 and carbofuran, Bacillus amyloliquefaciens strain AP-136 and carbosulfan, Bacillus amyloliquefaciens strain AP-136 and fosthiazate, Bacillus amyloliquefaciens strain AP-136 and cadusafos, Bacillus amyloliquefaciens strain AP-136 and oxamyl, Bacillus amyloliquefaciens strain AP-136 and thiodicarb, Bacillus amyloliquefaciens strain AP-136 and dimethoate, Bacillus amyloliquefaciens strain AP-136 and ethoprophos, Bacillus amyloliquefaciens strain AP-136 and terbufos, Bacillus amyloliquefaciens strain AP-136 and abamectin, Bacillus amyloliquefaciens strain AP-136 and methyl bromide and other alkyl halides, Bacillus amyloliquefaciens strain AP-136 and methyl isocyanate generators selected from diazomet and metam, Bacillus amyloliquefaciens strain AP-136 and fluazaindolizine, Bacillus amyloliquefaciens strain AP-136 and fluensulfone, Bacillus amyloliquefaciens strain AP-136 and fluopyram, Bacillus amyloliquefaciens strain AP-136 and tioxazafen, Bacillus amyloliquefaciens strain AP-136 and N-[1-(2,6-difluorophenyl)-1H-pyrazol-3-yl]-2-trifluoromethylbenzamide, Bacillus amyloliquefaciens strain AP-136 and cis-Jasmone, Bacillus amyloliquefaciens strain AP-136 and harpin, Bacillus amyloliquefaciens strain AP-136 and Azadirachta indica oil, Bacillus amyloliquefaciens strain AP-136 and Azadirachtin, Bacillus amyloliquefaciens strain AP-188 and alanycarb, Bacillus amyloliquefaciens strain AP-188 and aldicarb, Bacillus amyloliquefaciens strain AP-188 and carbofuran, Bacillus amyloliquefaciens strain AP-188 and carbosulfan, Bacillus amyloliquefaciens strain AP-188 and fosthiazate, Bacillus amyloliquefaciens strain AP-188 and cadusafos, Bacillus amyloliquefaciens strain AP-188 and oxamyl, Bacillus amyloliquefaciens strain AP-188 and thiodicarb, Bacillus amyloliquefaciens strain AP-188 and dimethoate, Bacillus amyloliquefaciens strain AP-188 and ethoprophos, Bacillus amyloliquefaciens strain AP-188 and terbufos, Bacillus amyloliquefaciens strain AP-188 and abamectin, Bacillus amyloliquefaciens strain AP-188 and methyl bromide and other alkyl halides, Bacillus amyloliquefaciens strain AP-188 and methyl isocyanate generators selected from diazomet and metam, Bacillus amyloliquefaciens strain AP-188 and fluazaindolizine, Bacillus amyloliquefaciens strain AP-188 and fluensulfone, Bacillus amyloliquefaciens strain AP-188 and fluopyram, Bacillus amyloliquefaciens strain AP-188 and tioxazafen, Bacillus amyloliquefaciens strain AP-188 and N-[1-(2,6-difluorophenyl)-1H-pyrazol-3-yl]-2-trifluoromethylbenzamide, Bacillus amyloliquefaciens strain AP-188 and cis-Jasmone, Bacillus amyloliquefaciens strain AP-188 and harpin, Bacillus amyloliquefaciens strain AP-188 and Azadirachta indica oil, Bacillus amyloliquefaciens strain AP-188 and Azadirachtin, Bacillus amyloliquefaciens strain AP-218 and alanycarb, Bacillus amyloliquefaciens strain AP-218 and aldicarb, Bacillus amyloliquefaciens strain AP-218 and carbofuran, Bacillus amyloliquefaciens strain AP-218 and carbosulfan, Bacillus amyloliquefaciens strain AP-218 and fosthiazate, Bacillus amyloliquefaciens strain AP-218 and cadusafos, Bacillus amyloliquefaciens strain AP-218 and oxamyl, Bacillus amyloliquefaciens strain AP-218 and thiodicarb, Bacillus amyloliquefaciens strain AP-218 and dimethoate, Bacillus amyloliquefaciens strain AP-218 and ethoprophos, Bacillus amyloliquefaciens strain AP-218 and terbufos, Bacillus amyloliquefaciens strain AP-218 and abamectin, Bacillus amyloliquefaciens strain AP-218 and methyl bromide and other alkyl halides, Bacillus amyloliquefaciens strain AP-218 and methyl isocyanate generators selected from diazomet and metam, Bacillus amyloliquefaciens strain AP-218 and fluazaindolizine, Bacillus amyloliquefaciens strain AP-218 and fluensulfone, Bacillus amyloliquefaciens strain AP-218 and fluopyram, Bacillus amyloliquefaciens strain AP-218 and tioxazafen, Bacillus amyloliquefaciens strain AP-218 and N-[1-(2,6-difluorophenyl)-1H-pyrazol-3-yl]-2-trifluoromethylbenzamide, Bacillus amyloliquefaciens strain AP-218 and cis-Jasmone, Bacillus amyloliquefaciens strain AP-218 and harpin, Bacillus amyloliquefaciens strain AP-218 and Azadirachta indica oil, Bacillus amyloliquefaciens strain AP-218 and Azadirachtin, Bacillus amyloliquefaciens strain AP-219 and alanycarb, Bacillus amyloliquefaciens strain AP-219 and aldicarb, Bacillus amyloliquefaciens strain AP-219 and carbofuran, Bacillus amyloliquefaciens strain AP-219 and carbosulfan, Bacillus amyloliquefaciens strain AP-219 and fosthiazate, Bacillus amyloliquefaciens strain AP-219 and cadusafos, Bacillus amyloliquefaciens strain AP-219 and oxamyl, Bacillus amyloliquefaciens strain AP-219 and thiodicarb, Bacillus amyloliquefaciens strain AP-219 and dimethoate, Bacillus amyloliquefaciens strain AP-219 and ethoprophos, Bacillus amyloliquefaciens strain AP-219 and terbufos, Bacillus amyloliquefaciens strain AP-219 and abamectin, Bacillus amyloliquefaciens strain AP-219 and methyl bromide and other alkyl halides, Bacillus amyloliquefaciens strain AP-219 and methyl isocyanate generators selected from diazomet and metam, Bacillus amyloliquefaciens strain AP-219 and fluazaindolizine, Bacillus amyloliquefaciens strain AP-219 and fluensulfone, Bacillus amyloliquefaciens strain AP-219 and fluopyram, Bacillus amyloliquefaciens strain AP-219 and tioxazafen, Bacillus amyloliquefaciens strain AP-219 and N-[1-(2,6-difluorophenyl)-1H-pyrazol-3-yl]-2-trifluoromethylbenzamide, Bacillus amyloliquefaciens strain AP-219 and cis-Jasmone, Bacillus amyloliquefaciens strain AP-219 and harpin, Bacillus amyloliquefaciens strain AP-219 and Azadirachta indica oil, Bacillus amyloliquefaciens strain AP-219 and Azadirachtin, Bacillus amyloliquefaciens strain AP-295 and alanycarb, Bacillus amyloliquefaciens strain AP-295 and aldicarb, Bacillus amyloliquefaciens strain AP-295 and carbofuran, Bacillus amyloliquefaciens strain AP-295 and carbosulfan, Bacillus amyloliquefaciens strain AP-295 and fosthiazate, Bacillus amyloliquefaciens strain AP-295 and cadusafos, Bacillus amyloliquefaciens strain AP-295 and oxamyl, Bacillus amyloliquefaciens strain AP-295 and thiodicarb, Bacillus amyloliquefaciens strain AP-295 and dimethoate, Bacillus amyloliquefaciens strain AP-295 and ethoprophos, Bacillus amyloliquefaciens strain AP-295 and terbufos, Bacillus amyloliquefaciens strain AP-295 and abamectin, Bacillus amyloliquefaciens strain AP-295 and methyl bromide and other alkyl halides, Bacillus amyloliquefaciens strain AP-295 and methyl isocyanate generators selected from diazomet and metam, Bacillus amyloliquefaciens strain AP-295 and fluazaindolizine, Bacillus amyloliquefaciens strain AP-295 and fluensulfone, Bacillus amyloliquefaciens strain AP-295 and fluopyram, Bacillus amyloliquefaciens strain AP-295 and tioxazafen, Bacillus amyloliquefaciens strain AP-295 and N-[1-(2,6-difluorophenyl)-1H-pyrazol-3-yl]-2-trifluoromethylbenzamide, Bacillus amyloliquefaciens strain AP-295 and cis-Jasmone, Bacillus amyloliquefaciens strain AP-295 and harpin, Bacillus amyloliquefaciens strain AP-295 and Azadirachta indica oil, Bacillus amyloliquefaciens strain AP-295 and Azadirachtin, Bacillus amyloliquefaciens strain MBI 600 and alanycarb, Bacillus amyloliquefaciens strain MBI 600 and aldicarb, Bacillus amyloliquefaciens strain MBI 600 and carbofuran, Bacillus amyloliquefaciens strain MBI 600 and carbosulfan, Bacillus amyloliquefaciens strain MBI 600 and fosthiazate, Bacillus amyloliquefaciens strain MBI 600 and cadusafos, Bacillus amyloliquefaciens strain MBI 600 and oxamyl, Bacillus amyloliquefaciens strain MBI 600 and thiodicarb, Bacillus amyloliquefaciens strain MBI 600 and dimethoate, Bacillus amyloliquefaciens strain MBI 600 and ethoprophos, Bacillus amyloliquefaciens strain MBI 600 and terbufos, Bacillus amyloliquefaciens strain MBI 600 and abamectin, Bacillus amyloliquefaciens strain MBI 600 and methyl bromide and other alkyl halides, Bacillus amyloliquefaciens strain MBI 600 and methyl isocyanate generators selected from diazomet and metam, Bacillus amyloliquefaciens strain MBI 600 and fluazaindolizine, Bacillus amyloliquefaciens strain MBI 600 and fluensulfone, Bacillus amyloliquefaciens strain MBI 600 and fluopyram, Bacillus amyloliquefaciens strain MBI 600 and tioxazafen, Bacillus amyloliquefaciens strain MBI 600 and N-[1-(2,6-difluorophenyl)-1H-pyrazol-3-yl]-2-trifluoromethylbenzamide, Bacillus amyloliquefaciens strain MBI 600 and cis-Jasmone, Bacillus amyloliquefaciens strain MBI 600 and harpin, Bacillus amyloliquefaciens strain MBI 600 and Azadirachta indica oil, Bacillus amyloliquefaciens strain MBI 600 and Azadirachtin, Bacillus amyloliquefaciens strain PTA-4838 and alanycarb, Bacillus amyloliquefaciens strain PTA-4838 and aldicarb, Bacillus amyloliquefaciens strain PTA-4838 and carbofuran, Bacillus amyloliquefaciens strain PTA-4838 and carbosulfan, Bacillus amyloliquefaciens strain PTA-4838 and fosthiazate, Bacillus amyloliquefaciens strain PTA-4838 and cadusafos, Bacillus amyloliquefaciens strain PTA-4838 and oxamyl, Bacillus amyloliquefaciens strain PTA-4838 and thiodicarb, Bacillus amyloliquefaciens strain PTA-4838 and dimethoate, Bacillus amyloliquefaciens strain PTA-4838 and ethoprophos, Bacillus amyloliquefaciens strain PTA-4838 and terbufos, Bacillus amyloliquefaciens strain PTA-4838 and abamectin, Bacillus amyloliquefaciens strain PTA-4838 and methyl bromide and other alkyl halides, Bacillus amyloliquefaciens strain PTA-4838 and methyl isocyanate generators selected from diazomet and metam, Bacillus amyloliquefaciens strain PTA-4838 and fluazaindolizine, Bacillus amyloliquefaciens strain PTA-4838 and fluensulfone, Bacillus amyloliquefaciens strain PTA-4838 and fluopyram, Bacillus amyloliquefaciens strain PTA-4838 and tioxazafen, Bacillus amyloliquefaciens strain PTA-4838 and N-[1-(2,6-difluorophenyl)-1H-pyrazol-3-yl]-2-trifluoromethylbenzamide, Bacillus amyloliquefaciens strain PTA-4838 and cis-Jasmone, Bacillus amyloliquefaciens strain PTA-4838 and harpin, Bacillus amyloliquefaciens strain PTA-4838 and Azadirachta indica oil, Bacillus amyloliquefaciens strain PTA-4838 and Azadirachtin, Bacillus amyloliquefaciens strain F727 and alanycarb, Bacillus amyloliquefaciens strain F727 and aldicarb, Bacillus amyloliquefaciens strain F727 and carbofuran, Bacillus amyloliquefaciens strain F727 and carbosulfan, Bacillus amyloliquefaciens strain F727 and fosthiazate, Bacillus amyloliquefaciens strain F727 and cadusafos, Bacillus amyloliquefaciens strain F727 and oxamyl, Bacillus amyloliquefaciens strain F727 and thiodicarb, Bacillus amyloliquefaciens strain F727 and dimethoate, Bacillus amyloliquefaciens strain F727 and ethoprophos, Bacillus amyloliquefaciens strain F727 and terbufos, Bacillus amyloliquefaciens strain F727 and abamectin, Bacillus amyloliquefaciens strain F727 and methyl bromide and other alkyl halides, Bacillus amyloliquefaciens strain F727 and methyl isocyanate generators selected from diazomet and metam, Bacillus amyloliquefaciens strain F727 and fluazaindolizine, Bacillus amyloliquefaciens strain F727 and fluensulfone, Bacillus amyloliquefaciens strain F727 and fluopyram, Bacillus amyloliquefaciens strain F727 and tioxazafen, Bacillus amyloliquefaciens strain F727 and N-[1-(2,6-difluorophenyl)-1H-pyrazol-3-yl]-2-trifluoromethylbenzamide, Bacillus amyloliquefaciens strain F727 and cis-Jasmone, Bacillus amyloliquefaciens strain F727 and harpin, Bacillus amyloliquefaciens strain F727 and Azadirachta indica oil, Bacillus amyloliquefaciens strain F727 and Azadirachtin, Bacillus firmus strain I-1582 and alanycarb, Bacillus firmus strain I-1582 and aldicarb, Bacillus firmus strain I-1582 and carbofuran, Bacillus firmus strain I-1582 and carbosulfan, Bacillus firmus strain I-1582 and fosthiazate, Bacillus firmus strain I-1582 and cadusafos, Bacillus firmus strain I-1582 and oxamyl, Bacillus firmus strain I-1582 and thiodicarb, Bacillus firmus strain I-1582 and dimethoate, Bacillus firmus strain I-1582 and ethoprophos, Bacillus firmus strain I-1582 and terbufos, Bacillus firmus strain I-1582 and abamectin, Bacillus firmus strain I-1582 and methyl bromide and other alkyl halides, Bacillus firmus strain I-1582 and methyl isocyanate generators selected from diazomet and metam, Bacillus firmus strain I-1582 and fluazaindolizine, Bacillus firmus strain I-1582 and fluensulfone, Bacillus firmus strain I-1582 and fluopyram, Bacillus firmus strain I-1582 and tioxazafen, Bacillus firmus strain I-1582 and N-[1-(2,6-difluorophenyl)-1H-pyrazol-3-yl]-2-trifluoromethylbenzamide, Bacillus firmus strain I-1582 and cis-Jasmone, Bacillus firmus strain I-1582 and harpin, Bacillus firmus strain I-1582 and Azadirachta indica oil, Bacillus firmus strain I-1582 and Azadirachtin, Bacillus firmus strain NRRL B-67003 and alanycarb, Bacillus firmus strain NRRL B-67003 and aldicarb, Bacillus firmus strain NRRL B-67003 and carbofuran, Bacillus firmus strain NRRL B-67003 and carbosulfan, Bacillus firmus strain NRRL B-67003 and fosthiazate, Bacillus firmus strain NRRL B-67003 and cadusafos, Bacillus firmus strain NRRL B-67003 and oxamyl, Bacillus firmus strain NRRL B-67003 and thiodicarb, Bacillus firmus strain NRRL B-67003 and dimethoate, Bacillus firmus strain NRRL B-67003 and ethoprophos, Bacillus firmus strain NRRL B-67003 and terbufos, Bacillus firmus strain NRRL B-67003 and abamectin, Bacillus firmus strain NRRL B-67003 and methyl bromide and other alkyl halides, Bacillus firmus strain NRRL B-67003 and methyl isocyanate generators selected from diazomet and metam, Bacillus firmus strain NRRL B-67003 and fluazaindolizine, Bacillus firmus strain NRRL B-67003 and fluensulfone, Bacillus firmus strain NRRL B-67003 and fluopyram, Bacillus firmus strain NRRL B-67003 and tioxazafen, Bacillus firmus strain NRRL B-67003 and N-[1-(2,6-difluorophenyl)-1H-pyrazol-3-yl]-2-trifluoromethylbenzamide, Bacillus firmus strain NRRL B-67003 and cis-Jasmone, Bacillus firmus strain NRRL B-67003 and harpin, Bacillus firmus strain NRRL B-67003 and Azadirachta indica oil, Bacillus firmus strain NRRL B-67003 and Azadirachtin, Bacillus firmus strain NRRL B-67518 and alanycarb, Bacillus firmus strain NRRL B-67518 and aldicarb, Bacillus firmus strain NRRL B-67518 and carbofuran, Bacillus firmus strain NRRL B-67518 and carbosulfan, Bacillus firmus strain NRRL B-67518 and fosthiazate, Bacillus firmus strain NRRL B-67518 and cadusafos, Bacillus firmus strain NRRL B-67518 and oxamyl, Bacillus firmus strain NRRL B-67518 and thiodicarb, Bacillus firmus strain NRRL B-67518 and dimethoate, Bacillus firmus strain NRRL B-67518 and ethoprophos, Bacillus firmus strain NRRL B-67518 and terbufos, Bacillus firmus strain NRRL B-67518 and abamectin, Bacillus firmus strain NRRL B-67518 and methyl bromide and other alkyl halides, Bacillus firmus strain NRRL B-67518 and methyl isocyanate generators selected from diazomet and metam, Bacillus firmus strain NRRL B-67518 and fluazaindolizine, Bacillus firmus strain NRRL B-67518 and fluensulfone, Bacillus firmus strain NRRL B-67518 and fluopyram, Bacillus firmus strain NRRL B-67518 and tioxazafen, Bacillus firmus strain NRRL B-67518 and N-[1-(2,6-difluorophenyl)-1H-pyrazol-3-yl]-2-trifluoromethylbenzamide, Bacillus firmus strain NRRL B-67518 and cis-Jasmone, Bacillus firmus strain NRRL B-67518 and harpin, Bacillus firmus strain NRRL B-67518 and Azadirachta indica oil, Bacillus firmus strain NRRL B-67518 and Azadirachtin, Bacillus firmus strain GB126 and alanycarb, Bacillus firmus strain GB126 and aldicarb, Bacillus firmus strain GB126 and carbofuran, Bacillus firmus strain GB126 and carbosulfan, Bacillus firmus strain GB126 and fosthiazate, Bacillus firmus strain GB126 and cadusafos, Bacillus firmus strain GB126 and oxamyl, Bacillus firmus strain GB126 and thiodicarb, Bacillus firmus strain GB126 and dimethoate, Bacillus firmus strain GB126 and ethoprophos, Bacillus firmus strain GB126 and terbufos, Bacillus firmus strain GB126 and abamectin, Bacillus firmus strain GB126 and methyl bromide and other alkyl halides, Bacillus firmus strain GB126 and methyl isocyanate generators selected from diazomet and metam, Bacillus firmus strain GB126 and fluazaindolizine, Bacillus firmus strain GB126 and fluensulfone, Bacillus firmus strain GB126 and fluopyram, Bacillus firmus strain GB126 and tioxazafen, Bacillus firmus strain GB126 and N-[1-(2,6-difluorophenyl)-1H-pyrazol-3-yl]-2-trifluoromethylbenzamide, Bacillus firmus strain GB126 and cis-Jasmone, Bacillus firmus strain GB126 and harpin, Bacillus firmus strain GB126 and Azadirachta indica oil, Bacillus firmus strain GB126 and Azadirachtin, Bacillus laterosporus strain ATCC PTA-3952 and alanycarb, Bacillus laterosporus strain ATCC PTA-3952 and aldicarb, Bacillus laterosporus strain ATCC PTA-3952 and carbofuran, Bacillus laterosporus strain ATCC PTA-3952 and carbosulfan, Bacillus laterosporus strain ATCC PTA-3952 and fosthiazate, Bacillus laterosporus strain ATCC PTA-3952 and cadusafos, Bacillus laterosporus strain ATCC PTA-3952 and oxamyl, Bacillus laterosporus strain ATCC PTA-3952 and thiodicarb, Bacillus laterosporus strain ATCC PTA-3952 and dimethoate, Bacillus laterosporus strain ATCC PTA-3952 and ethoprophos, Bacillus laterosporus strain ATCC PTA-3952 and terbufos, Bacillus laterosporus strain ATCC PTA-3952 and abamectin, Bacillus laterosporus strain ATCC PTA-3952 and methyl bromide and other alkyl halides, Bacillus laterosporus strain ATCC PTA-3952 and methyl isocyanate generators selected from diazomet and metam, Bacillus laterosporus strain ATCC PTA-3952 and fluazaindolizine, Bacillus laterosporus strain ATCC PTA-3952 and fluensulfone, Bacillus laterosporus strain ATCC PTA-3952 and fluopyram, Bacillus laterosporus strain ATCC PTA-3952 and tioxazafen, Bacillus laterosporus strain ATCC PTA-3952 and N-[1-(2,6-difluorophenyl)-1H-pyrazol-3-yl]-2-trifluoromethylbenzamide, Bacillus laterosporus strain ATCC PTA-3952 and cis-Jasmone, Bacillus laterosporus strain ATCC PTA-3952 and harpin, Bacillus laterosporus strain ATCC PTA-3952 and Azadirachta indica oil, Bacillus laterosporus strain ATCC PTA-3952 and Azadirachtin, Bacillus laterosporus strain ATCC PTA-3593 and alanycarb, Bacillus laterosporus strain ATCC PTA-3593 and aldicarb, Bacillus laterosporus strain ATCC PTA-3593 and carbofuran, Bacillus laterosporus strain ATCC PTA-3593 and carbosulfan, Bacillus laterosporus strain ATCC PTA-3593 and fosthiazate, Bacillus laterosporus strain ATCC PTA-3593 and cadusafos, Bacillus laterosporus strain ATCC PTA-3593 and oxamyl, Bacillus laterosporus strain ATCC PTA-3593 and thiodicarb, Bacillus laterosporus strain ATCC PTA-3593 and dimethoate, Bacillus laterosporus strain ATCC PTA-3593 and ethoprophos, Bacillus laterosporus strain ATCC PTA-3593 and terbufos, Bacillus laterosporus strain ATCC PTA-3593 and abamectin, Bacillus laterosporus strain ATCC PTA-3593 and methyl bromide and other alkyl halides, Bacillus laterosporus strain ATCC PTA-3593 and methyl isocyanate generators selected from diazomet and metam, Bacillus laterosporus strain ATCC PTA-3593 and fluazaindolizine, Bacillus laterosporus strain ATCC PTA-3593 and fluensulfone, Bacillus laterosporus strain ATCC PTA-3593 and fluopyram, Bacillus laterosporus strain ATCC PTA-3593 and tioxazafen, Bacillus laterosporus strain ATCC PTA-3593 and N-[1-(2,6-difluorophenyl)-1H-pyrazol-3-yl]-2-trifluoromethylbenzamide, Bacillus laterosporus strain ATCC PTA-3593 and cis-Jasmone, Bacillus laterosporus strain ATCC PTA-3593 and harpin, Bacillus laterosporus strain ATCC PTA-3593 and Azadirachta indica oil, Bacillus laterosporus strain ATCC PTA-3593 and Azadirachtin, Bacillus licheniformis strain ATCC PTA-6175 and alanycarb, Bacillus licheniformis strain ATCC PTA-6175 and aldicarb, Bacillus licheniformis strain ATCC PTA-6175 and carbofuran, Bacillus licheniformis strain ATCC PTA-6175 and carbosulfan, Bacillus licheniformis strain ATCC PTA-6175 and fosthiazate, Bacillus licheniformis strain ATCC PTA-6175 and cadusafos, Bacillus licheniformis strain ATCC PTA-6175 and oxamyl, Bacillus licheniformis strain ATCC PTA-6175 and thiodicarb, Bacillus licheniformis strain ATCC PTA-6175 and dimethoate, Bacillus licheniformis strain ATCC PTA-6175 and ethoprophos, Bacillus licheniformis strain ATCC PTA-6175 and terbufos, Bacillus licheniformis strain ATCC PTA-6175 and abamectin, Bacillus licheniformis strain ATCC PTA-6175 and methyl bromide and other alkyl halides, Bacillus licheniformis strain ATCC PTA-6175 and methyl isocyanate generators selected from diazomet and metam, Bacillus licheniformis strain ATCC PTA-6175 and fluazaindolizine, Bacillus licheniformis strain ATCC PTA-6175 and fluensulfone, Bacillus licheniformis strain ATCC PTA-6175 and fluopyram, Bacillus licheniformis strain ATCC PTA-6175 and tioxazafen, Bacillus licheniformis strain ATCC PTA-6175 and N-[1-(2,6-difluorophenyl)-1H-pyrazol-3-yl]-2-trifluoromethylbenzamide, Bacillus licheniformis strain ATCC PTA-6175 and cis-Jasmone, Bacillus licheniformis strain ATCC PTA-6175 and harpin, Bacillus licheniformis strain ATCC PTA-6175 and Azadirachta indica oil, Bacillus licheniformis strain ATCC PTA-6175 and Azadirachtin, Bacillus licheniformis SB3086 and alanycarb, Bacillus licheniformis SB3086 and aldicarb, Bacillus licheniformis SB3086 and carbofuran, Bacillus licheniformis SB3086 and carbosulfan, Bacillus licheniformis SB3086 and fosthiazate, Bacillus licheniformis SB3086 and cadusafos, Bacillus licheniformis SB3086 and oxamyl, Bacillus licheniformis SB3086 and thiodicarb, Bacillus licheniformis SB3086 and dimethoate, Bacillus licheniformis SB3086 and ethoprophos, Bacillus licheniformis SB3086 and terbufos, Bacillus licheniformis SB3086 and abamectin, Bacillus licheniformis SB3086 and methyl bromide and other alkyl halides, Bacillus licheniformis SB3086 and methyl isocyanate generators selected from diazomet and metam, Bacillus licheniformis SB3086 and fluazaindolizine, Bacillus licheniformis SB3086 and fluensulfone, Bacillus licheniformis SB3086 and fluopyram, Bacillus licheniformis SB3086 and tioxazafen, Bacillus licheniformis SB3086 and N-[1-(2,6-difluorophenyl)-1H-pyrazol-3-yl]-2-trifluoromethylbenzamide, Bacillus licheniformis SB3086 and cis-Jasmone, Bacillus licheniformis SB3086 and harpin, Bacillus licheniformis SB3086 and Azadirachta indica oil, Bacillus licheniformis SB3086 and Azadirachtin, Bacillus licheniformis CH200 and alanycarb, Bacillus licheniformis CH200 and aldicarb, Bacillus licheniformis CH200 and carbofuran, Bacillus licheniformis CH200 and carbosulfan, Bacillus licheniformis CH200 and fosthiazate, Bacillus licheniformis CH200 and cadusafos, Bacillus licheniformis CH200 and oxamyl, Bacillus licheniformis CH200 and thiodicarb, Bacillus licheniformis CH200 and dimethoate, Bacillus licheniformis CH200 and ethoprophos, Bacillus licheniformis CH200 and terbufos, Bacillus licheniformis CH200 and abamectin, Bacillus licheniformis CH200 and methyl bromide and other alkyl halides, Bacillus licheniformis CH200 and methyl isocyanate generators selected from diazomet and metam, Bacillus licheniformis CH200 and fluazaindolizine, Bacillus licheniformis CH200 and fluensulfone, Bacillus licheniformis CH200 and fluopyram, Bacillus licheniformis CH200 and tioxazafen, Bacillus licheniformis CH200 and N-[1-(2,6-difluorophenyl)-1H-pyrazol-3-yl]-2-trifluoromethylbenzamide, Bacillus licheniformis CH200 and cis-Jasmone, Bacillus licheniformis CH200 and harpin, Bacillus licheniformis CH200 and Azadirachta indica oil, Bacillus licheniformis CH200 and Azadirachtin, Bacillus licheniformis RTI 184 and alanycarb, Bacillus licheniformis RTI 184 and aldicarb, Bacillus licheniformis RTI 184 and carbofuran, Bacillus licheniformis RTI 184 and carbosulfan, Bacillus licheniformis RTI 184 and fosthiazate, Bacillus licheniformis RTI 184 and cadusafos, Bacillus licheniformis RTI 184 and oxamyl, Bacillus licheniformis RTI 184 and thiodicarb, Bacillus licheniformis RTI 184 and dimethoate, Bacillus licheniformis RTI 184 and ethoprophos, Bacillus licheniformis RTI 184 and terbufos, Bacillus licheniformis RTI 184 and abamectin, Bacillus licheniformis RTI 184 and methyl bromide and other alkyl halides, Bacillus licheniformis RTI 184 and methyl isocyanate generators selected from diazomet and metam, Bacillus licheniformis RTI 184 and fluazaindolizine, Bacillus licheniformis RTI 184 and fluensulfone, Bacillus licheniformis RTI 184 and fluopyram, Bacillus licheniformis RTI 184 and tioxazafen, Bacillus licheniformis RTI 184 and N-[1-(2,6-difluorophenyl)-1H-pyrazol-3-yl]-2-trifluoromethylbenzamide, Bacillus licheniformis RTI 184 and cis-Jasmone, Bacillus licheniformis RTI 184 and harpin, Bacillus licheniformis RTI 184 and Azadirachta indica oil, Bacillus licheniformis RTI 184 and Azadirachtin, a combination of Bacillus licheniformis CH200 and Bacillus licheniformis RTI 184 and alanycarb, a combination of Bacillus licheniformis CH200 and Bacillus licheniformis RTI 184 and aldicarb, a combination of Bacillus licheniformis CH200 and Bacillus licheniformis RTI 184 and carbofuran, a combination of Bacillus licheniformis CH200 and Bacillus licheniformis RTI 184 and carbosulfan, a combination of Bacillus licheniformis CH200 and Bacillus licheniformis RTI 184 and fosthiazate, a combination of Bacillus licheniformis CH200 and Bacillus licheniformis RTI 184 and cadusafos, a combination of Bacillus licheniformis CH200 and Bacillus licheniformis RTI 184 and oxamyl, a combination of Bacillus licheniformis CH200 and Bacillus licheniformis RTI 184 and thiodicarb, a combination of Bacillus licheniformis CH200 and Bacillus licheniformis RTI 184 and dimethoate, a combination of Bacillus licheniformis CH200 and Bacillus licheniformis RTI 184 and ethoprophos, a combination of Bacillus licheniformis CH200 and Bacillus licheniformis RTI 184 and terbufos, a combination of Bacillus licheniformis CH200 and Bacillus licheniformis RTI 184 and abamectin, a combination of Bacillus licheniformis CH200 and Bacillus licheniformis RTI 184 and methyl bromide and other alkyl halides, a combination of Bacillus licheniformis CH200 and Bacillus licheniformis RTI 184 and methyl isocyanate generators selected from diazomet and metam, a combination of Bacillus licheniformis CH200 and Bacillus licheniformis RTI 184 and fluazaindolizine, a combination of Bacillus licheniformis CH200 and Bacillus licheniformis RTI 184 and fluensulfone, a combination of Bacillus licheniformis CH200 and Bacillus licheniformis RTI 184 and fluopyram, a combination of Bacillus licheniformis CH200 and Bacillus licheniformis RTI 184 and tioxazafen, a combination of Bacillus licheniformis CH200 and Bacillus licheniformis RTI 184 and N-[1-(2,6-difluorophenyl)-1H-pyrazol-3-yl]-2-trifluoromethylbenzamide, a combination of Bacillus licheniformis CH200 and Bacillus licheniformis RTI 184 and cis-Jasmone, a combination of Bacillus licheniformis CH200 and Bacillus licheniformis RTI 184 and harpin, a combination of Bacillus licheniformis CH200 and Bacillus licheniformis RTI 184 and Azadirachta indica oil, a combination of Bacillus licheniformis CH200 and Bacillus licheniformis RTI 184 and Azadirachtin, Bacillus subtilis strain GB03 and alanycarb, Bacillus subtilis strain GB03 and aldicarb, Bacillus subtilis strain GB03 and carbofuran, Bacillus subtilis strain GB03 and carbosulfan, Bacillus subtilis strain GB03 and fosthiazate, Bacillus subtilis strain GB03 and cadusafos, Bacillus subtilis strain GB03 and oxamyl, Bacillus subtilis strain GB03 and thiodicarb, Bacillus subtilis strain GB03 and dimethoate, Bacillus subtilis strain GB03 and ethoprophos, Bacillus subtilis strain GB03 and terbufos, Bacillus subtilis strain GB03 and abamectin, Bacillus subtilis strain GB03 and methyl bromide and other alkyl halides, Bacillus subtilis strain GB03 and methyl isocyanate generators selected from diazomet and metam, Bacillus subtilis strain GB03 and fluazaindolizine, Bacillus subtilis strain GB03 and fluensulfone, Bacillus subtilis strain GB03 and fluopyram, Bacillus subtilis strain GB03 and tioxazafen, Bacillus subtilis strain GB03 and N-[1-(2,6-difluorophenyl)-1H-pyrazol-3-yl]-2-trifluoromethylbenzamide, Bacillus subtilis strain GB03 and cis-Jasmone, Bacillus subtilis strain GB03 and harpin, Bacillus subtilis strain GB03 and Azadirachta indica oil, Bacillus subtilis strain GB03 and Azadirachtin, Bacillus subtilis strain QST713/AQ713 and alanycarb, Bacillus subtilis strain QST713/AQ713 and aldicarb, Bacillus subtilis strain QST713/AQ713 and carbofuran, Bacillus subtilis strain QST713/AQ713 and carbosulfan, Bacillus subtilis strain QST713/AQ713 and fosthiazate, Bacillus subtilis strain QST713/AQ713 and cadusafos, Bacillus subtilis strain QST713/AQ713 and oxamyl, Bacillus subtilis strain QST713/AQ713 and thiodicarb, Bacillus subtilis strain QST713/AQ713 and dimethoate, Bacillus subtilis strain QST713/AQ713 and ethoprophos, Bacillus subtilis strain QST713/AQ713 and terbufos, Bacillus subtilis strain QST713/AQ713 and abamectin, Bacillus subtilis strain QST713/AQ713 and methyl bromide and other alkyl halides, Bacillus subtilis strain QST713/AQ713 and methyl isocyanate generators selected from diazomet and metam, Bacillus subtilis strain QST713/AQ713 and fluazaindolizine, Bacillus subtilis strain QST713/AQ713 and fluensulfone, Bacillus subtilis strain QST713/AQ713 and fluopyram, Bacillus subtilis strain QST713/AQ713 and tioxazafen, Bacillus subtilis strain QST713/AQ713 and N-[1-(2,6-difluorophenyl)-1H-pyrazol-3-yl]-2-trifluoromethylbenzamide, Bacillus subtilis strain QST713/AQ713 and cis-Jasmone, Bacillus subtilis strain QST713/AQ713 and harpin, Bacillus subtilis strain QST713/AQ713 and Azadirachta indica oil, Bacillus subtilis strain QST713/AQ713 and Azadirachtin, Bacillus subtilis strain AQ 153 and alanycarb, Bacillus subtilis strain AQ 153 and aldicarb, Bacillus subtilis strain AQ 153 and carbofuran, Bacillus subtilis strain AQ 153 and carbosulfan, Bacillus subtilis strain AQ 153 and fosthiazate, Bacillus subtilis strain AQ 153 and cadusafos, Bacillus subtilis strain AQ 153 and oxamyl, Bacillus subtilis strain AQ 153 and thiodicarb, Bacillus subtilis strain AQ 153 and dimethoate, Bacillus subtilis strain AQ 153 and ethoprophos, Bacillus subtilis strain AQ 153 and terbufos, Bacillus subtilis strain AQ 153 and abamectin, Bacillus subtilis strain AQ 153 and methyl bromide and other alkyl halides, Bacillus subtilis strain AQ 153 and methyl isocyanate generators selected from diazomet and metam, Bacillus subtilis strain AQ 153 and fluazaindolizine, Bacillus subtilis strain AQ 153 and fluensulfone, Bacillus subtilis strain AQ 153 and fluopyram, Bacillus subtilis strain AQ 153 and tioxazafen, Bacillus subtilis strain AQ 153 and N-[1-(2,6-difluorophenyl)-1H-pyrazol-3-yl]-2-trifluoromethylbenzamide, Bacillus subtilis strain AQ 153 and cis-Jasmone, Bacillus subtilis strain AQ 153 and harpin, Bacillus subtilis strain AQ 153 and Azadirachta indica oil, Bacillus subtilis strain AQ 153 and Azadirachtin, Bacillus subtilis strain AQ743 and alanycarb, Bacillus subtilis strain AQ743 and aldicarb, Bacillus subtilis strain AQ743 and carbofuran, Bacillus subtilis strain AQ743 and carbosulfan, Bacillus subtilis strain AQ743 and fosthiazate, Bacillus subtilis strain AQ743 and cadusafos, Bacillus subtilis strain AQ743 and oxamyl, Bacillus subtilis strain AQ743 and thiodicarb, Bacillus subtilis strain AQ743 and dimethoate, Bacillus subtilis strain AQ743 and ethoprophos, Bacillus subtilis strain AQ743 and terbufos, Bacillus subtilis strain AQ743 and abamectin, Bacillus subtilis strain AQ743 and methyl bromide and other alkyl halides, Bacillus subtilis strain AQ743 and methyl isocyanate generators selected from diazomet and metam, Bacillus subtilis strain AQ743 and fluazaindolizine, Bacillus subtilis strain AQ743 and fluensulfone, Bacillus subtilis strain AQ743 and fluopyram, Bacillus subtilis strain AQ743 and tioxazafen, Bacillus subtilis strain AQ743 and N-[1-(2,6-difluorophenyl)-1H-pyrazol-3-yl]-2-trifluoromethylbenzamide, Bacillus subtilis strain AQ743 and cis-Jasmone, Bacillus subtilis strain AQ743 and harpin, Bacillus subtilis strain AQ743 and Azadirachta indica oil, Bacillus subtilis strain AQ743 and Azadirachtin, Bacillus subtilis strain DB 101 and alanycarb, Bacillus subtilis strain DB 101 and aldicarb, Bacillus subtilis strain DB 101 and carbofuran, Bacillus subtilis strain DB 101 and carbosulfan, Bacillus subtilis strain DB 101 and fosthiazate, Bacillus subtilis strain DB 101 and cadusafos, Bacillus subtilis strain DB 101 and oxamyl, Bacillus subtilis strain DB 101 and thiodicarb, Bacillus subtilis strain DB 101 and dimethoate, Bacillus subtilis strain DB 101 and ethoprophos, Bacillus subtilis strain DB 101 and terbufos, Bacillus subtilis strain DB 101 and abamectin, Bacillus subtilis strain DB 101 and methyl bromide and other alkyl halides, Bacillus subtilis strain DB 101 and methyl isocyanate generators selected from diazomet and metam, Bacillus subtilis strain DB 101 and fluazaindolizine, Bacillus subtilis strain DB 101 and fluensulfone, Bacillus subtilis strain DB 101 and fluopyram, Bacillus subtilis strain DB 101 and tioxazafen, Bacillus subtilis strain DB 101 and N-[1-(2,6-difluorophenyl)-1H-pyrazol-3-yl]-2-trifluoromethylbenzamide, Bacillus subtilis strain DB 101 and cis-Jasmone, Bacillus subtilis strain DB 101 and harpin, Bacillus subtilis strain DB 101 and Azadirachta indica oil, Bacillus subtilis strain DB 101 and Azadirachtin, Bacillus subtilis strain DB 102 and alanycarb, Bacillus subtilis strain DB 102 and aldicarb, Bacillus subtilis strain DB 102 and carbofuran, Bacillus subtilis strain DB 102 and carbosulfan, Bacillus subtilis strain DB 102 and fosthiazate, Bacillus subtilis strain DB 102 and cadusafos, Bacillus subtilis strain DB 102 and oxamyl, Bacillus subtilis strain DB 102 and thiodicarb, Bacillus subtilis strain DB 102 and dimethoate, Bacillus subtilis strain DB 102 and ethoprophos, Bacillus subtilis strain DB 102 and terbufos, Bacillus subtilis strain DB 102 and abamectin, Bacillus subtilis strain DB 102 and methyl bromide and other alkyl halides, Bacillus subtilis strain DB 102 and methyl isocyanate generators selected from diazomet and metam, Bacillus subtilis strain DB 102 and fluazaindolizine, Bacillus subtilis strain DB 102 and fluensulfone, Bacillus subtilis strain DB 102 and fluopyram, Bacillus subtilis strain DB 102 and tioxazafen, Bacillus subtilis strain DB 102 and N-[1-(2,6-difluorophenyl)-1H-pyrazol-3-yl]-2-trifluoromethylbenzamide, Bacillus subtilis strain DB 102 and cis-Jasmone, Bacillus subtilis strain DB 102 and harpin, Bacillus subtilis strain DB 102 and Azadirachta indica oil, Bacillus subtilis strain DB 102 and Azadirachtin, Bacillus subtilis strain MBI 600 and alanycarb, Bacillus subtilis strain MBI 600 and aldicarb, Bacillus subtilis strain MBI 600 and carbofuran, Bacillus subtilis strain MBI 600 and carbosulfan, Bacillus subtilis strain MBI 600 and fosthiazate, Bacillus subtilis strain MBI 600 and cadusafos, Bacillus subtilis strain MBI 600 and oxamyl, Bacillus subtilis strain MBI 600 and thiodicarb, Bacillus subtilis strain MBI 600 and dimethoate, Bacillus subtilis strain MBI 600 and ethoprophos, Bacillus subtilis strain MBI 600 and terbufos, Bacillus subtilis strain MBI 600 and abamectin, Bacillus subtilis strain MBI 600 and methyl bromide and other alkyl halides, Bacillus subtilis strain MBI 600 and methyl isocyanate generators selected from diazomet and metam, Bacillus subtilis strain MBI 600 and fluazaindolizine, Bacillus subtilis strain MBI 600 and fluensulfone, Bacillus subtilis strain MBI 600 and fluopyram, Bacillus subtilis strain MBI 600 and tioxazafen, Bacillus subtilis strain MBI 600 and N-[1-(2,6-difluorophenyl)-1H-pyrazol-3-yl]-2-trifluoromethylbenzamide, Bacillus subtilis strain MBI 600 and cis-Jasmone, Bacillus subtilis strain MBI 600 and harpin, Bacillus subtilis strain MBI 600 and Azadirachta indica oil, Bacillus subtilis strain MBI 600 and Azadirachtin, Bacillus subtilis strain Y1336 and alanycarb, Bacillus subtilis strain Y1336 and aldicarb, Bacillus subtilis strain Y1336 and carbofuran, Bacillus subtilis strain Y1336 and carbosulfan, Bacillus subtilis strain Y1336 and fosthiazate, Bacillus subtilis strain Y1336 and cadusafos, Bacillus subtilis strain Y1336 and oxamyl, Bacillus subtilis strain Y1336 and thiodicarb, Bacillus subtilis strain Y1336 and dimethoate, Bacillus subtilis strain Y1336 and ethoprophos, Bacillus subtilis strain Y1336 and terbufos, Bacillus subtilis strain Y1336 and abamectin, Bacillus subtilis strain Y1336 and methyl bromide and other alkyl halides, Bacillus subtilis strain Y1336 and methyl isocyanate generators selected from diazomet and metam, Bacillus subtilis strain Y1336 and fluazaindolizine, Bacillus subtilis strain Y1336 and fluensulfone, Bacillus subtilis strain Y1336 and fluopyram, Bacillus subtilis strain Y1336 and tioxazafen, Bacillus subtilis strain Y1336 and N-[1-(2,6-difluorophenyl)-1H-pyrazol-3-yl]-2-trifluoromethylbenzamide, Bacillus subtilis strain Y1336 and cis-Jasmone, Bacillus subtilis strain Y1336 and harpin, Bacillus subtilis strain Y1336 and Azadirachta indica oil, Bacillus subtilis strain Y1336 and Azadirachtin, Bacillus subtilis var. amyloliquefaciens strain FZB24 and alanycarb, Bacillus subtilis var. amyloliquefaciens strain FZB24 and aldicarb, Bacillus subtilis var. amyloliquefaciens strain FZB24 and carbofuran, Bacillus subtilis var. amyloliquefaciens strain FZB24 and carbosulfan, Bacillus subtilis var. amyloliquefaciens strain FZB24 and fosthiazate, Bacillus subtilis var. amyloliquefaciens strain FZB24 and cadusafos, Bacillus subtilis var. amyloliquefaciens strain FZB24 and oxamyl, Bacillus subtilis var. amyloliquefaciens strain FZB24 and thiodicarb, Bacillus subtilis var. amyloliquefaciens strain FZB24 and dimethoate, Bacillus subtilis var. amyloliquefaciens strain FZB24 and ethoprophos, Bacillus subtilis var. amyloliquefaciens strain FZB24 and terbufos, Bacillus subtilis var. amyloliquefaciens strain FZB24 and abamectin, Bacillus subtilis var. amyloliquefaciens strain FZB24 and methyl bromide and other alkyl halides, Bacillus subtilis var. amyloliquefaciens strain FZB24 and methyl isocyanate generators selected from diazomet and metam, Bacillus subtilis var. amyloliquefaciens strain FZB24 and fluazaindolizine, Bacillus subtilis var. amyloliquefaciens strain FZB24 and fluensulfone, Bacillus subtilis var. amyloliquefaciens strain FZB24 and fluopyram, Bacillus subtilis var. amyloliquefaciens strain FZB24 and tioxazafen, Bacillus subtilis var. amyloliquefaciens strain FZB24 and N-[1-(2,6-difluorophenyl)-1H-pyrazol-3-yl]-2-trifluoromethylbenzamide, Bacillus subtilis var. amyloliquefaciens strain FZB24 and cis-Jasmone, Bacillus subtilis var. amyloliquefaciens strain FZB24 and harpin, Bacillus subtilis var. amyloliquefaciens strain FZB24 and Azadirachta indica oil, Bacillus subtilis var. amyloliquefaciens strain FZB24 and Azadirachtin, Bacillus thuringiensis strain EX297512 and alanycarb, Bacillus thuringiensis strain EX297512 and aldicarb, Bacillus thuringiensis strain EX297512 and carbofuran, Bacillus thuringiensis strain EX297512 and carbosulfan, Bacillus thuringiensis strain EX297512 and fosthiazate, Bacillus thuringiensis strain EX297512 and cadusafos, Bacillus thuringiensis strain EX297512 and oxamyl, Bacillus thuringiensis strain EX297512 and thiodicarb, Bacillus thuringiensis strain EX297512 and dimethoate, Bacillus thuringiensis strain EX297512 and ethoprophos, Bacillus thuringiensis strain EX297512 and terbufos, Bacillus thuringiensis strain EX297512 and abamectin, Bacillus thuringiensis strain EX297512 and methyl bromide and other alkyl halides, Bacillus thuringiensis strain EX297512 and methyl isocyanate generators selected from diazomet and metam, Bacillus thuringiensis strain EX297512 and fluazaindolizine, Bacillus thuringiensis strain EX297512 and fluensulfone, Bacillus thuringiensis strain EX297512 and fluopyram, Bacillus thuringiensis strain EX297512 and tioxazafen, Bacillus thuringiensis strain EX297512 and N-[1-(2,6-difluorophenyl)-1H-pyrazol-3-yl]-2-trifluoromethylbenzamide, Bacillus thuringiensis strain EX297512 and cis-Jasmone, Bacillus thuringiensis strain EX297512 and harpin, Bacillus thuringiensis strain EX297512 and Azadirachta indica oil, Bacillus thuringiensis strain EX297512 and Azadirachtin, Bacillus thuringiensis strain CR-371 and alanycarb, Bacillus thuringiensis strain CR-371 and aldicarb, Bacillus thuringiensis strain CR-371 and carbofuran, Bacillus thuringiensis strain CR-371 and carbosulfan, Bacillus thuringiensis strain CR-371 and fosthiazate, Bacillus thuringiensis strain CR-371 and cadusafos, Bacillus thuringiensis strain CR-371 and oxamyl, Bacillus thuringiensis strain CR-371 and thiodicarb, Bacillus thuringiensis strain CR-371 and dimethoate, Bacillus thuringiensis strain CR-371 and ethoprophos, Bacillus thuringiensis strain CR-371 and terbufos, Bacillus thuringiensis strain CR-371 and abamectin, Bacillus thuringiensis strain CR-371 and methyl bromide and other alkyl halides, Bacillus thuringiensis strain CR-371 and methyl isocyanate generators selected from diazomet and metam, Bacillus thuringiensis strain CR-371 and fluazaindolizine, Bacillus thuringiensis strain CR-371 and fluensulfone, Bacillus thuringiensis strain CR-371 and fluopyram, Bacillus thuringiensis strain CR-371 and tioxazafen, Bacillus thuringiensis strain CR-371 and N-[1-(2,6-difluorophenyl)-1H-pyrazol-3-yl]-2-trifluoromethylbenzamide, Bacillus thuringiensis strain CR-371 and cis-Jasmone, Bacillus thuringiensis strain CR-371 and harpin, Bacillus thuringiensis strain CR-371 and Azadirachta indica oil, Bacillus thuringiensis strain CR-371 and Azadirachtin, Bacillus thuringiensis strain AQ52 and alanycarb, Bacillus thuringiensis strain AQ52 and aldicarb, Bacillus thuringiensis strain AQ52 and carbofuran, Bacillus thuringiensis strain AQ52 and carbosulfan, Bacillus thuringiensis strain AQ52 and fosthiazate, Bacillus thuringiensis strain AQ52 and cadusafos, Bacillus thuringiensis strain AQ52 and oxamyl, Bacillus thuringiensis strain AQ52 and thiodicarb, Bacillus thuringiensis strain AQ52 and dimethoate, Bacillus thuringiensis strain AQ52 and ethoprophos, Bacillus thuringiensis strain AQ52 and terbufos, Bacillus thuringiensis strain AQ52 and abamectin, Bacillus thuringiensis strain AQ52 and methyl bromide and other alkyl halides, Bacillus thuringiensis strain AQ52 and methyl isocyanate generators selected from diazomet and metam, Bacillus thuringiensis strain AQ52 and fluazaindolizine, Bacillus thuringiensis strain AQ52 and fluensulfone, Bacillus thuringiensis strain AQ52 and fluopyram, Bacillus thuringiensis strain AQ52 and tioxazafen, Bacillus thuringiensis strain AQ52 and N-[1-(2,6-difluorophenyl)-1H-pyrazol-3-yl]-2-trifluoromethylbenzamide, Bacillus thuringiensis strain AQ52 and cis-Jasmone, Bacillus thuringiensis strain AQ52 and harpin, Bacillus thuringiensis strain AQ52 and Azadirachta indica oil, Bacillus thuringiensis strain AQ52 and Azadirachtin, Bacillus pumilus strain GB34 and alanycarb, Bacillus pumilus strain GB34 and aldicarb, Bacillus pumilus strain GB34 and carbofuran, Bacillus pumilus strain GB34 and carbosulfan, Bacillus pumilus strain GB34 and fosthiazate, Bacillus pumilus strain GB34 and cadusafos, Bacillus pumilus strain GB34 and oxamyl, Bacillus pumilus strain GB34 and thiodicarb, Bacillus pumilus strain GB34 and dimethoate, Bacillus pumilus strain GB34 and ethoprophos, Bacillus pumilus strain GB34 and terbufos, Bacillus pumilus strain GB34 and abamectin, Bacillus pumilus strain GB34 and methyl bromide and other alkyl halides, Bacillus pumilus strain GB34 and methyl isocyanate generators selected from diazomet and metam, Bacillus pumilus strain GB34 and fluazaindolizine, Bacillus pumilus strain GB34 and fluensulfone, Bacillus pumilus strain GB34 and fluopyram, Bacillus pumilus strain GB34 and tioxazafen, Bacillus pumilus strain GB34 and N-[1-(2,6-difluorophenyl)-1H-pyrazol-3-yl]-2-trifluoromethylbenzamide, Bacillus pumilus strain GB34 and cis-Jasmone, Bacillus pumilus strain GB34 and harpin, Bacillus pumilus strain GB34 and Azadirachta indica oil, Bacillus pumilus strain GB34 and Azadirachtin, Bacillus pumilus strain QST2808 and alanycarb, Bacillus pumilus strain QST2808 and aldicarb, Bacillus pumilus strain QST2808 and carbofuran, Bacillus pumilus strain QST2808 and carbosulfan, Bacillus pumilus strain QST2808 and fosthiazate, Bacillus pumilus strain QST2808 and cadusafos, Bacillus pumilus strain QST2808 and oxamyl, Bacillus pumilus strain QST2808 and thiodicarb, Bacillus pumilus strain QST2808 and dimethoate, Bacillus pumilus strain QST2808 and ethoprophos, Bacillus pumilus strain QST2808 and terbufos, Bacillus pumilus strain QST2808 and abamectin, Bacillus pumilus strain QST2808 and methyl bromide and other alkyl halides, Bacillus pumilus strain QST2808 and methyl isocyanate generators selected from diazomet and metam, Bacillus pumilus strain QST2808 and fluazaindolizine, Bacillus pumilus strain QST2808 and fluensulfone, Bacillus pumilus strain QST2808 and fluopyram, Bacillus pumilus strain QST2808 and tioxazafen, Bacillus pumilus strain QST2808 and N-[1-(2,6-difluorophenyl)-1H-pyrazol-3-yl]-2-trifluoromethylbenzamide, Bacillus pumilus strain QST2808 and cis-Jasmone, Bacillus pumilus strain QST2808 and harpin, Bacillus pumilus strain QST2808 and Azadirachta indica oil, Bacillus pumilus strain QST2808 and Azadirachtin, Bacillus pumilus strain BU F-33 and alanycarb, Bacillus pumilus strain BU F-33 and aldicarb, Bacillus pumilus strain BU F-33 and carbofuran, Bacillus pumilus strain BU F-33 and carbosulfan, Bacillus pumilus strain BU F-33 and fosthiazate, Bacillus pumilus strain BU F-33 and cadusafos, Bacillus pumilus strain BU F-33 and oxamyl, Bacillus pumilus strain BU F-33 and thiodicarb, Bacillus pumilus strain BU F-33 and dimethoate, Bacillus pumilus strain BU F-33 and ethoprophos, Bacillus pumilus strain BU F-33 and terbufos, Bacillus pumilus strain BU F-33 and abamectin, Bacillus pumilus strain BU F-33 and methyl bromide and other alkyl halides, Bacillus pumilus strain BU F-33 and methyl isocyanate generators selected from diazomet and metam, Bacillus pumilus strain BU F-33 and fluazaindolizine, Bacillus pumilus strain BU F-33 and fluensulfone, Bacillus pumilus strain BU F-33 and fluopyram, Bacillus pumilus strain BU F-33 and tioxazafen, Bacillus pumilus strain BU F-33 and N-[1-(2,6-difluorophenyl)-1H-pyrazol-3-yl]-2-trifluoromethylbenzamide, Bacillus pumilus strain BU F-33 and cis-Jasmone, Bacillus pumilus strain BU F-33 and harpin, Bacillus pumilus strain BU F-33 and Azadirachta indica oil, Bacillus pumilus strain BU F-33 and Azadirachtin, Bacillus pumilus strain AQ717 and alanycarb, Bacillus pumilus strain AQ717 and aldicarb, Bacillus pumilus strain AQ717 and carbofuran, Bacillus pumilus strain AQ717 and carbosulfan, Bacillus pumilus strain AQ717 and fosthiazate, Bacillus pumilus strain AQ717 and cadusafos, Bacillus pumilus strain AQ717 and oxamyl, Bacillus pumilus strain AQ717 and thiodicarb, Bacillus pumilus strain AQ717 and dimethoate, Bacillus pumilus strain AQ717 and ethoprophos, Bacillus pumilus strain AQ717 and terbufos, Bacillus pumilus strain AQ717 and abamectin, Bacillus pumilus strain AQ717 and methyl bromide and other alkyl halides, Bacillus pumilus strain AQ717 and methyl isocyanate generators selected from diazomet and metam, Bacillus pumilus strain AQ717 and fluazaindolizine, Bacillus pumilus strain AQ717 and fluensulfone, Bacillus pumilus strain AQ717 and fluopyram, Bacillus pumilus strain AQ717 and tioxazafen, Bacillus pumilus strain AQ717 and N-[1-(2,6-difluorophenyl)-1H-pyrazol-3-yl]-2-trifluoromethylbenzamide, Bacillus pumilus strain AQ717 and cis-Jasmone, Bacillus pumilus strain AQ717 and harpin, Bacillus pumilus strain AQ717 and Azadirachta indica oil, Bacillus pumilus strain AQ717 and Azadirachtin, Brevibacillus laterosporus strain ATCC 64 and alanycarb, Brevibacillus laterosporus strain ATCC 64 and aldicarb, Brevibacillus laterosporus strain ATCC 64 and carbofuran, Brevibacillus laterosporus strain ATCC 64 and carbosulfan, Brevibacillus laterosporus strain ATCC 64 and fosthiazate, Brevibacillus laterosporus strain ATCC 64 and cadusafos, Brevibacillus laterosporus strain ATCC 64 and oxamyl, Brevibacillus laterosporus strain ATCC 64 and thiodicarb, Brevibacillus laterosporus strain ATCC 64 and dimethoate, Brevibacillus laterosporus strain ATCC 64 and ethoprophos, Brevibacillus laterosporus strain ATCC 64 and terbufos, Brevibacillus laterosporus strain ATCC 64 and abamectin, Brevibacillus laterosporus strain ATCC 64 and methyl bromide and other alkyl halides, Brevibacillus laterosporus strain ATCC 64 and methyl isocyanate generators selected from diazomet and metam, Brevibacillus laterosporus strain ATCC 64 and fluazaindolizine, Brevibacillus laterosporus strain ATCC 64 and fluensulfone, Brevibacillus laterosporus strain ATCC 64 and fluopyram, Brevibacillus laterosporus strain ATCC 64 and tioxazafen, Brevibacillus laterosporus strain ATCC 64 and N-[1-(2,6-difluorophenyl)-1H-pyrazol-3-yl]-2-trifluoromethylbenzamide, Brevibacillus laterosporus strain ATCC 64 and cis-Jasmone, Brevibacillus laterosporus strain ATCC 64 and harpin, Brevibacillus laterosporus strain ATCC 64 and Azadirachta indica oil, Brevibacillus laterosporus strain ATCC 64 and Azadirachtin, Brevibacillus laterosporus strain NRS 1111 and alanycarb, Brevibacillus laterosporus strain NRS 1111 and aldicarb, Brevibacillus laterosporus strain NRS 1111 and carbofuran, Brevibacillus laterosporus strain NRS 1111 and carbosulfan, Brevibacillus laterosporus strain NRS 1111 and fosthiazate, Brevibacillus laterosporus strain NRS 1111 and cadusafos, Brevibacillus laterosporus strain NRS 1111 and oxamyl, Brevibacillus laterosporus strain NRS 1111 and thiodicarb, Brevibacillus laterosporus strain NRS 1111 and dimethoate, Brevibacillus laterosporus strain NRS 1111 and ethoprophos, Brevibacillus laterosporus strain NRS 1111 and terbufos, Brevibacillus laterosporus strain NRS 1111 and abamectin, Brevibacillus laterosporus strain NRS 1111 and methyl bromide and other alkyl halides, Brevibacillus laterosporus strain NRS 1111 and methyl isocyanate generators selected from diazomet and metam, Brevibacillus laterosporus strain NRS 1111 and fluazaindolizine, Brevibacillus laterosporus strain NRS 1111 and fluensulfone, Brevibacillus laterosporus strain NRS 1111 and fluopyram, Brevibacillus laterosporus strain NRS 1111 and tioxazafen, Brevibacillus laterosporus strain NRS 1111 and N-[1-(2,6-difluorophenyl)-1H-pyrazol-3-yl]-2-trifluoromethylbenzamide, Brevibacillus laterosporus strain NRS 1111 and cis-Jasmone, Brevibacillus laterosporus strain NRS 1111 and harpin, Brevibacillus laterosporus strain NRS 1111 and Azadirachta indica oil, Brevibacillus laterosporus strain NRS 1111 and Azadirachtin, Brevibacillus laterosporus strain NRS 1645 and alanycarb, Brevibacillus laterosporus strain NRS 1645 and aldicarb, Brevibacillus laterosporus strain NRS 1645 and carbofuran, Brevibacillus laterosporus strain NRS 1645 and carbosulfan, Brevibacillus laterosporus strain NRS 1645 and fosthiazate, Brevibacillus laterosporus strain NRS 1645 and cadusafos, Brevibacillus laterosporus strain NRS 1645 and oxamyl, Brevibacillus laterosporus strain NRS 1645 and thiodicarb, Brevibacillus laterosporus strain NRS 1645 and dimethoate, Brevibacillus laterosporus strain NRS 1645 and ethoprophos, Brevibacillus laterosporus strain NRS 1645 and terbufos, Brevibacillus laterosporus strain NRS 1645 and abamectin, Brevibacillus laterosporus strain NRS 1645 and methyl bromide and other alkyl halides, Brevibacillus laterosporus strain NRS 1645 and methyl isocyanate generators selected from diazomet and metam, Brevibacillus laterosporus strain NRS 1645 and fluazaindolizine, Brevibacillus laterosporus strain NRS 1645 and fluensulfone, Brevibacillus laterosporus strain NRS 1645 and fluopyram, Brevibacillus laterosporus strain NRS 1645 and tioxazafen, Brevibacillus laterosporus strain NRS 1645 and N-[1-(2,6-difluorophenyl)-1H-pyrazol-3-yl]-2-trifluoromethylbenzamide, Brevibacillus laterosporus strain NRS 1645 and cis-Jasmone, Brevibacillus laterosporus strain NRS 1645 and harpin, Brevibacillus laterosporus strain NRS 1645 and Azadirachta indica oil, Brevibacillus laterosporus strain NRS 1645 and Azadirachtin, Brevibacillus laterosporus strain NRS 1647 and alanycarb, Brevibacillus laterosporus strain NRS 1647 and aldicarb, Brevibacillus laterosporus strain NRS 1647 and carbofuran, Brevibacillus laterosporus strain NRS 1647 and carbosulfan, Brevibacillus laterosporus strain NRS 1647 and fosthiazate, Brevibacillus laterosporus strain NRS 1647 and cadusafos, Brevibacillus laterosporus strain NRS 1647 and oxamyl, Brevibacillus laterosporus strain NRS 1647 and thiodicarb, Brevibacillus laterosporus strain NRS 1647 and dimethoate, Brevibacillus laterosporus strain NRS 1647 and ethoprophos, Brevibacillus laterosporus strain NRS 1647 and terbufos, Brevibacillus laterosporus strain NRS 1647 and abamectin, Brevibacillus laterosporus strain NRS 1647 and methyl bromide and other alkyl halides, Brevibacillus laterosporus strain NRS 1647 and methyl isocyanate generators selected from diazomet and metam, Brevibacillus laterosporus strain NRS 1647 and fluazaindolizine, Brevibacillus laterosporus strain NRS 1647 and fluensulfone, Brevibacillus laterosporus strain NRS 1647 and fluopyram, Brevibacillus laterosporus strain NRS 1647 and tioxazafen, Brevibacillus laterosporus strain NRS 1647 and N-[1-(2,6-difluorophenyl)-1H-pyrazol-3-yl]-2-trifluoromethylbenzamide, Brevibacillus laterosporus strain NRS 1647 and cis-Jasmone, Brevibacillus laterosporus strain NRS 1647 and harpin, Brevibacillus laterosporus strain NRS 1647 and Azadirachta indica oil, Brevibacillus laterosporus strain NRS 1647 and Azadirachtin, Brevibacillus laterosporus strain BPM3 and alanycarb, Brevibacillus laterosporus strain BPM3 and aldicarb, Brevibacillus laterosporus strain BPM3 and carbofuran, Brevibacillus laterosporus strain BPM3 and carbosulfan, Brevibacillus laterosporus strain BPM3 and fosthiazate, Brevibacillus laterosporus strain BPM3 and cadusafos, Brevibacillus laterosporus strain BPM3 and oxamyl, Brevibacillus laterosporus strain BPM3 and thiodicarb, Brevibacillus laterosporus strain BPM3 and dimethoate, Brevibacillus laterosporus strain BPM3 and ethoprophos, Brevibacillus laterosporus strain BPM3 and terbufos, Brevibacillus laterosporus strain BPM3 and abamectin, Brevibacillus laterosporus strain BPM3 and methyl bromide and other alkyl halides, Brevibacillus laterosporus strain BPM3 and methyl isocyanate generators selected from diazomet and metam, Brevibacillus laterosporus strain BPM3 and fluazaindolizine, Brevibacillus laterosporus strain BPM3 and fluensulfone, Brevibacillus laterosporus strain BPM3 and fluopyram, Brevibacillus laterosporus strain BPM3 and tioxazafen, Brevibacillus laterosporus strain BPM3 and N-[1-(2,6-difluorophenyl)-1H-pyrazol-3-yl]-2-trifluoromethylbenzamide, Brevibacillus laterosporus strain BPM3 and cis-Jasmone, Brevibacillus laterosporus strain BPM3 and harpin, Brevibacillus laterosporus strain BPM3 and Azadirachta indica oil, Brevibacillus laterosporus strain BPM3 and Azadirachtin, Brevibacillus laterosporus strain G4 and alanycarb, Brevibacillus laterosporus strain G4 and aldicarb, Brevibacillus laterosporus strain G4 and carbofuran, Brevibacillus laterosporus strain G4 and carbosulfan, Brevibacillus laterosporus strain G4 and fosthiazate, Brevibacillus laterosporus strain G4 and cadusafos, Brevibacillus laterosporus strain G4 and oxamyl, Brevibacillus laterosporus strain G4 and thiodicarb, Brevibacillus laterosporus strain G4 and dimethoate, Brevibacillus laterosporus strain G4 and ethoprophos, Brevibacillus laterosporus strain G4 and terbufos, Brevibacillus laterosporus strain G4 and abamectin, Brevibacillus laterosporus strain G4 and methyl bromide and other alkyl halides, Brevibacillus laterosporus strain G4 and methyl isocyanate generators selected from diazomet and metam, Brevibacillus laterosporus strain G4 and fluazaindolizine, Brevibacillus laterosporus strain G4 and fluensulfone, Brevibacillus laterosporus strain G4 and fluopyram, Brevibacillus laterosporus strain G4 and tioxazafen, Brevibacillus laterosporus strain G4 and N-[1-(2,6-difluorophenyl)-1H-pyrazol-3-yl]-2-trifluoromethylbenzamide, Brevibacillus laterosporus strain G4 and cis-Jasmone, Brevibacillus laterosporus strain G4 and harpin, Brevibacillus laterosporus strain G4 and Azadirachta indica oil, Brevibacillus laterosporus strain G4 and Azadirachtin, Brevibacillus laterosporus strain NCIMB 41419 and alanycarb, Brevibacillus laterosporus strain NCIMB 41419 and aldicarb, Brevibacillus laterosporus strain NCIMB 41419 and carbofuran, Brevibacillus laterosporus strain NCIMB 41419 and carbosulfan, Brevibacillus laterosporus strain NCIMB 41419 and fosthiazate, Brevibacillus laterosporus strain NCIMB 41419 and cadusafos, Brevibacillus laterosporus strain NCIMB 41419 and oxamyl, Brevibacillus laterosporus strain NCIMB 41419 and thiodicarb, Brevibacillus laterosporus strain NCIMB 41419 and dimethoate, Brevibacillus laterosporus strain NCIMB 41419 and ethoprophos, Brevibacillus laterosporus strain NCIMB 41419 and terbufos, Brevibacillus laterosporus strain NCIMB 41419 and abamectin, Brevibacillus laterosporus strain NCIMB 41419 and methyl bromide and other alkyl halides, Brevibacillus laterosporus strain NCIMB 41419 and methyl isocyanate generators selected from diazomet and metam, Brevibacillus laterosporus strain NCIMB 41419 and fluazaindolizine, Brevibacillus laterosporus strain NCIMB 41419 and fluensulfone, Brevibacillus laterosporus strain NCIMB 41419 and fluopyram, Brevibacillus laterosporus strain NCIMB 41419 and tioxazafen, Brevibacillus laterosporus strain NCIMB 41419 and N-[1-(2,6-difluorophenyl)-1H-pyrazol-3-yl]-2-trifluoromethylbenzamide, Brevibacillus laterosporus strain NCIMB 41419 and cis-Jasmone, Brevibacillus laterosporus strain NCIMB 41419 and harpin, Brevibacillus laterosporus strain NCIMB 41419 and Azadirachta indica oil, Brevibacillus laterosporus strain NCIMB 41419 and Azadirachtin, Burkholderia rinojensis strain A396 and alanycarb, Burkholderia rinojensis strain A396 and aldicarb, Burkholderia rinojensis strain A396 and carbofuran, Burkholderia rinojensis strain A396 and carbosulfan, Burkholderia rinojensis strain A396 and fosthiazate, Burkholderia rinojensis strain A396 and cadusafos, Burkholderia rinojensis strain A396 and oxamyl, Burkholderia rinojensis strain A396 and thiodicarb, Burkholderia rinojensis strain A396 and dimethoate, Burkholderia rinojensis strain A396 and ethoprophos, Burkholderia rinojensis strain A396 and terbufos, Burkholderia rinojensis strain A396 and abamectin, Burkholderia rinojensis strain A396 and methyl bromide and other alkyl halides, Burkholderia rinojensis strain A396 and methyl isocyanate generators selected from diazomet and metam, Burkholderia rinojensis strain A396 and fluazaindolizine, Burkholderia rinojensis strain A396 and fluensulfone, Burkholderia rinojensis strain A396 and fluopyram, Burkholderia rinojensis strain A396 and tioxazafen, Burkholderia rinojensis strain A396 and N-[1-(2,6-difluorophenyl)-1H-pyrazol-3-yl]-2-trifluoromethylbenzamide, Burkholderia rinojensis strain A396 and cis-Jasmone, Burkholderia rinojensis strain A396 and harpin, Burkholderia rinojensis strain A396 and Azadirachta indica oil, Burkholderia rinojensis strain A396 and Azadirachtin, Lysobacter antibioticus strain 13-1 and alanycarb, Lysobacter antibioticus strain 13-1 and aldicarb, Lysobacter antibioticus strain 13-1 and carbofuran, Lysobacter antibioticus strain 13-1 and carbosulfan, Lysobacter antibioticus strain 13-1 and fosthiazate, Lysobacter antibioticus strain 13-1 and cadusafos, Lysobacter antibioticus strain 13-1 and oxamyl, Lysobacter antibioticus strain 13-1 and thiodicarb, Lysobacter antibioticus strain 13-1 and dimethoate, Lysobacter antibioticus strain 13-1 and ethoprophos, Lysobacter antibioticus strain 13-1 and terbufos, Lysobacter antibioticus strain 13-1 and abamectin, Lysobacter antibioticus strain 13-1 and methyl bromide and other alkyl halides, Lysobacter antibioticus strain 13-1 and methyl isocyanate generators selected from diazomet and metam, Lysobacter antibioticus strain 13-1 and fluazaindolizine, Lysobacter antibioticus strain 13-1 and fluensulfone, Lysobacter antibioticus strain 13-1 and fluopyram, Lysobacter antibioticus strain 13-1 and tioxazafen, Lysobacter antibioticus strain 13-1 and N-[1-(2,6-difluorophenyl)-1H-pyrazol-3-yl]-2-trifluoromethylbenzamide, Lysobacter antibioticus strain 13-1 and cis-Jasmone, Lysobacter antibioticus strain 13-1 and harpin, Lysobacter antibioticus strain 13-1 and Azadirachta indica oil, Lysobacter antibioticus strain 13-1 and Azadirachtin, Lysobacter enzymogenes strain C3 and alanycarb, Lysobacter enzymogenes strain C3 and aldicarb, Lysobacter enzymogenes strain C3 and carbofuran, Lysobacter enzymogenes strain C3 and carbosulfan, Lysobacter enzymogenes strain C3 and fosthiazate, Lysobacter enzymogenes strain C3 and cadusafos, Lysobacter enzymogenes strain C3 and oxamyl, Lysobacter enzymogenes strain C3 and thiodicarb, Lysobacter enzymogenes strain C3 and dimethoate, Lysobacter enzymogenes strain C3 and ethoprophos, Lysobacter enzymogenes strain C3 and terbufos, Lysobacter enzymogenes strain C3 and abamectin, Lysobacter enzymogenes strain C3 and methyl bromide and other alkyl halides, Lysobacter enzymogenes strain C3 and methyl isocyanate generators selected from diazomet and metam, Lysobacter enzymogenes strain C3 and fluazaindolizine, Lysobacter enzymogenes strain C3 and fluensulfone, Lysobacter enzymogenes strain C3 and fluopyram, Lysobacter enzymogenes strain C3 and tioxazafen, Lysobacter enzymogenes strain C3 and N-[1-(2,6-difluorophenyl)-1H-pyrazol-3-yl]-2-trifluoromethylbenzamide, Lysobacter enzymogenes strain C3 and cis-Jasmone, Lysobacter enzymogenes strain C3 and harpin, Lysobacter enzymogenes strain C3 and Azadirachta indica oil, Lysobacter enzymogenes strain C3 and Azadirachtin, Myrothecium verrucaria strain AARC-0255 and alanycarb, Myrothecium verrucaria strain AARC-0255 and aldicarb, Myrothecium verrucaria strain AARC-0255 and carbofuran, Myrothecium verrucaria strain AARC-0255 and carbosulfan, Myrothecium verrucaria strain AARC-0255 and fosthiazate, Myrothecium verrucaria strain AARC-0255 and cadusafos, Myrothecium verrucaria strain AARC-0255 and oxamyl, Myrothecium verrucaria strain AARC-0255 and thiodicarb, Myrothecium verrucaria strain AARC-0255 and dimethoate, Myrothecium verrucaria strain AARC-0255 and ethoprophos, Myrothecium verrucaria strain AARC-0255 and terbufos, Myrothecium verrucaria strain AARC-0255 and abamectin, Myrothecium verrucaria strain AARC-0255 and methyl bromide and other alkyl halides, Myrothecium verrucaria strain AARC-0255 and methyl isocyanate generators selected from diazomet and metam, Myrothecium verrucaria strain AARC-0255 and fluazaindolizine, Myrothecium verrucaria strain AARC-0255 and fluensulfone, Myrothecium verrucaria strain AARC-0255 and fluopyram, Myrothecium verrucaria strain AARC-0255 and tioxazafen, Myrothecium verrucaria strain AARC-0255 and N-[1-(2,6-difluorophenyl)-1H-pyrazol-3-yl]-2-trifluoromethylbenzamide, Myrothecium verrucaria strain AARC-0255 and cis-Jasmone, Myrothecium verrucaria strain AARC-0255 and harpin, Myrothecium verrucaria strain AARC-0255 and Azadirachta indica oil, Myrothecium verrucaria strain AARC-0255 and Azadirachtin, Paecilomyces lilacinus strain 251 and alanycarb, Paecilomyces lilacinus strain 251 and aldicarb, Paecilomyces lilacinus strain 251 and carbofuran, Paecilomyces lilacinus strain 251 and carbosulfan, Paecilomyces lilacinus strain 251 and fosthiazate, Paecilomyces lilacinus strain 251 and cadusafos, Paecilomyces lilacinus strain 251 and oxamyl, Paecilomyces lilacinus strain 251 and thiodicarb, Paecilomyces lilacinus strain 251 and dimethoate, Paecilomyces lilacinus strain 251 and ethoprophos, Paecilomyces lilacinus strain 251 and terbufos, Paecilomyces lilacinus strain 251 and abamectin, Paecilomyces lilacinus strain 251 and methyl bromide and other alkyl halides, Paecilomyces lilacinus strain 251 and methyl isocyanate generators selected from diazomet and metam, Paecilomyces lilacinus strain 251 and fluazaindolizine, Paecilomyces lilacinus strain 251 and fluensulfone, Paecilomyces lilacinus strain 251 and fluopyram, Paecilomyces lilacinus strain 251 and tioxazafen, Paecilomyces lilacinus strain 251 and N-[1-(2,6-difluorophenyl)-1H-pyrazol-3-yl]-2-trifluoromethylbenzamide, Paecilomyces lilacinus strain 251 and cis-Jasmone, Paecilomyces lilacinus strain 251 and harpin, Paecilomyces lilacinus strain 251 and Azadirachta indica oil, Paecilomyces lilacinus strain 251 and Azadirachtin, Paecilomyces variotii strain Q-09 and alanycarb, Paecilomyces variotii strain Q-09 and aldicarb, Paecilomyces variotii strain Q-09 and carbofuran, Paecilomyces variotii strain Q-09 and carbosulfan, Paecilomyces variotii strain Q-09 and fosthiazate, Paecilomyces variotii strain Q-09 and cadusafos, Paecilomyces variotii strain Q-09 and oxamyl, Paecilomyces variotii strain Q-09 and thiodicarb, Paecilomyces variotii strain Q-09 and dimethoate, Paecilomyces variotii strain Q-09 and ethoprophos, Paecilomyces variotii strain Q-09 and terbufos, Paecilomyces variotii strain Q-09 and abamectin, Paecilomyces variotii strain Q-09 and methyl bromide and other alkyl halides, Paecilomyces variotii strain Q-09 and methyl isocyanate generators selected from diazomet and metam, Paecilomyces variotii strain Q-09 and fluazaindolizine, Paecilomyces variotii strain Q-09 and fluensulfone, Paecilomyces variotii strain Q-09 and fluopyram, Paecilomyces variotii strain Q-09 and tioxazafen, Paecilomyces variotii strain Q-09 and N-[1-(2,6-difluorophenyl)-1H-pyrazol-3-yl]-2-trifluoromethylbenzamide, Paecilomyces variotii strain Q-09 and cis-Jasmone, Paecilomyces variotii strain Q-09 and harpin, Paecilomyces variotii strain Q-09 and Azadirachta indica oil, Paecilomyces variotii strain Q-09 and Azadirachtin, Trichoderma asperellum strain ICC 012 and alanycarb, Trichoderma asperellum strain ICC 012 and aldicarb, Trichoderma asperellum strain ICC 012 and carbofuran, Trichoderma asperellum strain ICC 012 and carbosulfan, Trichoderma asperellum strain ICC 012 and fosthiazate, Trichoderma asperellum strain ICC 012 and cadusafos, Trichoderma asperellum strain ICC 012 and oxamyl, Trichoderma asperellum strain ICC 012 and thiodicarb, Trichoderma asperellum strain ICC 012 and dimethoate, Trichoderma asperellum strain ICC 012 and ethoprophos, Trichoderma asperellum strain ICC 012 and terbufos, Trichoderma asperellum strain ICC 012 and abamectin, Trichoderma asperellum strain ICC 012 and methyl bromide and other alkyl halides, Trichoderma asperellum strain ICC 012 and methyl isocyanate generators selected from diazomet and metam, Trichoderma asperellum strain ICC 012 and fluazaindolizine, Trichoderma asperellum strain ICC 012 and fluensulfone, Trichoderma asperellum strain ICC 012 and fluopyram, Trichoderma asperellum strain ICC 012 and tioxazafen, Trichoderma asperellum strain ICC 012 and N-[1-(2,6-difluorophenyl)-1H-pyrazol-3-yl]-2-trifluoromethylbenzamide, Trichoderma asperellum strain ICC 012 and cis-Jasmone, Trichoderma asperellum strain ICC 012 and harpin, Trichoderma asperellum strain ICC 012 and Azadirachta indica oil, Trichoderma asperellum strain ICC 012 and Azadirachtin, Trichoderma asperellum strain SKT-1 and alanycarb, Trichoderma asperellum strain SKT-1 and aldicarb, Trichoderma asperellum strain SKT-1 and carbofuran, Trichoderma asperellum strain SKT-1 and carbosulfan, Trichoderma asperellum strain SKT-1 and fosthiazate, Trichoderma asperellum strain SKT-1 and cadusafos, Trichoderma asperellum strain SKT-1 and oxamyl, Trichoderma asperellum strain SKT-1 and thiodicarb, Trichoderma asperellum strain SKT-1 and dimethoate, Trichoderma asperellum strain SKT-1 and ethoprophos, Trichoderma asperellum strain SKT-1 and terbufos, Trichoderma asperellum strain SKT-1 and abamectin, Trichoderma asperellum strain SKT-1 and methyl bromide and other alkyl halides, Trichoderma asperellum strain SKT-1 and methyl isocyanate generators selected from diazomet and metam, Trichoderma asperellum strain SKT-1 and fluazaindolizine, Trichoderma asperellum strain SKT-1 and fluensulfone, Trichoderma asperellum strain SKT-1 and fluopyram, Trichoderma asperellum strain SKT-1 and tioxazafen, Trichoderma asperellum strain SKT-1 and N-[1-(2,6-difluorophenyl)-1H-pyrazol-3-yl]-2-trifluoromethylbenzamide, Trichoderma asperellum strain SKT-1 and cis-Jasmone, Trichoderma asperellum strain SKT-1 and harpin, Trichoderma asperellum strain SKT-1 and Azadirachta indica oil, Trichoderma asperellum strain SKT-1 and Azadirachtin, Trichoderma asperellum strain T34 and alanycarb, Trichoderma asperellum strain T34 and aldicarb, Trichoderma asperellum strain T34 and carbofuran, Trichoderma asperellum strain T34 and carbosulfan, Trichoderma asperellum strain T34 and fosthiazate, Trichoderma asperellum strain T34 and cadusafos, Trichoderma asperellum strain T34 and oxamyl, Trichoderma asperellum strain T34 and thiodicarb, Trichoderma asperellum strain T34 and dimethoate, Trichoderma asperellum strain T34 and ethoprophos, Trichoderma asperellum strain T34 and terbufos, Trichoderma asperellum strain T34 and abamectin, Trichoderma asperellum strain T34 and methyl bromide and other alkyl halides, Trichoderma asperellum strain T34 and methyl isocyanate generators selected from diazomet and metam, Trichoderma asperellum strain T34 and fluazaindolizine, Trichoderma asperellum strain T34 and fluensulfone, Trichoderma asperellum strain T34 and fluopyram, Trichoderma asperellum strain T34 and tioxazafen, Trichoderma asperellum strain T34 and N-[1-(2,6-difluorophenyl)-1H-pyrazol-3-yl]-2-trifluoromethylbenzamide, Trichoderma asperellum strain T34 and cis-Jasmone, Trichoderma asperellum strain T34 and harpin, Trichoderma asperellum strain T34 and Azadirachta indica oil, Trichoderma asperellum strain T34 and Azadirachtin, Trichoderma asperellum strain T25 and alanycarb, Trichoderma asperellum strain T25 and aldicarb, Trichoderma asperellum strain T25 and carbofuran, Trichoderma asperellum strain T25 and carbosulfan, Trichoderma asperellum strain T25 and fosthiazate, Trichoderma asperellum strain T25 and cadusafos, Trichoderma asperellum strain T25 and oxamyl, Trichoderma asperellum strain T25 and thiodicarb, Trichoderma asperellum strain T25 and dimethoate, Trichoderma asperellum strain T25 and ethoprophos, Trichoderma asperellum strain T25 and terbufos, Trichoderma asperellum strain T25 and abamectin, Trichoderma asperellum strain T25 and methyl bromide and other alkyl halides, Trichoderma asperellum strain T25 and methyl isocyanate generators selected from diazomet and metam, Trichoderma asperellum strain T25 and fluazaindolizine, Trichoderma asperellum strain T25 and fluensulfone, Trichoderma asperellum strain T25 and fluopyram, Trichoderma asperellum strain T25 and tioxazafen, Trichoderma asperellum strain T25 and N-[1-(2,6-difluorophenyl)-1H-pyrazol-3-yl]-2-trifluoromethylbenzamide, Trichoderma asperellum strain T25 and cis-Jasmone, Trichoderma asperellum strain T25 and harpin, Trichoderma asperellum strain T25 and Azadirachta indica oil, Trichoderma asperellum strain T25 and Azadirachtin, Trichoderma asperellum strain SF04 and alanycarb, Trichoderma asperellum strain SF04 and aldicarb, Trichoderma asperellum strain SF04 and carbofuran, Trichoderma asperellum strain SF04 and carbosulfan, Trichoderma asperellum strain SF04 and fosthiazate, Trichoderma asperellum strain SF04 and cadusafos, Trichoderma asperellum strain SF04 and oxamyl, Trichoderma asperellum strain SF04 and thiodicarb, Trichoderma asperellum strain SF04 and dimethoate, Trichoderma asperellum strain SF04 and ethoprophos, Trichoderma asperellum strain SF04 and terbufos, Trichoderma asperellum strain SF04 and abamectin, Trichoderma asperellum strain SF04 and methyl bromide and other alkyl halides, Trichoderma asperellum strain SF04 and methyl isocyanate generators selected from diazomet and metam, Trichoderma asperellum strain SF04 and fluazaindolizine, Trichoderma asperellum strain SF04 and fluensulfone, Trichoderma asperellum strain SF04 and fluopyram, Trichoderma asperellum strain SF04 and tioxazafen, Trichoderma asperellum strain SF04 and N-[1-(2,6-difluorophenyl)-1H-pyrazol-3-yl]-2-trifluoromethylbenzamide, Trichoderma asperellum strain SF04 and cis-Jasmone, Trichoderma asperellum strain SF04 and harpin, Trichoderma asperellum strain SF04 and Azadirachta indica oil, Trichoderma asperellum strain SF04 and Azadirachtin, Trichoderma asperellum strain TV1 and alanycarb, Trichoderma asperellum strain TV1 and aldicarb, Trichoderma asperellum strain TV1 and carbofuran, Trichoderma asperellum strain TV1 and carbosulfan, Trichoderma asperellum strain TV1 and fosthiazate, Trichoderma asperellum strain TV1 and cadusafos, Trichoderma asperellum strain TV1 and oxamyl, Trichoderma asperellum strain TV1 and thiodicarb, Trichoderma asperellum strain TV1 and dimethoate, Trichoderma asperellum strain TV1 and ethoprophos, Trichoderma asperellum strain TV1 and terbufos, Trichoderma asperellum strain TV1 and abamectin, Trichoderma asperellum strain TV1 and methyl bromide and other alkyl halides, Trichoderma asperellum strain TV1 and methyl isocyanate generators selected from diazomet and metam, Trichoderma asperellum strain TV1 and fluazaindolizine, Trichoderma asperellum strain TV1 and fluensulfone, Trichoderma asperellum strain TV1 and fluopyram, Trichoderma asperellum strain TV1 and tioxazafen, Trichoderma asperellum strain TV1 and N-[1-(2,6-difluorophenyl)-1H-pyrazol-3-yl]-2-trifluoromethylbenzamide, Trichoderma asperellum strain TV1 and cis-Jasmone, Trichoderma asperellum strain TV1 and harpin, Trichoderma asperellum strain TV1 and Azadirachta indica oil, Trichoderma asperellum strain TV1 and Azadirachtin, Trichoderma asperellum strain T11 and alanycarb, Trichoderma asperellum strain T11 and aldicarb, Trichoderma asperellum strain T11 and carbofuran, Trichoderma asperellum strain T11 and carbosulfan, Trichoderma asperellum strain T11 and fosthiazate, Trichoderma asperellum strain T11 and cadusafos, Trichoderma asperellum strain T11 and oxamyl, Trichoderma asperellum strain T11 and thiodicarb, Trichoderma asperellum strain T11 and dimethoate, Trichoderma asperellum strain T11 and ethoprophos, Trichoderma asperellum strain T11 and terbufos, Trichoderma asperellum strain T11 and abamectin, Trichoderma asperellum strain T11 and methyl bromide and other alkyl halides, Trichoderma asperellum strain T11 and methyl isocyanate generators selected from diazomet and metam, Trichoderma asperellum strain T11 and fluazaindolizine, Trichoderma asperellum strain T11 and fluensulfone, Trichoderma asperellum strain T11 and fluopyram, Trichoderma asperellum strain T11 and tioxazafen, Trichoderma asperellum strain T11 and N-[1-(2,6-difluorophenyl)-1H-pyrazol-3-yl]-2-trifluoromethylbenzamide, Trichoderma asperellum strain T11 and cis-Jasmone, Trichoderma asperellum strain T11 and harpin, Trichoderma asperellum strain T11 and Azadirachta indica oil, Trichoderma asperellum strain T11 and Azadirachtin, Trichoderma harzianum strain ICC012 and alanycarb, Trichoderma harzianum strain ICC012 and aldicarb, Trichoderma harzianum strain ICC012 and carbofuran, Trichoderma harzianum strain ICC012 and carbosulfan, Trichoderma harzianum strain ICC012 and fosthiazate, Trichoderma harzianum strain ICC012 and cadusafos, Trichoderma harzianum strain ICC012 and oxamyl, Trichoderma harzianum strain ICC012 and thiodicarb, Trichoderma harzianum strain ICC012 and dimethoate, Trichoderma harzianum strain ICC012 and ethoprophos, Trichoderma harzianum strain ICC012 and terbufos, Trichoderma harzianum strain ICC012 and abamectin, Trichoderma harzianum strain ICC012 and methyl bromide and other alkyl halides, Trichoderma harzianum strain ICC012 and methyl isocyanate generators selected from diazomet and metam, Trichoderma harzianum strain ICC012 and fluazaindolizine, Trichoderma harzianum strain ICC012 and fluensulfone, Trichoderma harzianum strain ICC012 and fluopyram, Trichoderma harzianum strain ICC012 and tioxazafen, Trichoderma harzianum strain ICC012 and N-[1-(2,6-difluorophenyl)-1H-pyrazol-3-yl]-2-trifluoromethylbenzamide, Trichoderma harzianum strain ICC012 and cis-Jasmone, Trichoderma harzianum strain ICC012 and harpin, Trichoderma harzianum strain ICC012 and Azadirachta indica oil, Trichoderma harzianum strain ICC012 and Azadirachtin, Trichoderma harzianum rifai T39 and alanycarb, Trichoderma harzianum rifai T39 and aldicarb, Trichoderma harzianum rifai T39 and carbofuran, Trichoderma harzianum rifai T39 and carbosulfan, Trichoderma harzianum rifai T39 and fosthiazate, Trichoderma harzianum rifai T39 and cadusafos, Trichoderma harzianum rifai T39 and oxamyl, Trichoderma harzianum rifai T39 and thiodicarb, Trichoderma harzianum rifai T39 and dimethoate, Trichoderma harzianum rifai T39 and ethoprophos, Trichoderma harzianum rifai T39 and terbufos, Trichoderma harzianum rifai T39 and abamectin, Trichoderma harzianum rifai T39 and methyl bromide and other alkyl halides, Trichoderma harzianum rifai T39 and methyl isocyanate generators selected from diazomet and metam, Trichoderma harzianum rifai T39 and fluazaindolizine, Trichoderma harzianum rifai T39 and fluensulfone, Trichoderma harzianum rifai T39 and fluopyram, Trichoderma harzianum rifai T39 and tioxazafen, Trichoderma harzianum rifai T39 and N-[1-(2,6-difluorophenyl)-1H-pyrazol-3-yl]-2-trifluoromethylbenzamide, Trichoderma harzianum rifai T39 and cis-Jasmone, Trichoderma harzianum rifai T39 and harpin, Trichoderma harzianum rifai T39 and Azadirachta indica oil, Trichoderma harzianum rifai T39 and Azadirachtin, Trichoderma harzianum rifai strain KRL-AG2 and alanycarb, Trichoderma harzianum rifai strain KRL-AG2 and aldicarb, Trichoderma harzianum rifai strain KRL-AG2 and carbofuran, Trichoderma harzianum rifai strain KRL-AG2 and carbosulfan, Trichoderma harzianum rifai strain KRL-AG2 and fosthiazate, Trichoderma harzianum rifai strain KRL-AG2 and cadusafos, Trichoderma harzianum rifai strain KRL-AG2 and oxamyl, Trichoderma harzianum rifai strain KRL-AG2 and thiodicarb, Trichoderma harzianum rifai strain KRL-AG2 and dimethoate, Trichoderma harzianum rifai strain KRL-AG2 and ethoprophos, Trichoderma harzianum rifai strain KRL-AG2 and terbufos, Trichoderma harzianum rifai strain KRL-AG2 and abamectin, Trichoderma harzianum rifai strain KRL-AG2 and methyl bromide and other alkyl halides, Trichoderma harzianum rifai strain KRL-AG2 and methyl isocyanate generators selected from diazomet and metam, Trichoderma harzianum rifai strain KRL-AG2 and fluazaindolizine, Trichoderma harzianum rifai strain KRL-AG2 and fluensulfone, Trichoderma harzianum rifai strain KRL-AG2 and fluopyram, Trichoderma harzianum rifai strain KRL-AG2 and tioxazafen, Trichoderma harzianum rifai strain KRL-AG2 and N-[1-(2,6-difluorophenyl)-1H-pyrazol-3-yl]-2-trifluoromethylbenzamide, Trichoderma harzianum rifai strain KRL-AG2 and cis-Jasmone, Trichoderma harzianum rifai strain KRL-AG2 and harpin, Trichoderma harzianum rifai strain KRL-AG2 and Azadirachta indica oil, Trichoderma harzianum rifai strain KRL-AG2 and Azadirachtin, Trichoderma viride strain TV1 and alanycarb, Trichoderma viride strain TV1 and aldicarb, Trichoderma viride strain TV1 and carbofuran, Trichoderma viride strain TV1 and carbosulfan, Trichoderma viride strain TV1 and fosthiazate, Trichoderma viride strain TV1 and cadusafos, Trichoderma viride strain TV1 and oxamyl, Trichoderma viride strain TV1 and thiodicarb, Trichoderma viride strain TV1 and dimethoate, Trichoderma viride strain TV1 and ethoprophos, Trichoderma viride strain TV1 and terbufos, Trichoderma viride strain TV1 and abamectin, Trichoderma viride strain TV1 and methyl bromide and other alkyl halides, Trichoderma viride strain TV1 and methyl isocyanate generators selected from diazomet and metam, Trichoderma viride strain TV1 and fluazaindolizine, Trichoderma viride strain TV1 and fluensulfone, Trichoderma viride strain TV1 and fluopyram, Trichoderma viride strain TV1 and tioxazafen, Trichoderma viride strain TV1 and N-[1-(2,6-difluorophenyl)-1H-pyrazol-3-yl]-2-trifluoromethylbenzamide, Trichoderma viride strain TV1 and cis-Jasmone, Trichoderma viride strain TV1 and harpin, Trichoderma viride strain TV1 and Azadirachta indica oil, Trichoderma viride strain TV1 and Azadirachtin, Trichoderma viride strain TV25 and alanycarb, Trichoderma viride strain TV25 and aldicarb, Trichoderma viride strain TV25 and carbofuran, Trichoderma viride strain TV25 and carbosulfan, Trichoderma viride strain TV25 and fosthiazate, Trichoderma viride strain TV25 and cadusafos, Trichoderma viride strain TV25 and oxamyl, Trichoderma viride strain TV25 and thiodicarb, Trichoderma viride strain TV25 and dimethoate, Trichoderma viride strain TV25 and ethoprophos, Trichoderma viride strain TV25 and terbufos, Trichoderma viride strain TV25 and abamectin, Trichoderma viride strain TV25 and methyl bromide and other alkyl halides, Trichoderma viride strain TV25 and methyl isocyanate generators selected from diazomet and metam, Trichoderma viride strain TV25 and fluazaindolizine, Trichoderma viride strain TV25 and fluensulfone, Trichoderma viride strain TV25 and fluopyram, Trichoderma viride strain TV25 and tioxazafen, Trichoderma viride strain TV25 and N-[1-(2,6-difluorophenyl)-1H-pyrazol-3-yl]-2-trifluoromethylbenzamide, Trichoderma viride strain TV25 and cis-Jasmone, Trichoderma viride strain TV25 and harpin, Trichoderma viride strain TV25 and Azadirachta indica oil, Trichoderma viride strain TV25 and Azadirachtin, Trichoderma atroviride strain CNCM I-1237 and alanycarb, Trichoderma atroviride strain CNCM I-1237 and aldicarb, Trichoderma atroviride strain CNCM I-1237 and carbofuran, Trichoderma atroviride strain CNCM I-1237 and carbosulfan, Trichoderma atroviride strain CNCM I-1237 and fosthiazate, Trichoderma atroviride strain CNCM I-1237 and cadusafos, Trichoderma atroviride strain CNCM I-1237 and oxamyl, Trichoderma atroviride strain CNCM I-1237 and thiodicarb, Trichoderma atroviride strain CNCM I-1237 and dimethoate, Trichoderma atroviride strain CNCM I-1237 and ethoprophos, Trichoderma atroviride strain CNCM I-1237 and terbufos, Trichoderma atroviride strain CNCM I-1237 and abamectin, Trichoderma atroviride strain CNCM I-1237 and methyl bromide and other alkyl halides, Trichoderma atroviride strain CNCM I-1237 and methyl isocyanate generators selected from diazomet and metam, Trichoderma atroviride strain CNCM I-1237 and fluazaindolizine, Trichoderma atroviride strain CNCM I-1237 and fluensulfone, Trichoderma atroviride strain CNCM I-1237 and fluopyram, Trichoderma atroviride strain CNCM I-1237 and tioxazafen, Trichoderma atroviride strain CNCM I-1237 and N-[1-(2,6-difluorophenyl)-1H-pyrazol-3-yl]-2-trifluoromethylbenzamide, Trichoderma atroviride strain CNCM I-1237 and cis-Jasmone, Trichoderma atroviride strain CNCM I-1237 and harpin, Trichoderma atroviride strain CNCM I-1237 and Azadirachta indica oil, Trichoderma atroviride strain CNCM I-1237 and Azadirachtin, Trichoderma atroviride strain CNCM I-1237 and alanycarb, Trichoderma atroviride strain CNCM I-1237 and aldicarb, Trichoderma atroviride strain CNCM I-1237 and carbofuran, Trichoderma atroviride strain CNCM I-1237 and carbosulfan, Trichoderma atroviride strain CNCM I-1237 and fosthiazate, Trichoderma atroviride strain CNCM I-1237 and cadusafos, Trichoderma atroviride strain CNCM I-1237 and oxamyl, Trichoderma atroviride strain CNCM I-1237 and thiodicarb, Trichoderma atroviride strain CNCM I-1237 and dimethoate, Trichoderma atroviride strain CNCM I-1237 and ethoprophos, Trichoderma atroviride strain CNCM I-1237 and terbufos, Trichoderma atroviride strain CNCM I-1237 and abamectin, Trichoderma atroviride strain CNCM I-1237 and methyl bromide and other alkyl halides, Trichoderma atroviride strain CNCM I-1237 and methyl isocyanate generators selected from diazomet and metam, Trichoderma atroviride strain CNCM I-1237 and fluazaindolizine, Trichoderma atroviride strain CNCM I-1237 and fluensulfone, Trichoderma atroviride strain CNCM I-1237 and fluopyram, Trichoderma atroviride strain CNCM I-1237 and tioxazafen, Trichoderma atroviride strain CNCM I-1237 and N-[1-(2,6-difluorophenyl)-1H-pyrazol-3-yl]-2-trifluoromethylbenzamide, Trichoderma atroviride strain CNCM I-1237 and cis-Jasmone, Trichoderma atroviride strain CNCM I-1237 and harpin, Trichoderma atroviride strain CNCM I-1237 and Azadirachta indica oil, Trichoderma atroviride strain CNCM I-1237 and Azadirachtin, Trichoderma atroviride strain NMI No. V08/002387 and alanycarb, Trichoderma atroviride strain NMI No. V08/002387 and aldicarb, Trichoderma atroviride strain NMI No. V08/002387 and carbofuran, Trichoderma atroviride strain NMI No. V08/002387 and carbosulfan, Trichoderma atroviride strain NMI No. V08/002387 and fosthiazate, Trichoderma atroviride strain NMI No. V08/002387 and cadusafos, Trichoderma atroviride strain NMI No. V08/002387 and oxamyl, Trichoderma atroviride strain NMI No. V08/002387 and thiodicarb, Trichoderma atroviride strain NMI No. V08/002387 and dimethoate, Trichoderma atroviride strain NMI No. V08/002387 and ethoprophos, Trichoderma atroviride strain NMI No. V08/002387 and terbufos, Trichoderma atroviride strain NMI No. V08/002387 and abamectin, Trichoderma atroviride strain NMI No. V08/002387 and methyl bromide and other alkyl halides, Trichoderma atroviride strain NMI No. V08/002387 and methyl isocyanate generators selected from diazomet and metam, Trichoderma atroviride strain NMI No. V08/002387 and fluazaindolizine, Trichoderma atroviride strain NMI No. V08/002387 and fluensulfone, Trichoderma atroviride strain NMI No. V08/002387 and fluopyram, Trichoderma atroviride strain NMI No. V08/002387 and tioxazafen, Trichoderma atroviride strain NMI No. V08/002387 and N-[1-(2,6-difluorophenyl)-1H-pyrazol-3-yl]-2-trifluoromethylbenzamide, Trichoderma atroviride strain NMI No. V08/002387 and cis-Jasmone, Trichoderma atroviride strain NMI No. V08/002387 and harpin, Trichoderma atroviride strain NMI No. V08/002387 and Azadirachta indica oil, Trichoderma atroviride strain NMI No. V08/002387 and Azadirachtin, Trichoderma atroviride strain NMI No. V08/002388 and alanycarb, Trichoderma atroviride strain NMI No. V08/002388 and aldicarb, Trichoderma atroviride strain NMI No. V08/002388 and carbofuran, Trichoderma atroviride strain NMI No. V08/002388 and carbosulfan, Trichoderma atroviride strain NMI No. V08/002388 and fosthiazate, Trichoderma atroviride strain NMI No. V08/002388 and cadusafos, Trichoderma atroviride strain NMI No. V08/002388 and oxamyl, Trichoderma atroviride strain NMI No. V08/002388 and thiodicarb, Trichoderma atroviride strain NMI No. V08/002388 and dimethoate, Trichoderma atroviride strain NMI No. V08/002388 and ethoprophos, Trichoderma atroviride strain NMI No. V08/002388 and terbufos, Trichoderma atroviride strain NMI No. V08/002388 and abamectin, Trichoderma atroviride strain NMI No. V08/002388 and methyl bromide and other alkyl halides, Trichoderma atroviride strain NMI No. V08/002388 and methyl isocyanate generators selected from diazomet and metam, Trichoderma atroviride strain NMI No. V08/002388 and fluazaindolizine, Trichoderma atroviride strain NMI No. V08/002388 and fluensulfone, Trichoderma atroviride strain NMI No. V08/002388 and fluopyram, Trichoderma atroviride strain NMI No. V08/002388 and tioxazafen, Trichoderma atroviride strain NMI No. V08/002388 and N-[1-(2,6-difluorophenyl)-1H-pyrazol-3-yl]-2-trifluoromethylbenzamide, Trichoderma atroviride strain NMI No. V08/002388 and cis-Jasmone, Trichoderma atroviride strain NMI No. V08/002388 and harpin, Trichoderma atroviride strain NMI No. V08/002388 and Azadirachta indica oil, Trichoderma atroviride strain NMI No. V08/002388 and Azadirachtin, Trichoderma atroviride strain NMI No. V08/002389 and alanycarb, Trichoderma atroviride strain NMI No. V08/002389 and aldicarb, Trichoderma atroviride strain NMI No. V08/002389 and carbofuran, Trichoderma atroviride strain NMI No. V08/002389 and carbosulfan, Trichoderma atroviride strain NMI No. V08/002389 and fosthiazate, Trichoderma atroviride strain NMI No. V08/002389 and cadusafos, Trichoderma atroviride strain NMI No. V08/002389 and oxamyl, Trichoderma atroviride strain NMI No. V08/002389 and thiodicarb, Trichoderma atroviride strain NMI No. V08/002389 and dimethoate, Trichoderma atroviride strain NMI No. V08/002389 and ethoprophos, Trichoderma atroviride strain NMI No. V08/002389 and terbufos, Trichoderma atroviride strain NMI No. V08/002389 and abamectin, Trichoderma atroviride strain NMI No. V08/002389 and methyl bromide and other alkyl halides, Trichoderma atroviride strain NMI No. V08/002389 and methyl isocyanate generators selected from diazomet and metam, Trichoderma atroviride strain NMI No. V08/002389 and fluazaindolizine, Trichoderma atroviride strain NMI No. V08/002389 and fluensulfone, Trichoderma atroviride strain NMI No. V08/002389 and fluopyram, Trichoderma atroviride strain NMI No. V08/002389 and tioxazafen, Trichoderma atroviride strain NMI No. V08/002389 and N-[1-(2,6-difluorophenyl)-1H-pyrazol-3-yl]-2-trifluoromethylbenzamide, Trichoderma atroviride strain NMI No. V08/002389 and cis-Jasmone, Trichoderma atroviride strain NMI No. V08/002389 and harpin, Trichoderma atroviride strain NMI No. V08/002389 and Azadirachta indica oil, Trichoderma atroviride strain NMI No. V08/002389 and Azadirachtin, Trichoderma atroviride strain NMI No. V08/002390 and alanycarb, Trichoderma atroviride strain NMI No. V08/002390 and aldicarb, Trichoderma atroviride strain NMI No. V08/002390 and carbofuran, Trichoderma atroviride strain NMI No. V08/002390 and carbosulfan, Trichoderma atroviride strain NMI No. V08/002390 and fosthiazate, Trichoderma atroviride strain NMI No. V08/002390 and cadusafos, Trichoderma atroviride strain NMI No. V08/002390 and oxamyl, Trichoderma atroviride strain NMI No. V08/002390 and thiodicarb, Trichoderma atroviride strain NMI No. V08/002390 and dimethoate, Trichoderma atroviride strain NMI No. V08/002390 and ethoprophos, Trichoderma atroviride strain NMI No. V08/002390 and terbufos, Trichoderma atroviride strain NMI No. V08/002390 and abamectin, Trichoderma atroviride strain NMI No. V08/002390 and methyl bromide and other alkyl halides, Trichoderma atroviride strain NMI No. V08/002390 and methyl isocyanate generators selected from diazomet and metam, Trichoderma atroviride strain NMI No. V08/002390 and fluazaindolizine, Trichoderma atroviride strain NMI No. V08/002390 and fluensulfone, Trichoderma atroviride strain NMI No. V08/002390 and fluopyram, Trichoderma atroviride strain NMI No. V08/002390 and tioxazafen, Trichoderma atroviride strain NMI No. V08/002390 and N-[1-(2,6-difluorophenyl)-1H-pyrazol-3-yl]-2-trifluoromethylbenzamide, Trichoderma atroviride strain NMI No. V08/002390 and cis-Jasmone, Trichoderma atroviride strain NMI No. V08/002390 and harpin, Trichoderma atroviride strain NMI No. V08/002390 and Azadirachta indica oil, Trichoderma atroviride strain NMI No. V08/002390 and Azadirachtin, Trichoderma atroviride strain ATCC 20476 and alanycarb, Trichoderma atroviride strain ATCC 20476 and aldicarb, Trichoderma atroviride strain ATCC 20476 and carbofuran, Trichoderma atroviride strain ATCC 20476 and carbosulfan, Trichoderma atroviride strain ATCC 20476 and fosthiazate, Trichoderma atroviride strain ATCC 20476 and cadusafos, Trichoderma atroviride strain ATCC 20476 and oxamyl, Trichoderma atroviride strain ATCC 20476 and thiodicarb, Trichoderma atroviride strain ATCC 20476 and dimethoate, Trichoderma atroviride strain ATCC 20476 and ethoprophos, Trichoderma atroviride strain ATCC 20476 and terbufos, Trichoderma atroviride strain ATCC 20476 and abamectin, Trichoderma atroviride strain ATCC 20476 and methyl bromide and other alkyl halides, Trichoderma atroviride strain ATCC 20476 and methyl isocyanate generators selected from diazomet and metam, Trichoderma atroviride strain ATCC 20476 and fluazaindolizine, Trichoderma atroviride strain ATCC 20476 and fluensulfone, Trichoderma atroviride strain ATCC 20476 and fluopyram, Trichoderma atroviride strain ATCC 20476 and tioxazafen, Trichoderma atroviride strain ATCC 20476 and N-[1-(2,6-difluorophenyl)-1H-pyrazol-3-yl]-2-trifluoromethylbenzamide, Trichoderma atroviride strain ATCC 20476 and cis-Jasmone, Trichoderma atroviride strain ATCC 20476 and harpin, Trichoderma atroviride strain ATCC 20476 and Azadirachta indica oil, Trichoderma atroviride strain ATCC 20476 and Azadirachtin, Trichoderma atroviride strain T11 and alanycarb, Trichoderma atroviride strain T11 and aldicarb, Trichoderma atroviride strain T11 and carbofuran, Trichoderma atroviride strain T11 and carbosulfan, Trichoderma atroviride strain T11 and fosthiazate, Trichoderma atroviride strain T11 and cadusafos, Trichoderma atroviride strain T11 and oxamyl, Trichoderma atroviride strain T11 and thiodicarb, Trichoderma atroviride strain T11 and dimethoate, Trichoderma atroviride strain T11 and ethoprophos, Trichoderma atroviride strain T11 and terbufos, Trichoderma atroviride strain T11 and abamectin, Trichoderma atroviride strain T11 and methyl bromide and other alkyl halides, Trichoderma atroviride strain T11 and methyl isocyanate generators selected from diazomet and metam, Trichoderma atroviride strain T11 and fluazaindolizine, Trichoderma atroviride strain T11 and fluensulfone, Trichoderma atroviride strain T11 and fluopyram, Trichoderma atroviride strain T11 and tioxazafen, Trichoderma atroviride strain T11 and N-[1-(2,6-difluorophenyl)-1H-pyrazol-3-yl]-2-trifluoromethylbenzamide, Trichoderma atroviride strain T11 and cis-Jasmone, Trichoderma atroviride strain T11 and harpin, Trichoderma atroviride strain T11 and Azadirachta indica oil, Trichoderma atroviride strain T11 and Azadirachtin, Trichoderma atroviride strain LC52 and alanycarb, Trichoderma atroviride strain LC52 and aldicarb, Trichoderma atroviride strain LC52 and carbofuran, Trichoderma atroviride strain LC52 and carbosulfan, Trichoderma atroviride strain LC52 and fosthiazate, Trichoderma atroviride strain LC52 and cadusafos, Trichoderma atroviride strain LC52 and oxamyl, Trichoderma atroviride strain LC52 and thiodicarb, Trichoderma atroviride strain LC52 and dimethoate, Trichoderma atroviride strain LC52 and ethoprophos, Trichoderma atroviride strain LC52 and terbufos, Trichoderma atroviride strain LC52 and abamectin, Trichoderma atroviride strain LC52 and methyl bromide and other alkyl halides, Trichoderma atroviride strain LC52 and methyl isocyanate generators selected from diazomet and metam, Trichoderma atroviride strain LC52 and fluazaindolizine, Trichoderma atroviride strain LC52 and fluensulfone, Trichoderma atroviride strain LC52 and fluopyram, Trichoderma atroviride strain LC52 and tioxazafen, Trichoderma atroviride strain LC52 and N-[1-(2,6-difluorophenyl)-1H-pyrazol-3-yl]-2-trifluoromethylbenzamide, Trichoderma atroviride strain LC52 and cis-Jasmone, Trichoderma atroviride strain LC52 and harpin, Trichoderma atroviride strain LC52 and Azadirachta indica oil, Trichoderma atroviride strain LC52 and Azadirachtin, Trichoderma atroviride strain SC1 and alanycarb, Trichoderma atroviride strain SC1 and aldicarb, Trichoderma atroviride strain SC1 and carbofuran, Trichoderma atroviride strain SC1 and carbosulfan, Trichoderma atroviride strain SC1 and fosthiazate, Trichoderma atroviride strain SC1 and cadusafos, Trichoderma atroviride strain SC1 and oxamyl, Trichoderma atroviride strain SC1 and thiodicarb, Trichoderma atroviride strain SC1 and dimethoate, Trichoderma atroviride strain SC1 and ethoprophos, Trichoderma atroviride strain SC1 and terbufos, Trichoderma atroviride strain SC1 and abamectin, Trichoderma atroviride strain SC1 and methyl bromide and other alkyl halides, Trichoderma atroviride strain SC1 and methyl isocyanate generators selected from diazomet and metam, Trichoderma atroviride strain SC1 and fluazaindolizine, Trichoderma atroviride strain SC1 and fluensulfone, Trichoderma atroviride strain SC1 and fluopyram, Trichoderma atroviride strain SC1 and tioxazafen, Trichoderma atroviride strain SC1 and N-[1-(2,6-difluorophenyl)-1H-pyrazol-3-yl]-2-trifluoromethylbenzamide, Trichoderma atroviride strain SC1 and cis-Jasmone, Trichoderma atroviride strain SC1 and harpin, Trichoderma atroviride strain SC1 and Azadirachta indica oil, Trichoderma atroviride strain SC1 and Azadirachtin, Trichoderma atroviride strain SKT-1 and alanycarb, Trichoderma atroviride strain SKT-1 and aldicarb, Trichoderma atroviride strain SKT-1 and carbofuran, Trichoderma atroviride strain SKT-1 and carbosulfan, Trichoderma atroviride strain SKT-1 and fosthiazate, Trichoderma atroviride strain SKT-1 and cadusafos, Trichoderma atroviride strain SKT-1 and oxamyl, Trichoderma atroviride strain SKT-1 and thiodicarb, Trichoderma atroviride strain SKT-1 and dimethoate, Trichoderma atroviride strain SKT-1 and ethoprophos, Trichoderma atroviride strain SKT-1 and terbufos, Trichoderma atroviride strain SKT-1 and abamectin, Trichoderma atroviride strain SKT-1 and methyl bromide and other alkyl halides, Trichoderma atroviride strain SKT-1 and methyl isocyanate generators selected from diazomet and metam, Trichoderma atroviride strain SKT-1 and fluazaindolizine, Trichoderma atroviride strain SKT-1 and fluensulfone, Trichoderma atroviride strain SKT-1 and fluopyram, Trichoderma atroviride strain SKT-1 and tioxazafen, Trichoderma atroviride strain SKT-1 and N-[1-(2,6-difluorophenyl)-1H-pyrazol-3-yl]-2-trifluoromethylbenzamide, Trichoderma atroviride strain SKT-1 and cis-Jasmone, Trichoderma atroviride strain SKT-1 and harpin, Trichoderma atroviride strain SKT-1 and Azadirachta indica oil, Trichoderma atroviride strain SKT-1 and Azadirachtin, Trichoderma atroviride strain SKT-2 and alanycarb, Trichoderma atroviride strain SKT-2 and aldicarb, Trichoderma atroviride strain SKT-2 and carbofuran, Trichoderma atroviride strain SKT-2 and carbosulfan, Trichoderma atroviride strain SKT-2 and fosthiazate, Trichoderma atroviride strain SKT-2 and cadusafos, Trichoderma atroviride strain SKT-2 and oxamyl, Trichoderma atroviride strain SKT-2 and thiodicarb, Trichoderma atroviride strain SKT-2 and dimethoate, Trichoderma atroviride strain SKT-2 and ethoprophos, Trichoderma atroviride strain SKT-2 and terbufos, Trichoderma atroviride strain SKT-2 and abamectin, Trichoderma atroviride strain SKT-2 and methyl bromide and other alkyl halides, Trichoderma atroviride strain SKT-2 and methyl isocyanate generators selected from diazomet and metam, Trichoderma atroviride strain SKT-2 and fluazaindolizine, Trichoderma atroviride strain SKT-2 and fluensulfone, Trichoderma atroviride strain SKT-2 and fluopyram, Trichoderma atroviride strain SKT-2 and tioxazafen, Trichoderma atroviride strain SKT-2 and N-[1-(2,6-difluorophenyl)-1H-pyrazol-3-yl]-2-trifluoromethylbenzamide, Trichoderma atroviride strain SKT-2 and cis-Jasmone, Trichoderma atroviride strain SKT-2 and harpin, Trichoderma atroviride strain SKT-2 and Azadirachta indica oil, Trichoderma atroviride strain SKT-2 and Azadirachtin, Trichoderma atroviride strain SKT-3 and alanycarb, Trichoderma atroviride strain SKT-3 and aldicarb, Trichoderma atroviride strain SKT-3 and carbofuran, Trichoderma atroviride strain SKT-3 and carbosulfan, Trichoderma atroviride strain SKT-3 and fosthiazate, Trichoderma atroviride strain SKT-3 and cadusafos, Trichoderma atroviride strain SKT-3 and oxamyl, Trichoderma atroviride strain SKT-3 and thiodicarb, Trichoderma atroviride strain SKT-3 and dimethoate, Trichoderma atroviride strain SKT-3 and ethoprophos, Trichoderma atroviride strain SKT-3 and terbufos, Trichoderma atroviride strain SKT-3 and abamectin, Trichoderma atroviride strain SKT-3 and methyl bromide and other alkyl halides, Trichoderma atroviride strain SKT-3 and methyl isocyanate generators selected from diazomet and metam, Trichoderma atroviride strain SKT-3 and fluazaindolizine, Trichoderma atroviride strain SKT-3 and fluensulfone, Trichoderma atroviride strain SKT-3 and fluopyram, Trichoderma atroviride strain SKT-3 and tioxazafen, Trichoderma atroviride strain SKT-3 and N-[1-(2,6-difluorophenyl)-1H-pyrazol-3-yl]-2-trifluoromethylbenzamide, Trichoderma atroviride strain SKT-3 and cis-Jasmone, Trichoderma atroviride strain SKT-3 and harpin, Trichoderma atroviride strain SKT-3 and Azadirachta indica oil, Trichoderma atroviride strain SKT-3 and Azadirachtin, Tsukamurella paurometabola strain C-924 and alanycarb, Tsukamurella paurometabola strain C-924 and aldicarb, Tsukamurella paurometabola strain C-924 and carbofuran, Tsukamurella paurometabola strain C-924 and carbosulfan, Tsukamurella paurometabola strain C-924 and fosthiazate, Tsukamurella paurometabola strain C-924 and cadusafos, Tsukamurella paurometabola strain C-924 and oxamyl, Tsukamurella paurometabola strain C-924 and thiodicarb, Tsukamurella paurometabola strain C-924 and dimethoate, Tsukamurella paurometabola strain C-924 and ethoprophos, Tsukamurella paurometabola strain C-924 and terbufos, Tsukamurella paurometabola strain C-924 and abamectin, Tsukamurella paurometabola strain C-924 and methyl bromide and other alkyl halides, Tsukamurella paurometabola strain C-924 and methyl isocyanate generators selected from diazomet and metam, Tsukamurella paurometabola strain C-924 and fluazaindolizine, Tsukamurella paurometabola strain C-924 and fluensulfone, Tsukamurella paurometabola strain C-924 and fluopyram, Tsukamurella paurometabola strain C-924 and tioxazafen, Tsukamurella paurometabola strain C-924 and N-[1-(2,6-difluorophenyl)-1H-pyrazol-3-yl]-2-trifluoromethylbenzamide, Tsukamurella paurometabola strain C-924 and cis-Jasmone, Tsukamurella paurometabola strain C-924 and harpin, Tsukamurella paurometabola strain C-924 and Azadirachta indica oil, Tsukamurella paurometabola strain C-924 and Azadirachtin, Pasteuria nishizawae oyacyst LF/ST and alanycarb, Pasteuria nishizawae oyacyst LF/ST and aldicarb, Pasteuria nishizawae oyacyst LF/ST and carbofuran, Pasteuria nishizawae oyacyst LF/ST and carbosulfan, Pasteuria nishizawae oyacyst LF/ST and fosthiazate, Pasteuria nishizawae oyacyst LF/ST and cadusafos, Pasteuria nishizawae oyacyst LF/ST and oxamyl, Pasteuria nishizawae oyacyst LF/ST and thiodicarb, Pasteuria nishizawae oyacyst LF/ST and dimethoate, Pasteuria nishizawae oyacyst LF/ST and ethoprophos, Pasteuria nishizawae oyacyst LF/ST and terbufos, Pasteuria nishizawae oyacyst LF/ST and abamectin, Pasteuria nishizawae oyacyst LF/ST and methyl bromide and other alkyl halides, Pasteuria nishizawae oyacyst LF/ST and methyl isocyanate generators selected from diazomet and metam, Pasteuria nishizawae oyacyst LF/ST and fluazaindolizine, Pasteuria nishizawae oyacyst LF/ST and fluensulfone, Pasteuria nishizawae oyacyst LF/ST and fluopyram, Pasteuria nishizawae oyacyst LF/ST and tioxazafen, Pasteuria nishizawae oyacyst LF/ST and N-[1-(2,6-difluorophenyl)-1H-pyrazol-3-yl]-2-trifluoromethylbenzamide, Pasteuria nishizawae oyacyst LF/ST and cis-Jasmone, Pasteuria nishizawae oyacyst LF/ST and harpin, Pasteuria nishizawae oyacyst LF/ST and Azadirachta indica oil, Pasteuria nishizawae oyacyst LF/ST and Azadirachtin, Pasteuria nishizawae Pn1 and alanycarb, Pasteuria nishizawae Pn1 and aldicarb, Pasteuria nishizawae Pn1 and carbofuran, Pasteuria nishizawae Pn1 and carbosulfan, Pasteuria nishizawae Pn1 and fosthiazate, Pasteuria nishizawae Pn1 and cadusafos, Pasteuria nishizawae Pn1 and oxamyl, Pasteuria nishizawae Pn1 and thiodicarb, Pasteuria nishizawae Pn1 and dimethoate, Pasteuria nishizawae Pn1 and ethoprophos, Pasteuria nishizawae Pn1 and terbufos, Pasteuria nishizawae Pn1 and abamectin, Pasteuria nishizawae Pn1 and methyl bromide and other alkyl halides, Pasteuria nishizawae Pn1 and methyl isocyanate generators selected from diazomet and metam, Pasteuria nishizawae Pn1 and fluazaindolizine, Pasteuria nishizawae Pn1 and fluensulfone, Pasteuria nishizawae Pn1 and fluopyram, Pasteuria nishizawae Pn1 and tioxazafen, Pasteuria nishizawae Pn1 and N-[1-(2,6-difluorophenyl)-1H-pyrazol-3-yl]-2-trifluoromethylbenzamide, Pasteuria nishizawae Pn1 and cis-Jasmone, Pasteuria nishizawae Pn1 and harpin, Pasteuria nishizawae Pn1 and Azadirachta indica oil, Pasteuria nishizawae Pn1 and Azadirachtin
118. The plant, cell, plant part or seed of any one of these numbered, or the soil in which they are grown or are intended to be grown, also treated with an insecticide selected from IAN1, IAN2, IAN3, IAN4, IAN5, IAN6, IAN7, IAN8, IAN9, IAN10, IAN11, IAN12, IAN13, IAN14, IAN15, IAN16, IAN17, IAN18, IAN19, IAN20, IAN21, IAN22, IAN23, IAN24, IAN25, IAN26, IAN27, IAN28, IAN29, IAN30, or SIAN1.
119. The plant, cell, plant part or seed of any one of these numbered paragraphs, or the soil in which they are grown or are intended to be grown, also treated with a fungicide selected from F1, F2, F3, F4, F5, F6, F7, F8, F9, F9, F10, F11, F12, F13, F14, F15, F16, or SF1.
120. The plant, cell, plant part or seed of any one of these numbered paragraphs, or the soil in which they are grown or are intended to be grown, treated with a combination selected from the group consisting of: combination of Clothianidin and Bacillus firmus (such as B. firmus GB126), combination of Clothianidin, Bacillus thuringiensis (such as B. thuringiensis strain EX297512) and Bacillus firmus (such as B. firmus GB126), combination of Imidacloprid and Thiodicarb, combination of Imidacloprid and Prothioconazole, combination of Clothianidin and Carboxin and Metalaxyl and Trifloxystrobin, combination of Metalaxyl and Prothioconazole and Tebuconazole, combination of Clothianidin and beta-Cyfluthrin, combination of Prothioconazole and Tebuconazole, combination of Clothianidin and Imidacloprid and Prothioconazole and Tebuconazole, combination of Carbendazim and Thiram, combination of Imidacloprid and Methiocarb, combination of Metalaxyl, Penflufen and Prothioconazole, combination of Metalaxyl, Penflufen, Prothioconazole and imidacloprid, combination of Metalaxyl, Penflufen, Prothioconazole, imidacloprid and fluopyram, combination of Metalaxyl, Penflufen, Prothioconazole, imidacloprid, fluopyram and Bacillus firmus (such as B. firmus GB126), combination of Metalaxyl, Penflufen, Prothioconazole, imidacloprid, fluopyram, Bacillus firmus (such as B. firmus GB126) and Bacillus thuringiensis (such as B. thuringiensis strain EX297512), combination of Metalaxyl, Penflufen, Prothioconazole and Clothianidin, combination of Metalaxyl, Penflufen, Prothioconazole, Clothianidin and Bacillus firmus (such as B. firmus GB126), combination of Metalaxyl, Penflufen, Prothioconazole, Clothianidin, Bacillus firmus (such as B. firmus GB126) and Bacillus thuringiensis (such as B. thuringiensis strain EX297512), combination of Metalaxyl, Penflufen, Prothioconazole, Clothianidin, Bacillus firmus (such as B. firmus GB126) and fluopyram, combination of Metalaxyl, Penflufen, Prothioconazole, Clothianidin, Bacillus firmus (such as B. firmus GB126), fluopyram and Bacillus thuringiensis (such as B. thuringiensis strain EX297512), combination of Imidacloprid and Prothioconazole, combination of Imidacloprid and Tefluthrin, combination of Imidacloprid and Pencycuron, combination of Imidacloprid and Penflufen, combination of Fluoxastrobin, Prothioconazole and Tebuconazole, combination of Fluoxastrobin, Prothioconazole and metalaxyl, combination of Fluoxastrobin, Prothioconazole, metalaxyl and imdiacloprid, combination of Fluoxastrobin, Prothioconazole, metalaxyl, imdiacloprid and fluopyram, combination of Fluoxastrobin, Prothioconazole, metalaxyl, imdiacloprid, fluopyram and Bacillus firmus (such as B. firmus GB126), and combination of Fluoxastrobin, Prothioconazole, metalaxyl, imdiacloprid, fluopyram, Bacillus firmus (such as B. firmus GB126), and Bacillus thuringiensis (such as B. thuringiensis strain EX297512), combination of Fluoxastrobin, Prothioconazole, metalaxyl, clothianidin and fluopyram, combination of Fluoxastrobin, Prothioconazole, metalaxyl, clothianidin and Bacillus firmus (such as B. firmus GB126), combination of Fluoxastrobin, Prothioconazole, metalaxyl, clothianidin, Bacillus firmus (such as B. firmus GB126), and Bacillus thuringiensis (such as B. thuringiensis strain EX297512), combination of Fluoxastrobin, Prothioconazole, metalaxyl, clothianidin and Bacillus firmus (such as B. firmus GB126), combination of Fluoxastrobin, Prothioconazole, metalaxyl, clothianidin, Bacillus firmus (such as B. firmus GB126), and Bacillus thuringiensis (such as B. thuringiensis strain EX297512), combination of Metalaxyl and Trifloxystrobin, combination of Penflufen and Trifloxystrobin, combination of Prothioconazole and Tebuconazole, combination of Fluoxastrobin and Prothioconazole and Tebuconazole and Triazoxide, combination of Imidacloprid and Methiocarb and Thiram, combination of Clothianidin and beta-Cyfluthrin, combination of Clothianidin and Fluoxastrobin and Prothioconazole and Tebuconazole, combination of Fluopyram and Fluoxastrobin and Triadimenol, combination of Metalaxyl and Trifloxystrobin, combination of Imidacloprid and Ipconacole, combination of Difenoconazol and Fludioxonil and Tebuconazole, combination of Imidacloprid and Tebuconazole, combination of Imidacloprid, Prothioconazole and Tebuconazole, combination of Metalaxyl, Prothioconazole and Tebuconazole, combination of fluopyram and Bacillus firmus (such as B. firmus GB126), combination of fluopyram, Bacillus firmus (such as B. firmus GB126), and Bacillus thuringiensis (such as B. thuringiensis strain EX297512), Combination of Pasteria nishazawae (such as P. nishizawae Pn1), thiamethoxam, sedexane, fludioxinil and mefonaxam, Combination of thiamethoxam, sedexane, fludioninil and mefonaxam, Combination of thiamethoxam, fludioxinil and mefonaxam, Combination of fludioxinil and mefonaxam, Combination of pyraclostrobin and fluoxayprad, Combination of abamectin and thiamethoxam, Combination of Burkholderia spp. strain (such as strain A396) and imidacloprid, Combination of Bacillus amyloliquefaciens (such as B. amyloliquefaciens strain PTA-4838) and clothianidin, Combination of tioxazafen, imidacloprid, prothioconazole, fluoxastrobin and metalaxyl, Combination of tioxazafen, clothiandiin, prothioconazole, fluoxastroin and metalaxyl, Combination of tioxazafen, clothianidin, Bacillus firmus (such as B. firmus GB126), prothioconazole, fluoxastrobin and metalaxyl, Combination of tioxazafen, clothianidin, Bacillus firmus (such as B. firmus GB126), Bacillus thuringiensis (such as B. thuringiensis strain EX297512), prothioconazole, fluoxastrobin and metalaxyl, combination of tioxazafen, prothioconazole, fluoxastrobin and metalaxyl, combination of tioxazafen, pyraclostrobin, fluoxyprad, metalaxyl and imidacloprid, a combination of Sedaxane, Thiamethoxam, Fludioxonil, and Mefenoxam, and a combination of Pasteuria nishizawae, Sedaxane, Thiamethoxam, Fludioxonil, and Mefenoxam, combination of clothianidin, fluopyram and Bacillus firmus (such as B. firmus GB126), combination of clothianidin, fluopyram, tioxazafen and Bacillus firmus (such as B. firmus GB126); combination of clothianidin, fluopyram, Bacillus firmus (such as B. firmus GB126) and Bacillus thuringiensis (such as B. thuringiensis strain EX297512), combination of clothianidin, fluopyram, tioxazafen, Bacillus firmus (such as B. firmus GB126) and Bacillus thuringiensis (such as B. thuringiensis strain EX297512), combination of pyraclostrobin and thiophanate-methyl and fipronil such as a BASF product called Standak Top, combination of imidacloprid and thiodicarb such as a product called Cropstar from Bayer CropScience.
121. The plant, cell, plant part or seed of any one of these numbered paragraphs, also comprising another soybean transformation event such as a soybean transformation event providing tolerance to additional herbicides, a soybean transformation event providing tolerance to nematodes via another mode of action, or a soybean transformation event providing insect control, or any one of the following soybean transformation events: Event MON87751, Event pDAB8264.42.32.1, Event DAS-81419-2, Event FG-072, Event SYHTOH2, Event DAS-68416-4, Event DAS-81615-9, Event DAS-44406-6, Event MON87708, Event MON89788, Event DAS-14536-7, Event GTS 40-3-2, Event A2704-12, Event BPS-CV127-9, Event A5547-127, Event MON87754, Event DP-3Ø5423-1, Event MON87701, Event MON87705, Event MON87712, Event pDAB4472-1606, Event DP-356043-5, Event MON87769, Event IND-Ø527 41Ø-5, Event DP305423, or any of the following Event combinations: MON89788 x MON87708, HOS×GTS 40-3-2, FG-072×A5547-127, MON87701×MON 89788, DAS-81419-2×DAS-44406-6, DAS-81419-2×DAS-68416-4, DAS-68416-4×MON 89788, MON 87705×MON 89788, MON 87769×MON 89788, DP305423×GTS 40-3-2, DP305423 x MON87708, Event DP305423×MON87708×Event MON89788, DP305423×MON89788, MON87705×MON87708, MON87705×MON87708×MON89788, MON89788 x MON87708×A5547-127, MON87751×MON87701×MON87708×MON89788, SYHTOH2×MON89788, SYHTOH2×GTS 40-3-2, SYHTOH2×MON89788×MON87708.

In one embodiment, the above paragraphs that refer to the soil or growth medium in which said plant, cell, tissue, plant part or seed are intended to be grown, also include the step of planting or sowing of said plant, cell, tissue, part or seed comprising EE-GM4 (with or without one or more native SCN resistance genes/loci as described herein and/or one or more other soybean transformation events as described herein) in said pre-treated soil or growth medium.

In one embodiment, the compound(s) or the biological control agent(s), or mixtures, in any one of the above numbered paragraphs are selected from any one of the groups as described herein, such as H1, H2, H3, H4, H5, IAN1, IAN2, IAN3, IAN4, IAN5, IAN6, IAN7, IAN8, IAN9, IAN10, IAN11, IAN12, IAN13, IAN14, IAN15, IAN16, IAN17, IAN18, IAN19, IAN20, IAN21, IAN22, IAN23, IAN24, IAN25, IAN26, IAN27, IAN28, IAN29, IAN30, SIAN1, F1, F2, F3, F4, F5, F6, F7, F8, F9, F9, F10, F11, F12, F13, F14, F15, F16, SF1, P1, BCA1, BCA2, BCA3, BCA4, BCA5, BCA6, BCA7, BCA8, BCA9, BCA10, NC1, NC2, NC3, SBCA1, SAI1, SCI and SC2, or are selected from any one of the groups SIAN1, SF1, BCA8, BCA9, BCA10, NC1, NC2, NC3, SBCA1, SAI1, SCI and SC2, such as for seed treatment.

### BRIEF DESCRIPTION OF THE DRAWINGS

The following Examples, not intended to limit the invention to the specific embodiments described, may be understood in conjunction with the accompanying Figures, incorporated herein by reference, in which:

FIG. 1: Schematic representation of the relationship between the cited nucleotide sequences and primers. Black bar: inserted T-DNA; hatched bar: DNA flanking the T-DNA. Black arrows: oligonucleotide primers, checkered arrow (a): chimeric cry14Ab-1.b gene (see Table 2 for composition of the chimeric gene); hatched arrow (b): chimeric hppdPf-4 Pa gene (see Table 2 for composition of the chimeric gene); black arrows: oligonucleotide primers; (c) refers to complement of the indicated nucleotide sequence; black line: oligonucleotide probes (the number below is the representative SEQ ID No.). The numbers below the bars representing SEQ ID No. 5 and 6 are the nucleotide positions of the different elements in said sequences. Note: the scheme is not drawn to scale.

FIG. 2: End-Point method for EE-GM4 identity analysis.

FIG. 2 shows an example of the result of the method described in Example 2.1 for a series of soybean samples containing EE-GM4 and conventional soybean samples. For each sample the S/B ratios for both the EE-GM4 specific reaction and the endogenous reaction are displayed. In this figure, samples marked “1-15” are soybean samples containing EE-GM4, samples marked “WT” are wild-type soybean samples (not containing EE-GM4), and samples marked “NTC” are the No Template Controls. Vertical (white) bars marked with “a” show the signal obtained for event EE-GM4, vertical (dark) bars marked with “b” show the signal obtained for the endogenous soybean gene. The horizontal line marked with “1” is the minimal Signal to Background ratio to detect event EE-GM4, the horizontal line marked with “2” is the minimal Signal to Background ratio to detect the endogenous soybean sequence, the horizontal line marked with “3” is the maximum Signal to Background ratio for non-target (no DNA) sample (background fluorescence).

FIG. 3: End-Point method for EE-GM4 identity and zygosity.

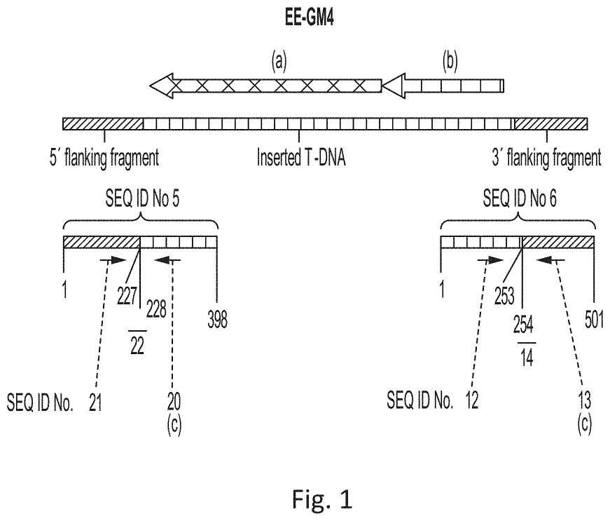

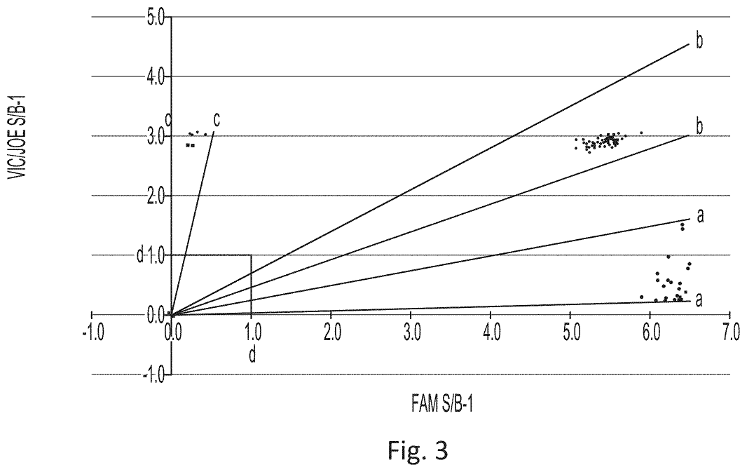

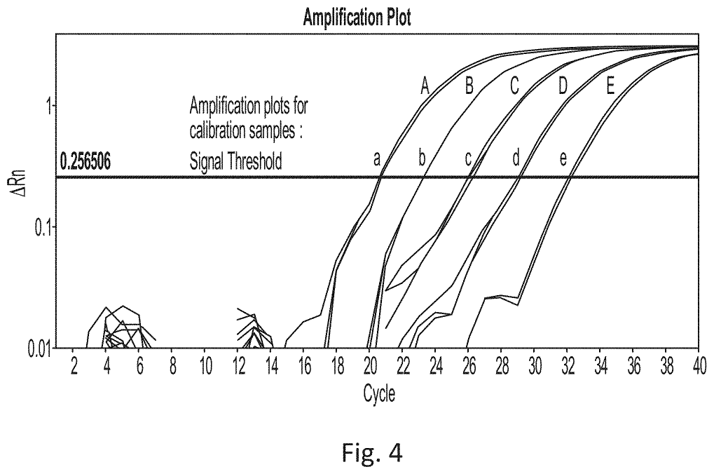

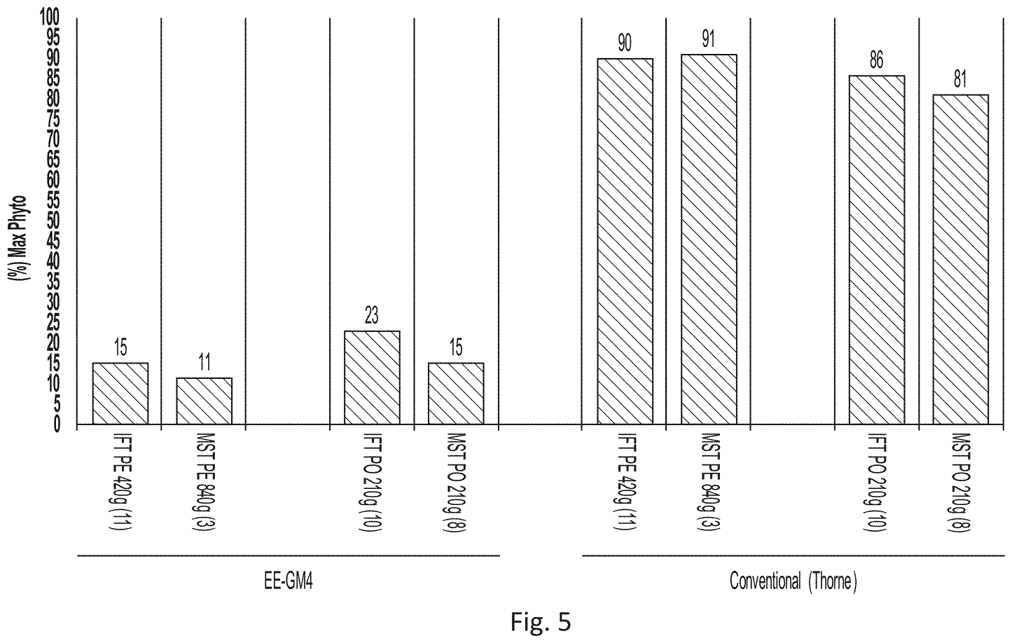

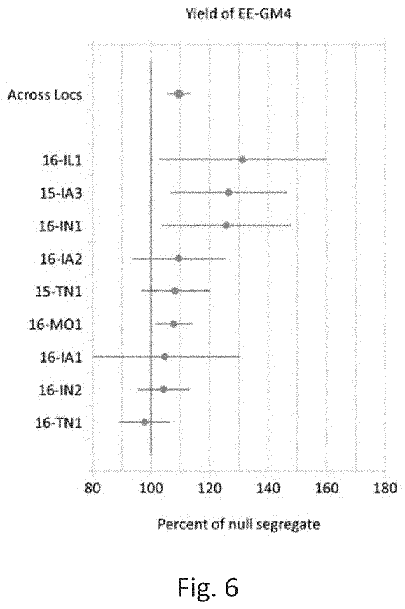

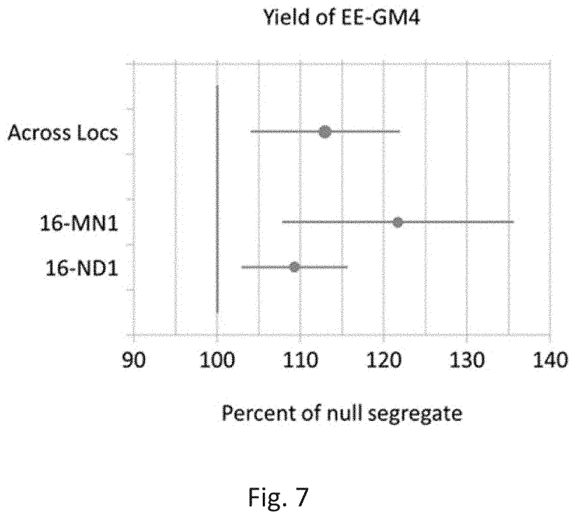

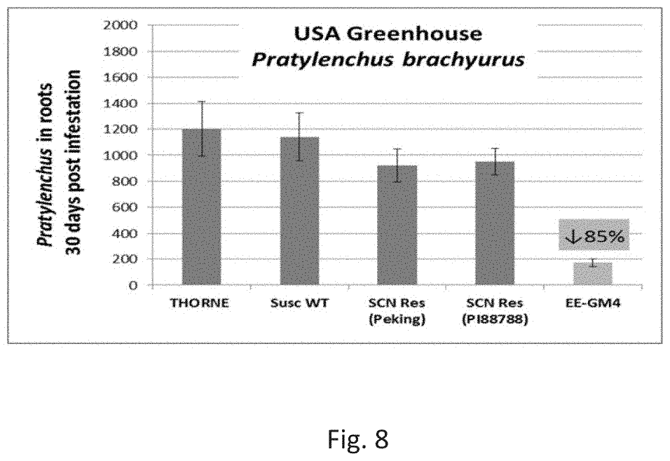

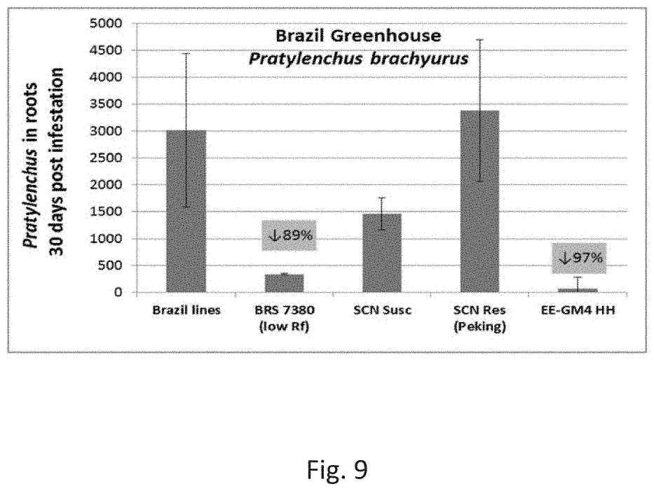

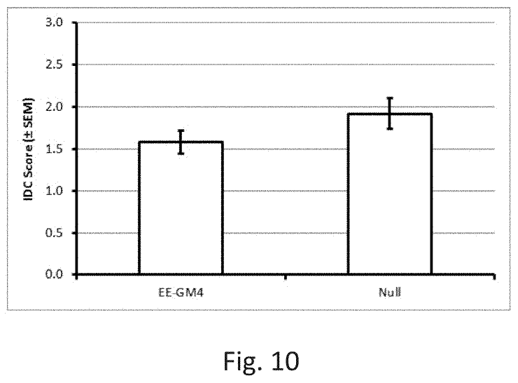

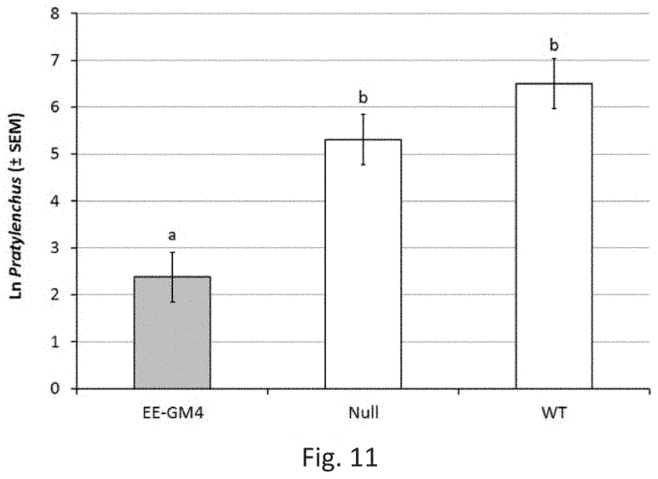

FIG. 3 shows an example of the result of the method described in Example 2.2 for a series of soybean samples containing EE-GM4 in a homozygous state, soybean samples containing EE-GM4 in a hemizygous state and conventional soybean samples. In this figure:
Samples within the lines marked with “a”: soybean samples containing EE-GM4 in a homozygous state.
Samples within the lines marked with “b”: soybean samples containing EE-GM4 in a hemizygous state
Samples within the lines marked with “c”: soybean samples not containing EE-GM4
Samples within the box formed by the lines marked with “d”: inconclusive samples

FIG. 4: Real-Time PCR method for EE-GM4 Low Level Presence analysis

FIG. 4 shows an example of the results of the RT-PCR method described in Example 2.3 for low level presence analysis as performed on the calibration samples. “a”, “b”, “c”, “d”, “e” indicate the Ct values for calibration samples “A”, “B”, “C”, “D”, “E”, respectively. Calibration samples “A”, “B”, “C”, “D”, “E” have decreasing amounts of EE-GM4 DNA.

FIG. 5: Average max phyto results for herbicide treatments

FIG. 5 shows the average of the maximum plant phytotoxicity data recorded for herbicide treatment in several field trials across 2 years, for soybean plants containing event EE-GM4 as compared to untransformed/conventional soybean plants (Thorne). Numbers in ( ) below a treatment give the number of trials included in the bar, the number on top of each bar gives the average maximum phytotoxicity value for that treatment. Treatments applied were: IFT=isoxaflutole, MST=mesotrione, PE=pre-emergence, PO=post-emergence (at V2-V3 stage, with adjuvants crop oil concentrate and ammonium sulfate added to increase herbicide activity). Rates shown are in gram active ingredient/hectare (4× dose in pre-emergence, 2× dose in post-emergence).

FIG. 6. Grain yield of EE-GM4 in Thorne in SCN infested fields.

EE-GM4 in original transformant background (Thorne) was tested in 9 different locations throughout Iowa, Illinois, Indiana, Missouri and Tennessee in 2015 and 2016, in SCN infested fields (ranging from low to high SCN infestation). The dot is the estimated yield of the homozygous event for each trial (as percent difference to the null), the horizontal lines represent the 95% confidence limits of the contrast between the homozygous event and the null segregant (if the line does not overlap the vertical line at 100 percent yield of null segregant, then the event was significantly different from the null segregant). “Across Locs” is the estimated yield of a combined analysis across all 9 locations.

FIG. 7. Grain yield of EE-GM4 in elite susceptible background in SCN infested fields.

EE-GM4 was introgressed (BC2F3) into an elite MG I (maturity group I) line that is susceptible to SCN and was tested at one location in Minnesota and one location in North Dakota in 2016 (each with a high SCN infestation level). The dot is the estimated yield of the homozygous event for each trial (as percent difference to the null), the horizontal line around the dot represents the 95% confidence limits of the contrast between the homozygous event and the null segregate (if the line does not overlap the vertical line at 100 percent of null segregate (i.e., no difference), then the event was significantly different from the null segregate). “Across Locs” is the estimated yield of a combined analysis across both locations.

FIG. 8. Pratylenchus resistance greenhouse assay in the USA

Elite soybean plants with EE-GM4 control Pratylenchus brachyurus in US greenhouse assays. Plants with EE-GM4 (“EE-GM4”) were compared to other elite soybean lines: one SCN susceptible Maturity Group (MG) 3 line (“THORNE”), one MG3 SCN susceptible line, one MG 6.2 SCN susceptible line and one MG9 SCN susceptible line (“Susc WT” shows the average for these 3 lines), one MG3 SCN resistant line (with the rhg1 resistance allele from PI88788, “SCN Res (PI88788)”), and one MG 6.2 SCN resistant line with the rhg1 and Rhg4 SCN resistance from Peking (“SCN Res (Peking)”). Plotted are the average numbers of Pratylenchus in roots 30 days after infestation (5 plants per entry), also showing the variation observed across variaties (as typically seen in greenhouse assays). Results show 85% control of Pratylenchus across EE-GM4 lines. Soybean lines with native SCN resistance (from Peking or PI88788) do not control Pratylenchus brachyurus.

FIG. 9. Pratylenchus resistance greenhouse assay in Brazil

Soybean plants with EE-GM4 (“EE-GM4”) significantly reduce Pratylenchus brachyurus in soybean roots. Pratylenchus brachyurus were isolated from local fields in Brazil. EE-GM4 plants (in two different US elite lines (both maturity group 6.2, one SCN-susceptible and one with Peking SCN-resistance (“EE-GM4”)) and five Brazilian soybean lines, with limited Pratylenchus control (“Brazil lines”), one Brazilian line, labeled as low Rf (reproductive factor) for Pratylenchus (“BRS 7380 (low Rf)”), one US elite line (maturity group 6.2) that is SCN-susceptible (“SCN Susc”) and one US elite line of MG 6.2 with Peking SCN-resistance (“SCN Res (Peking)”) were evaluated for Pratylenchus control in a greenhouse assay in Brazil. Plotted are the averages of those entries, also showing the variation observed across varieties (as typically seen in greenhouse assays). One Brazilian soybean line (BRS 7380), showed 89% reduction of Pratylenchus. EE-GM4 lines gave 79% control of Pratylenchus. Soybean lines that carry Peking native resistance to SCN do not control Pratylenchus brachyurus.

FIG. 10. Iron Deficiency Chlorosis (IDC) scores for EE-GM4 plants compared to nulls

FIG. 10 shows the IDC scores of soybean plants with EE-GM4 at one location (with high SCN infestation). The trial was a split-plot design (4 plots per entry) looking at the effect of the event in 3 different backgrounds (2 susceptible soybean lines and 1 with SCN resistance from PI88788). Shown are the averages of IDC scores for plants with event EE-GM4 (“EE-GM4”) and the corresponding null segregant (“Null”, lacking EE-GM4) across three genetic backgrounds (1 SCN-resistant, 1 SCN-susceptible, and the SCN-susceptible Thorne background). One bar represents 12 total plots. The vertical lines indicate the standard error (“SEM” is the Standard Error of the Mean).

FIG. 11. Pratylenchus efficacy field trial in Brazil

Soybean plants homozygous for EE-GM4 (“HH”) showed significantly lower Pratylenchus brachyurus nematodes in soybean roots. The EE-GM4 event in the elite maturity group IX background was compared to segregating sister lines having lost the transgene (“Null”), as well as to the wild-type elite maturity group IX parent line (“WT”) in fields naturally infested with P. brachyurus. The data were compiled from two trial locations that were sampled at −98 days after planting. Plotted are the natural log of the averages of those entries, and the variation. The vertical lines indicate the standard error (“SEM” is the Standard Error of the Mean). Treatment means with different letters are significantly different at P<0.05.

### DETAILED DESCRIPTION OF THE PREFERRED EMBODIMENTS OF THE INVENTION

In this invention, EE-GM4 has been identified as an elite event from a population of transgenic soybean plants in the development of nematode resistant soybean (Glycine max) comprising a gene coding for 4-hydroxy phenylpyruvate dioxygenase (HPPD) inhibitor tolerance combined with a gene conferring resistance to nematodes, each under control of a plant-expressible promoter. Specific tools for use in the identification of elite event EE-GM4 in biological samples are described herein.

The incorporation of a recombinant DNA molecule in the plant genome typically results from transformation of a cell or tissue. The particular site of incorporation is usually due to random integration.

The DNA introduced into the plant genome as a result of transformation of a plant cell or tissue with a recombinant DNA or “transforming DNA”, and originating from such transforming DNA is hereinafter referred to as “inserted T-DNA” comprising one or more “transgenes”. The transgenes of EE-GM4 are the nematode resistance and HPPD inhibitor herbicide tolerance genes. “Plant DNA” in the context of the present invention will refer to DNA originating from the plant which is transformed. Plant DNA will usually be found in the same genetic locus in the corresponding wild-type plant. The inserted T-DNA can be characterized by the location and the configuration at the site of incorporation of the recombinant DNA molecule in the plant genome.

The site in the plant genome where a recombinant DNA has been inserted is also referred to as the “insertion site” or “target site”. Insertion of the recombinant DNA into the region of the plant genome referred to as “pre-insertion plant DNA” (also named “pre-insertion locus” herein) can be associated with a deletion of plant DNA, referred to as “target site deletion”. A “flanking region” or “flanking sequence” as used herein refers to a sequence of at least 10 bp, at least 20 bp, at least 50 bp, and up to 5000 bp of DNA different from the introduced T-DNA, preferably DNA from the plant genome which is located either immediately upstream of and contiguous with or immediately downstream of and contiguous with the inserted T-DNA. Transformation procedures leading to random integration of the inserted T-DNA will result in transformants with different flanking regions, which are characteristic and unique for each transformant. When the recombinant DNA is introduced into a plant through traditional crossing, its insertion site in the plant genome, or its flanking regions, will generally not be changed.

An “isolated nucleic acid (sequence/molecule)” or “isolated DNA (sequence/molecule)”, as used herein, refers to a nucleic acid or DNA (sequence/molecule) which is no longer in the natural environment it was isolated from, e.g., the nucleic acid sequence in another bacterial host or in a plant genome, or a nucleic acid or DNA (sequence/molecule) fused to DNA or nucleic acid (sequence/molecule) from another origin, such as when contained in a chimeric gene under the control of a (heterologous) plant-expressible promoter. Any nucleic acid or DNA of this invention, including any primer, can also be non-naturally-occurring, such as a nucleic acid or DNA with a sequence identical to a sequence occurring in nature, but having a label (missing from the naturally-occurring counterpart), or with a sequence having at least one nucleotide addition or replacement or at least one internal nucleotide deletion compared to a naturally-existing nucleotide, or with a sequence having a sequence identity below 100% (not identical) to a naturally-existing nucleic acid or DNA or a fragment thereof, or a nucleic acid or DNA with a sequence consisting of nucleotide sequences from different origins that do not occur together in nature (a chimeric or hybrid DNA), or a man-made synthetic nucleic acid or DNA with a sequence different from the natural nucleic acid or DNA or a fragment thereof.

An event is defined as a (artificial) genetic locus that, as a result of genetic engineering, carries an inserted T-DNA or transgene comprising at least one copy of a gene of interest or of the genes of interest. The typical allelic states of an event are the presence or absence of the inserted T-DNA. An event is characterized phenotypically by the expression of the transgene or transgenes. At the genetic level, an event is part of the genetic make-up of a plant. At the molecular level, an event can be characterized by the restriction map (e.g., as determined by Southern blotting), by the upstream and/or downstream flanking sequences of the transgene, the location of molecular markers and/or the molecular configuration of the transgene. Usually transformation of a plant with a transforming DNA comprising at least one gene of interest leads to a population of transformants comprising a multitude of separate events, each of which is unique. An event is characterized by the inserted T-DNA and at least one of the flanking sequences.

An elite event, as used herein, is an event which is selected from a group of events, obtained by transformation with the same transforming DNA, based on an optimal trait efficacy and superior expression, stability of the transgene(s) and its compatibility with optimal agronomic characteristics of the plant comprising it. Thus the criteria for elite event selection are one or more, preferably two or more, advantageously all of the following:
a) trait efficacy;
b) that the presence of the inserted T-DNA does not compromise other desired characteristics of the plant, such as those relating to agronomic performance or commercial value;
c) that the event is characterized by a well-defined molecular configuration which is stably inherited and for which appropriate tools for identity control can be developed;
d) that the gene(s) of interest show(s) a correct, appropriate and stable spatial and temporal phenotypic expression, at a commercially acceptable level in a range of environmental conditions in which the plants carrying the event are likely to be exposed in normal agronomic use.

It is preferred that the inserted T-DNA is associated with a position in the plant genome that allows easy introgression into desired commercial genetic backgrounds.

The status of an event as an elite event is confirmed by introgression of the elite event in different relevant genetic backgrounds and observing compliance with one, two, three or all of the criteria e.g. a), b), c) and d) above.

An “elite event” thus refers to a genetic locus comprising an inserted T-DNA, which meets the above-described criteria. A plant, plant material or progeny such as seeds can comprise one or more different elite events in its genome.

The tools developed to identify an elite event or the plant or plant material comprising an elite event, or products which comprise plant material comprising the elite event, are based on the specific genomic characteristics of the elite event, such as, a specific restriction map of the genomic region comprising the inserted T-DNA, molecular markers or the sequence of the flanking region(s) of the inserted T-DNA.

Once one or both of the flanking regions of the inserted T-DNA have been sequenced, primers and/or probes can be developed which specifically recognize this (these) sequence(s) in the nucleic acid (DNA or RNA) of a sample by way of a molecular biological technique. For instance a PCR method can be developed to identify the elite event in biological samples (such as samples of plants, plant material or products comprising plant material). Such a PCR is based on at least two specific “primers”, one recognizing a sequence within the 5′ or 3′ T-DNA flanking region of the elite event and the other recognizing a sequence within the inserted T-DNA. The primers preferably have a sequence of between 15 and 35 nucleotides which under optimized PCR conditions “specifically recognize” a sequence within the 5′ or 3′ T-DNA flanking region of the elite event and the inserted T-DNA of the elite event respectively, so that a specific fragment (“integration fragment” or discriminating amplicon) is amplified from a nucleic acid sample comprising the elite event. This means that only the targeted integration fragment, and no other sequence in the plant genome or inserted T-DNA, is amplified under optimized PCR conditions.

PCR primers suitable for the invention may be the following:
oligonucleotides ranging in length from 17 nt to about 200 nt, comprising a nucleotide sequence of at least 17 consecutive nucleotides, preferably 20 consecutive nucleotides, selected from the 5′ T-DNA flanking sequence (SEQ ID No. 5 from nucleotide 1 to nucleotide 227 or of SEQ ID No. 24 from nucleotide 1 to nucleotide 1058 or plant genomic sequences upstream thereof and contiguous therewith) at their 3′ end (primers recognizing 5′ T-DNA flanking sequences); or
oligonucleotides ranging in length from 17 nt to about 200 nt, comprising a nucleotide sequence of at least 17 consecutive nucleotides, preferably 20 consecutive nucleotides, selected from the 3′ T-DNA flanking sequence (complement of SEQ ID No. 6 from nucleotide 254 to nucleotide 501 or the nucleotide sequence of the complement of SEQ ID No. 25 from nucleotide 254 to nucleotide 1339 or plant genomic sequences downstream thereof and contiguous therewith) at their 3′ end (primers recognizing 3′ T-DNA flanking sequences); or
oligonucleotides ranging in length from 17 nt to about 200 nt, comprising a nucleotide sequence of at least 17 consecutive nucleotides, preferably 20 consecutive nucleotides, selected from the inserted T-DNA sequences (complement of SEQ ID No. 5 from nucleotide 228 to nucleotide 398 or sequence of SEQ ID No. 6 from nucleotide 1 to nucleotide 253, or the sequence of SEQ ID No. 11 from nucleotide 17 to nucleotide 7621, or the sequence of SEQ ID No. 23 from nucleotide 1059 to nucleotide 8663, or its complement) at their 3′ end (primers recognizing inserted T-DNA).

It will be understood that primers recognizing the 5′ T-DNA flanking sequences can be used in a PCR reaction together with primers recognizing the inserted T-DNA which are selected from the complement of SEQ ID No. 5 from nucleotide 228 to nucleotide 398 or T-DNA sequences downstream thereof and contiguous therewith, whereas primers recognizing the 3′ T-DNA flanking sequences can be used in a PCR reaction together with primers recognizing the inserted T-DNA which are selected from the sequence of SEQ ID No. 6 from nucleotide 1 to nucleotide 253, or T-DNA upstream thereof and contiguous therewith. Primers recognizing inserted T-DNA can also be selected from the sequence of SEQ ID No. 11 from nucleotide 17 to nucleotide 7621, or the sequence of SEQ ID No. 23 from nucleotide 1059 to nucleotide 8663, or the complement thereof.

The primers may of course be longer than the mentioned 17 consecutive nucleotides, and may, e.g., be 20, 21, 30, 35, 50, 75, 100, 150, 200 nt long or even longer. The primers may entirely consist of nucleotide sequence selected from the mentioned nucleotide sequences of flanking sequences and inserted T-DNA sequences. However, the nucleotide sequence of the primers at their 5′ end (i.e., outside of the 17 consecutive nucleotides at the 3′ end) is less critical. Thus, the 5′ sequence of the primers may comprise or consist of a nucleotide sequence selected from the flanking sequences or inserted T-DNA, as appropriate, but may contain several (e.g., 1, 2, 5, or 10) mismatches in comparison with the T-DNA or T-DNA flanking DNA. The 5′ sequence of the primers may even entirely be a nucleotide sequence unrelated to the flanking sequences or inserted T-DNA, such as, e.g., a nucleotide sequence representing one or more restriction enzyme recognition sites, or such as nucleotide sequences capable of binding other oligonucleotides, such as labelled oligonucleotides, such as FRET cassettes (LGC genomics; see Semagn et al., 2014, Mol Breeding 33:1-14, and U.S. Pat. No. 7,615,620). Such unrelated sequences or flanking DNA sequences with mismatches should preferably not be longer than 100, more preferably not longer than 50 or even 25 nucleotides. The primers can also be modified with a label, such as a fluorescent label.

Moreover, suitable primers may comprise or consist (essentially) of a nucleotide sequence at their 3′ end spanning the joining region between the 5′ or 3′ T-DNA flanking region-derived sequences and the inserted T-DNA sequences (located at nucleotides 227 and 228 in SEQ ID No. 5 and nucleotides 253 and 254 in SEQ ID No. 6, or nucleotides 1058 and 1059 in SEQ ID No. 24 and nucleotides 253 and 254 in SEQ ID No. 25) provided the mentioned 3′-located 17 consecutive nucleotides are not derived exclusively from either the inserted T-DNA or the T-DNA flanking sequences in SEQ ID No. 5 or 6 or SEQ ID No. 24 or 25.

It will also be immediately clear to the skilled artisan that properly selected PCR primer pairs should also not comprise sequences complementary to each other.

For the purpose of the invention, the “complement of a nucleotide sequence represented in SEQ ID No: X” is the nucleotide sequence which can be derived from the represented nucleotide sequence by replacing the nucleotides with their complementary nucleotide according to Chargaff's rules (A⇔T; G⇔C) and reading the sequence in the 5′ to 3′ direction, i.e., in opposite direction of the represented nucleotide sequence.

Examples of suitable primers are the oligonucleotide sequences of SEQ ID no. 13 or SEQ ID No. 21 or SEQ ID No. 26 or 27 (3′ or 5′ T-DNA flanking sequence recognizing primer), or SEQ ID No. 14 or SEQ ID No. 20 or SEQ ID No. 28 or 29 (inserted T-DNA recognizing primer for use with the 3′ or 5′ T-DNA flanking sequence recognizing primers).

Preferably, the amplified fragment has a length of between 50 and 500 nucleotides, such as a length between 50 and 150 nucleotides. The specific primers may have a sequence which is between 80 and 100% identical to a sequence within the 5′ or 3′ T-DNA flanking region of the elite event and the inserted T-DNA of the elite event, respectively, provided the mismatches still allow specific identification of the elite event with these primers under optimized PCR conditions. The range of allowable mismatches however, can easily be determined experimentally and are known to a person skilled in the art.

Detection of integration fragments can occur in various ways, e.g., via size estimation after gel analysis. The integration fragments may also be directly sequenced. Other sequence specific methods for detection of amplified DNA fragments are also known in the art. Amplified DNA fragments can also be detected using labelled sequences and detection of the label. For example, a labelled probe can be included in the reaction mixture which specifically binds to the amplified fragment. In one embodiment, the labelled probe (FRET hybridization probe) can comprise a fluorescent label and a quencher, such that the FRET cassette is no longer quenched and emits fluorescence when bound to the PCR product. Alternatively, a labelled FRET cassette, i.e., an oligonucleotide labeled with a fluorescent label and a quencher, can be included in the reaction mixture which specifically binds one of the primers in the reaction mixture, such as a FRET cassette directed to a 5′ extension of the primer used in the reaction mixture (see, e.g., Semagn et al., 2014, Mol Breeding 33:1-14, and U.S. Pat. No. 7,615,620). Fluorescence can be measured using methods known in the art. Fluorescence can be measured real-time, i.e., during each cycle of the PCR reaction. Fluorescence can also be measured at the end of the PCR reaction.

As the sequence of the primers and their relative location in the genome are unique for the elite event, amplification of the integration fragment will occur only in biological samples comprising (the nucleic acid of) the elite event. Preferably when performing a PCR to identify the presence of EE-GM4 in unknown samples, a control is included of a set of primers with which a fragment within a “housekeeping gene” of the plant species of the event can be amplified. Housekeeping genes are genes that are expressed in most cell types and which are concerned with basic metabolic activities common to all cells. Preferably, the fragment amplified from the housekeeping gene is a fragment which is larger than the amplified integration fragment. Depending on the samples to be analyzed, other controls can be included.

Standard PCR protocols are described in the art, such as in “PCR Applications Manual” (Roche Molecular Biochemicals, 2nd Edition, 1999, or 3^{rd} Edition, 2006) and other references. The optimal conditions for the PCR, including the sequence of the specific primers, are specified in a “PCR (or Polymerase Chain Reaction) Identification Protocol” for each elite event. It is however understood that a number of parameters in the PCR Identification Protocol may need to be adjusted to specific laboratory conditions, and may be modified slightly to obtain similar results. For instance, use of a different method for preparation of DNA may require adjustment of, for instance, the amount of primers, polymerase and annealing conditions used. Similarly, the selection of other primers may dictate other optimal conditions for the PCR Identification Protocol. These adjustments will however be apparent to a person skilled in the art, and are furthermore detailed in current PCR application manuals such as the one cited above.

Alternatively, specific primers can be used to amplify an integration fragment that can be used as a “specific probe” for identifying EE-GM4 in biological samples. Contacting nucleic acid of a biological sample, with the probe, under conditions which allow hybridization of the probe with its corresponding fragment in the nucleic acid, results in the formation of a nucleic acid/probe hybrid. The formation of this hybrid can be detected (e.g., via labeling of the nucleic acid or probe), whereby the formation of this hybrid indicates the presence of EE-GM4. Such identification methods based on hybridization with a specific probe (either on a solid phase carrier or in solution) have been described in the art. The specific probe is preferably a sequence which, under optimized conditions, hybridizes specifically to a region comprising part of the 5′ or 3′ T-DNA flanking region of the elite event and part of the inserted T-DNA contiguous therewith (hereinafter referred to as “specific region”). Preferably, the specific probe comprises a sequence of between 50 and 500 bp, or of 100 to 350 bp which is at least 80%, or between 80 and 85%, or between 85 and 90%, or between 90 and 95%, or between 95% and 100% identical (or complementary), or is identical (or complementary) to the nucleotide sequence of a specific region of EE-GM4. Preferably, the specific probe will comprise a sequence of about 15 to about 100 contiguous nucleotides identical (or complementary) to a specific region of the elite event.

Oligonucleotides suitable as PCR primers for detection of the elite event EE-GM4 can also be used to develop a PCR-based protocol to determine the zygosity status of plants containing the elite event. To this end, two primers recognizing the wild-type locus before integration are designed in such a way that they are directed towards each other and have the insertion site located in between the primers. These primers may contain primers specifically recognizing the 5′ and/or 3′ T-DNA flanking sequences of EE-GM4. This set of primers recognizing the wild-type locus before integration, together with a third primer complementary to transforming DNA sequences (inserted T-DNA) allows simultaneous diagnostic PCR amplification of the EE-GM4 specific locus, as well as of the wild type locus. If the plant is homozygous for the transgenic locus or the corresponding wild type locus, the diagnostic PCR will give rise to a single PCR product typical, preferably typical in length, for either the transgenic or wild type locus. If the plant is hemizygous for the transgenic locus, two locus-specific PCR products will appear, reflecting both the amplification of the transgenic and wild type locus.

Alternatively, to determine the zygosity status of plants containing the elite event, two primers recognizing the wild-type locus before integration are designed in such a way that they are directed towards each other, and that one primer specifically recognizes the 5′ or the 3′ T-DNA flanking sequences contained in SEQ ID No. 5 or 6 or in SEQ ID No. 24 or 25, and that one primer specifically recognizes the 3′ or the 5′ T-DNA flanking sequences contained within SEQ ID No. 6 or 5 or within SEQ ID No. 25 or 24, or specifically recognizes the pre-insertion locus. For the current invention, a suitable primer pair recognizing the wild type locus before integration is a primer pair containing one primer comprising or consisting (essentially) of the nucleotide sequence of SEQ ID No. 18, and one primer comprising or consisting (essentially) of the nucleotide sequence of SEQ ID No. 13. This set of primers, together with a third primer complementary to transforming DNA sequences (inserted T-DNA), or complementary to transforming DNA sequences and the 5′ or 3′ T-DNA flanking sequences contiguous therewith, and in a direction towards the primer which specifically recognizes the 5′ or the 3′ T-DNA flanking sequences (such as a primer comprising or consisting (essentially) of the nucleotide sequence of SEQ ID No. 12, which is in a direction towards the primer comprising or consisting (essentially) of the nucleotide sequence of SEQ ID No. 13) allow simultaneous diagnostic PCR amplification of the EE-GM4 specific locus, as well as of the wild type locus. If the plant is homozygous for the transgenic locus or the corresponding wild type locus, the diagnostic PCR will give rise to a single PCR product typical for either the transgenic or wild type locus. If the plant is hemizygous for the transgenic locus, two locus-specific PCR products will appear, reflecting both the amplification of the transgenic and wild type locus.

Detection of the PCR products typical for the wild-type and transgenic locus can be based on determination of the length of the PCR products which can be typical for the wild-type and transgenic locus. Alternatively, detection of the PCR products typical for the wild-type and transgenic locus can be performed by modification of the primer specific for the pre-insertion locus and by modification of the primer specific for the inserted T-DNA, and detection of incorporation into a PCR product of the modified primers. For example, the primer specific for the pre-insertion locus and the primer specific for the inserted T-DNA can be labeled using a fluorescent label, wherein the labels are different for the two primers. Fluorescence can be detected when the primer is incorporated into a PCR product. If the plant is homozygous for the transgenic locus or the corresponding wild type locus, fluorescence can be detected of the label of the primer specific for the inserted T-DNA only or of the primer specific for the pre-insertion locus only. If the plant is hemizygous for the transgenic locus, fluorescence can be detected of both the label of the primer specific for the inserted T-DNA and of the primer specific for the pre-insertion locus, reflecting both the amplification of the transgenic and wild type locus.

Alternatively, the primer specific for the pre-insertion locus and the primer specific for the inserted T-DNA can have a 5′ extension which specifically binds a labeled FRET cassette, i.e. an oligonucleotide labelled with a fluorescent label and a quencher, wherein the 5′ extension and the corresponding FRET cassettes are different for the two primers (see, e.g., Semagn et al., 2014, Mol Breeding 33:1-14, and U.S. Pat. No. 7,615,620). Fluorescence can be detected when the primer is incorporated into a PCR product and, subsequently, the FRET cassette is incorporated in the PCR product. If the plant is homozygous for the transgenic locus or the corresponding wild type locus, fluorescence can be detected of the FRET cassette specifically binding to the primer specific for the inserted T-DNA only or of the FRET cassette specifically binding to the primer specific for the pre-insertion locus only. If the plant is hemizygous for the transgenic locus, fluorescence can be detected of both of the FRET cassette specifically binding to the primer specific for the inserted T-DNA and of the FRET cassette specifically binding to the primer specific for the pre-insertion locus, reflecting both the amplification of the transgenic and wild type locus.

If the plant is homozygous for the transgenic locus or the corresponding wild type locus, the diagnostic PCR will give rise to a single PCR product typical, preferably typical in length, for either the transgenic or wild type locus. If the plant is hemizygous for the transgenic locus, two locus-specific PCR products will appear, reflecting both the amplification of the transgenic and wild type locus.

Alternatively, to determine the zygosity status of plants containing the elite event, presence of the event can be determined in a PCR reaction in a quantitative way as described in the Examples. To this end, two primers recognizing the event EE-GM4 are designed in such a way that they are directed towards each other, wherein one primer specifically recognizes the 5′ or 3′ T-DNA flanking sequence contained within SEQ ID No. 5 or 6 or within SEQ ID No. 24 or 25, and wherein one primer specifically recognizes the inserted T-DNA within SEQ ID no. 5 or 6 or within SEQ ID No. 24 or 25 or within SEQ ID No. 11 or 23. This set of primers allows PCR amplification of the EE-GM4 specific locus. The amplified DNA fragment can quantitatively be detected using a labeled probe which is included in the reaction mixture which specifically binds to the amplified fragment. The labeled probe can comprise a fluorescent label and a quencher, such that label is no longer quenched and emits fluorescence when bound to the PCR product. Fluorescence can be measured real-time, i.e. during each cycle of the PCR reaction, using methods known in the art. The PCR cycle at which the fluorescence exceeds a certain threshold level is a measure for the amount of EE-GM4 specific locus in the biological sample which is analyzed, and the zygosity status can be calculated based on reference homozygous and heterozygous samples.

Alternatively, zygosity status of plants comprising EE-GM4 can also be determined based on copy number analysis, using the Taqman chemistry and principles of Real-Time PCR. The alternative method will typically include a EE-GM4 specific reaction to quantify the EE-GM4 copy number, and a endogenous gene-specific reaction for normalization of the EE-GM4 copy number. Samples containing the EE-GM4 event in a homozygous state will have a relative copy number that is two-fold higher than hemizygous samples. Azygous samples will not amplify the EE-GM4 sequence in such a method.

Furthermore, detection methods specific for elite event EE-GM4 which differ from PCR based amplification methods can also be developed using the elite event specific sequence information provided herein. Such alternative detection methods include linear signal amplification detection methods based on invasive cleavage of particular nucleic acid structures, also known as Invader™ technology, (as described e.g. in U.S. Pat. No. 5,985,557 “Invasive Cleavage of Nucleic Acids”, U.S. Pat. No. 6,001,567 “Detection of Nucleic Acid sequences by Invader Directed Cleavage”, incorporated herein by reference). To this end, the target sequence is hybridized with a labeled first nucleic acid oligonucleotide comprising the nucleotide sequence of SEQ ID No. 5 from nucleotide position 228 to nucleotide position 245 or its complement or comprising the nucleotide sequence of SEQ ID No. 6 from nucleotide position 236 to nucleotide position 253 or its complement, and is further hybridized with a second nucleic acid oligonucleotide comprising the nucleotide sequence of SEQ ID No. 5 from nucleotide 210 to nucleotide 227 or its complement or comprising the nucleotide sequence of SEQ ID No. 6 from nucleotide 254 to nucleotide 271 or its complement, wherein said first and second oligonucleotide overlap by at least one nucleotide. The duplex or triplex structure which is produced by this hybridization allows selective probe cleavage with an enzyme (Cleavase®) leaving the target sequence intact. The cleaved labeled probe is subsequently detected, potentially via an intermediate step resulting in further signal amplification.

In one embodiment is provided a method of detecting the presence of elite event EE-GM4 in biological samples through hybridization with a substantially complementary labeled nucleic acid probe in which the probe:target nucleic acid ratio is amplified through recycling of the target nucleic acid sequence, said method comprising:

a) hybridizing said target nucleic acid sequence to a first nucleic acid oligonucleotide comprising the nucleotide sequence of SEQ ID No. 5 from nucleotide position 228 to nucleotide position 245 or its complement or said first nucleic acid oligonucleotide comprising the nucleotide sequence of SEQ ID No. 6 from nucleotide position 236 to nucleotide position 253 or its complement;
b) hybridizing said target nucleic acid sequence to a second nucleic acid oligonucleotide comprising the nucleotide sequence of SEQ ID No. 5 from nucleotide 210 to nucleotide 227 or its complement or said second nucleic acid oligonucleotide comprising the nucleotide sequence of SEQ ID No. 6 from nucleotide 254 to nucleotide 271 or its complement, wherein said first and second oligonucleotide overlap by at least one nucleotide and wherein either said first or said second oligonucleotide is labeled to be said labeled nucleic acid probe;
c) cleaving only the labeled probe within the probe:target nucleic acid sequence duplex with an enzyme which causes selective probe cleavage resulting in duplex disassociation, leaving the target sequence intact;
d) recycling of the target nucleic acid sequence by repeating steps (a) to (c); and
e) detecting cleaved labeled probe, thereby determining the presence of said target nucleic acid sequence, and detecting the presence of elite event EE-GM4 in said biological samples.

Two nucleic acids are “substantially complementary” as used herein, when they are not the full complement of each other (as defined herein), such as when their sequences are at least 80%, at least 85%, at least 90%, at least 95%, or at least 99% complementary to each other.

A “kit” as used herein refers to a set of reagents for the purpose of performing the method of the invention, more particularly, the identification of the elite event EE-GM4 in biological samples or the determination of the zygosity status of EE-GM4 containing plant material. More particularly, a preferred embodiment of the kit of the invention comprises at least one or two specific primers, as described above for identification of the elite event, or three specific primers, or two specific primers and one specific probe, as described above for the determination of the zygosity status. Optionally, the kit can further comprise any other reagent described herein in the PCR Identification Protocol or any of the other protocols as described herein for EE-GM4 detection. Alternatively, according to another embodiment of this invention, the kit can comprise a specific probe, as described above, which specifically hybridizes with nucleic acid of biological samples to identify the presence of EE-GM4 therein. Optionally, the kit can further comprise any other reagent (such as but not limited to hybridizing buffer, label) for identification of EE-GM4 in biological samples, using the specific probe.

The kit of the invention can be used, and its components can be specifically adjusted, for purposes of quality control (e.g., purity of seed lots), detection of the presence or absence of the elite event in plant material or material comprising or derived from plant material, such as but not limited to food or feed products.

As used herein, “sequence identity” with regard to nucleotide sequences (DNA or RNA), refers to the number of positions with identical nucleotides divided by the number of nucleotides in the shorter of the two sequences. The alignment of the two nucleotide sequences is performed by the Wilbur and Lipmann algorithm (Wilbur and Lipmann, 1983, Proc. Nat. Acad. Sci. USA 80:726) using a window-size of 20 nucleotides, a word length of 4 nucleotides, and a gap penalty of 4. Computer-assisted analysis and interpretation of sequence data, including sequence alignment as described above, can, e.g., be conveniently performed using the sequence analysis software package of the Genetics Computer Group (GCG, University of Wisconsin Biotechnology Center). Sequences are indicated as “essentially similar” when such sequences have a sequence identity of at least about 75%, at least about 80%, at least about 85%, at least about 90%, at least about 95%, at least about 98%, or at least about 99%, or at least 95%, at least 96%, at least 97%, at least 98%, at least 99%, at least 99.5% or at least 99.9%. It is clear that when RNA sequences are said to be essentially similar or have a certain degree of sequence identity with DNA sequences, thymidine (T) in the DNA sequence is considered equal to uracil (U) in the RNA sequence. Also, it is clear that small differences or mutations may appear in DNA sequences over time and that some mismatches can be allowed for the event-specific primers or probes of the invention, so any DNA sequence indicated herein in any embodiment of this invention for any 3′ or 5′ T-DNA flanking DNA or for any insert or inserted T-DNA or any primer or probe of this invention, also includes sequences essentially similar to the sequences provided herein, such as sequences hybridizing to or with at least 90%, 95%, 96%, 97%, 98%, or at least 99% sequence identity to the sequence given for any 3′ or 5′ T-DNA flanking DNA, for any primer or probe or for any insert or inserted T-DNA of this invention, such as a nucleotide sequence differing in 1 to 200, 1 to 150, 1 to 100, 1 to 75, 1 to 50, 1 to 30, 1 to 20, 1 to 10, 1 to 5, or 1 to 3 nucleotides from any given sequence.

The term “primer” as used herein encompasses any nucleic acid that is capable of priming the synthesis of a nascent nucleic acid in a template-dependent process, such as PCR. Typically, primers are oligonucleotides from 10 to 30 nucleotides, but longer sequences can be employed. Primers may be provided in double-stranded form, though the single-stranded form is preferred. Probes can be used as primers, but are designed to bind to the target DNA or RNA and need not be used in an amplification process.

The term “recognizing” as used herein when referring to specific primers or probes, refers to the fact that the specific primers or probes specifically hybridize to a nucleic acid sequence in the elite event under the conditions set forth in the method (such as the conditions of the PCR Identification Protocol), whereby the specificity is determined by the presence of positive and negative controls.

The term “hybridizing” as used herein when referring to specific probes, refers to the fact that the probe binds to a specific region in the nucleic acid sequence of the elite event under standard stringency conditions. Standard stringency conditions as used herein refers to the conditions for hybridization described herein or to the conventional hybridizing conditions as described by Sambrook et al., 1989 (Molecular Cloning: A Laboratory Manual, Second Edition, Cold Spring Harbor Laboratory Press, NY) which for instance can comprise the following steps: 1) immobilizing plant genomic DNA fragments on a filter, 2) prehybridizing the filter for 1 to 2 hours at 42° C. in 50% formamide, 5×SSPE, 2×Denhardt's reagent and 0.1% SDS, or for 1 to 2 hours at 68° C. in 6×SSC, 2×Denhardt's reagent and 0.1% SDS, 3) adding the hybridization probe which has been labeled, 4) incubating for 16 to 24 hours, 5) washing the filter for 20 min. at room temperature in 1×SSC, 0.1% SDS, 6) washing the filter three times for 20 min. each at 68° C. in 0.2×SSC, 0.1% SDS, and 7) exposing the filter for 24 to 48 hours to X-ray film at −70° C. with an intensifying screen.

As used in herein, a biological sample is a sample of a plant, plant material or products comprising plant material. The term “plant” is intended to encompass soybean (Glycine max) plant tissues, at any stage of maturity, as well as any cells, tissues, or organs taken from or derived from any such plant, including without limitation, any seeds, leaves, stems, flowers, roots, single cells, gametes, cell cultures, tissue cultures or protoplasts. “Plant material”, as used herein refers to material which is obtained or derived from a plant. Products comprising plant material relate to food, feed or other products which are produced using plant material or can be contaminated by plant material. It is understood that, in the context of the present invention, such biological samples are tested for the presence of nucleic acids specific for EE-GM4, implying the presence of nucleic acids in the samples. Thus the methods referred to herein for identifying elite event EE-GM4 in biological samples, relate to the identification in biological samples of nucleic acids which comprise the elite event.

As used herein “comprising” is to be interpreted as specifying the presence of the stated features, integers, steps, reagents or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA sequence which is functionally or structurally defined, may comprise additional DNA sequences, such as promoter, leader, trailer, and/or transcript termination sequences (possibly also including a DNA encoding a targeting or transit peptide).

The present invention also relates to the development of an elite event EE-GM4 in soybean plants comprising this event, the progeny plants and seeds comprising elite event EE-GM4 obtained from these plants and to the plant cells, or plant material derived from plants comprising this event, as well as to such plants or seeds treated with one or more of the compound(s) and/or biological control agent(s) of the invention, or a mixture as described herein. Plants comprising elite event EE-GM4 can be obtained as described in the Examples. This invention also relates to seed comprising elite event EE-GM4 deposited at the ATCC under deposit number PTA-123624 or derivatives therefrom comprising elite event EE-GM4. “Derivatives (of seed)” as used herein, refers to plants which can be grown from such seed, progeny resulting from selfing, crossing or backcrossing, as well as plant cells, organs, parts, tissue, cell cultures, protoplasts, and plant material of same. This includes plants or seeds treated with one or more of the compound(s) and/or biological control agent(s) of the invention, or a mixture as described herein.

Soybean plants or plant material comprising EE-GM4 can be identified according to any one of the identification protocols for EE-GM4 as described in the Examples, including the End-Point method for EE-GM4 identity analysis in Example 2.1, the End-Point method for EE-GM4 identity and zygosity analysis as described in Example 2.2, or the Real-Time PCR method for EE-GM4 Low Level Presence analysis as described in Example 2.3. Briefly, soybean genomic DNA present in the biological sample is amplified by PCR using a primer which specifically recognizes a sequence within the 5′ or 3′ T-DNA flanking sequence of EE-GM4 such as the primer with the sequence of SEQ ID NO: 13 or SEQ ID No. 21, and a primer which recognizes a sequence in the inserted T-DNA, such as the primer with the sequence of SEQ ID No. 12 or SEQ ID No. 20, or with a primer which recognizes the 5′ or 3′ T-DNA flanking sequence of EE-GM4 and the inserted T-DNA contiguous therewith. DNA primers which amplify part of an endogenous soybean sequence are used as positive control for the PCR amplification. If upon PCR amplification, the material yields a fragment of the expected size or gives rise to fluorescence of the expected fluorescent label, the material contains plant material from a soybean plant harboring elite event EE-GM4.

Plants harboring EE-GM4 are characterized by their nematode resistance, particularly SCN, lesion nematode and/or root-knot (“RKN”) and/or reniform nematode resistance, as well as by their tolerance to HPPD inhibitors such as isoxaflutole, topramezone or mesotrione. Soybean plants in different commercially available varieties harboring EE-GM4 are also characterized by having agronomical characteristics that are comparable to the corresponding non-transgenic isogenic commercially available varieties, in the absence of HPPD inhibitor herbicide application and SCN infestation. It has been observed that the presence of an inserted T-DNA in the insertion region of the soybean plant genome described herein, confers particularly interesting phenotypic and molecular characteristics to the plants comprising this event.

Also provided herein is a method for producing a soybean plant resistant to SCN and tolerant to HPPD inhibitor herbicides, comprising introducing resistance to SCN and tolerance to HPPD inhibitor herbicides into the genome of a soybean plant by crossing a first soybean plant lacking a Cry14Ab-1-encoding gene and lacking an HPPD-4-encoding gene with an EE-GM4-containing soybean plant, and selecting a progeny plant resistant to SCN and tolerant to HPPD inhibitor herbicides. In one embodiment, this method includes the treatment of said plant with one or more of the compound(s) and/or biological control agent(s) of the invention, or a mixture as described herein, such as one or more of the nematicidal compound(s) and/or nematicidal biological control agent(s) as described herein. Resistance to SCN can be measured using standard SCN greenhouse assay, e.g., on the world wide web at plantpath.iastate.edu/tylkalab/greenhouse-resistance-screening and plantmanagementnetwork.org/pub/php/review/2009/sce08/.

One embodiment of this invention provides an elite event in soybean plants, obtainable by insertion of 2 transgenes at a specific location in the soybean genome, which elite event confers resistance to nematodes and tolerance to an HPPD inhibitor herbicide such as isoxaflutole, topramezone or mesotrione on such soybean plants, and wherein such elite event has an agronomic performance essentially similar to isogenic lines (as used herein, “isogenic lines” or “near-isogenic lines” are soybean lines of the same genetic background but lacking the transgenes, such as plants of the same genetic background as the plant used for transformation, or segregating sister lines (“nulls”) having lost the transgenes). Particularly, the current invention provides an elite event in soybean plants, wherein the insertion or presence of said elite event in the genome of such soybean plants does not appear to cause an increased susceptibility to disease, does not cause a yield penalty, or does not cause increased lodging, as compared to isogenic lines or to commercial soybean lines. Hence, the current invention provides an elite event in soybean plants, designated as EE-GM4, which results in soybean plants that have improved resistance to nematodes and can tolerate the application of an HPPD inhibitor herbicide such as isoxaflutole, topramezone or mesotrione without negatively affecting the yield of said soybean plants compared to isogenic lines, which soybean plants are not statistically significantly different in their disease susceptibility, or lodging, from isogenic soybean plants or from commercial soybean cultivars. These characteristics make the current elite event a valuable tool in a nematode control and weed resistance management program. In one embodiment, event EE-GM4 is combined with one or more soybean transformation events containing a herbicide tolerance gene, such as one or more soybean transformation events providing tolerance to any one or a combination of glyphosate-based, glufosinate-based, HPPD inhibitor-based, PPO inhibitor-based, sulfonylurea- or imidazolinone-based, AHAS- or ALS-inhibiting and/or auxin-type (e.g., dicamba, 2,4-D) herbicides, including but not limited to Event EE-GM3 (aka FG-072, MST-FG072-3, described in WO2011063411, USDA-APHIS Petition 09-328-O1p), Event SYHTOH2 (aka 0H2, SYN-ØØØH2-5, described in WO2012/082548 and 12-215-01p), Event DAS-68416-4 (aka Enlist Soybean, described in WO2011/066384 and WO2011/066360, USDA-APHIS Petition 09-349-O1p), Event DAS-44406-6 (aka Enlist E3, DAS-444Ø6-6, described in WO2012/075426 and USDA-APHIS 11-234-O1p), Event MON87708 (dicamba-tolerant event of Roundup Ready 2 Xtend Soybeans, described in WO2011/034704 and USDA-APHIS Petition 10-188-01p, MON-877Ø8-9), Event MON89788 (aka Genuity Roundup Ready 2 Yield, MON-89788-1, described in WO2006/130436 and USDA-APHIS Petition 06-178-O1p), Event 40-3-2 (aka Roundup Ready, GTS 40-3-2, MON-Ø4Ø32-6, described in USDA-APHIS Petition 93-258-01), Event A2704-12 (aka LL27, ACS-GMØØ5-3, described in WO2006108674 and USDA-APHIS Petition 96-068-O1p), Event 127 (aka BPS-CV127-9, described in WO2010/080829), Event A5547-127 (aka LL55, EE-GM2, ACS-GMØØ6-4, described in WO2006108675 and in USDA-APHIS Petition 96-068-O1p), event MON87705 (MON-877Ø5-6, Vistive Gold, published PCT patent application, USDA-APHIS Petition 09-201-01p), Event IND-ØØ410-5 (aka HB4 Soybean, US FDA BNF No. 000155, USDA-APHIS Docket No. APHIS-2017-0075, www.aphis.usda.gov/brs/aphisdocs/17_22301p.pdf), Event DP305423 (aka DP-3Ø5423-1, published PCT patent application, USDA-APHIS Petition 06-354-01p), Event DAS-81419-2 (aka Conkesta™ Soybean, described in WO2013016527 and USDA-APHIS Petition 12-272-01p), Event 3560.4.3.5 (aka DP-356043-5, described in WO2008/002872), Event MON87712 (aka MON-87712-4, described in WO2012/051199, USDA-APHIS Petition 11-202-01p), Event MON87769 (aka MON-87769-7, described in WO2009102873 and in USDA-APHIS Petition 09-183-01p, or EE-GM4 is combined with a combination of the following events. Event MON89788×MON87708 (aka Roundup Ready 2 Xtend Soybeans, MON-877Ø8-9×MON-89788-1), Event HOS×Event 40-3-2 (aka Plenish High Oleic Soybeans×Roundup Ready Soybeans), Event EE-GM3×EE-GM2 (aka FG-072×LL55, described in WO2011063413), Event MON 87701×MON 89788 (aka Intacta RR2 Pro Soybean, MON-877Ø1-2×MON-89788-1), DAS-81419-2×DAS-444Ø6-6 (aka Conkesta™ Enlist E3™ Soybean, DAS-81419-2×DAS-444Ø6-6), Event DAS-68416-4×Event MON 89788 (aka Enlist™ RoundUp Ready® 2 Soybean, DAS-68416-4 X MON-89788-1), Event MON 87705 x Event MON 89788 (aka Vistive Gold, MON-877Ø5-6×MON-89788-1), Event DP305423 x Event 40-3-2 (DP-3Ø5423-1×MON-Ø4Ø32-6), Event DP305423×MON87708 (DP-3Ø5423-1 x MON-877Ø8-9), Event DP305423×MON87708×Event MON89788 (DP-3Ø5423-1×MON-877Ø8-9×MON-89788-1), Event DP305423×Event MON89788 (DP-3Ø5423-1×MON-89788-1), Event MON87705×MON87708 (MON-877Ø5-6×MON-877Ø8-9), Event MON87705 x MON87708×MON89788 (MON-877Ø5-6×MON-877Ø8-9×MON-89788-1), Event MON89788×MON87708×A5547-127 (MON-89788-1×MON-877Ø8-9×ACS-GMØØ6-4), Event MON87751×MON87701×MON87708×MON89788 (MON-87751-7×MON-877Ø1-2 x MON-877Ø8-9×MON-89788-1), or Event MON87769×Event MON89788 (aka Omega-3 x Genuity Roundup Ready 2 Yield Soybeans, MON-87769-7×MON-89788-1). This includes such plants or seeds containing such combination of soybean GM events treated with one or more of the compound(s) and/or biological control agent(s) of the invention, or a mixture, as described herein. In another embodiment such plants or seeds also comprise one or more native soybean SCN resistance loci or genes, such as one or more of the SCN resistance genes or loci from the resistance sources of Table 1, or one or more of the SCN resistance genes or loci from the resistance sources PI 88788, PI 548402, PI 209332, or PI 437654.

In one embodiment of the invention, plants or seeds comprising event EE-GM4 also comprise another transformation event providing nematode control, such as a transformation event that produces double-stranded RNA interfering with one or more nematode genes critical for nematode feeding, development, or reproduction, or a transformation event that produces another nematicidal toxin (different from Cry14Ab-1). In another embodiment such plants or seeds also comprise one or more native soybean SCN resistance loci or genes, such as one or more of the SCN resistance genes or loci from the resistance sources of Table 1, or one or more of the SCN resistance genes or loci from the resistance sources PI 88788, PI 548402, PI 209332, or PI 437654.

Provided herein is also a soybean plant or part thereof comprising event EE-GM4, wherein representative soybean seed comprising event EE-GM4 has been deposited under ATCC accession number PTA-123624. Further provided herein are seeds of such plants, comprising such event, as well as a soybean product produced from such seeds, wherein said soybean product comprises event EE-GM4. Such soybean product can be or can comprise soybean meal, ground soybean grain, soybean flakes, soybean flour, or a product comprising any of these processed soybean products. Particularly, such soybean product comprises a nucleic acid that produces an amplicon diagnostic of or specific for event EE-GM4, such amplicon comprising the sequence of any one of SEQ ID No. 1 or 3 or SEQ ID No. 2 or 4. Also provided herein is a method for producing a soybean product, comprising obtaining a soybean seed or grain comprising event EE-GM4, and producing such soybean product therefrom. Also provided herein is a method of obtaining processed food, feed or industrial products derived from soybean grain, such as soybean oil, soybean protein, lecithin, soybean milk, tofu, margarine, biodiesel, biocomposites, adhesives, solvents, lubricants, cleaners, foam, paint, ink, candles, soybean-oil or soybean protein-containing food or (animal) feed products, said method comprising obtaining grain comprising EE-GM4 and producing said processed food, feed or industrial product. In one embodiment, this process can also include the step of a obtaining a soybean seed or plant comprising event EE-GM4, growing said seed or plant in a field, and harvesting soybean grain. Optionally, this method includes application of an HPPD inhibitor herbicide such as IFT, topramezone or mesotrione before planting, before emergence, after emergence or over the top of plants comprising EE-GM4. In one embodiment, the above soybean-derived processed food, feed or industrial products are included in this invention, such as such processed products that produce an EE-GM4 event-specific amplicon using the methods described herein, or that comprise the nucleotide sequence of any one of SEQ ID No. 1, 3 or 5 to SEQ ID No. 2, 4, or 6.

Also provided herein is a soybean plant, which is progeny of any of the above soybean plants, and which comprises event EE-GM4, such as a progeny plant or seed of any one of the above soybean plants that comprises the sequence of SEQ ID No. 1 or 3 or the sequence of SEQ ID No. 2 or 4, or a progeny plant or seed of any one of the above soybean plants that comprises the sequence of SEQ ID No. 1 or 3 and the sequence of SEQ ID No. 2 or 4, or a progeny plant or seed of any one of the above soybean plants that comprises the sequence of SEQ ID No. 5 or SEQ ID No. 24 or the sequence of SEQ ID No. 6 or SEQ ID No. 25, or a progeny plant or seed of any one of the above soybean plants that comprises the sequence of SEQ ID No. 5 or SEQ ID No. 24 and the sequence of SEQ ID No. 6 or SEQ ID No. 25. In one embodiment, such plant or seed is treated with one or more of the compound(s) and/or biological control agent(s) of the invention, or a mixture as described herein. In another embodiment such plants or seeds also comprise one or more native soybean SCN resistance loci or genes, such as one or more of the SCN resistance genes or loci from the resistance sources of Table 1, or one or more of the SCN resistance genes or loci from the resistance sources PI 88788, PI 548402, PI 209332, or PI 437654.

Further provided herein is a method for producing a soybean plant resistant to nematodes and tolerant to isoxaflutole and/or topramezone and/or mesotrione herbicide, comprising introducing into the genome of such plant event EE-GM4, particularly by crossing a first soybean plant lacking event EE-GM4 with a soybean plant comprising EE-GM4, and selecting a progeny plant resistant to nematodes and tolerant to isoxaflutole and/or topramezone and/or mesotrione herbicide. In one embodiment, such method comprises treating the plant or seed of the invention with one or more of the compound(s) and/or biological control agent(s) of the invention, or a mixture as described herein. In another embodiment such plants or seeds also comprise one or more native soybean SCN resistance loci or genes, such as one or more of the SCN resistance genes or loci from the resistance sources of Table 1, or one or more of the SCN resistance genes or loci from the resistance sources PI 88788, PI 548402, PI 209332, or PI 437654.

Also provided herein is a soybean plant resistant to nematodes and tolerant to isoxaflutole, topramezone or mesotrione herbicide with acceptable agronomical characteristics, comprising a Cry14Ab-1 and HPPD-4 protein, and capable of producing an amplicon diagnostic for event EE-GM4. Also provided herein are the specific isolated amplicons (DNA sequence fragments) as such, that can be obtained using the specific detection tools described herein, particularly amplicons including in their sequence a DNA fragment originating from 5′ or 3′ T-DNA flanking DNA and the T-DNA inserted in the plant genome by transformation, as defined herein.

Further provided herein is a method for controlling weeds in a field of soybean plants comprising event EE-GM4, or a field to be planted with such soybean plants (wherein said plants are planted in said field after treatment), comprising treating the field with an effective amount of an HPPD inhibitor herbicide such as an isoxaflutole-based or topramezone-based or mesotrione-based herbicide, wherein such plants are tolerant to such herbicide. In one embodiment, such method comprises treating the soybean plants or seeds, or the soil in which the soybean plants or seeds are grown or are intended to be grown, with one or more of the compound(s) and/or biological control agent(s) or a mixture comprising them, as described herein, such as wherein said compound or biological control agent is nematicidal, insecticidal, or fungicidal.

Further provided herein is a DNA comprising the sequence of SEQ ID No. 5 or 6 or a sequence essentially similar thereto, and any plant, cell, tissue or seed, particularly of soybean, comprising such DNA sequence, such as a plant, cell, tissue, or seed comprising EE-GM4. Also included herein is any soybean plant, cell, tissue or seed, comprising the DNA sequence (heterologous or foreign to a conventional soybean plant, seed, tissue or cell) of SEQ ID No. 5 or 6, or comprising a DNA sequence with at least 99% or 99.5% sequence identity to the sequence of SEQ ID No. 5 or 24 or SEQ ID No. 6 or 25.

Also described is a chimeric DNA comprising an inserted T-DNA, wherein the sequence of said inserted T-DNA comprises the sequence of SEQ ID No. 11 from nucleotide 17 to nucleotide 7621 or SEQ ID No. 23 from nucleotide 1059 to nucleotide 8663, or a sequence with at least 97, 98, 99, 99.5 or at least 99.9% sequence identity thereto, flanked by a 5′ and a 3′ T-DNA flanking region, wherein the 5′ T-DNA flanking region immediately upstream of and contiguous with said inserted T-DNA is characterized by a sequence comprising the sequence of SEQ ID No. 5 from nucleotide 1 to nucleotide 227 or of SEQ ID No. 24 from nucleotide 1 to nucleotide 1058, and wherein the 3′ T-DNA flanking region immediately downstream of and contiguous with said inserted T-DNA is characterized by a sequence comprising the sequence of SEQ ID No. 6 from nucleotide 254 to nucleotide 501 or the nucleotide sequence of the complement of SEQ ID No. 25 from nucleotide 254 to nucleotide 1339. In one embodiment, the sequence of said inserted T-DNA consists of the sequence of SEQ ID No. 11 from nucleotide 17 to nucleotide 7621 or SEQ ID No. 23 from nucleotide 1059 to nucleotide 8663, or a sequence with at least 97, 98, 99, 99.5 or at least 99.9% sequence identity thereto, flanked by part of a 5′ and a 3′ T-DNA flanking region, wherein said part of the 5′ T-DNA flanking region immediately upstream of and contiguous with said inserted T-DNA is characterized by a sequence consisting of the sequence of SEQ ID No. 5 from nucleotide 1 to nucleotide 227 or of SEQ ID No. 24 from nucleotide 1 to nucleotide 1058 or a sequence with at least 97, 98, 99, 99.5 or at least 99.9% sequence identity thereto, and wherein said part of the 3′ T-DNA flanking region immediately downstream of and contiguous with said inserted T-DNA is characterized by a sequence consisting of the sequence of SEQ ID No. 6 from nucleotide 254 to nucleotide 501 or the nucleotide sequence of the complement of SEQ ID No. 25 from nucleotide 254 to nucleotide 1339 or a sequence with at least 97, 98, 99, 99.5 or at least 99.9% sequence identity thereto.

Chimeric DNA refers to DNA sequences, including regulatory and coding sequences that are not found together in nature. Accordingly, a chimeric DNA may comprise DNA regions adjacent to each other that are derived from different sources, or which are arranged in a manner different from that found in nature. Examples of a chimeric DNA are the sequences of SEQ ID No. 5 or 6.

Also provided herein is a transgenic soybean plant, plant cell, tissue, or seed, comprising in their genome event EE-GM4 characterized by a nucleic acid molecule comprising a nucleotide sequence essentially similar to SEQ ID No. 1 or 3 and a nucleic acid molecule comprising a nucleotide sequence essentially similar to SEQ ID No. 2 or 4, or the complement of said sequences, as well as a soybean plant, plant cell, tissue, or seed, comprising in their genome event EE-GM4 characterized by a nucleic acid molecule comprising a nucleotide sequence essentially similar to SEQ ID No. 5 or 24 and SEQ ID No. 6 or 25, or the complement of said sequences.

Even further provided herein is a soybean plant, cell, tissue or seed, comprising EE-GM4, characterized by comprising in the genome of its cells a nucleic acid sequence with at least 80%, at least 85%, at least 90%, at least 95%, at least 96%, at least 97%, at least 98%, at least 99% or 100% sequence identity to any one of SEQ ID No. 1, 3, 5, or 24 and a nucleic acid sequence with at least 80%, at least 90%, at least 95%, at least 96%, at least 97%, at least 98%, at least 99% or 100% sequence identity to any one of SEQ ID No. 2, 4, 6, or 25, or the complement of said sequences.

In one embodiment, the above plant or seed comprising EE-GM4 is treated with one or more of the compound(s) and/or biological control agent(s) of the invention, or a mixture/combination as described herein.

The term “isoxaflutole”, as used herein, refers to the herbicide isoxaflutole [i.e. (5-cyclopropyl-4-isoxazolyl)[2-(methylsulfonyl)-4-(trifluoromethyl)phenyl]methanone], the active metabolite thereof, diketonitrile, and any mixtures or solutions comprising said compound. HPPD inhibiting herbicides useful for application on the event of this invention are the diketonitriles, e.g., 2-cyano-3-cyclopropyl-1-(2-methylsulphonyl-4-trifluoromethylphenyl)-propane-1,3-dione and 2-cyano-1-[4-(methylsulphonyl)-2-trifluoromethylphenyl]-3-(1-methylcyclopropyl)propane-1,3-fione; other isoxazoles; and the pyrazolinates, e.g. topramezone [i.e. [3-(4,5-dihydro-3-isoxazolyl)-2-methyl-4-(methylsulfonyl) phenyl](5-hydroxy-1-methyl-H-pyrazol-4-yl)methanone], and pyrasulfotole [(5-hydroxy-1,3-dimethylpyrazol-4-yl(2-mesyl-4-trifluaromethylphenyl) methanone]; or mesotrione [2-[4-(Methylsulfonyl)-2-nitrobenzoyl]cyclohexane-1,3-dione]; or 2-chloro-3-(methylsulfanyl)-N-(1-methyl-1H-tetrazol-5-yl)-4-(trifluoromethyl)benzamide]; or 2-methyl-N-(5-methyl-1,3,4-oxadiazol-2-yl)-3-(methylsulfonyl)-4-(trifluoromethyl)benzamide; or pyrazofen [2-[4-(2,4-dichlorobenzoyl)-1,3-dimethylpyrazol-5-yloxy]acetophenone].

In one embodiment of this invention, a field to be planted with soybean plants containing the EE-GM4 event, can be treated with an HPPD inhibitor herbicide, such as isoxaflutole (‘IFT’), topramezone or mesotrione, or with both an HPPD inhibitor herbicide and glyphosate, before the soybean is sown, which cleans the field of weeds that are killed by the HPPD inhibitor and/or glyphosate, allowing for no-till practices, followed by planting or sowing of the soybeans in that same pre-treated field later on (burn-down application using an HPPD inhibitor herbicide). The residual activity of IFT will also protect the emerging and growing soybean plants from competition by weeds in the early growth stages. Once the soybean plants have a certain size, and weeds tend to re-appear, an HPPD inhibitor or a mixture of an HPPD inhibitor with a selective (conventional) soybean herbicide or a mixture of an HPPD inhibitor with a herbicide that is non-selective in soybean (e.g., glyphosate or glufosinate) but for which the plants contain a tolerance gene/locus so that they are tolerant to said herbicide, can be applied as post-emergent herbicide over the top of the plants.

In another embodiment of this invention, a field in which seeds containing the EE-GM4 event were sown, can be treated with an HPPD inhibitor herbicide, such as IFT, topramezone or mesotrione, before the soybean plants emerge but after the seeds are sown (the field can be made weed-free before sowing using other means, including conventional tillage practices such as ploughing, chisel ploughing, or seed bed preparation), where residual activity will keep the field free of weeds killed by the herbicide so that the emerging and growing soybean plants have no competition by weeds (pre-emergence application of an HPPD inhibitor herbicide). Once the soybean plants have a certain size, and weeds tend to re-appear, an HPPD inhibitor—or an HPPD inhibitor-soybean selective (conventional) herbicide mixture or a mixture of an HPPD inhibitor with a herbicide that is non-selective in soybean (e; g., glyphosate or glufosinate) but for which the plants contain a tolerance gene/locus so that said plants are tolerant to said herbicide—can be applied as post-emergent herbicide over the top of the plants. In one embodiment of the invention is provided a process for weed control comprising sowing in a field EE-GM4-containing soybean seeds, and treating said field with an HPPD inhibitor herbicide before plants emerge from said seed, but after the seeds are sown.

In another embodiment of this invention, plants containing the EE-GM4 event can be treated with an HPPD inhibitor herbicide, such as IFT, topramezone or mesotrione, over the top of the soybean plants that have emerged from the seeds that were sown, which cleans the field of weeds killed by the HPPD inhibitor, which application can be together with (e.g., in a spray tank mix), followed by or preceded by a treatment with a selective (conventional) soybean post-emergent herbicide, or a herbicide that is non-selective in soybean (e.g., glyphosate or glufosinate) but for which the plants contain a tolerance gene/locus so that said plants are tolerant to said herbicide, over the top of the plants (post-emergence application of an HPPD inhibitor herbicide (with or without said soybean selective or non-selective herbicide)).

In one embodiment, such methods involving use of an HAPPD inhibitor herbicide, also comprise treating the soybean plants or seeds, or the soil in which the soybean plants or seeds are grown or are intended to be grown, with one or more of the compound(s) and/or biological control agent(s) or a mixture comprising them, as described herein, such as wherein said compound or biological control agent is nematicidal, insecticidal, or fungicidal. In another embodiment such plants or seeds also comprise one or more native soybean SCN resistance loci or genes, such as one or more of the SCN resistance genes or loci from the resistance sources of Table 1, or one or more of the SCN resistance genes or loci from the resistance sources PI 88788, PI 548402, PI 209332, or PI 437654.

Also, in accordance with the current invention, soybean plants harboring EE-GM4 (which may also contain another herbicide tolerance soybean event/trait as described herein) may be treated with, or soybean seeds harboring EE-GM4 may be coated with, any soybean insectide, herbicide or fungicide. In one embodiment, such plants or seeds also contain one or more soybean SCN resistance genes from from the resistance sources of Table 1, or or one or more of the SCN resistance genes or loci from the resistance sources PI 88788, PI 548402, PI 209332 or PI 437654, or comprise one or more of the soybean SCN resistance loci or genes selected from the group consisting of: rhg1, rhg1-b, rhg2, rhg3, Rhg4, Rhg5, qSCN11, cqSCN-003, cqSCN-005, cqSCN-006, and cqSCN-007.

The compounds (such as pesticides or herbicides), biological control agents, or plant growth regulators of the invention as referred to herein are described below, as well as formulations, mixtures and types of treatment. In one embodiment, the compound(s) and/or biological control agent(s) of the invention, or a mixture comprising them, is/are used as seed treatment on the seeds of the invention, as described below.

### Insecticides/Acaricides/Nematicides

The active ingredients specified herein by their Common Name are known and described, for example, in The Pesticide Manual (17th Ed., British Crop Protection Council, updated version at on the world wide web at bcpc.org/product/bcpc-online-pesticide-manual-latest-version) or can be searched on the internet (e.g., on the world wide web at alanwood.net/pesticides). The classification is based on the current IRAC Mode of Action Classification Scheme at the time of filing of this patent application.

In one embodiment, an insecticide/acaricide/nematicide used in this invention is from the following groups IAN1 to IAN30: IAN1: Acetylcholinesterase (AChE) inhibitors, preferably carbamates selected from alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb, or organophosphates selected from acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.

IAN2: GABA-gated chloride channel blockers, preferably cyclodiene-organochlorines selected from chlordane and endosulfan or phenylpyrazoles (fiproles), for example ethiprole and fipronil.

IAN3: Sodium channel modulators, preferably pyrethroids selected from acrinathrin, allethrin, d-cis-trans allethrin, d-trans allethrin, bifenthrin, bioallethrin, bioallethrin s-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans-isomer], deltamethrin, empenthrin [(EZ)-(1R)-isomer], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans-isomer], prallethrin, pyrethrins (pyrethrum), resmethrin, silafluofen, tefluthrin, tetramethrin, tetramethrin [(1R)-isomer)], tralomethrin and transfluthrin or DDT or methoxychlor.

IAN4: Nicotinic acetylcholine receptor (nAChR) competitive modulators, preferably neonicotinoids selected from acetamiprid, clothianidin, dinotefuran, imidacloprid, nitenpyram, thiacloprid and thiamethoxam or nicotine or sulfoxaflor or flupyradifurone.

IAN5: Nicotinic acetylcholine receptor (nAChR) allosteric modulators, preferably spinosyns selected from spinetoram and spinosad.

IAN6: Glutamate-gated chloride channel (GluCl) allosteric modulators, preferably avermectins/milbemycins selected from abamectin, emamectin benzoate, lepimectin and milbemectin.

IAN7: Juvenile hormone mimics, preferably juvenile hormone analogues selected from hydroprene, kinoprene and methoprene, or fenoxycarb or pyriproxyfen.

IAN8: Miscellaneous non-specific (multi-site) inhibitors, preferably alkyl halides selected from methyl bromide and other alkyl halides, or chloropicrine or sulphuryl fluoride or borax or tartar emetic or methyl isocyanate generators selected from diazomet and metam.

IAN9: Chordotonal organ TRPV channel modulators selected from pymetrozine, afidopyropen and pyrifluquinazone.

IAN10: Mite growth inhibitors selected from clofentezine, hexythiazox, diflovidazin and etoxazole.

IAN11: Microbial disruptors of the insect gut membrane selected from Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and B.t. plant proteins selected from Cry1Ab, Cry1Ac, Cry1Fa, Cry1A.105, Cry2Ab, Vip3A, mCry3A, Cry3Ab, Cry3Bb and Cry34Abl/35Ab1.

IAN12: Inhibitors of mitochondrial ATP synthase, preferably ATP disruptors selected from diafenthiuron, or organotin compounds selected from azocyclotin, cyhexatin and fenbutatin oxide, or propargite or tetradifon.

IAN13: Uncouplers of oxidative phosphorylation via disruption of the proton gradient selected from chlorfenapyr, DNOC and sulfluramid.

IAN14: Nicotinic acetylcholine receptor channel blockers selected from bensultap, cartap hydrochloride, thiocylam and thiosultap-sodium.

IAN15: Inhibitors of chitin biosynthesis, type 0, selected from bistrifluron, chlorfluazuron, diflubenzuron, flucycloxuron, flufenoxuron, hexaflumuron, lufenuron, novaluron, noviflumuron, teflubenzuron and triflumuron.

IAN16: Inhibitors of chitin biosynthesis, type 1 selected from buprofezin.

IAN17: Moulting disruptor (in particular for Diptera, i.e. dipterans) selected from cyromazine.

IAN18: Ecdysone receptor agonists selected from chromafenozide, halofenozide, methoxyfenozide and tebufenozide.

IAN19: Octopamine receptor agonists selected from amitraz.

IAN20: Mitochondrial complex III electron transport inhibitors selected from hydramethylnone, acequinocyl and fluacrypyrim.

IAN21: Mitochondrial complex I electron transport inhibitors, preferably METI acaricides selected from fenazaquin, fenpyroximate, pyrimidifen, pyridaben, tebufenpyrad and tolfenpyrad, or rotenone (Derris).

IAN22: Voltage-dependent sodium channel blockers selected from indoxacarb and metaflumizone.

IAN23: Inhibitors of acetyl CoA carboxylase, preferably tetronic and tetramic acid derivatives selected from spirodiclofen, spiromesifen and spirotetramat.

IAN24: Mitochondrial complex IV electron transport inhibitors, preferably phosphines selected from aluminium phosphide, calcium phosphide, phosphine and zinc phosphide, or cyanides selected from calcium cyanide, potassium cyanide and sodium cyanide.

IAN25: Mitochondrial complex II electron transport inhibitors, preferably beta-ketonitrile derivatives selected from cyenopyrafen and cyflumetofen, and carboxanilides selected from pyflubumide.

IAN28: Ryanodine receptor modulators, preferably diamides selected from chlorantraniliprole, cyantraniliprole, tetranaliiprole and flubendiamide.

IAN29: Chordotonal organ Modulators (with undefined target site) selected from flonicamid.

IAN30: further active compounds selected from the group consisting of: Afidopyropen, Afoxolaner, Azadirachtin, Benclothiaz, Benzoximate, Bifenazate, Broflanilide, Bromopropylate, Chinomethionat, Chloroprallethrin, Cryolite, Cyclaniliprole, Cycloxaprid, Cyhalodiamide, Dicloromezotiaz, Dicofol, epsilon-Metofluthrin, epsilon-Momfluthrin, Flometoquin, Fluazaindolizine, Fluensulfone, Flufenerim, Flufenoxystrobin, Flufiprole, Fluhexafon, Fluopyram (including the formulations described in WO2018046381, incorporated herein by reference), Fluralaner, Fluxametamide, Fufenozide, Guadipyr, Heptafluthrin, Imidaclothiz, Iprodione, kappa-Bifenthrin, kappa-Tefluthrin, Lotilaner, Meperfluthrin, Paichongding, Pyridalyl, Pyrifluquinazon, Pyriminostrobin, Spirobudiclofen, Tetramethylfluthrin, Tetraniliprole, Tetrachlorantraniliprole, Tigolaner, Tioxazafen (Nemastrike™), Thiofluoximate, Triflumezopyrim and iodomethane; furthermore preparations based on Bacillus firmus (I-1582, BioNeem, Votivo), and also the following compounds: 1-{2-fluoro-4-methyl-5-[(2,2,2-trifluoroethyl)sulphinyl]phenyl}-3-(trifluoromethyl)-1H-1,2,4-triazole-5-amine (known from WO2006/043635) (CAS 885026-50-6), {1′-[(2E)-3-(4-chlorophenyl)prop-2-en-1-yl]-5-fluorospiro[indol-3,4′-piperidin]-1(2H)-yl}(2-chloropyridin-4-yl)methanone (known from WO2003/106457) (CAS 637360-23-7), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494) (CAS 872999-66-1), 3-(4-chloro-2,6-dimethylphenyl)-4-hydroxy-8-methoxy-1,8-diazaspiro[4.5]dec-3-en-2-one (known from WO2010052161) (CAS 1225292-17-0), 3-(4-chloro-2,6-dimethylphenyl)-8-methoxy-2-oxo-1,8-diazaspiro[4.5]dec-3-en-4-yl ethyl carbonate (known from EP2647626) (CAS 1440516-42-6), 4-(but-2-yn-1-yloxy)-6-(3,5-dimethylpiperidin-1-yl)-5-fluoropyrimidine (known from WO2004/099160) (CAS 792914-58-0), PF1364 (known from JP2010/018586) (CAS 1204776-60-2), N-[(2E)-1-[(6-chloropyridin-3-yl)methyl]pyridin-2(1H)-ylidene]-2,2,2-trifluoroacetamide (known from WO2012/029672) (CAS 1363400-41-2), (3E)-3-[1-[(6-chloro-3-pyridyl)methyl]-2-pyridylidene]-1,1,1-trifluoro-propan-2-one (known from WO2013/144213) (CAS 1461743-15-6), N-[3-(benzylcarbamoyl)-4-chlorophenyl]-1-methyl-3-(pentafluoroethyl)-4-(trifluoromethyl)-1H-pyrazole-5-carboxamide (known from WO2010/051926) (CAS 1226889-14-0), 5-bromo-4-chloro-N-[4-chloro-2-methyl-6-(methylcarbamoyl)phenyl]-2-(3-chloro-2-pyridyl)pyrazole-3-carboxamide (known from CN103232431) (CAS 1449220-44-3), 4-[5-(3,5-dichlorophenyl)-4,5-dihydro-5-(trifluoromethyl)-3-isoxazolyl]-2-methyl-N-(cis-1-oxido-3-thietanyl)-benzamide, 4-[5-(3,5-dichlorophenyl)-4,5-dihydro-5-(trifluoromethyl)-3-isoxazolyl]-2-methyl-N-(trans-1-oxido-3-thietanyl)-benzamide and 4-[(5S)-5-(3,5-dichlorophenyl)-4,5-dihydro-5-(trifluoromethyl)-3-isoxazolyl]-2-methyl-N-(cis-1-oxido-3-thietanyl)benzamide (known from WO 2013/050317 A1) (CAS 1332628-83-7), N-[3-chloro-1-(3-pyridinyl)-1H-pyrazol-4-yl]-N-ethyl-3-[(3,3,3-trifluoropropyl)sulfinyl]-propanamide, (+)-N-[3-chloro-1-(3-pyridinyl)-1H-pyrazo-4-yl]-N-ethyl-3-[(3,3,3-trifluoropropyl)sulfinyl]-propanamide and (−)-N-[3-chloro-1-(3-pyridinyl)-1H-pyrazol-4-yl]-N-ethyl-3-[(3,3,3-trifluoropropyl)sulfinyl]-propanamide (known from WO 2013/162715 A2, WO 2013/162716 A2, US 2014/0213448 A1) (CAS 1477923-37-7), 5-[[(2E)-3-chloro-2-propen-1-yl]amino]-1-[2,6-dichloro-4-(trifluoromethyl)phenyl]-4-[(trifluoromethyl)sulfinyl]-1H-pyrazole-3-carbonitrile (known from CN 101337937 A) (CAS 1105672-77-2), 3-bromo-N-[4-chloro-2-methyl-6-[(methylamino)thioxomethyl]phenyl]-1-(3-chloro-2-pyridinyl)-1H-pyrazole-5-carboxamide, (Liudaibenjiaxuanan, known from CN 103109816 A) (CAS 1232543-85-9); N-[4-chloro-2-[[(1,1-dimethylethyl)amino]carbonyl]-6-methylphenyl]-1-(3-chloro-2-pyridinyl)-3-(fluoromethoxy)-1H-Pyrazole-5-carboxamide (known from WO 2012/034403 A1) (CAS 1268277-22-0), N-[2-(5-amino-1,3,4-thiadiazol-2-yl)-4-chloro-6-methylphenyl]-3-bromo-1-(3-chloro-2-pyridinyl)-1H-pyrazole-5-carboxamide (known from WO 2011/085575 A1) (CAS 1233882-22-8), 4-[3-[2,6-dichloro-4-[(3,3-dichloro-2-propen-1-yl) oxy]phenoxy]propoxy]-2-methoxy-6-(trifluoromethyl)-pyrimidine (known from CN 101337940 A) (CAS 1108184-52-6); (2E)- and 2(Z)-2-[2-(4-cyanophenyl)-1-[3-(trifluoromethyl)phenyl]ethylidene]-N-[4-(difluoromethoxy)phenyl]-hydrazinecarboxamide (known from CN 101715774 A) (CAS 1232543-85-9); 3-(2,2-dichloroethenyl)-2,2-dimethyl-4-(1H-benzimidazol-2-yl)phenyl-cyclopropanecarboxylic acid ester (known from CN 103524422 A) (CAS 1542271-46-4); (4aS)-7-chloro-2,5-dihydro-2-[[(methoxycarbonyl)[4-[(trifluoromethyl)thio]phenyl]amino]carbonyl]-indeno[1,2-e][1,3,4]oxadiazine-4a(3H)-carboxylic acid methyl ester (known from CN 102391261 A) (CAS 1370358-69-2); 6-deoxy-3-O-ethyl-2,4-di-O-methyl-, 1-[N-[4-[1-[4-(1,1,2,2,2-pentafluoroethoxy)phenyl]-1H-1,2,4-triazol-3-yl]phenyl]carbamate]-α-L-mannopyranose (known from US 2014/0275503 A1) (CAS 1181213-14-8); 8-(2-cyclopropylmethoxy-4-trifluoromethyl-phenoxy)-3-(6-trifluoromethyl-pyridazin-3-yl)-3-aza-bicyclo[3.2.1]octane (CAS 1253850-56-4), (8-anti)-8-(2-cyclopropylmethoxy-4-trifluoromethyl-phenoxy)-3-(6-trifluoromethyl-pyridazin-3-yl)-3-aza-bicyclo[3.2.1]octane (CAS 933798-27-7), (8-syn)-8-(2-cyclopropylmethoxy-4-trifluoromethyl-phenoxy)-3-(6-trifluoromethyl-pyridazin-3-yl)-3-aza-bicyclo[3.2.1]octane (known from WO 2007040280 A1, WO 2007040282 A1) (CAS 934001-66-8), N-[3-chloro-1-(3-pyridinyl)-1H-pyrazol-4-yl]-N-ethyl-3-[(3,3,3-trifluoropropyl)thio]-propanamide (known from WO 2015/058021 A1, WO 2015/058028 A1) (CAS 1477919-27-9) and N-[4-(aminothioxomethyl)-2-methyl-6-[(methylamino)carbonyl]phenyl]-3-bromo-1-(3-chloro-2-pyridinyl)-1H-pyrazole-5-carboxamide (known from CN 103265527 A) (CAS 1452877-50-7), 5-(1,3-dioxan-2-yl)-4-[[4-(trifluoromethyl) phenyl]methoxy]-pyrimidine (known from WO 2013/115391 A1) (CAS 1449021-97-9), 3-(4-chloro-2,6-dimethylphenyl)-4-hydroxy-8-methoxy-1-methyl-1,8-diazaspiro[4.5]dec-3-en-2-one (known from WO 2010/066780 A1, WO 2011/151146 A1) (CAS 1229023-34-0), 3-(4-chloro-2,6-dimethylphenyl)-8-methoxy-1-methyl-1,8-diazaspiro[4.5]decane-2,4-dione (known from WO 2014/187846 A1) (CAS 1638765-58-8), 3-(4-chloro-2,6-dimethylphenyl)-8-methoxy-1-methyl-2-oxo-1,8-diazaspiro[4.5]dec-3-en-4-yl-carbonic acid ethyl ester (known from WO 2010/066780 A1, WO 2011151146 A1) (CAS 1229023-00-0), N-[1-[(6-chloro-3-pyridinyl)methyl]-2(1H)-pyridinylidene]-2,2,2-trifluoro-acetamide (known from DE 3639877 A1, WO 2012029672 A1) (CAS 1363400-41-2), [N(E)]-N-[1-[(6-chloro-3-pyridinyl)methyl]-2(1H)-pyridinylidene]-2,2,2-trifluoro-acetamide, (known from WO 2016005276 A1) (CAS 1689566-03-7), [N(Z)]—N-[1-[(6-chloro-3-pyridinyl)methyl]-2(1H)-pyridinylidene]-2,2,2-trifluoro-acetamide, (CAS 1702305-40-5), 3-endo-3-[2-propoxy-4-(trifluoromethyl)phenoxy]-9-[[5-(trifluoromethyl)-2-pyridinyl]oxy]-9-azabicyclo[3.3.1]nonane (known from WO 2011/105506 A1, WO 2016/133011 A1) (CAS 1332838-17-1), the compounds described in WO2018087036 or WO2015169776, flupyrimin or mixtures thereof (see EP0268915, WO2012/029672, WO2013/129688), Spiropidion, Benzpyrimoxan, Pyrifluramide, the nematicides described in U.S. Pat. No. 9,433,218 or 9,701,673 (incorporated herein by reference), and N-[1-(2,6-difluorophenyl)-1H-pyrazol-3-yl]-2-trifluoromethylbenzamide (described in WO2014/053450).

Also included as insecticide/acaricide/nematicides used in this invention are pyridazinamides including but not limited to 1-isopropyl-N,5-dimethyl-N-pyridazin-4-yl-pyrazole-4-carboxamide; 1-(1,2-dimethylpropyl)-N-ethyl-5-methyl-N-pyridazin-4-yl-pyrazole-4-carboxamide; N,5-dimethyl-N-pyridazin-4-yl-1-(2,2,2-trifluoro-1-methyl-ethyl)pyrazole-4-carboxamide; 1-[1-(1-cyanocyclopropyl)ethyl]-N-ethyl-5-methyl-N-pyridazin-4-yl-pyrazole-4-carboxamide; N-ethyl-1-(2-fluoro-1-methyl-propyl)-5-methyl-N-pyridazin-4-yl-pyrazole-4-carboxamide; 1-(1,2-dimethylpropyl)-N,5-dimethyl-N-pyridazin-4-yl-pyrazole-4-carboxamide; 1-[1-(1-cyanocyclopropyl)ethyl]-N,5-dimethyl-N-pyridazin-4-yl-pyrazole-4-carboxamide; N-methyl-1-(2-fluoro-1-methyl-propyl]-5-methyl-N-pyridazin-4-yl-pyrazole-4-carboxamide; 1-(4,4-difluorocyclohexyl)-N-ethyl-5-methyl-N-pyridazin-4-yl-pyrazole-4-carboxamide; or 1-(4,4-difluorocyclohexyl)-N,5-dimethyl-N-pyridazin-4-yl-pyrazole-4-carboxamide. In some embodiments, the pyridazinamide used in the invention is 1-(1,2-dimethylpropyl)-N-ethyl-5-methyl-N-pyridazin-4-yl-pyrazole-4-carboxamide.

Also included as insecticide/acaricide/nematicides used in this invention are mesoionics including but not limited to (3R)-3-(2-chlorothiazol-5-yl)-8-methyl-5-oxo-6-phenyl-2,3-dihydrothiazolo[3,2-a]pyrimidin-8-ium-7-olate; (3S)-3-(6-chloro-3-pyridyl)-8-methyl-5-oxo-6-phenyl-2,3-dihydrothiazolo[3,2-a]pyrimidin-8-ium-7-olate; (3S)-8-methyl-5-oxo-6-phenyl-3-pyrimidin-5-yl-2,3-dihydrothiazolo[3,2-a]pyrimidin-8-ium-7-olate; (3R)-3-(2-chlorothiazol-5-yl)-8-methyl-5-oxo-6-[3-(trifluoromethyl)phenyl]-2,3-dihydrothiazolo[3,2-a]pyrimidin-8-ium-7-olate; (3R)-3-(2-chlorothiazol-5-yl)-6-(3,5-dichlorophenyl)-8-methyl-5-oxo-2,3-dihydrothiazolo[3,2-a]pyrimidin-8-ium-7-olate; (3R)-3-(2-chlorothiazol-5-yl)-8-ethyl-5-oxo-6-phenyl-2,3-dihydrothiazolo[3,2-a]pyrimidin-8-ium-7-olate. In some embodiments, the mesoionic used in the invention is (3R)-3-(2-chlorothiazol-5-yl)-8-methyl-5-oxo-6-phenyl-2,3-dihydrothiazolo[3,2-a]pyrimidin-8-ium-7-olate.

Further nematicidal compound used in this invention include trifluoroethyl sulfoxide (known from Kumiai/Bayer); N-[1-(2,6-difluorophenyl)-1H-pyrazol-3-yl]-2-(trifluoromethyl)benzamide (compound (1a or Ia; described in WO15144681); N-[1-(3,5-difluoropyridin-2-yl)-1H-pyrazol-3-yl]-2-(trifluoromethyl)benzamide (compound 1b or Ib); N-[2-(2, 6-Difluorophenyl)-2H-1, 2,3-triazol-4-yl]-2-(trifluoromethyl)benzamide (compound 1c or Ic).

An insecticide/insecticidal agent in crop protection, as described herein for use on or with the plants or seeds of the invention, is capable of controlling insects. The term “controlling insects”, or “insect-contolling” as used herein, means killing the insects (in any stage, preferably at least in the larval stage) or preventing or impeding their development, their reproduction, or their growth or preventing or impeding their penetration into or their sucking/feeding on plant tissue.

A nematicide/nematicidal agent in crop protection, as described herein for use on or with the plants or seeds of the invention, is capable of controlling nematodes. The term “controlling nematodes”, or “nematode-controlling”, as used herein, means killing the nematodes or preventing or impeding their development, their reproduction, or their growth or preventing or impeding their penetration into or their sucking/feeding on plant tissue. Hence, a “nematicide” or “nematicidal agent”, as used herein, is an agent that can kill nematodes (such as plant-pathogenic nematodes) or can prevent or impede their development, their reproduction, or their growth, or can prevent or impede their penetration into or their sucking/feeding on plant tissue.

The efficacy of nematicidal compounds or biological control agents is determined by comparing mortalities, gall formation, cyst formation, nematode density per volume of soil, nematode density per root, number of nematode eggs per soil volume, mobility of the nematodes between a treated plant or plant part or the treated soil and an untreated plant or plant part or the untreated soil. In one embodiment the reduction achieved is 25-50% in comparison to an untreated plant, plant part or the untreated soil, in another embodiment 51-79% reduction in comparison to an untreated plant, plant part or the untreated soil and in yet another embodiment refers to the complete kill or the complete prevention of development and growth of the nematodes by a reduction of 80 to 100%. The control of nematodes as described herein also comprises the control of proliferation of the nematodes (development of cysts and/or eggs). Nematicidal compounds and/or or biological control agents can also be used to keep the soybean plants of the invention more healthy, and they can be employed curatively, preventatively or systemically for the control of nematodes.

The use of the nematode-controlling compounds or biological control agents can further increase the yield of the plants of the invention.

The nematode-controlling compounds or biological control agents as mentioned herein can be used to control plant nematodes such as Aglenchus agricola, Anguina tritici, Aphelenchoides arachidis, Aphelenchoides fragaria, and the stem and leaf endoparasites Aphelenchoides spp., Belonolaimus gracilis, Belonolaimus longicaudatus, Belonolaimus nortoni, Bursaphelenchus cocophilus, Bursaphelenchus eremus, Bursaphelenchus xylophilus and Bursaphelenchus spp., Cacopaurus pestis, Criconemella curvata, Criconemella onoensis, Criconemella ornata, Criconemella rusium, Criconemella xenoplax (Mesocriconema xenoplax) and Criconemella spp., Criconemoides ferniae, Criconemoides onoense, Criconemoides ornatum and Criconemoides spp., Ditylenchus destructor, Ditylenchus dipsaci, Ditylenchus myceliophagus and also the stem and leaf endoparasites Ditylenchus spp., Dolichodorus heterocephalus, Globodera pallida (Heterodera pallida), Globodera rostochiensis (yellow potato cyst nematode), Globodera solanacearum, Globodera tabacum, Globodera virginia and the non-migratory cyst-forming parasites Globodera spp., Helicotylenchus digonicus, Helicotylenchus dihystera, Helicotylenchus erythrine, Helicotylenchus multicinctus, Helicotylenchus nannus, Helicotylenchus pseudorobustus and Helicotylenchus spp., Hemicriconemoides, Hemicycliophora arenaria, Hemicycliophora nudata, Hemicycliophora parvana, Heterodera avenae, Heterodera cruciferae, Heterodera glycines, Heterodera oryzae, Heterodera schachtii, Heterodera zeae and the non-migratory cyst-forming parasites Heterodera spp., Hirschmaniella gracilis, Hirschmaniella oryzae, Hirschmaniella spinicaudata and the stem and leaf endoparasites Hirschmaniella spp., Hoplolaimus aegyptii, Hoplolaimus calfornicus, Hoplolaimus columbus, Hoplolaimus galeatus, Hoplolaimus indicus, Hoplolaimus magnistylus, Hoplolaimus pararobustus, Longidorus africanus, Longidorus breviannulatus, Longidorus elongatus, Longidorus laevicapitatus, Longidorus vineacola and the ectoparasites Longidorus spp., Meloidogyne acronea, Meloidogyne africana, Meloidogyne arenaria, Meloidogyne arenaria thamesi, Meloidogyne artiella, Meloidogyne chitwoodi, Meloidogyne coffeicola, Meloidogyne ethiopica, Meloidogyne exigua, Meloidogyne fallax, Meloidogyne graminicola, Meloidogyne graminis, Meloidogyne hapla, Meloidogyne incognita, Meloidogyne incognita acrita, Meloidogyne javanica, Meloidogyne kikuyensis, Meloidogyne minor, Meloidogyne naasi, Meloidogyne paranaensis, Meloidogyne thamesi and the non-migratory parasites Meloidogyne spp., Meloinema spp., Nacobbus aberrans, Neotylenchus vigissi, Paraphelenchus pseudoparietinus, Paratrichodorus allius, Paratrichodorus lobatus, Paratrichodorus minor, Paratrichodorus nanus, Paratrichodorus porosus, Paratrichodorus teres and Paratrichodorus spp., Paratylenchus hamatus, Paratylenchus minutus, Paratylenchus projectus and Paratylenchus spp., Pratylenchus agilis, Pratylenchus alleni, Pratylenchus andinus, Pratylenchus brachyurus, Pratylenchus cerealis, Pratylenchus coffeae, Pratylenchus crenatus, Pratylenchus delattrei, Pratylenchus giibbicaudatus, Pratylenchus goodeyi, Pratylenchus hamatus, Pratylenchus hexincisus, Pratylenchus loosi, Pratylenchus neglectus, Pratylenchus penetrans, Pratylenchus pratensis, Pratylenchus scribneri, Pratylenchus teres, Pratylenchus thornei, Pratylenchus vulnus, Pratylenchus zeae and the migratory endoparasites Pratylenchus spp., Pseudohalenchus minutus, Psilenchus magnidens, Psilenchus tumidus, Punctodera chalcoensis, Quinisulcius acutus, Radopholus citrophilus, Radopholus similis, the migratory endoparasites Radopholus spp., Rotylenchulus borealis, Rotylenchulus parvus, Rotylenchulus reniformis and Rotylenchulus spp., Rotylenchus laurentinus, Rotylenchus macrodoratus, Rotylenchus robustus, Rotylenchus uniformis and Rotylenchus spp., Scutellonema brachyurum, Scutellonema bradys, Scutellonema clathricaudatum and the migratory endoparasites Scutellonema spp., Subanguina radiciola, Tetylenchus nicotianae, Trichodorus cylindricus, Trichodorus minor, Trichodorus primitivus, Trichodorus proximus, Trichodorus similis, Trichodorus sparsus and the ectoparasites Trichodorus spp., Tylenchorhynchus agri, Tylenchorhynchus brassicae, Tylenchorhynchus clarus, Tylenchorhynchus claytoni, Tylenchorhynchus digitatus, Tylenchorhynchus ebriensis, Tylenchorhynchus maximus, Tylenchorhynchus nudus, Tylenchorhynchus vulgaris and Tylenchorhynchus spp., Tylenchulus semipenetrans and the semiparasites Tylenchulus spp., Xiphinema americanum, Xiphinema brevicolle, Xiphinema dimorphicaudatum, Xiphinema index and the ectoparasites Xiphinema spp.

The above compounds with nematode-controlling activity are particularly suitable for controlling soybean nematodes, in particular nematodes selected from the group consisting of: Pratylenchus brachyurus, Pratylenchus pratensis, Pratylenchus penetrans, Pratylenchus scribneri, Belonolaimus longicaudatus, Heterodera glycines, Hoplolaimus columbus and also Pratylenchus coffeae, Pratylenchus hexincisus, Pratylenchus neglectus, Pratylenchus crenatus, Pratylenchus alleni, Pratylenchus agilis, Pratylenchus zeae, Pratylenchus vulnus, Belonolaimus gracilis, Meloidogyne arenaria, Meloidogyne incognita, Meloidogyne javanica, Meloidogyne hapla, Hoplolaimus columbus, Hoplolaimus galeatus and Rotylenchulus reniformis.

In one embodiment, nematicidal compounds to be used for treating seeds or plants of the invention, or the soil wherein said seeds or plants are growing or are intended to be grown, include these of group NC1: alanycarb, aldicarb, carbofuran, carbosulfan, fosthiazate, cadusafos, oxamyl, thiodicarb, dimethoate, ethoprophos, terbufos, abamectin, methyl bromide and other alkyl halides, methyl isocyanate generators selected from diazomet and metam, fluazaindolizine, fluensulfone, fluopyram, tioxazafen, N-[1-(2,6-difluorophenyl)-1H-pyrazol-3-yl]-2-trifluoromethylbenzamide, cis-Jasmone, harpin, Azadirachta indica oil, or Azadirachtin. In another embodiment, the plants or seeds of the invention, or the soil in which they are grown or are intended to be grown, are treated with any of the following nematicidal agents of group NC2: fosthiazate, cadusafos, thiodicarb, abamectin, fluazaindolizine, fluopyram, tioxazafen, N-[1-(2,6-difluorophenyl)-1H-pyrazol-3-yl]-2-trifluoromethylbenzamide, cis-Jasmone, harpin, Azadirachta indica oil, or Azadirachtin, particularly the seeds of the invention are treated with said nematicidal compounds.

In one embodiment, the plant, cell, plant part or seed of the invention, comprising EE-GM4, is treated with a combination selected from the group NC3 consisting of: fosthiazate and cadusafos, fosthiazate and thiodicarb, fosthiazate and abamectin, fosthiazate and fluazaindolizine, fosthiazate and fluopyram, fosthiazate and tioxazafen, fosthiazate and N-[1-(2,6-difluorophenyl)-1H-pyrazol-3-yl]-2-trifluoromethylbenzamide, fosthiazate and cis-Jasmone, fosthiazate and harpin, fosthiazate and Azadirachta indica oil, fosthiazate and Azadirachtin, cadusafos and fosthiazate, cadusafos and thiodicarb, cadusafos and abamectin, cadusafos and fluazaindolizine, cadusafos and fluopyram, cadusafos and tioxazafen, cadusafos and N-[1-(2,6-difluorophenyl)-1H-pyrazol-3-yl]-2-trifluoromethylbenzamide, cadusafos and cis-Jasmone, cadusafos and harpin, cadusafos and Azadirachta indica oil, cadusafos and Azadirachtin, thiodicarb and fosthiazate, thiodicarb and cadusafos, thiodicarb and abamectin, thiodicarb and fluazaindolizine, thiodicarb and fluopyram, thiodicarb and tioxazafen, thiodicarb and N-[1-(2,6-difluorophenyl)-1H-pyrazol-3-yl]-2-trifluoromethylbenzamide, thiodicarb and cis-Jasmone, thiodicarb and harpin, thiodicarb and Azadirachta indica oil, abamectin and cadusafos, abamectin and thiodicarb, abamectin and fluazaindolizine, abamectin and fluopyram, abamectin and tioxazafen, abamectin and N-[1-(2,6-difluorophenyl)-1H-pyrazol-3-yl]-2-trifluoromethylbenzamide, abamectin and cis-Jasmone, abamectin and harpin, abamectin and Azadirachta indica oil, abamectin and Azadirachtin, fluazaindolizine and abamectin, fluazaindolizine and fluopyram, fluazaindolizine and tioxazafen, fluazaindolizine and N-[1-(2,6-difluorophenyl)-1H-pyrazol-3-yl]-2-trifluoromethylbenzamide, fluazaindolizine and cis-Jasmone, fluazaindolizine and harpin, fluazaindolizine and Azadirachta indica oil, fluazaindolizine and Azadirachtin, fluopyram and fluazaindolizine, fluopyram and tioxazafen, fluopyram and N-[1-(2,6-difluorophenyl)-1H-pyrazol-3-yl]-2-trifluoromethylbenzamide, fluopyram and cis-Jasmone, fluopyram and harpin, fluopyram and Azadirachta indica oil, fluopyram and Azadirachtin, tioxazafen and N-[1-(2,6-difluorophenyl)-1H-pyrazol-3-yl]-2-trifluoromethylbenzamide, tioxazafen and cis-Jasmone, tioxazafen and harpin, tioxazafen and Azadirachta indica oil, tioxazafen and Azadirachtin, N-[1-(2,6-difluorophenyl)-1H-pyrazol-3-yl]-2-trifluoromethylbenzamide and cis-Jasmone, N-[1-(2,6-difluorophenyl)-1H-pyrazol-3-yl]-2-trifluoromethylbenzamide and harpin, N-[1-(2,6-difluorophenyl)-1H-pyrazol-3-yl]-2-trifluoromethylbenzamide and Azadirachta indica oil, N-[1-(2,6-difluorophenyl)-1H-pyrazol-3-yl]-2-trifluoromethylbenzamide and Azadirachtin, cis-Jasmone and harpin, cis-Jasmone and Azadirachta indica oil, cis-Jasmone and Azadirachtin, harpin and Azadirachta indica oil, and harpin and Azadirachtin.

### Fungicides

The active ingredients specified herein by their Common Name are known and described, for example, in The Pesticide Manual (17th Ed.British Crop Protection Council, updated version at on the world wide web at bcpc.org/product/bcpc-online-pesticide-manual-latest-version) or can be searched on the internet (e.g. on the world wide web at alanwood.net/pesticides).

All named fungicidal mixing partners of the classes F1 to F15 can, if their functional groups enable this, optionally form salts with suitable bases or acids. All named mixing partners of the classes F1 to F15 can include tautomeric forms, where applicable.

F1. Inhibitors of the ergosterol biosynthesis, for example (1.001) cyproconazole, (1.002) difenoconazole, (1.003) epoxiconazole, (1.004) fenhexamid, (1.005) fenpropidin, (1.006) fenpropimorph, (1.007) fenpyrazamine, (1.008) fluquinconazole, (1.009) flutriafol, (1.010) imazalil, (1.011) imazalil sulfate, (1.012) ipconazole, (1.013) metconazole, (1.014) myclobutanil, (1.015) paclobutrazol, (1.016) prochloraz, (1.017) propiconazole, (1.018) prothioconazole, (1.019) Pyrisoxazole, (1.020) spiroxamine, (1.021) tebuconazole, (1.022) tetraconazole, (1.023) triadimenol, (1.024) tridemorph, (1.025) triticonazole, (1.026) (1R,2S,5S)-5-(4-chlorobenzyl)-2-(chloromethyl)-2-methyl-1-(1H-1,2,4-triazol-1-ylmethyl)cyclopentanol, (1.027) (1S,2R,5R)-5-(4-chlorobenzyl)-2-(chloromethyl)-2-methyl-1-(1H-1,2,4-triazol-1-ylmethyl)cyclopentanol, (1.028) (2R)-2-(1-chlorocyclopropyl)-4-[(1R)-2,2-dichlorocyclopropyl]-1-(1H-1,2,4-triazol-1-yl)butan-2-ol, (1.029) (2R)-2-(1-chlorocyclopropyl)-4-[(1S)-2,2-dichlorocyclopropyl]-1-(1H-1,2,4-triazol-1-yl)butan-2-ol, (1.030) (2R)-2-[4-(4-chlorophenoxy)-2-(trifluoromethyl)phenyl]-1-(1H-1,2,4-triazol-1-yl)propan-2-ol, (1.031) (2S)-2-(1-chlorocyclopropyl)-4-[(1R)-2,2-dichlorocyclopropyl]-1-(1H-1,2,4-triazol-1-yl)butan-2-ol, (1.032) (2S)-2-(1-chlorocyclopropyl)-4-[(1S)-2,2-dichlorocyclopropyl]-1-(1H-1,2,4-triazol-1-yl)butan-2-ol, (1.033) (2S)-2-[4-(4-chlorophenoxy)-2-(trifluoromethyl)phenyl]-1-(1H-1,2,4-triazol-1-yl)propan-2-ol, (1.034) (R)-[3-(4-chloro-2-fluorophenyl)-5-(2,4-difluorophenyl)-1,2-oxazol-4-yl](pyridin-3-yl)methanol, (1.035) (S)-[3-(4-chloro-2-fluorophenyl)-5-(2,4-difluorophenyl)-1,2-oxazol-4-yl](pyridin-3-yl)methanol, (1.036) [3-(4-chloro-2-fluorophenyl)-5-(2,4-difluorophenyl)-1,2-oxazol-4-yl](pyridin-3-yl)methanol, (1.037) 1-({(2R,4S)-2-[2-chloro-4-(4-chlorophenoxy)phenyl]-4-methyl-1,3-dioxolan-2-yl}methyl)-1H-1,2,4-triazole, (1.038) 1-({(2S,4S)-2-[2-chloro-4-(4-chlorophenoxy)phenyl]-4-methyl-1,3-dioxolan-2-yl}methyl)-1H-1,2,4-triazole, (1.039) 1-{[3-(2-chlorophenyl)-2-(2,4-difluorophenyl)oxiran-2-yl]methyl}-1H-1,2,4-triazol-5-yl thiocyanate, (1.040) 1-{[rel(2R,3R)-3-(2-chlorophenyl)-2-(2,4-difluorophenyl)oxiran-2-yl]methyl}-1H-1,2,4-triazol-5-yl thiocyanate, (1.041) 1-{[rel(2R,3S)-3-(2-chlorophenyl)-2-(2,4-difluorophenyl)oxiran-2-yl]methyl}-1H-1,2,4-triazol-5-yl thiocyanate, (1.042) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.043) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.044) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.045) 2-[(2R,4S,5S)-1-(2,4-dichlorophenyl)-5-hydroxy-2,6,6-trimethylheptan-4-yl]-2,4-dihydro-3H-1,2,4-triazole-3-thione, (1.046) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.047) 2-[(2S,4R,5S)-1-(2,4-dichlorophenyl)-5-hydroxy-2,6,6-trimethylheptan-4-yl]-2,4-dihydro-3H-1,2,4-triazole-3-thione, (1.048) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.049) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.050) 2-[1-(2,4-dichlorophenyl)-5-hydroxy-2,6,6-trimethylheptan-4-yl]-2,4-dihydro-3H-1,2,4-triazole-3-thione, (1.051) 2-[2-chloro-4-(2,4-dichlorophenoxy)phenyl]-1-(1H-1,2,4-triazol-1-yl)propan-2-ol, (1.052) 2-[2-chloro-4-(4-chlorophenoxy)phenyl]-1-(1H-1,2,4-triazol-1-yl)butan-2-ol, (1.053) 2-[4-(4-chlorophenoxy)-2-(trifluoromethyl)phenyl]-1-(1H-1,2,4-triazol-1-yl)butan-2-ol, (1.054) 2-[4-(4-chlorophenoxy)-2-(trifluoromethyl)phenyl]-1-(1H-1,2,4-triazol-1-yl)pentan-2-ol, (1.055) 2-[4-(4-chlorophenoxy)-2-(trifluoromethyl)phenyl]-1-(1H-1,2,4-triazol-1-yl)propan-2-ol, (1.056) 2-{[3-(2-chlorophenyl)-2-(2,4-difluorophenyl)oxiran-2-yl]methyl}-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulfanyl)-1-{[3-(2-chlorophenyl)-2-(2,4-difluorophenyl)oxiran-2-yl]methyl}-1H-1,2,4-triazole, (1.061) 5-(allylsulfanyl)-1-{[rel(2R,3R)-3-(2-chlorophenyl)-2-(2,4-difluorophenyl)oxiran-2-yl]methyl}-1H-1,2,4-triazole, (1.062) 5-(allylsulfanyl)-1-{[rel(2R,3S)-3-(2-chlorophenyl)-2-(2,4-difluorophenyl)oxiran-2-yl]methyl}-1H-1,2,4-triazole, (1.063) N′-(2,5-dimethyl-4-{[3-(1,1,2,2-tetrafluoroethoxy)phenyl]sulfanyl}phenyl)-N-ethyl-N-methylimidoformamide, (1.064) N′-(2,5-dimethyl-4-{[3-(2,2,2-trifluoroethoxy)phenyl]sulfanyl}phenyl)-N-ethyl-N-methylimidoformamide, (1.065) N′-(2,5-dimethyl-4-{[3-(2,2,3,3-tetrafluoropropoxy)phenyl]sulfanyl}phenyl)-N-ethyl-N-methylimidoformamide, (1.066) N′-(2,5-dimethyl-4-{[3-(pentafluoroethoxy)phenyl]sulfanyl}phenyl)-N-ethyl-N-methylimidoformamide, (1.067) N′-(2,5-dimethyl-4-{3-[(1,1,2,2-tetrafluoroethyl)sulfanyl]phenoxy}phenyl)-N-ethyl-N-methylimidoformamide, (1.068) N′-(2,5-dimethyl-4-{3-[(2,2,2-trifluoroethyl)sulfanyl]phenoxy}phenyl)-N-ethyl-N-methylimidoformamide, (1.069) N′-(2,5-dimethyl-4-{3-[(2,2,3,3-tetrafluoropropyl)sulfanyl]phenoxy}phenyl)-N-ethyl-N-methylimidoformamide, (1.070) N′-(2,5-dimethyl-4-{3-[(pentafluoroethyl)sulfanyl]phenoxy}phenyl)-N-ethyl-N-methylimidoformamide, (1.071) N′-(2,5-dimethyl-4-phenoxyphenyl)-N-ethyl-N-methylimidoformamide, (1.072) N′-(4-{[3-(difluoromethoxy)phenyl]sulfanyl}-2,5-dimethylphenyl)-N-ethyl-N-methylimidoformamide, (1.073) N′-(4-{3-[(difluoromethyl)sulfanyl]phenoxy}-2,5-dimethylphenyl)-N-ethyl-N-methylimidoformamide, (1.074) N′-[5-bromo-6-(2,3-dihydro-1H-inden-2-yloxy)-2-methylpyridin-3-yl]-N-ethyl-N-methylimidoformamide, (1.075) N′-{4-[(4,5-dichloro-1,3-thiazol-2-yl)oxy]-2,5-dimethylphenyl}-N-ethyl-N-methylimidoformamide, (1.076) N′-{5-bromo-6-[(1R)-1-(3,5-difluorophenyl)ethoxy]-2-methylpyridin-3-yl}-N-ethyl-N-methylimidoformamide, (1.077) N′-{5-bromo-6-[(1S)-1-(3,5-difluorophenyl)ethoxy]-2-methylpyridin-3-yl}-N-ethyl-N-methylimidoformamide, (1.078) N′-{5-bromo-6-[(cis-4-isopropylcyclohexyl)oxy]-2-methylpyridin-3-yl}-N-ethyl-N-methylimidoformamide, (1.079) N′-{5-bromo-6-[(trans-4-isopropylcyclohexyl)oxy]-2-methylpyridin-3-yl}-N-ethyl-N-methylimidoformamide, (1.080) N′-{5-bromo-6-[1-(3,5-difluorophenyl)ethoxy]-2-methylpyridin-3-yl}-N-ethyl-N-methylimidoformamide, (1.081) Mefentrifluconazole, (1.082) Ipfentrifluconazole, (1.083) Azaconazole, (1.084) Hexaconazole, (1.085) Triadimefon, (1.086) Oxpoconazole, (1.087) Penconazole, (1.088) Prochloraz, (1.089) Triflumizole, (1.090) Triforine, (1.091) Pyrifenox, (1.092) Fenarimol, (1.093) Nuarimol, (1.094) Bitertanol, (1.095) Bromuconazole, (1.096) Diniconazole, (1.097) Epoxiconazole, (1.098) Fenfuconazole, (1.099) Imibenconazole.

F2: Inhibitors of the respiratory chain at complex I or II, for example (2.001) benzovindiflupyr, (2.002) bixafen, (2.003) boscalid, (2.004) carboxin, (2.005) fluopyram, (2.006) flutolanil, (2.007) fluxapyroxad, (2.008) furametpyr, (2.009) Isofetamid, (2.010) isopyrazam (anti-epimeric enantiomer 1R,4S,9S), (2.011) isopyrazam (anti-epimeric enantiomer 1S,4R,9R), (2.012) isopyrazam (anti-epimeric racemate 1RS,4SR,9SR), (2.013) isopyrazam (mixture of syn-epimeric racemate 1RS,4SR,9RS and anti-epimeric racemate 1RS,4SR,9SR), (2.014) isopyrazam (syn-epimeric enantiomer 1R,4S,9R), (2.015) isopyrazam (syn-epimeric enantiomer 1S,4R,9S), (2.016) isopyrazam (syn-epimeric racemate 1RS,4SR,9RS), (2.017) penflufen, (2.018) penthiopyrad, (2.019) pydiflumetofen, (2.020) Pyraziflumid, (2.021) sedaxane, (2.022) 1,3-dimethyl-N-(1,1,3-trimethyl-2,3-dihydro-1H-inden-4-yl)-1H-pyrazole-4-carboxamide, (2.023) 1,3-dimethyl-N-[(3R)-1,1,3-trimethyl-2,3-dihydro-1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) 1-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3R)-1,1,3-trimethyl-2,3-dihydro-1H-inden-4-yl]-1H-pyrazole-4-carboxamide, (2.029) 3-(difluoromethyl)-1-methyl-N-[(3S)-1,1,3-trimethyl-2,3-dihydro-1H-inden-4-yl]-1H-pyrazole-4-carboxamide, (2.030) 3-(difluoromethyl)-N-(7-fluoro-1,1,3-trimethyl-2,3-dihydro-1H-inden-4-yl)-1-methyl-1H-pyrazole-4-carboxamide, (2.031) 3-(difluoromethyl)-N-[(3R)-7-fluoro-1,1,3-trimethyl-2,3-dihydro-1H-inden-4-yl]-1-methyl-1H-pyrazole-4-carboxamide, (2.032) 3-(difluoromethyl)-N-[(3S)-7-fluoro-1,1,3-trimethyl-2,3-dihydro-1H-inden-4-yl]-1-methyl-1H-pyrazole-4-carboxamide, (2.033) 5,8-difluoro-N-[2-(2-fluoro-4-{[4-(trifluoromethyl)pyridin-2-yl]oxy}phenyl)ethyl]quinazolin-4-amine, (2.034)N-(2-cyclopentyl-5-fluorobenzyl)-N-cyclopropyl-3-(difluoromethyl)-5-fluoro-1-methyl-H-pyrazole-4-carboxamide, (2.035)N-(2-tert-butyl-5-methylbenzyl)-N-cyclopropyl-3-(difluoromethyl)-5-fluoro-1-methyl-1H-pyrazole-4-carboxamide, (2.036)N-(2-tert-butylbenzyl)-N-cyclopropyl-3-(difluoromethyl)-5-fluoro-1-methyl-1H-pyrazole-4-carboxamide, (2.037)N-(5-chloro-2-ethylbenzyl)-N-cyclopropyl-3-(difluoromethyl)-5-fluoro-1-methyl-1H-pyrazole-4-carboxamide, (2.038)N-(5-chloro-2-isopropylbenzyl)-N-cyclopropyl-3-(difluoromethyl)-5-fluoro-1-methyl-1H-pyrazole-4-carboxamide, (2.039)N-[(1R,4S)-9-(dichloromethylene)-1,2,3,4-tetrahydro-1,4-methanonaphthalen-5-yl]-3-(difluoromethyl)-1-methyl-H-pyrazole-4-carboxamide, (2.040)N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, (2.041)N-[1-(2,4-dichlorophenyl)-1-methoxypropan-2-yl]-3-(difluoromethyl)-1-methyl-H-pyrazole-4-carboxamide, (2.042)N-[2-chloro-6-(trifluoromethyl)benzyl]-N-cyclopropyl-3-(difluoromethyl)-5-fluoro-1-methyl-1H-pyrazole-4-carboxamide, (2.043)N-[3-chloro-2-fluoro-6-(trifluoromethyl)benzyl]-N-cyclopropyl-3-(difluoromethyl)-5-fluoro-1-methyl-H-pyrazole-4-carboxamide, (2.044)N-[5-chloro-2-(trifluoromethyl)benzyl]-N-cyclopropyl-3-(difluoromethyl)-5-fluoro-1-methyl-1H-pyrazole-4-carboxamide, (2.045)N-cyclopropyl-3-(difluoromethyl)-5-fluoro-1-methyl-N-[5-methyl-2-(trifluoromethyl)benzyl]-1H-pyrazole-4-carboxamide, (2.046)N-cyclopropyl-3-(difluoromethyl)-5-fluoro-N-(2-fluoro-6-isopropylbenzyl)-1-methyl-H-pyrazole-4-carboxamide, (2.047)N-cyclopropyl-3-(difluoromethyl)-5-fluoro-N-(2-isopropyl-5-methylbenzyl)-1-methyl-1H-pyrazole-4-carboxamide, (2.048)N-cyclopropyl-3-(difluoromethyl)-5-fluoro-N-(2-isopropylbenzyl)-1-methyl-1H-pyrazole-4-carbothioamide, (2.049)N-cyclopropyl-3-(difluoromethyl)-5-fluoro-N-(2-isopropylbenzyl)-1-methyl-H-pyrazole-4-carboxamide, (2.050)N-cyclopropyl-3-(difluoromethyl)-5-fluoro-N-(5-fluoro-2-isopropylbenzyl)-1-methyl-1H-pyrazole-4-carboxamide, (2.051)N-cyclopropyl-3-(difluoromethyl)-N-(2-ethyl-4,5-dimethylbenzyl)-5-fluoro-1-methyl-1H-pyrazole-4-carboxamide, (2.052)N-cyclopropyl-3-(difluoromethyl)-N-(2-ethyl-5-fluorobenzyl)-5-fluoro-1-methyl-H-pyrazole-4-carboxamide, (2.053)N-cyclopropyl-3-(difluoromethyl)-N-(2-ethyl-5-methylbenzyl)-5-fluoro-1-methyl-1H-pyrazole-4-carboxamide, (2.054)N-cyclopropyl-N-(2-cyclopropyl-5-fluorobenzyl)-3-(difluoromethyl)-5-fluoro-1-methyl-1H-pyrazole-4-carboxamide, (2.055)N-cyclopropyl-N-(2-cyclopropyl-5-methylbenzyl)-3-(difluoromethyl)-5-fluoro-1-methyl-1H-pyrazole-4-carboxamide, (2.056)N-cyclopropyl-N-(2-cyclopropylbenzyl)-3-(difluoromethyl)-5-fluoro-1-methyl-1H-pyrazole-4-carboxamide, (2.057) Benzovindiflupyr, (2.058) Isoflucypram.

F3: Inhibitors of the respiratory chain at complex III, for example (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadone, (3.010) fenamidone, (3.011) flufenoxystrobin, (3.012) fluoxastrobin, (3.013) kresoxim-methyl, (3.014) metominostrobin, (3.015) orysastrobin, (3.016) picoxystrobin, (3.017) pyraclostrobin, (3.018) pyrametostrobin, (3.019) pyraoxystrobin, (3.020) trifloxystrobin, (3.021) (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, (3.022) (2E,3Z)-5-{[1-(4-chlorophenyl)-1H-pyrazol-3-yl]oxy}-2-(methoxyimino)-N,3-dimethylpent-3-enamide, (3.023) (2R)-2-{2-[(2,5-dimethylphenoxy)methyl]phenyl}-2-methoxy-N-methylacetamide, (3.024) (2S)-2-{2-[(2,5-dimethylphenoxy)methyl]phenyl}-2-methoxy-N-methylacetamide, (3.025) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (3.026) 2-{2-[(2,5-dimethylphenoxy)methyl]phenyl}-2-methoxy-N-methylacetamide, (3.027)N-(3-ethyl-3,5,5-trimethylcyclohexyl)-3-formamido-2-hydroxybenzamide, (3.028) (2E,3Z)-5-{[1-(4-chloro-2-fluorophenyl)-1H-pyrazol-3-yl]oxy}-2-(methoxyimino)-N,3-dimethylpent-3-enamide, (3.029) methyl {5-[3-(2,4-dimethylphenyl)-1H-pyrazol-1-yl]-2-methylbenzyl}carbamate, (3.030) mandestrobin.

F4: Inhibitors of the mitosis and cell division, for example (4.001) carbendazim, (4.002) diethofencarb, (4.003) ethaboxam, (4.004) fluopicolide, (4.005) pencycuron, (4.006) thiabendazole, (4.007) thiophanate-methyl, (4.008) zoxamide, (4.009) 3-chloro-4-(2,6-difluorophenyl)-6-methyl-5-phenylpyridazine, (4.010) 3-chloro-5-(4-chlorophenyl)-4-(2,6-difluorophenyl)-6-methylpyridazine, (4.011) 3-chloro-5-(6-chloropyridin-3-yl)-6-methyl-4-(2,4,6-trifluorophenyl)pyridazine, (4.012) 4-(2-bromo-4-fluorophenyl)-N-(2,6-difluorophenyl)-1,3-dimethyl-1H-pyrazol-5-amine, (4.013) 4-(2-bromo-4-fluorophenyl)-N-(2-bromo-6-fluorophenyl)-1,3-dimethyl-1H-pyrazol-5-amine, (4.014) 4-(2-bromo-4-fluorophenyl)-N-(2-bromophenyl)-1,3-dimethyl-1H-pyrazol-5-amine, (4.015) 4-(2-bromo-4-fluorophenyl)-N-(2-chloro-6-fluorophenyl)-1,3-dimethyl-1H-pyrazol-5-amine, (4.016) 4-(2-bromo-4-fluorophenyl)-N-(2-chlorophenyl)-1,3-dimethyl-1H-pyrazol-5-amine, (4.017) 4-(2-bromo-4-fluorophenyl)-N-(2-fluorophenyl)-1,3-dimethyl-1H-pyrazol-5-amine, (4.018) 4-(2-chloro-4-fluorophenyl)-N-(2,6-difluorophenyl)-1,3-dimethyl-1H-pyrazol-5-amine, (4.019) 4-(2-chloro-4-fluorophenyl)-N-(2-chloro-6-fluorophenyl)-1,3-dimethyl-1H-pyrazol-5-amine, (4.020) 4-(2-chloro-4-fluorophenyl)-N-(2-chlorophenyl)-1,3-dimethyl-1H-pyrazol-5-amine, (4.021) 4-(2-chloro-4-fluorophenyl)-N-(2-fluorophenyl)-1,3-dimethyl-1H-pyrazol-5-amine, (4.022) 4-(4-chlorophenyl)-5-(2,6-difluorophenyl)-3,6-dimethylpyridazine, (4.023)N-(2-bromo-6-fluorophenyl)-4-(2-chloro-4-fluorophenyl)-1,3-dimethyl-1H-pyrazol-5-amine, (4.024)N-(2-bromophenyl)-4-(2-chloro-4-fluorophenyl)-1,3-dimethyl-1H-pyrazol-5-amine, (4.025)N-(4-chloro-2,6-difluorophenyl)-4-(2-chloro-4-fluorophenyl)-1,3-dimethyl-1H-pyrazol-5-amine.

F5: Compounds capable to have a multisite action, for example (5.001) bordeaux mixture, (5.002) captafol, (5.003) captan, (5.004) chlorothalonil, (5.005) copper hydroxide, (5.006) copper naphthenate, (5.007) copper oxide, (5.008) copper oxychloride, (5.009) copper(2+) sulfate, (5.010) dithianon, (5.011) dodine, (5.012) folpet, (5.013) mancozeb, (5.014) maneb, (5.015) metiram, (5.016) metiram zinc, (5.017) oxine-copper, (5.018) propineb, (5.019) sulfur and sulfur preparations including calcium polysulfide, (5.020) thiram, (5.021) zineb, (5.022) ziram, (5.023) 6-ethyl-5,7-dioxo-6,7-dihydro-5H-pyrrolo[3′,4′:5,6][1,4]dithiino[2,3-c][1,2]thiazole-3-carbonitrile.

F6: Compounds capable to induce a host defence, for example (6.001) acibenzolar-S-methyl, (6.002) isotianil, (6.003) probenazole, (6.004) tiadinil.

F7: Inhibitors of the amino acid and/or protein biosynthesis, for example (7.001) cyprodinil, (7.002) kasugamycin, (7.003) kasugamycin hydrochloride hydrate, (7.004) oxytetracycline, (7.005) pyrimethanil, (7.006) 3-(5-fluoro-3,3,4,4-tetramethyl-3,4-dihydroisoquinolin-1-yl)quinoline.

F8: Inhibitors of the ATP production, for example (8.001) silthiofam.

F9: Inhibitors of the cell wall synthesis, for example (9.001) benthiavalicarb, (9.002) dimethomorph, (9.003) flumorph, (9.004) iprovalicarb, (9.005) mandipropamid, (9.006) pyrimorph, (9.007) valifenalate, (9.008) (2E)-3-(4-tert-butylphenyl)-3-(2-chloropyridin-4-yl)-1-(morpholin-4-yl)prop-2-en-1-one, (9.009) (2Z)-3-(4-tert-butylphenyl)-3-(2-chloropyridin-4-yl)-1-(morpholin-4-yl)prop-2-en-1-one.

F10: Inhibitors of the lipid and membrane synthesis, for example (10.001) propamocarb, (10.002) propamocarb hydrochloride, (10.003) tolclofos-methyl.

F1: Inhibitors of the melanin biosynthesis, for example (11.001) tricyclazole, (11.002) 2,2,2-trifluoroethyl {3-methyl-1-[(4-methylbenzoyl)amino]butan-2-yl}carbamate.

F12: Inhibitors of the nucleic acid synthesis, for example (12.001) benalaxyl, (12.002) benalaxyl-M (kiralaxyl), (12.003) metalaxyl, (12.004) metalaxyl-M (mefenoxam), (12.005) furalaxyl.

F13: Inhibitors of the signal transduction, for example (13.001) fludioxonil, (13.002) iprodione, (13.003) procymidone, (13.004) proquinazid, (13.005) quinoxyfen, (13.006) vinclozolin.

F14: Compounds capable to act as an uncoupler, for example (14.001) fluazinam, (14.002) meptyldinocap.

F15: Further compounds, for example (15.001) Abscisic acid, (15.002) benthiazole, (15.003) bethoxazin, (15.004) capsimycin, (15.005) carvone, (15.006) chinomethionat, (15.007) cufraneb, (15.008) cyflufenamid, (15.009) cymoxanil, (15.010) cyprosulfamide, (15.011) flutianil, (15.012) fosetyl-aluminium, (15.013) fosetyl-calcium, (15.014) fosetyl-sodium, (15.015) methyl isothiocyanate, (15.016) metrafenone, (15.017) mildiomycin, (15.018) natamycin, (15.019) nickel dimethyldithiocarbamate, (15.020) nitrothal-isopropyl, (15.021) oxamocarb, (15.022) Oxathiapiprolin, (15.023) oxyfenthiin, (15.024) pentachlorophenol and salts, (15.025) phosphorous acid and its salts, (15.026) propamocarb-fosetylate, (15.027) pyriofenone (chlazafenone), (15.028) tebufloquin, (15.029) tecloftalam, (15.030) tolnifanide, (15.031) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.032) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.033) 2-(6-benzylpyridin-2-yl)quinazoline, (15.034) 2,6-dimethyl-1H,5H-[1,4]dithiino[2,3-c:5,6-c′]dipyrrole-1,3,5,7(2H,6H)-tetrone, (15.035) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (15.036) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (15.037) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.038) 2-[6-(3-fluoro-4-methoxyphenyl)-5-methylpyridin-2-yl]quinazoline, (15.039) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.040) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.041) 2-{2-[(7,8-difluoro-2-methylquinolin-3-yl)oxy]-6-fluorophenyl}propan-2-ol, (15.042) 2-{2-fluoro-6-[(8-fluoro-2-methylquinolin-3-yl)oxy]phenyl}propan-2-ol, (15.043) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.044) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, (15.045) 2-phenylphenol and salts, (15.046) 3-(4,4,5-trifluoro-3,3-dimethyl-3,4-dihydroisoquinolin-1-yl)quinoline, (15.047) 3-(4,4-difluoro-3,3-dimethyl-3,4-dihydroisoquinolin-1-yl)quinoline, (15.048) 4-amino-5-fluoropyrimidin-2-ol (tautomeric form: 4-amino-5-fluoropyrimidin-2(1H)-one), (15.049) 4-oxo-4-[(2-phenylethyl)amino]butanoic acid, (15.050) 5-amino-1,3,4-thiadiazole-2-thiol, (15.051) 5-chloro-N′-phenyl-N′-(prop-2-yn-1-yl)thiophene-2-sulfonohydrazide, (15.052) 5-fluoro-2-[(4-fluorobenzyl)oxy]pyrimidin-4-amine, (15.053) 5-fluoro-2-[(4-methylbenzyl)oxy]pyrimidin-4-amine, (15.054) 9-fluoro-2,2-dimethyl-5-(quinolin-3-yl)-2,3-dihydro-1,4-benzoxazepine, (15.055) but-3-yn-1-yl {6-[({[(Z)-(1-methyl-1H-tetrazol-5-yl)(phenyl)methylene]amino}oxy)methyl]pyridin-2-yl}carbamate, (15.056) ethyl (2Z)-3-amino-2-cyano-3-phenylacrylate, (15.057) phenazine-1-carboxylic acid, (15.058) propyl 3,4,5-trihydroxybenzoate, (15.059) quinolin-8-ol, (15.060) quinolin-8-ol sulfate (2:1), (15.061) tert-butyl {6-[({[(1-methyl-1H-tetrazol-5-yl)(phenyl)methylene]amino}oxy)methyl]pyridin-2-yl}carbamate, (15.062) 5-fluoro-4-imino-3-methyl-1-[(4-methylphenyl)sulfonyl]-3,4-dihydropyrimidin-2(1H)-one, (15.063) any of the pesticides described in US20140221362 (incorporated herein by reference), such as any of the compounds of claim 1 therein, (15.064) fenpicoxamid, (15.065) tolprocarb, (15.066) picarbutrazox, (15.067) pyraziflumid, (15.068) Inpyrfluxam (WO2011162397), (15.069) quinofumelin, (15.070) fluindapyr, (15.071) pyrapropoyne, (15.072) 5-fluoropyrimidone, (15.073) any fungicidal compounds from published patent applications WO2014/201326, WO2014/201327, WO2013/071169, WO2014/182951, WO2014/182945, WO2014/182950, incorporated herein by reference.

Particularly preferred mixtures of fungicides used in the context of the present invention are selected from the group F16 consisting of: mixtures of Prothioconazole and Fluopyram, mixtures of Prothioconazole and Tebuconazole, mixtures of Prothioconazole, Bixafen, and Tebuconazole, mixtures of Prothioconazole, Bixafen, and Trifloxystrobin, mixtures of Prothioconazole, Bixafen, and Spiroxamine, mixtures of Prothioconazole, Bixafen, and Chlorothalonil, mixtures of Prothioconazole and Tebuconazole and Metalaxyl, mixtures of Prothioconazole, Bixafen, and Fluopyram, mixtures of Prothioconazole and Fluoxastrobin, mixtures of Prothioconazole and Trifloxystrobin, mixtures of Prothioconazole and Pencycuron, mixtures of Prothioconazole and Spiroxamine, mixtures of Tebuconazole and Bixafen, mixtures of Tebuconazole and Metalaxyl, mixtures of Tebuconazole and Fluoxastrobin, mixtures of Tebuconazole and Trifloxystrobin, mixtures of Tebuconazole and Fenhexamid, mixtures of Tebuconazole and Fluopicolide, mixtures of Tebuconazole and Spiroxamine, mixtures of Tebuconazole and Pencycuron, mixtures of Fluopyram and Tebuconazole, mixtures of Fluopyram and Spiroxamine, mixtures of Fluopyram and Pyrimethanil, mixtures of Fluopyram and Trifloxystrobin, mixtures of Fluopyram and Triadimenol, mixtures of Fluopyram and Fosetyl or salts and esters thereof, in particular Fosetyl-aluminium, mixtures of Fluopyram and Imidacloprid, mixtures of Fluopyram and Spiroxamine, mixtures of Fluopyram and Fluoxastrobin, mixtures of Bixafen and Spiroxamine, mixtures of Bixafen and Trifloxystrobin, mixtures of Bixafen and Chlorothalonil, mixtures of Bixafen and Fluoxastrobin, mixtures of Trifloxystrobin and Isotianil, mixtures of Trifloxystrobin and Metalaxyl, mixtures of Trifloxystrobin and Propineb, mixtures of Trifloxystrobin and Pyrimethanil, mixtures of Trifloxystrobin and Fosetyl or salts and esters thereof, in particular Fosetyl-aluminium, mixtures of Trifloxystrobin and Fluopicolide, mixtures of Propineb and Fluopicolide, mixtures of Fosetyl or salts and esters thereof, in particular Fosetyl-aluminium, and Fluopicolide, mixtures of Bixafen and Ipfentrifluconazole, mixtures of Bixafen and Mefentrifluconazole, mixtures of Metalaxyl and Ipfentrifluconazole, mixtures of Metalaxyl and Mefentrifluconazole, mixtures of Prothioconazole and Ipfentrifluconazole, mixtures of Prothioconazole and Mefentrifluconazole, mixtures of Tebuconazole and Ipfentrifluconazole, mixtures of Tebuconazole and Mefentrifluconazole, mixtures of Trifloxystrobin and Ipfentrifluconazole, mixtures of Trifloxystrobin and Mefentrifluconazole, mixtures of Fluoxastrobin and Ipfentrifluconazole, mixtures of Fluoxastrobin and Mefentrifluconazole, mixtures of Fluopyram and Ipfentrifluconazole, mixtures of Fluopyram and Mefentrifluconazole, mixtures of Spiroxamine and Ipfentrifluconazole, mixtures of Spiroxamine and Mefentrifluconazole, mixtures of Pydiflumetofen and Ipfentrifluconazole, mixtures of Pydiflumetofen and Mefentrifluconazole, mixtures of Pyraclostrobin and Ipfentrifluconazole, mixtures of Pyraclostrobin and Mefentrifluconazole, mixtures of Fluxapyroxad and Ipfentrifluconazole, mixtures of Fluxapyroxad and Mefentrifluconazole.

A fungicide/fungicidal agent in crop protection, as described herein for use on or with the plants or seeds of the invention, is capable of controlling fungi or oomycetes. The term “controlling fungi/oomycetes”, as used herein, means killing the fungi/oomycetes or preventing or impeding their development or their growth or preventing or impeding their penetration into or their feeding on plant tissue.

### Herbicides

Examples for herbicides useful in the context of the present invention are disclosed in group H1:

Acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, amitrole, ammoniumsulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate, and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyrimorate, cyclosulfamuron, cycloxydim, cyhalofop, cyhalofop-butyl, cyprazine, 2,4-D, 2,4-D-butotyl, -butyl, -dimethylammonium, -diolamin, -ethyl, -2-ethylhexyl, -isobutyl, -isooctyl, -isopropylammonium, -potassium, -triisopropanolammonium, and -trolamine, 2,4-DB, 2,4-DB-butyl, -dimethylammonium, -isooctyl, -potassium, and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, dimetrasulfuron, dinitramine, dinoterb, diphenamid, diquat, diquat-dibromid, dithiopyr, diuron, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-9600, F-5231, i.e. N-{2-chloro-4-fluoro-5-[4-(3-fluoropropyl)-5-oxo-4,5-dihydro-1H-tetrazol-1-yl]phenyl}ethanesulfonamide, F-7967, i. e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium, and -trimesium, H-9201, i.e. 0-(2,4-dimethyl-6-nitrophenyl)O-ethyl isopropylphosphoramidothioate, halauxifen, halauxifen-methyl, halosafen, halosulfuron, halosulfuron-methyl,haloxyfop,haloxyfop-P,haloxyfop-ethoxyethyl,haloxyfop-P-ethoxyethyl, haloxyfop-methyl, haloxyfop-P-methyl, hexazinone, HW-02, i.e. 1-(dimethoxyphosphoryl) ethyl-(2,4-dichlorophenoxy)acetate,imazamethabenz,imazamethabenz-methyl,imazamox,imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and -sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium, and -sodium, MCPB, MCPB-methyl, -ethy,l and -sodium, mecoprop, mecoprop-sodium, and -butotyl, mecoprop-P, mecoprop-P-butotyl, -dimethylammonium, -2-ethylhexyl, and -potassium, mefenacet, mefluidide, mesosulfuron, mesosulfuron-methyl, mesotrione, methabenzthiazuron, metam, metamifop, metamitron, metazachlor, metazosulfuron, methabenz-thiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinat, monolinuron, monosulfuron, monosulfuron-ester, MT-5950, i.e. N-(3-chloro-4-isopropylphenyl)-2-methylpentan amide, NGGC-011, napropamide, NC-310, i.e. [5-(benzyloxy)-1-methyl-1H-pyrazol-4-yl](2,4-dichlorophenyl)methanone, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxyfluorfen, paraquat, paraquat dichloride, pebulate, pendimethalin, penoxsulam, pentachlorphenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, SL-261, sulcotrione, sulfentrazone, sulfo-meturon, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxo-imidazolidine-4,5-dione, 2,3,6-TBA, TCA (trichloroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, trito-sulfuron, urea sulfate, vernolate, XDE-848, ZJ-0862, i.e. 3,4-dichloro-N-{2-[(4,6-dimethoxypyrimidin-2-yl)oxy]benzyl}aniline, and the following compounds:

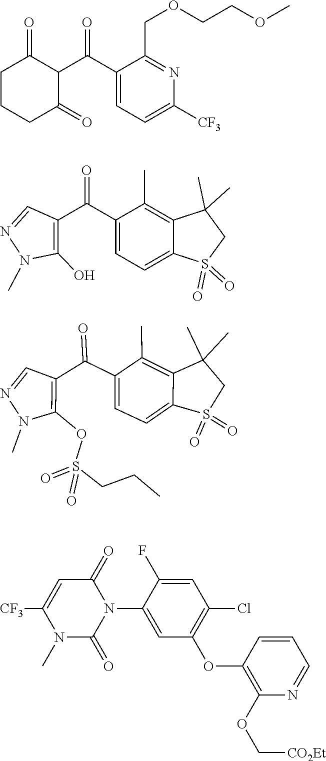

Group H2 discloses herbicides especially used in the context of the present invention are selected from the group consisting of:

Acetochlor, Alachlor, Dicamba, Diflufenican, Flufenacet, Fluroxypyr, Foramsulfuron, Glufosinate, L-Glufosinate, Glyphosate, Hexazinone, Imazamox, Imazethapyr, Indaziflam, Iodosulfuron, Isoxaflutole, Mesosulfuron, Mesotrione, Metolachlor, Metribuzin, Metsulfuron, Pendimethalin, Penoxsulam, Picloram, Pinoxaden, Pyroxsulam, Quinmerac, Rimsulfuron, Thiencarbazone, Tembotrione, Thifensulfuron, and/or Tribenuron, and the esters and/or agronomically acceptable salts of these herbicides.

Group H3 discloses preferred herbicides are selected from the group consisting of:

Acetochlor, Dicamba, Diflufenican, Flufenacet, Foramsulfuron, Glufosinate, L-Glufosinate, Glyphosate, Iodosulfuron, Isoxaflutole, Mesosulfuron, Mesotrione, Metribuzin, Pinoxaden, Tembotrione, and/or Thiencarbazone, and the esters and/or agronomically acceptable salts of these herbicides.

If Glyphosate salts are used as herbicide, preference is given to Glyphosate isopropylamine salt, Glyphosate potassium salt, Glyphosate sodium salt, Glyphosate trimethylsulfonium salt.

If (L-)Glufosinate salts are used as herbicide, preference is given to (L-)Glufosinate-ammonium salt, (L-) Glufosinate potassium salt and (L-)Glufosinate sodium salt.

If Foramsulfuron salts are used as herbicide, preference is given to Foramsulfuron-sodium.

If Iodosulfuron esters and/or salts thereof are used as herbicide, preference is given to Iodosulfuron-methyl and Iodosulfuron-methyl-sodium.

If Mesosulfuron esters and/or salts thereof are used as herbicide, preference is given to Mesosulfuron-methyl and Mesosulfuron-methyl-sodium.

If Thiencarbazone esters and/or salts thereof are used as herbicide, preference is given to Thiencarbazone-methyl and Thiencarbazone-methyl-sodium.

Preferred mixtures of herbicides used in the context of the present invention are disclosed in group H4: mixtures of Acetochlor and other herbicides mentioned herein, mixtures of Dicamba, esters and/or salts thereof and other herbicides mentioned herein, mixtures of Diflufenican and other herbicides mentioned herein, mixtures of Flufenacet and other herbicides mentioned herein, mixtures of Glufosinate and/or salts thereof (preferably Glufosinate-ammonium salt, Glufosinate potassium salt and Glufosinate sodium salt) and other herbicides mentioned herein, mixtures of L-Glufosinate and/or salts thereof (preferably L-Glufosinate-ammonium salt, L-Glufosinate potassium salt and L-Glufosinate sodium salt) and other herbicides mentioned herein, mixtures of Indaziflam and other herbicides mentioned herein, mixtures of Isoxaflutole and other herbicides mentioned herein, mixtures of Mesosulfuron esters and/or salts thereof (preferably Mesosulfuron-methyl(-sodium)) and other herbicides mentioned herein, mixtures of Metribuzin and other herbicides mentioned herein, mixtures of Thiencarbazone esters and/or salts thereof (preferably Thiencarbazone-methyl(-sodium)) and other herbicides mentioned herein, mixtures of Tembotrione and other herbicides mentioned herein.

Particularly preferred mixtures of herbicides used in the context of the present invention are disclosed in group H5:

mixtures of Acetochlor and Isoxaflutole, mixtures of Acetochlor and Tembotrione mixtures of Diflufenican and Flufenacet, mixtures of Diflufenican and Metribuzin, mixtures of Glufosinate and/or salts thereof and Indaziflam, in particular mixtures of Glufosinate-ammonium and Indaziflam, mixtures of L-Glufosinate and/or salts thereof and Indaziflam, in particular mixtures of L-Glufosinate-ammonium and Indaziflam, mixtures of Mesosulfuron-methyl(-sodium) and Iodosulfuron-methyl(-sodium), mixtures of Mesosulfuron-methyl(-sodium), Iodosulfuron-methyl(-sodium) and Flufenacet, mixtures of Tembotrione and Isoxaflutole, mixtures of Tembotrione, Isoxaflutole, and Acetochlor.

The herbicides and the mixtures of herbicides mentioned herein may be used in pre-emergence applications and/or in post-emergence applications.

In accordance with the present invention, the herbicides and the mixtures of herbicides mentioned herein may be applied as a split application over time. Another possibility is the application of the individual active ingredients or the mixtures comprising the active ingredients in a plurality of portions (sequential application), for example pre-emergence applications, followed by post-emergence applications or early post-emergence applications, followed by applications at medium or late post-emergence.

In one embodiment, a herbicide as listed in any one of groups H1 to H5 is used on seeds or plants comprising elite event EE-GM4 of the invention, or on soil wherein said seeds or plants are to be planted/sown, and said herbicide is not isoxaflutole, topramezone or mesotrione. In another embodiment, a herbicide as listed in any one of groups H1 to H5 is used on seeds or plants comprising elite event EE-GM4 of the invention, or on soil wherein said seeds or plants are to be planted/sown, wherein said plant or seed comprising EE-GM4 also contains one or more soybean SCN resistance genes from PI 88788, PI 548402, PI 209332 or PI 437654, or comprises one or more of the soybean SCN resistance loci or genes selected from the group consisting of: rhg1, rhg1-b, rhg2, rhg3, Rhg4, Rhg5, qSCN11, cqSCN-003, cqSCN-005, cqSCN-006, and cqSCN-007.

Examples for plant growth regulators are useful in the context of the present invention are disclosed in group P1:

Acibenzolar, acibenzolar-S-methyl, 5-aminolevulinic acid, ancymidol, 6-benzylaminopurine, Brassinolid, catechine, chlormequat chloride, cloprop, cyclanilide, 3-(cycloprop-1-enyl) propionic acid, daminozide, dazomet, n-decanol, dikegulac, dikegulac-sodium, endothal, endothal-dipotassium, -disodium, and -mono(N,N-dimethylalkylammonium), ethephon, flumetralin, flurenol, flurenol-butyl, flurprimidol, forchlorfenuron, gibberellic acid, inabenfide, indol-3-acetic acid (IAA), 4-indol-3-ylbutyric acid, isoprothiolane, probenazole, jasmonic acid, maleic hydrazide, mepiquat chloride, 1-methylcyclopropene, methyl jasmonate, 2-(1-naphthyl)acetamide, 1-naphthylacetic acid, 2-naphthyloxyacetic acid, nitrophenolate-mixture, paclobutrazol, N-(2-phenylethyl)-beta-alanine, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

### Biological Control Agent (BCA) Groups:

Biological control agents are, in particular, bacteria, fungi or yeasts, protozoa, viruses, entomopathogenic nematodes, products produced by microorganisms including proteins or secondary metabolites and botanicals, especially botanical extracts, that support or enhance plant or seed growth or development so as to protect or increase plant yield, particularly when plants or seeds experience stresses such as drought or attack by plant pathogens/pests (e.g., by killing plant pathogens or plant pests or preventing or impeding their development or their growth or preventing or impeding their penetration into or their sucking/feeding on plant tissue). Therefore, the Biological Control Agent (BCA) Groups (1) to (7) according to the invention are:

BCA Group (1): bacteria

BCA Group (2): fungi or yeasts

BCA Group (3): protozoa

BCA Group (4): viruses

BCA Group (5): entomopathogenic nematodes

BCA Group (6): products produced by microorganisms including proteins or secondary metabolites

BCA Group (7): botanicals, especially botanical extracts.

These Biological Control Agent (“BCA”) Groups BCA1 to BCA7 are further characterized as follows:

BCA1: According to the invention biological control agents which are summarized under the term “bacteria” include but are not limited to spore-forming, root-colonizing bacteria, or bacteria useful as bioinsecticide, biofungicide or bionematicide. Such bacteria to be used or employed according to the invention include but are not limited to:

(1.1) Agrobacterium radiobacter, in particular strain K84 (product known as Galltrol-A from AgBioChem, CA) or strain K1026 (product known as Nogall from Becker Underwood, US), (1.2) Agrobacterium vitis, in particular the non-pathogenic strain VAR03-1, (1.3) Azorhizobium caulinodans, preferably strain ZB-SK-5, (1.4) Azospirillum amazonense, (1.5) Azospirillum brasilense, (1.6) Azospirillum halopraeference, (1.7) Azospirillum irakense, (1.8) Azospirillum lipoferum, (1.9) Azotobacter chroococcum, preferably strain H 23 (CECT 4435) (cf. Applied Soil Ecology 12 (1999) 51±59), (1.10) Azotobacter vinelandii, preferably strain ATCC 12837 (cf. Applied Soil Ecology 12 (1999) 51±59), (1.11) Bacillus sp. strain AQ175 (ATCC Accession No. 55608), (1.12) Bacillus sp. strain AQ177 (ATCC Accession No. 55609), (1.13) Bacillus sp. strain AQ178 (ATCC Accession No. 53522), (1.14) Bacillus acidocaldarius, (1.15) Bacillus acidoterrestris, (1.16) Bacillus agri (cf. WO 2012/140207), (1.17) Bacillus aizawai (cf. WO 2012/140207), (1.18) Bacillus albolactis (cf. WO 2012/140207), (1.19) Bacillus alcalophilus, (1.20) Bacillus alvei, (1.21) Bacillus aminoglucosidicus, (1.22) Bacillus aminovorans, (1.23) Bacillus amylolyticus (also known as Paenibacillus amylolyticus), (1.24) Bacillus amyloliquefaciens, in particular B. amyloliquefaciens strain IN937a (cf. WO 2012/140207), or B. amyloliquefaciens strain FZB42 (DSM 231179) (product known as RhizoVital® from ABiTEP, DE), or B. amyloliquefaciens strain B3, or B. amyloliquefaciens strain D747, (products known as Bacstar® from Etec Crop Solutions, NZ, or Double Nickel™ from Certis, US), B. amyloliquefaciens s strain APM-1 (ATCC accession number PTA-4838, known as Aveo EZ from Valent), Bacillus amyloliquefaciens TJ1000 (also known as Bacillus amyloliquefaciens (Fukumoto) Priest et al. (ATCC BAA-390), or the B. amyloliquefaciens strains from US2012/0149571, such as B. amyloliquefaciens AP-136 (NRRL B-50330; NRRL B-50614), B. amyloliquefaciens AP-188 (NRRL B-50331; NRRL B-50615), B. amyloliquefaciens AP-218 (NRRL B-50618), B. amyloliquefaciens AP-219 (NRRL B-50332, NRRL B-50619), and B. amyloliquefaciens AP-295 (NRRL B-50333, NRRL B-50620), or Bacillus amyloliquefaciens strain MBI 600 (previously Bacillus subtilis strain MBI 600), or Bacillus amyloliquefaciens strain MBI 600 in combination with cis-Jasmone, or Bacillus amyloliquefaciens strain F727 (1.25) Bacillus aneurinolyticus, (1.26) Bacillus atrophaeus, (1.27) Bacillus azotoformans, (1.28) Bacillus badius, (1.29) Bacillus cereus (synonyms: Bacillus endorhythmos, Bacillus medusa), in particular spores of B. cereus strain CNCM I-1562 (cf. U.S. Pat. No. 6,406,690), or strain BPO1 (ATCC 55675), (products known as Mepichlor from Arysta, US or Mepplus, Micro-Flo Company LLC, US), (1.30) Bacillus chitinosporus, in particular strain AQ746 (Accession No. NRRL B-21618), (1.31) Bacillus circulans (1.32) Bacillus coagulans, in particular strain TQ33, (1.33) Bacillusfastidiosus, (1.34) Bacillus firmus, in particular strain I-1582 (products known as Bionem, Flocter or VOTIVO from Bayer CropScience, CNCM 1-1582), Bacillus firmus strain NRRL B-67003, or Bacillus firmus strain NRRL B-67518, (1.35) Bacillus kurstaki, (1.36) Bacillus lacticola, (1.37) Bacillus lactimorbus, (1.38) Bacillus lactis, (1.39) Bacillus laterosporus (also known as Brevibacillus laterosporus), (product known as Bio-Tode from Agro-Organics, SA), Bacillus laterosporus ATCC PTA-3952, Bacillus laterosporus ATCC PTA-3593, (1.40) Bacillus lautus, (1.41) Bacillus lentimorbus, (1.42) Bacillus lentus, (1.43) Bacillus licheniformis, in particular strain SB3086 (product known as EcoGuard™ Biofungicide or Green Releaf from Novozymes Biologicals, US), lichenformis CH200, or licheniformis RTI 184, or a combination of licheniformis CH200 and lichenformis RTI 184, or Bacillus lichenformis ATCC PTA-6175, (1.44) Bacillus maroccanus, (1.45) Bacillus medusa, (1.46) Bacillus megaterium, (products known as Bioarc®, from BioArc), or B. megaterium strain YFM3.25, (1.47) Bacillus metiens, (1.48) Bacillus mojavensis, in particular strain SR11 (CECT-7666), or Bacillus mojavensis AP-209 (US 2012/0149, NRRL B-50616), (1.49) Bacillus mycoides, in particular strain AQ726 (Accession No. NRRL B21664) or isolate J, (product known as BmJ from Certis USA), (1.50) Bacillus nematocida, (1.51) Bacillus nigrificans, (1.52) Bacilluspopilliae, (product known as Cronox from Bio-Crop, CO), (1.53) Bacillus psychrosaccharolyticus, (1.54) Bacillus pumilus, in particular strain GB34 (Accession No. ATCC 700814), (products known as Yield Shield® from Bayer Crop Science, DE), and strain QST2808 (Accession No. NRRL B-30087), (products known as Sonata QST 2808® from Bayer Crop Science), or strain BU F-33, (product known as Integral F-33 from Becker Underwood, US), or strain AQ717 (Accession No. NRRL B21662), (1.55) Bacillus siamensis, in particular strain KCTC 13613T, (1.56) Bacillus smithii, (1.57) Bacillus sphaericus, in particular Serotype H5a5b strain 2362, (product known as VectoLex® from Valent BioSciences, US), (1.58) Bacillus subtilis, in particular strain GB03 (Accession No. ATCC SD-1397), (product known as Kodiak® from Bayer Crop Science, DE), and strain QST713/AQ713 (Accession No. NRRL B-21661), (products known as Serenade QST 713®, Serenade Soil and Serenade Max from Bayer Crop Science) and strain AQ 153 (ATCC accession No. 55614), and strain AQ743 (Accession No. NRRL B-21665), and strain DB 101, (products known as Shelter from Dagutat Bio lab, ZA), and strain DB 102, (product known as Artemis from Dagutat Bio lab, ZA), and Bacillus subtilis strain MBI 600 (now Bacillus amyloliquefaciens strain MBI 600), (e.g., products known as Subtilex from Becker Underwood, US), or Bacillus subtilis Y1336 (available as BIOBAC® WP from Bion-Tech, Taiwan, registered as a biological fungicide in Taiwan under Registration Nos. 4764, 5454, 5096 and 5277), or B. subtilis var. amyloliquefaciens strain FZB24, (product known as Taegro® from Novozymes, US), a mutant of FZB24 that was assigned Accession No. NRRL B-50349 by the Agricultural Research Service Culture Collection and is also described in U.S. Patent Publication No. 20110230345, Bacillus amyloliquefaciens FZB42, available from ABiTEP GMBH, Germany, as the plant strengthening product RHIZOVITAL and described in European Patent Publication No. EP2179652, mutants of FZB42 described in International Application Publication No. WO 2012/130221, including Bacillus amyloliquefaciens ABI01, which was assigned Accession No. DSM 10-1092 by the DSMZ—German Collection of Microorganisms and Cell Cultures, or B. subtilis subspecies natto (formerly B. natto), or B. subtilis isolate B246, (product known as Avogreen from RE at UP) or strain MBI600 (products known as Subtilex or HiStick N/T from Becker Underwood), or strain QST30002/AQ30002 (Accesion No. NRRL B-50421, cf. WO 2012/087980), or strain QST30004/AQ30004 (Accession No. NRRL B-50455, cf. WO 2012/087980), or Bacillus subtilis (including Bacillus subtilis var. amyloliquefaciens) strains and Bacillus amyloliquefaciens strains that produce a fungicidal combination of lipopeptides, including (a) fengycin or plipastatin-type compound(s), (an) iturin-type compound(s), and/or (a) surfactin-type compound(s) (Ongena et al., Trends in Microbiology, Vol 16, No. 3, March 2008, pp. 115-125), (1.59) Bacillus tequilensis, in particular strain NII-0943, (1.60) Bacillus thuringiensis, in particular B. thuringiensis subspecies israelensis (serotype H-14), strain AM65-52 (Accession No. ATCC 1276), (product known as VectoBac® from Valent BioSciences, US), or B. th. israelensis strain BMP 144, (product known as Aquabac from Becker Microbial Products IL), or B. thuringiensis subsp. aizawai, in particular strain ABTS-1857 (SD-1372), (products known as XenTari® from Bayer Crop Science, DE) or strain GC-91 (Accession No. NCTC 11821), or serotype H-7, (product known as Florbac WG from Valent BioSciences, US), or B. thuringiensis subsp. kurstaki strain HD-1, (product known as Dipel® ES from Valent BioSciences, US), or strain BMP 123 from Becker Microbial Products, IL, or strain ABTS 351 (Accession No. ATCC SD-1275), or strain PB 54 (Accession No. CECT 7209), or strain SA 11 (Accession No. NRRL B-30790), or strain SA 12 (Accession No. NRRL B-30791), or strain EG 2348 (Accession No. NRRL B-18208), or strain EG-7841 (product known as Crymax from Certis USA), or B. thuringiensis subsp. tenebrionis strain NB 176 (SD-5428), (product known as Novodor® FC from BioFa DE), or B. thuringiensis subspecies. aegypti, (product known as Agerin), or B. thuringiensis var. colmeri (product known as TianBaoBTc from Changzhou Jianghai Chemical Factory), or B. thuringiensis var. darmstadiensis strains 24-91 (product known as Baciturin), or B. thuringiensis var. dendrolimus (products known as Dendrobacillin), or B. thuringiensis subsp. galleriae (product known as GrubGone or BeetleGone from Phyllom BioProducts), or B. thuringiensis var. japonensis strain Buibui, or B. thuringiensis subsp. morrisoni, or B. thuringiensis var. san diego (product known as M-One® from Mycogen Corporation, US), or B. thuringiensis subsp. thuringiensis serotype 1, strain MPPL002, or B. thuringiensis var. thuringiensis, or B. thuringiensis var 7216 (product known as Amactic, Pethian), or B. thuringiensis var T36 (product known as Cahat) or B. thuringiensis strain BD #32 (Accession No. NRRL B-21530) from Bayer Crop Science, DE, or B. thuringiensis strain AQ52 (Accession No. NRRL B-21619) from Bayer Crop Science, DE, or B. thuringiensis strain CR-371 (Accession No. ATCC 55273), (1.61) Bacillus unflagellatus, (1.62) Bradyrhizobium japonicum (product known as Optimize from Novozymes), (1.63) Brevibacillus brevis (formerly Bacillus brevis), (product known as Brevisin), in particular strains SS86-3, SS86-4, SS86-5, 2904, (1.64) Brevibacillus laterosporus (formerly Bacillus laterosporus), in particular strains ATCC 64, NRS 1111, NRS 1645, NRS 1647, BPM3, G4, NCIMB 41419, (1.65) Burkholderia spp., in particular Burkholderia rinojensis strain A396 (Accession No. NRRL B-50319), (product known as MBI-206 TGAI from Marrone Bio Innovations), or B. cepacia (product known as Deny from Stine Microbial Products), (1.66) Chromobacterium subtsugae, in particular strain PRAA4-1T (MBI-203), (product known as Grandevo from Marrone Bio Innovations), (1.67) Corynebacterium paurometabolum, (1.68) Delftia acidovorans, in particular strain RAY209 (product known as BioBoost® from Brett Young Seeds), (1.69) Gluconacetobacter diazotrophicus, (1.70) Herbaspirilum rubrisubalbicans, (1.71) Herbaspirilum seropedicae, (1.72) Lactobacillus sp. (product known as Lactoplant from LactoPAFI), (1.73) Lactobacillus acidophilus (product known as Fruitsan from Inagrosa-Industrias Agrobiológicas, S. A), (1.74) Lysobacter antibioticus, in particular strain 13-1 (cf. Biological Control 2008, 45, 288-296), (1.75) Lysobacter enzymogenes, in particular strain C3 (cf. J Nematol. 2006 June; 38(2): 233-239), (1.76) Paenibacillus alvei, in particular strains III3DT-1A, 1112E, 46C3, 2771 (Bacillus genetic stock center, November 2001), (1.77) Paenibacillus macerans, (1.78) Paenibacillus polymyxa, in particular strain AC-1 (product known as Topseed from Green Biotech Company Ltd.), (1.79) Paenibacillus popilliae (formerly Bacillus popilliae) product known as Milky spore disease from St. Gabriel Laboratories), (1.80) Pantoea agglomerans, in particular strain E325 (Accession No. NRRL B-21856), (product known as Bloomtime Biological FD Biopesticide from Northwest Agricultural Products), (1.81) Pasteuria nishizawae, such as the product known as oyacyst LF/ST from Pasteuria Bioscience, or Pasteuria nishizawae Pn1 (Clariva from Syngenta), (1.82) Pasteuria penetrans (formerly Bacillus penetrans), (product known as Pasteuria wettable powder from Pasteuria Bioscience), (1.83) Pasteuria ramosa, (1.84) Pasteuria renformis, (1.85) Pasteuria thornei, (1.86) Pasteuria usgae (products known as Econem™ from Pasteuria Bioscience), (1.87) Pectobacterium carotovorum (formerly Erwinia carotovora), (product known as BioKeeper from Nissan, JP), (1.88) Pseudomonas aeruginosa, in particular strains WS-1 or PN1, (1.89) Pseudomonas aureofaciens, in particular strain TX-1 (product known as Spot-Less Biofungicide from Eco Soils Systems, CA), (1.90) Pseudomonas cepacia (formerly known as Burkholderia cepacia), in particular type Wisconsin, strains M54 or J82, (1.91) Pseudomonas chlororaphis, in particular strain MA 342 (products known as Cedomon from Bioagri, S), or strain 63-28 (product known as ATEze from EcoSoil Systems, US), (1.92) Pseudomonas fluorescens, in particular strain A506 (products known as Blightban from NuFarm or Frostban B from Frost Technology Corp), or strain 1629RS (product known as Frostban D from Frost Technology Corp), (1.93) Pseudomonas proradix (product known as Proradix® from Sourcon Padena), (1.94) Pseudomonas putida, (1.95) Pseudomonas resinovorans (product known as Solanacure from Agricultural Resaerch Council, SA), (1.96) Pseudomonas syringae, in particular strain MA-4 (product known as Biosave from EcoScience, US), or strain 742RS (product known as Frostban C from Frost Technology Corp, (1.97) Rhizobium fredii, (1.98) Rhizobium leguminosarum, in particular bv. viceae strain Z25 (Accession No. CECT 4585), (1.99) Rhizobium loti, (1.100) Rhizobium meliloti, (1.101) Rhizobium trifolii, (1.102) Rhizobium tropici, (1.103) Rhodococcus globerulus strain AQ719 (Accession No. NRRL B21663) from Bayer Crop Science, DE, (1.104) Serratia entomophila (product known as Invade® from Wrightson Seeds), (1.105) Serratia marcescens, in particular strain SRM (Accession No. MTCC 8708) or strain R35, (1.106) Streptomyces sp. strain NRRL B-30145 from Bayer Crop Science, DE, or strains WYE 20 (KCTC 0341BP) and WYE 324 (KCTC0342BP), (1.107) Streptomyces acidiscabies, in particular strain RL-110T, (product known as MBI-005EP from Marrone Bioinnovations, CA), (1.108) Streptomyces candidus, in particular strain Y21007-2, (products known as BioBac or BioAid from Biontech, TW), (1.109) Streptomyces colombiensis (1.110) Streptomyces galbus (=Streptomyces griseoviridis), in particular strain K61 (Accession No. DSM 7206) (product known as Mycostop® from Verdera, cf. Crop Protection 2006, 25, 468-475) or strain QST 6047 (=strain NRRL B-30232) (product known as Virtuoso from Bayer Crop Science, DE), (1.111) Streptomyces goshikiensis, (1.112) Streptomyces lavendulae, (1.113) Streptomyces lydicus, in particular strain WYCD108US) or strain WYEC108 (product known as Actinovate from Natural Industries, US), (1.114) Streptomyces microflavus, in particular strain AQ6121 (=QRD 31.013, NRRL B-50550) from Bayer Crop Science, or strain M (=AQ6121.002) (091013-02 deposited with the Canadian International Depository Authority) from Bayer Crop Science, (1.115) Streptomyces prasinus (cf. “Prasinons A and B: potent insecticides from Streptomyces prasinus”, Applied microbiology 1973 November), (1.116) Streptomyces rimosus, (1.117) Streptomyces saraceticus (product known as Clanda from A & A Group (Agro Chemical Corp.)), (1.118) Streptomyces venezuelae, (1.119) Thiobacillus sp. (product known as Cropaid from Cropaid Ltd UK), (1.120) Virgibacillus pantothenticus (formerly Bacillus pantothenticus), in particular strain ATCC 14576/DSM 491, (1.121) Xanthomonas campestris (herbicidal activity), in particular pv poae (product known as Camperico), (1.122) Xenorhabdus (=Photorhabdus) luminescens, (1.123) Xenorhabdus (=Photorhabdus) nematophila, and (1.124) Bacillus solisalsi, such as Bacillus solisalsi AP-217 (US 2012/0149571, NRRL B-50617), Bacillus thuringiensis strain EX297512, Bacillus lichenformis FMCH001 (contained in product known as “PRESENCE” by Chr. Hansen), Bacillus subtilis FMCH002 (contained in the product known as “PRESENCE” by Chr. Hansen), Bacillus amyloliquefaciens MBI-600 (contained in TRUNEMCO), wherein said mentioned bacteria are preferred.

BCA2: According to the invention biological control agents that are summarized under the term “fungi” or “yeasts” include but are not limited to:

(2.1) Ampelomyces quisqualis, in particular strain AQ 10 (Accession No. CNCM I-807) (product known as AQ 10® from IntrachemBio Italia), (2.2) Arkansas Fungus 18 (ARF18, cf. WO2012/140207), (2.3) Arthrobotrys dactyloides (cf. WO 2012/140207), (2.4) Arthrobotrys oligospora (cf. WO 2012/140207), (2.5) Arthrobotrys superba, (cf. WO 2012/140207), (2.6) Aschersonia aleyrodis (cf. Berger, 1921. Bull. State Pl. Bd. 5:141), (2.7) Aspergillus flavus, in particular strain NRRL 21882 (product known as Afla-Guard® from Syngenta) or strain AF36 (product known as AF36 from Arizona Cotton Research and Protection Council, US), (2.8) Aureobasidium pullulans, in particular blastospores of strain DSM14940 or blastospores of strain DSM 14941 or mixtures thereof (products known as Botector® or Blossom Protect® from bio-ferm, CH), (2.9) Beauveria bassiana, in particular strain ATCC 74040 (product known as Naturalis® from Intrachem Bio Italia) and strain GHA (Accession No. ATCC74250) (products known as BotaniGuard Es or Mycontrol-O from Laverlam International Corporation), or strain ATP02 (Accession No. DSM 24665, cf. WO/2011/117351), or strain CG 716 (product known as BoveMax® from Novozymes), or strain ANT-03 (from Anatis Bioprotection, CA), (2.10) Beauveria brongniartii (product known as Beaupro from Andermatt Biocontrol AG), (2.11) Candida oleophila, in particular strain O (product known as Nexy® from BioNext) or isolate I-182 (product known as Aspire® from Ecogen, US), (2.12) Candida saitoana, in particular strain NRRL Y-21022 (cf. U.S. Pat. No. 5,591,429), (2.13) Chaetomium cupreum, (2.14) Chaetomium globosum, (2.15) Cladosporium cladosporioides, in particular strain H39, (2.16) Colletotrichum gloeosporioides, in particular strain ATCC 20358, (2.17) Conidiobolus obscurus, (2.18) Coniothyrium minitans, in particular strain CON/M/91-8 (Accession No. DSM-9660), (product known as Contans® from Bayer Crop Science, DE), (2.19) Cryptococcus albidus (product known as YieldPlus® from Anchor Bio-Technologies, ZA), (2.20) Cryptococcus flavescens, in particular strain 3C (NRRL Y-50378) and strain 4C (NRRL Y-50379) (described in U.S. Pat. No. 8,241,889), (2.21) Cylindrocarpon heteronema, (2.22) Dactylaria candida, (2.23) Dilophosphora alopecuri (product known as Twist Fungus®), (2.24) Entomophthora virulenta (product known as Vektor), (2.25) Exophiala jeanselmei, (2.26) Exophilia pisciphila, (2.27) Fusarium oxysporum, in particular strain Fo47 (non-pathogenic) (product known as Fusaclean from Natural Plant Protection, FR), (2.28) Fusarium solani, for example strain Fs5 (non-pathogenic), (2.29) Gigaspora margarita, (2.30) Gigaspora monosporum, (2.31) Gliocladium catenulatum (Synonym: Clonostachys rosea f. catenulate) in particular strain J1446 (products known as Prestop® from AgBio Inc. or Primastop® from Kemira Agro Oy), (2.32) Gliocladium roseum, in particular strain 321U, (2.33) Glomus aggregatum, (2.34) Glomus brasilianum, (2.35) Glomus clarum, (2.36) Glomus deserticola, (2.37) Glomus etunicatum, (2.38) Glomus intraradices, (2.39) Glomus iranicum, (2.40) Glomus monosporum, (2.41) Glomus mosseae, (2.42) Harposporium anguillullae, (2.43) Hirsutella citriformis, (2.44) Hirsutella minnesotensis, (2.45) Hirsutella rhossiliensis, (2.46) Hirsutella thompsonii (products known as Mycohit or ABTEC from Agro Bio-tech Research Centre, IN), (2.47) Laccaria bicolor, (2.48) Laccaria laccata, (2.49) Lagenidium giganteum (product known as Laginex®, Bayer Crop Science, DE), (2.50) Lecanicillium spp., in particular strain HRO LEC 12 from Bayer Crop Science, DE, (2.51) Lecanicillium lecanii (formerly known as Verticillium lecanii) in particular conidia of strain KVO1 (products known as Mycotal® or Vertalec®, Koppert/Arysta), or strain DAOM198499, or strain DAOM216596, (2.52) Lecanicillium muscarium (formerly Verticillium lecanii), in particular strain 1/1 from Bayer Crop Science, DE, or strain VE 6/CABI(=IMI) 268317/CBS102071/ARSEF5128, (2.53) Meristacrum asterospermum (2.54) Metarhizium anisopliae, in particular strain F52 (DSM3884/ATCC 90448) (products known as BIO 1020, Bayer Crop Science, DE, or Met52, Novozymes), or M. anisopliae var acridum (product known as GreenGuard, Becker Underwood, US), or M. anisopliae var acridum isolate IMI 330189 (ARSEF7486), (product known as Green Muscle from Biological Control Products), (2.55) Metarhizium flavoviride, (2.56) Metschnikowia fructicola, in particular the strain NRRL Y-30752 (product known as Shemer® from Bayer Crop Science, DE), (2.57) Microdochium dimerum, in particular strain L13 (products known as ANTIBOT® from Agrauxine), (2.58) Microsphaeropsis ochracea (product known as Microx® from Bayer Crop Science, DE), (2.59) Monacrosporium cionopagum, (2.60) Monacrosporium psychrophilum, (2.61) Monacrosporium drechsleri, (2.62) Monacrosporium gephyropagum (2.63) Mucor haemelis (product known as BioAvard from Indore Biotech Inputs & Research), (2.64) Muscodor albus, in particular strain QST 20799 (Accession No. NRRL 30547) (products known as Arabesque™, Glissade™, or Andante™ from Bayer Crop Science, DE), (2.65) Muscodor roseus strains A3-5 (Accession No. NRRL 30548), (2.66) Myrothecium verrucaria, in particular strain AARC-0255 (product known as DiTera™ from Valent Biosciences), (2.67) Nematoctonus geogenius, (2.68) Nematoctonus leiosporus, (2.69) Neocosmospora vasinfecta, (2.70) Nomuraea rileyi, in particular strains SA86101, GU87401, SR86151, CG128 and VA9101, (2.71) Ophiostoma pilferum, in particular strain D97 (product known as Sylvanex), (2.72) Paecilomyces fumosoroseus (new: Isaria fumosorosea), in particular strain IFPC 200613, or strain apopka 97 (product known as PreFeRal® WG from Biobest) or strain FE 9901 (products known as NoFly® from Natural Industries Inc., US), (2.73) Paecilomyces lilacinus, in particular spores of P. lilacinus strain 251 (AGAL 89/030550), (product known as BioAct® from Bayer Crop Science, DE; cf. Crop Protection 2008, 27, 352-361), (2.74) Paecilomyces variotii, in particular strain Q-09 (product known as Nemaquim® from Quimia, MX), (2.75) Pandora delphacis, (2.76) Paraglomus sp, in particular P. brasilianum, (2.77) Penicillium bilaii, in particular strain ATCC 22348 (products known as JumpStart® from Novozymes, PB-50, Provide), (2.78) Penicillium vermiculatum, (2.79) Phlebiopsis (or Phlebia or Peniophora) gigantea, in particular the strains VRA 1835 (ATCC 90304), VRA 1984 (DSM16201), VRA 1985 (DSM16202), VRA 1986 (DSM16203), FOC PG B20/5 (IMI390096), FOC PG SP log 6 (IMI390097), FOC PG SP log 5 (IMI390098), FOC PG BU3 (IMI390099), FOC PG BU4 (IMI390100), FOC PG 410.3 (IMI390101), FOC PG 97/1062/116/1.1 (IMI390102), FOC PG B22/SP1287/3.1 (IMI390103), FOC PG SH1 (IMI390104), FOC PG B22/SP1190/3.2 (IMI390105), (products known as Rotstop® from Verdera, FIN, PG-Agromaster®, PG-Fungler®, PG-IBL, PG-Poszwald, Rotex® from e-nema, DE), (2.80) Phoma macrostroma, in particular strain 94-44B (products known as Phoma H or Phoma P from Scotts, US), (2.81) Pichia anomala, in particular strain WRL-076 (NRRL Y-30842), (2.82) Pisolithus tinctorius, (2.83) Pochonia chlamydosporia (for example the product known as Rizotec; the bacterial strain is also known as Vercillium chlamydosporium), in particular var catenulata (IMI SD 187) (product known as KlamiC from The National Center of Animal and Plant Health (CENSA), CU), or P. chlamydosporia var chlamydosporia (resp. V. chlamydosporium var chlamydosporium), (2.84) Pseudozyma aphidis (2.85), Pseudozyma flocculosa, in particular strain PF-A22 UL (product known as Sporodex® L from Plant Products Co., CA), (2.86) Pythium oligandrum, in particular strain DV74 or M1 (ATCC 38472), (product known as Polyversum from Bioprepraty, CZ), (2.87) Rhizopogon amylopogon, (2.88) Rhizopogon fulvigleba, (2.89) Rhizopogon luteolus, (2.90) Rhizopogon tinctorus, (2.91) Rhizopogon villosullus, (2.92) Saccharomyces cerevisae, in particular strain CNCM No. I-3936, strain CNCM No. I-3937, strain CNCM No. I-3938, strain CNCM No. I-3939 (patent application US 2011/0301030), (2.93) Scleroderma citrinum, (2.94) Sclerotinia minor, in particular strain IMI 344141 (product known as Sarritor), (2.95) Sporothrix insectorum (product known as Sporothrix Es from Biocerto, BR), (2.96) Stagonospora atriplicis, (2.97) Stagonospora heteroderae, (2.98) Stagonospora phaseoli, (2.99) Suillus granulatus, (2.100) Suillus punctatapies, (2.101) Talaromyces flavus, in particular strain V117b (product known as PROTUS® WG from Bayer Crop Science, DE), (2.102) Trichoderma album (product known as Bio Zeid® from Organic Biotechnology, EG), (2.103) Trichoderma asperellum, in particular strain ICC 012 (CABI CC IMI 392716) (also known as Trichoderma harzianum ICC012), or strain SKT-1 (product known as ECO-HOPE® from Kumiai Chemical Industry) or strain T34 (product known as T34 Biocontrol from Bioncontrol Technologies, ES) or isolate SF04 (URM-5911) or strain TV1 (MUCL 43093) (also known as Trichoderma viride TV1) or strain T11 (CECT 20178) (also known as Trichoderma viride T25), (2.104) Trichoderma atroviride, in particular strain CNCM I-1237 (product known as Esquive® WP from Agrauxine, FR,) or the strains NMI No. V08/002387, NMI No. V08/002388, NMI No. V08/002389, NMI No. V08/002390 (patent application US 2011/0009260) or strain ATCC 20476 (IMI 206040) or strain T11 (IMI352941/CECT20498) or strain LC52 (products known as Tenet® or Sentinel® from Agrimm Technologies, NZ), or strain SCI from Bayer Crop Science, DE, or the strains SKT-1 (FERM P-16510), SKT-2 (FERM P-16511) and SKT-3 (FERM P-17021), (2.105) Trichoderma gamsii (formerly T. viride), in particular strain ICC080 (IMI CC 392151 CABI) (product known as Bioderma), (2.106) Trichoderma harmatum, in particular strain TH382 (product known as Incept from Syngenta), (2.107) Trichoderma harzianum, in particular T. harzianum rifai T39 (product known as Trichodex® from Makhteshim, US), or T. harzianum rifai strain KRL-AG2 (strain T-22, /ATCC 208479) (products known as PLANTSHIELD T-22G, Rootshield® and TurfShield from BioWorks, US), or strain KD (products known as Trichoplus from Biological Control Products, SA, or Eco-T from Plant Health Products, SZ), or strain ITEM 908 (CBS 118749), or strain TH 35 (formerly known as Trichoderma lignorum), (product known as Root Pro from Mycontrol), or strain DB 103 (product known as T-Gro from Dagutat Biolab), or strain TSTh20 (Patent Deposit Designation number PTA-0317), or strain 1295-22, (2.108) Trichoderma koningii, (2.109) Trichoderma lignorum, in particular strain TL-0601 (product known as Mycotric from Futureco Bioscience, ES), (2.110) Trichoderma polysporum, in particular strain IMI 206039/ATCC 20475, (2.111) Trichoderma saturnisporium, in particular strain PBP-TH-001 from Bayer Crop Science, DE, (2.112) Trichoderma stromaticum (product known as TRICOVAB® from Ceclap, BR), (2.113) Trichoderma virens (also known as Gliocladium virens), in particular strain GL-21 (product known as SoilGard from Certis, US) or strain G41 or Trichoderma (Gliocladium) virens strain GI-3 (or G1-3, ATCC 58678), (2.114) Trichoderma viride, in particular strain TV1, (2.115) Tsukamurella paurometabola, in particular strain C-924 (product known as HeberNem®), (2.116) Ulocladium oudemansii, in particular strain HRU3 (product known as Botry-Zen® from Botry-Zen Ltd, NZ), (2.117) Verticillium albo-atrum (formerly V. dahliae), in particular strain WCS850 (CBS 276.92), (2.118) Verticillium chlamydosporium, (2.119) Verticillium dahlia, (2.120) Zoophtora radicans, and (2.121) a Penicillium strain, such as Penicillium steckii strain IBWF 104-06 (accession number DSM 27859).

wherein said mentioned fungi or yeasts are preferred.

BCA3: According to the invention biological control agents that are summarized under the term “protozoa” include but are not limited to: (3.1) Nosema locustae (product known as NoloBait), (3.2) Thelohania solenopsis and (3.3) Vairimorpha spp,

wherein said mentioned protozoa are preferred.

BCA4: According to the invention biological control agents that are summarized under the term “viruses” include but are not limited to:

(4.1) Adoxophyes orana (summer fruit tortrix) granulosis virus (GV), (product known as Capex® from BIOFA), (4.2) Agrotis segetum (turnip moth) nuclear polyhedrosis virus (NPV), (4.3) Anagrapha falcifera (celery looper) NPV, (4.4) Anticarsia gemmatalis (woolly pyrol moth) multiple NPV (product known as Coopervirus PM by Coodetec), (4.5) Autographa californica (alfalfa looper) mNPV (product known as VPN80 from Agricola El Sol; GT), (4.6) Biston suppressaria (tea looper) NPV, (4.7) Bombyx mori (silkworm) NPV, (4.8) Cryptophlebia leucotreta (false codling moth) GV (products known as Cryptex from Andermatt Biocontrol, CH), (4.9) Cydia pomonella (codling moth) granulosis virus (GV) (product known as Madex Plus from Andermatt Biocontrol, CH), (4.10) Dendrolimus punctatus (masson pine moth) CPV, (4.11) Helicoverpa armigera (cotton bollworm) NPV (product known as Helicovex from Andermatt Biocontrol, CH), (4.12) Helicoverpa (previously Heliothis) zea (corn earworm) NPV (product known as Elcar), (4.13) Leucoma salicis (satin moth) NPV, (4.14) Lymantria dispar (gypsy moth) NPV (product known as Gypcheck, US Forest Service), (4.15) Neodiprion abietis (balsam-fir sawfly) NPV (product known as Abietiv™), (4.16) Neodiprion lecontei (red-headed pine sawfly) NPV (product known as Lecontvirus from the Canadian Forestry Service), (4.17) Neodiprion sertifer (pine sawfly) NPV (product known as Neocheck-S from the US Forest service), (4.18) Orgyia pseudotsugata (douglas-fir tussock moth) NPV (product known as TM-BioControl-™) (4.19) Phthorimaea operculella (tobacco leaf miner) GV (product known as Matapol Plus), (4.20) Pieris rapae (small white butterfly) GV, (4.21) Plutella xylostella (diamondback moth) GV, (4.22) Spodoptera albula (gray-streaked armywom moth) mNPV (product known as VPN 82, Agricola El Sol, GT), (4.23) Spodoptera exempta (true armyworm) mNPV, (4.24) Spodoptera exigua (sugarbeet armyworm) mNPV (product known as Spexit from Andermatt Biocontrol), (4.25) Spodoptera frugiperda (fall armyworm) mNPV), (4.26) Spodoptera littoralis (tobacco cutworm) NPV (products known as Littovir from Andermatt Biocontrol, CH or Spodoptrin from NPP Calliope France), and (4.27) Spodoptera litura (oriental leafworm moth) NPV (products known as Littovir), wherein said mentioned viruses are preferred.

BCA5: According to the invention biological control agents that are summarized under the term “entomopathogenic nematodes” include but are not limited to:

(5.1) Abbreviata caucasica, (5.2) Acuaria spp., (5.3) Agamermis decaudata, (5.4) Allantonema spp., (5.5) Amphimermis spp., (5.6) Beddingia (=Deladenus) siridicola, (5.7) Bovienema spp., (5.8) Cameronia spp., (5.9) Chitwoodiella ovofilamenta, (5.10) Contortylenchus spp., (5.11) Culicimermis spp., (5.12) Diplotriaena spp., (5.13) Empidomermis spp., (5.14) Filipjevimermis leipsandra, (5.15) Gastromermis spp., (5.16) Gongylonema spp., (5.17) Gynopoecilia pseudovipara, (5.18) Heterorhabditis spp., in particular (5.19) Heterorhabditis bacteriophora (products known as B-Green® or Larvanem®, Koppert or Nemasys® G, Becker Underwood), or (5.20) Heterorhabditis baujardi, or (5.21) Heterorhabditis heliothidis (products known as Nematon®, biohelp GmbH), or (5.22) Heterorhabditis indica, (5.23) Heterorhabditis marelatus, (5.24) Heterorhabditis megidis (products known as Larvanem® M, Koppert or Meginem®, Andermatt Biocontrol AG or Nemasys-H®), (5.25) Heterorhabditis zealandica, (5.26) Hexamermis spp., (5.27) Hydromermis spp., (5.28) Isomermis spp., (5.29) Limnomermis spp., (5.30) Maupasina weissi, (5.31) Mermis nigrescens, (5.32) Mesomermis spp., (5.33) Neomesomermis spp., (5.34) Neoparasitylenchus rugulosi, (5.35) Octomyomermis spp., (5.36) Parasitaphelenchus spp., (5.37) Parasitorhabditis spp., (5.38) Parasitylenchus spp., (5.39) Perutilimermis culicis, (5.40) Phasmarhabditis hermaphrodita (product known as Nemaslug from BASF AG), (5.41) Physaloptera spp., (5.42) Protrellatus spp., (5.43) Pterygodermatites spp., (5.44) Romanomermis spp., (5.45) Seuratum cadarachense, (5.46) Sphaerulariopsis spp., (5.47) Spirura guianensis, (5.48) Steinernema spp. (=Neoaplectana spp.), in particular (5.49) Steinernema bibionis (product known as Nematoden gegen Trauermücken®), or (5.50) Steinernema carpocapsae (products known as Biocontrol, Nemasys-C®, NemAttack®), or (5.51) Steinernema feltiae (=Neoaplectana carpocapsae), (products known as Nemasys®, Nemaflor®, Nemaplus®, NemaShield®), or (5.52) Steinernema glaseri (products known as Biotopia®), or (5.53) Steinernema kraussei (products known as Exhibitline®, Grubsure®, Kraussei System®, Larvesure®), or (5.54) Steinernema riobrave (products known as Biovector®), or (5.55) Steinernema scapterisci (products known as Nematac® S), or (5.56) Steinernema scarabaei, or (5.57) Steinernema siamkayai, (5.58) Steinernema thailandse (products known as Nemanox®), (5.59) Strelkovimermis peterseni, (5.60) Subulura spp., (5.61) Sulphuretylenchus elongatus, and (5.62) Tetrameres spp.,

wherein said mentioned entomopathogenic nematodes are preferred.

BCA6: Biological control agents which are summarized under the term “proteins or secondary metabolites” include but are not limited to:

(6.1) Bacillus thuringiensis toxins (isolated from different subspecies of B. thuringiensis), (6.2) Gougerotin (isolated from Streptomyces microflavus strain AQ 6121, from Bayer Crop Science), (6.3) Harpin (isolated from Erwinia amylovora, products known as Harp-N-Tek™, Messenger®, Employ™, ProAct™), (6.4) the spider toxin GS-omega/kappa-Hxtx-Hv1a, product known as Versitude from Vestaron,
wherein said mentioned proteins or secondary metabolites are preferred.

BCA7: Biological control agents which are summarized under the term “botanical extracts” include but are not limited to:

(7.1) Thymol, extracted e. g. from thyme (Thymus vulgaris), (7.2) Neem tree (Azadirachta indica) oil, and therein Azadirachtin, (7.3) Pyrethrum, an extract made from the dried flower heads of different species of the genus Tanacetum, and therein Pyrethrins (the active components of the extract), (7.4) extract of Cassia nigricans, (7.5) wood extract of Quassia amara (bitterwood), (product known as Quassan from Andermatt Biocontrol AG), (7.6) Rotenon, an extract from the roots and stems of several tropical and subtropical plant species, especially those belonging to the genera Lonchocarpus and Derris, (7.7) extract of Allium sativum (garlic), (7.8) Quillaja extract, made from the concentrated purified extract of the outer cambium layer of the Quillaja Saponaria Molina tree, (7.9) Sabadilla (Sabadilla=Schoenocaulon officinale) seeds, in particular Veratrin (extracted from the seeds), (7.10) Ryania, an extract made from the ground stems of Ryania speciosa, in particular Ryanodine (the active component of the extract), (7.11) extract of Viscum album (mistletoe), (7.12) extract of Tanacetum vulgare (tansy), (7.13) extract of Artemisia absinthium (wormwood), (7.14) extract of Urtica dioica (stinging nettle), (7.15) extract of Symphytum officinale (common comfrey), (7.16) extract of Tropaeulum majus (monks cress), (7.17) leaves and bark of Quercus (oak tree) (7.18) Yellow mustard powder, (7.19) oil of the seeds of Chenopodium anthelminticum (wormseed goosefoot), (7.20) dried leaves of Dryopteris filixmas (male fern), (7.21) bark of Celastrus angulatus (chinese bittersweet), (7.22) extract of Equisetum arvense (field horsetail), (7.23) Chitin (7.24) natural extracts or simulated blend of Chenopodium ambrosioides (wormseed), (product known as Requiem® from Bayer Crop Science) which contains a mixture of three terpenes, i.e. α-terpinene (around 10%), p-cymene (around 3.75%) and limonene (around 3%) as pesticidally active ingredients; it is disclosed in US 2010/0316738 corresponding to WO 2010/144919), (7.25) Saponins of Chenopodium quinoa (quinoa goosefoot), (product known as Heads Up), (7.26) Maltodextrin (product known as Majestik from Certis Europe), (7.27) orange oil (product known as PREV-AM from Oro Agri B.V.), sesame oil (product known as Dragon-fire-CCP, U.S. Pat. No. 6,599,539), wherein said mentioned botanical extracts are preferred.

Also included are bacteria and fungi which are added as ‘inoculant’ to plants or plant parts or plant organs and which, by virtue of their particular properties, promote plant growth and plant health. Examples which may be mentioned are:

Agrobacterium spp., Azorhizobium caulinodans, Azospirillum spp., Azotobacter spp., Bradyrhizobium spp., Burkholderia spp., in particular Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., in particular Rhizobium triolii, Rhizopogon spp., Scleroderma spp., Suillus spp., Streptomyces spp., Rhizophagus irregularis (previously known as Glomus intraradices).

Also included herein are any of the biological or chemical control agents ((bio)pesticides) described in US2017/0188584 (incorporated by reference herein, such as any of the biological or chemical control agents from the groups A) to O), or the biopesticides listed in paragraph 127 in US2017/0188584). Biological control agents as used herein can be obtained from culture collections and deposition centers (often referred to by their acronym (on the world wide web at wfcc.info/ccinfo/collection/by_acronym/)) or strain prefix herein, such as strains with the following prefixes from the following collections: AGAL or NMI from: National Measurement Institute, 1/153 Bertie Street, Port Melbourne, Victoria, Australia 3207; ATCC from American Type Culture Collection, 10801 University Blvd., Manassas, Va. 20110-2209, USA; BR from Embrapa Agrobiology Diazothrophic Microbial Culture Collection, P.O. Box 74.505, Seropedica, Rio de Janeiro, 23.851-970, Brazil; CABI or IMI from CABI Europe—International Mycological Institute, Bakeham Lane, Egham, Surrey, TW20 9TYNRRL, UK; CB from The CB Rhizobium Collection, School of Environment and Agriculture, University of Western Sydney, Hawkesbury, Locked Bag 1797, South Penrith Distribution Centre, NSW 1797, Australia; CBS from Centraalbureau voor Schimmelcultures, Fungal Biodiversity Centre, Uppsalaan 8, PO Box 85167, 3508 AD Utrecht, Netherlands; CC from the Division of Plant Industry, CSIRO, Canberra, Australia; CNCM from Collection Nationale de Cultures de Microorganismes, Institute Pasteur, 25 rue du Docteur Roux, F-75724 PARIS Cedex 15; CPAC from Embrapa-Cerrados, CX. Postal 08223, Planaltina, DF, 73301-970, Brazil; DSM from Leibniz-Institut DSMZ-Deutsche Sammlung von Mikroorganismen and Zellkulturen GmbH, Inhoffenstra.beta.e 7 B, 38124 Braunschweig, Germany; IDAC from International Depositary Authority of Canada Collection, Canada; ICMP from Interntional Collection of Micro-organisms from Plants, Landcare Research, Private Bag 92170, Auckland Mail Centre, Auckland 1142, New Zealand; IITA from IITA, PMB 5320, Ibadan, Nigeria; INTA from Agriculture Collection Laboratory of the Instituto de Microbiologia y Zoologia Agricola (IMYZA), Instituto Nacional de Tecnologi'a Agropecuaria (INTA), Castelar, Argentina; MSDJ from Laboratoire de Microbiologie des Sols, INRA, Dijon, France; MUCL from Mycotheque de l'Universite catholique de Louvain, Croix du Sud 2, box L7.05.06, 1348 Louvain-la-Neuve, Belgium; NCIMB or NICB from The National Collections of Industrial and Marine Bacteria Ltd., Torry Research Station, P.O. Box 31, 135 Abbey Road, Aberdeen, AB9 8DG, Scotland; Nitragin from Nitragin strain collection, The Nitragin Company, Milwaukee, Wis., USA, NRRL or ARSEF (collection of entomopathogenic fungi) from ARS Culture Collection of the National Center for Agricultural Utilization Research, Agricultural Research Service, U.S. Department of Agriculture, 1815 North University Street, Peoria, Ill. 61604, USA; NZP from the Department of Scientific and Industrial Research Culture Collection, Applied Biochemistry Division, Palmerston North, New Zealand; PPRI from ARC-Plant Protection Research Institute, Private Bag X134, Queenswood Pretoria, Gauteng, 0121, South Africa; SEMIA from FEPAGRO-Fundacao Estadual de Pesquisa Agropecuaria, Rua Gonsalves Dias, 570, Bairro Menino Deus, Porto Alegre/RS, Brazil; SRDI from SARDI, Adelaide, South Australia; USDA from U.S. Department of Agriculture, Agricultural Research Service, Soybean and Alfalfa Research Laboratory, BARC-West, 10300 Baltimore Boulevard, Building 011, Beltsville, Md. 20705, USA (Beltsville Rhiz. Cult. Catalog: on the world wide web at pdf.usaid.gov/pdf_docs/PNAAW891.pdf); and WSM from Murdoch University, Perth, Western Australia.

Also included herein as biological control agent are any of the following biochemical pesticides: citral, (E,Z)-7,9-dodecadien-1-yl acetate, ethyl formate, (E,Z)-2,4-ethyl decadienoate (pear ester), (Z,Z,E)-7,11,13-hexadecatrienal, heptyl butyrate, isopropyl myristate, lavanulyl senecioate, 2-methyl 1-butanol, methyl eugenol, (E,Z)-2,13-octadecadien-1-ol, (E,Z)-2,13-octadecadien-1-ol acetate, (E,Z)-3,13-octadecadien-1-ol, R-1-octen-3-ol, pentatermanone, potassium silicate, sorbitol actanoate, (E,Z,Z)-3,8,11-tetradecatrienyl acetate, (Z,E)-9,12-tetradecadien-1-yl acetate, Z-7-tetradecen-2-one, Z-9-tetradecen-1-yl acetate, Z-11-tetradecenal, Z-11-tetradecen-1-ol, Acacia negra extract, and extract of grapefruit seeds and pulp.

Also included herein as biological control agent is any of the biopesticides mentioned at: (on the world wide web at sitem.herts.ac.uk/aeru/bpdb/atoz.htm, gcm.wfcc.info/, landcareresearch.co.nz/resources.collections/icmp, epa.gov/opp00001/biopesticides/, omri.org/omri-lists, and included as compound herein is any of the pesticides mentioned at sitem.herts.ac.uk/aeru/ppdb/en/atoz.htm.

In one embodiment, a biological control agent for use in the current invention includes one or more biological control agents selected from group BCA8: a Bacillus species strain, a Brevibacillus species strain, a Burkholderia species strain, a Lysobacter species strain, a Pasteuria species strain, an Arthrobotrys species strain, a Nematoctonus species strain, a Myrothecium species strain, a Paecilomyces species strain, a Trichoderma species strain, and a Tsukamurella species strain.

In another embodiment, the plant, cell, plant part or seed of the invention, or the soil in which they are grown or are intended to be grown, are treated with a biological control agent selected from group BCA9 consisting of: Bacillus amyloliquefaciens, Bacillus firmus, Bacillus laterosporus, Bacillus lentus, Bacillus lichenformis, Bacillus nematocida, Bacillus pumilus, Bacillus subtilis, Bacillus penetrans, Bacillus thuringiensis, Brevibacillus laterosporus, Burkholderia rinojensis, Lysobacter antibioticus, Lysobacter enzymogenes, Pasteuria nishizawae, Pasteuria penetrans, Pasteuria ramosa, Pasteuria renformis, Pasteuria thornei, Pasteuria usage, Arthrobotrys dactyloides, Arthrobotrys oligospora, Arthrobotrys superba, Nematoctonus geogenius, Nematoctonus leiosporus, Myrothecium verrucaria, Paecilomyces lilacinus, Paecilomyces variotii, Trichoderma asperellum, Trichoderma harzianum, Trichoderma viride, Trichoderma harzianum rifai, and Tsukamurella paurometabola.

In one embodiment, the plant, cell, plant part or seed of the invention, or the soil in which they are grown or are intended to be grown, are treated with a biological control agent selected from group BCA10 consisting of: Bacillus amyloliquefaciens strain IN937a, Bacillus amyloliquefaciens strain FZB42, Bacillus amyloliquefaciens strain FZB24, Bacillus amyloliquefaciens strain ABI01, Bacillus amyloliquefaciens strain B3, Bacillus amyloliquefaciens strain D747, Bacillus amyloliquefaciens strain APM-1, Bacillus amyloliquefaciens strain TJ1000, Bacillus amyloliquefaciens strain AP-136, Bacillus amyloliquefaciens strain AP-188, Bacillus amyloliquefaciens strain AP-218, Bacillus amyloliquefaciens strain AP-219, Bacillus amyloliquefaciens strain AP-295, Bacillus amyloliquefaciens strain MBI 600, Bacillus amyloliquefaciens strain PTA-4838, Bacillus amyloliquefaciens strain F727, Bacillus firmus strain I-1582, Bacillus firmus strain NRRL B-67003, Bacillus firmus strain NRRL B-67518, Bacillus firmus strain GB126, Bacillus laterosporus strain ATCC PTA-3952, Bacillus laterosporus strain ATCC PTA-3593, Bacillus lichenformis strain ATCC PTA-6175, Bacillus thuringiensis strain EX297512, Brevibacillus laterosporus strain ATCC 64, Brevibacillus laterosporus strain NRS 1111, Brevibacillus laterosporus strain NRS 1645, Brevibacillus laterosporus strain NRS 1647, Brevibacillus laterosporus strain BPM3, G4, Brevibacillus laterosporus strain NCIMB 41419, Burkholderia rinojensis strain A396, Lysobacter antibioticus strain 13-1, Lysobacter enzymogenes strain C3, Myrothecium verrucaria strain AARC-0255, Paecilomyces lilacinus strain 251, Paecilomyces variotii strain Q-09, Trichoderma asperellum strain ICC 012, Trichoderma asperellum strain SKT-1, Trichoderma asperellum strain T34, Trichoderma asperellum strain T25, Trichoderma asperellum strain SF04, Trichoderma asperellum strain TV1, Trichoderma asperellum strain T 11, Trichoderma harzianum strain ICC012, Trichoderma harzianum rifai T39, Trichoderma harzianum rifai strain KRL-AG2, Trichoderma viride strain TV1 or strain TV25, Trichoderma atroviride strain CNCM I-1237, and Tsukamurella paurometabola strain C-924.

All plants and plant parts can be treated with the compounds and/or biological control agents and/or mixtures in accordance with the invention. Plants of the invention can be soybean plants containing traits obtained by conventional breeding and optimization methods or by biotechnological methods or combinations of these methods. Plant parts should be understood to mean all parts and organs of the plants above and below ground, such as shoot, leaf, flower and root, examples given being leaves, stems, flowers, pods and seeds, and also roots. Parts of plants also include harvested plants or harvested plant parts and vegetative and generative propagation material, for example seedlings, cuttings or seeds.

Treatment according to the invention of the plants and plant parts of the invention with the compounds and/or biological control agents and/or mixtures in accordance with the invention is carried out directly or by allowing the compounds and/or biological control agents and/or mixtures to act on the surroundings, environment or storage space by the customary treatment methods, for example by immersion, spraying, evaporation, fogging, scattering, painting on, injection and, in the case of propagation material, in particular in the case of seeds, also by applying one or more coats.

The plants comprising the elite event of the invention which can be treated in accordance with the invention include also plants which, through genetic modification or breeding, received genetic material which imparts particular advantageous useful properties (“traits”) to these plants, besides the (soybean or engineered) traits contained in the event of the invention. Examples of such properties are better plant growth, increased tolerance to high or low temperatures, increased tolerance to drought or to levels of water or soil salinity, enhanced flowering performance, easier harvesting, accelerated ripening, higher yields, higher quality and/or a higher nutritional value of the harvested products, better storage life and/or processability of the harvested products. Further and particularly emphasized examples of such properties are increased resistance of the plants against pests, such as animal or microbial pests, such as against insects, arachnids, nematodes, mites, slugs and snails owing, for example, to toxins formed in the plants, in particular those toxins derived from Bacillus thuringiensis (for example the toxins known as Cry1Aa, Cry1Ab, Cry1Ac, Cry2Ab, Cry2Ae, Cry3Aa, Cry9c, Cry3Bb and Cry1Fa and also any mutants thereof such as Cry1A.105, or combinations of such toxins, such as Cry1Ac and Cry1F, or Cry1Ac, Cry1A.105, and Cry2Ab), furthermore increased resistance of the plants against phytopathogenic fungi, bacteria and/or viruses owing, for example, to systemic acquired resistance (SAR), systemin, phytoalexins, elicitors and also resistance genes and correspondingly expressed proteins and toxins, and also increased tolerance of the plants to certain herbicidally active compounds, for example imidazolinones, sulphonylureas, glyphosate, PPO inhibitors, metribuzin, or phosphinothricin (for example the “PAT” gene). The genes which impart the desired traits in question may also be present in combinations with one another in the transgenic plants.

### Crop Protection—Types of Treatment

The treatment of the plants and plant parts with the compounds and/or biological control agents and/or mixtures is carried out directly or by action on their surroundings, habitat (such as the soil or the field in which the plants of the invention were planted or sown or will be planted or sown) or storage space using customary treatment methods, for example by dipping, spraying, atomizing, irrigating, evaporating, dusting, fogging, broadcasting, foaming, painting, spreading-on, injecting, watering (drenching), drip irrigating and, in the case of propagation material, in particular in the case of seed, furthermore as a powder for dry seed treatment, a solution for liquid seed treatment, a water-soluble powder for slurry treatment, by incrusting, by coating with one or more coats, etc.. It is furthermore possible to apply the compounds and/or biological control agents and/or mixtures by the ultra-low volume method or to inject the application form or the compounds and/or biological control agents and/or mixtures itself into the soil (e.g., in furrow or pre-plant application).

One treatment of the plants of the invention is foliar application, i.e. the compounds and/or biological control agents and/or mixtures are applied to the foliage, where treatment frequency and the application rate should be adjusted according to the level of infestation with the pest in question.

In the case of systemically active compounds and/or biological agents, the compounds and/or biological control agents and/or mixtures also access the plants via the root system. The plants are then treated by the action of the compounds and/or biological control agents and/or mixtures on the habitat of the plant. This may be done, for example, by drenching, or by mixing into the soil or the nutrient solution, i.e. the locus of the plant (e.g., soil) is impregnated with a liquid form of the compounds and/or biological control agents and/or mixtures thereof, or by soil application, i.e. the compounds and/or biological control agents and/or mixtures according to the invention are introduced in solid form (e.g., in the form of granules) into the locus of the plants, or by drip application (often also referred to as “chemigation”), i.e. the liquid application of the compounds and/or biological control agents and/or mixtures according to the invention from surface or sub-surface driplines over a certain period of time together with varying amounts of water at defined locations in the vicinity of the plants.

### Seed Treatment

The control of pests by treating the seed of plants has been known for a long time and is the subject of continuous improvements. Methods for the treatment of seed can also take into consideration the intrinsic insecticidal or nematicidal properties of pest-resistant or -tolerant plants in order to achieve optimum protection of the seed and also the germinating plant with a minimum of pesticides being employed.

The present invention therefore in particular also relates to a method for the protection of seed and germinating plants containing the event of the invention, from attack by pests, by treating the seed with one or more of the compound(s) and/or biological control agent(s) and/or mixtures described herein. The method according to the invention for protecting seed and germinating plants against attack by pests furthermore comprises a method where the seed is treated simultaneously in one operation or sequentially with a compounds and/or biological control agents and one or more mixing components. It also comprises a method where the seed is treated at different times with a compound, a biological control agent and a mixing component.

The invention likewise relates to the use of the compounds and/or biological control agents and/or mixtures as described herein for the treatment of seed containing the elite event of the invention for protecting that seed and the resulting plant from pests.

Furthermore, the invention relates to seed containing the elite event of the invention which has been treated with a compound and/or biological control agent and/or mixture or combination according to the invention so as to afford protection from pests. The invention also relates to seed which has been treated simultaneously with a compound and/or biological control agent and a mixing component. The invention furthermore relates to seed which has been treated at different times with a compound and/or biological control agent and a mixing component. In the case of seed which has been treated at different points in time with compounds and/or biological control agents and/or mixtures as described herein, the individual substances may be present on the soybean seed of the invention in different layers. Here, the layers comprising a compound and/or biological control agent and/or mixture may optionally be separated by an intermediate layer. The invention also relates to seed of the invention where a compound and/or biological control agent and/or mixture have been applied as component of a coating or as a further layer or further layers in addition to a coating.

Furthermore, the invention relates to seed which, after the treatment with a compound and/or biological control agent and/or mixture as described herein, is subjected to a film-coating process to prevent dust abrasion on the seed.

One of the advantages encountered with a systemically acting compound is the fact that, by treating the seed, not only the seed itself but also the plants resulting therefrom are, after emergence, protected against pests. In this manner, the immediate treatment of the crop at the time of sowing or shortly thereafter can be dispensed with.

Also, treatment of the seed with a compound and/or biological control agent and/or mixture germination as described herein, and emergence of the treated seed may be enhanced.

Furthermore, compound or biological control agents or mixtures thereof can be employed in combination with compositions or compounds of signalling technology, leading to better colonization by symbionts such as, for example, rhizobia, mycorrhizae such as Rootella@mycorrhiza, and/or endophytic bacteria or fungi, and/or to optimized nitrogen fixation.

In the context of the present invention, the seed is treated in a state in which it is stable enough to avoid damage during treatment. In general, the seed may be treated at any point in time between harvest and sowing. The seed usually used has been separated from the plant and freed from the pods or other plant parts. For example, it is possible to use seed which has been harvested, cleaned and dried down to a moisture content which allows storage. Alternatively, it is also possible to use seed which, after drying, has been treated with, for example, water and then dried again, as in the case of primed seed.

When treating the seed, the amount of the compound or biological agent or mixture described herein applied to the seed and/or the amount of further additives is chosen in such a way that the germination of the seed is not adversely affected, or that the resulting plant is not damaged so that yield is negatively affected.

In general, the compounds or biological agents are applied to the seed in a suitable formulation. Suitable formulations and processes for seed treatment are known to the person skilled in the art.

The compounds and/or biological agents and/or mixtures thereof described herein can be converted to the customary seed dressing formulations, such as solutions, emulsions, suspensions, powders, foams, slurries or other coating compositions for seed, and also ULV formulations.

These formulations are prepared in a known manner, by mixing the compounds and/or biological agents and/or mixtures thereof with customary additives such as, for example, customary extenders and also solvents or diluents, colorants, wetting agents, dispersants, emulsifiers, antifoams, preservatives, secondary thickeners, adhesives, gibberellins and also water.

Colorants which may be present in the seed-dressing formulations which can be used in accordance with the invention are all colorants which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed dressing formulations usable in accordance with the invention are all substances which promote wetting and which are conventionally used for the formulation of agrochemically active compounds. Preference is given to using alkylnaphthalenesulphonates, such as diisopropyl- or diisobutylnaphthalenesulphonates.

Useful dispersants and/or emulsifiers which may be present in the seed dressing formulations usable in accordance with the invention are all nonionic, anionic and cationic dispersants conventionally used for the formulation of active agrochemical ingredients. Preference is given to using nonionic or anionic dispersants or mixtures of nonionic or anionic dispersants. Suitable nonionic dispersants include in particular ethylene oxide/propylene oxide block polymers, alkylphenol polyglycol ethers and tristryrylphenol polyglycol ethers, and the phosphated or sulphated derivatives thereof. Suitable anionic dispersants are in particular lignosulphonates, polyacrylic acid salts and arylsulphonate/formaldehyde condensates.

Antifoams which may be present in the seed dressing formulations usable in accordance with the invention are all foam-inhibiting substances conventionally used for the formulation of active agrochemical ingredients. Preference is given to using silicone antifoams and magnesium stearate.

Preservatives which may be present in the seed dressing formulations usable in accordance with the invention are all substances usable for such purposes in agrochemical compositions. Examples include dichlorophene and benzyl alcohol hemiformal.

Secondary thickeners which may be present in the seed dressing formulations usable in accordance with the invention are all substances which can be used for such purposes in agrochemical compositions. Cellulose derivatives, acrylic acid derivatives, xanthan, modified clays and finely divided silica are preferred.

Adhesives which may be present in the seed dressing formulations usable in accordance with the invention are all customary binders usable in seed dressing products. Polyvinylpyrrolidone, polyvinyl acetate, polyvinyl alcohol and tylose may be mentioned as being preferred.

Gibberellins which can be present in the seed-dressing formulations which can be used in accordance with the invention are preferably the gibberellins A1, A3 (=gibberellic acid), A4 and A7; gibberellic acid is especially preferably used.

For treatment of seed with the seed dressing formulations usable in accordance with the invention, or the use forms prepared therefrom by adding water, all mixing units usable customarily for the seed dressing are useful. Specifically, the procedure in the seed dressing is to place the seed into a mixer, operated batch-wise or continously, to add the particular desired amount of seed dressing formulations, either as such or after prior dilution with water, and to mix everything until the formulation is distributed homogeneously on the seed. If appropriate, this is followed by a drying operation.

The application rate of the seed dressing formulations usable in accordance with the invention can be varied within a relatively wide range. It is guided by the particular content of the compounds and/or biological agents and/or mixtures thereof in the formulations. The application rates of a chemical compound are generally between 0.001 and 50 g per kilogram of seed, preferably between 0.01 and 15 g per kilogram of seed.

Preferred fungicides for seed treatment of seeds containing the event of the invention are selected from the group named SF1 consisting of:

Benzovindiflupyr, Carbendazim, Carboxin, Difenoconazole, Ethaboxam, Fludioxonil, Fluquinconazole, Fluxapyroxad, Ipconazole, Ipfentrifluconazole,Isotianil, Mefenoxam, Mefentrifluconazole, Metalaxyl, Pencycuron, Penflufen, Penthiopyrad, Prothioconazole, Prochloraz, Pyraclostrobin, Sedaxane, Silthiofam, Tebuconazole, Trifloxystrobin, Triticonazole, Ethaboxam (SCC), Penthiopyrad (DPX pipeline), Benzovindiflupyr (SYN pipeline), Bixafen, (see biologicals), Dimethomorph, Fenamidone, Fluopicolide, Fluoxastrobin, Flutolanil, Tolclophos-methyl, Azoxystrobin, Chlorothalonil, Cyproconazole, Cyprodinil, Diniconazole, Fluopyram, Flutriafol, Fluxapyroxad, Imazalil, Isopyrazam, Isotianil, Iprodione, Metconazole, Myclobutanil, Picoxystrobin, Pyrimethanil, Tetraconazole, Thiabendazole, Thiophanate-methyl, Triadimenol, Thiram, Triflumizole, Ziram, 2,6-dimethyl-1H,5H-[1,4]dithiino[2,3-c:5,6-c′]dipyrrole-1,3,5,7(2H,6H)-tetrone, 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate (Thiazolylpiperidin, N-(5-chloro-2-isopropylbenzyl)-N-cyclopropyl-3-(difluoromethyl)-5-fluoro-1-methyl-1H-pyrazole-4-carboxamide Cypropamide, Picarbutrazox Oxathiapiprolin, Picoxystrobin, Isoflucypram, and Pydiflumetofen.

Preferred insecticides/acaricides/nematicides for seed treatment of seeds are selected from the group named SIAN1 consisting of:

Abamectin, Afidopyropen, Bifenthrin, Carbofuran, Carbendazim, Clothianidin, Cyazypyr, Cypermethrin, Deltamethrin, Difenoconazole, Ethoxysulfuron, Fenamidon, Fenoxaprop-P-Ethyl, Ethiprole, Fipronil, Fluazaindolizine, Flupyradifurone (Sivanto™), Flubendiamide, Fluopicolide, Fluopyram, Fluquinconazole, Fosetyl-A1, Imidacloprid, Prochloraz, Propineb, Lambda-Cyhalothrin, Methiocarb, Chlorantraniliprole, Spirotetramat, Spinosad, Tebuconazole, Thiacloprid, Thiametoxam, Thiodicarb, Tioxazafen, (Nemastrike™), Fluazaindolizine (nematicide), Lambda-Cyhalothrin, Bifenthrin, Cypermethrin, Acetamiprid, 13-Cyfluthrin, Flubendiamide, Thiacloprid, Chlorphyriphos, Metamidophos, Phorate, Sulfoxaflor, Tefluthrin, Triflumuron, Tetraniliprole, Triflumuron, Broflanilide (MCI 8007-Mitsui/BASF), Cycloxaprid, Fosthiazate, Fluensulfone and Spinetoram.

Preferred Biologicals and biological active ingredients for seed treatment are selected from the group named SBCA1, consisting of:

Pasteuria nishizawae, such as Pasteuria nishizawae Pn1 (product known as Clariva form Syngenta), a Burkholderia strain, in particular strain A396, Bacillus amyloliquefaciens, such as Bacillus amyloliquefaciens strain PTA-4838 (known as Aveo EZ from Valent), or Bacillus amyloliquefaciens TJ1000, Bacillus firmus, such as Bacillus firmus GB126, Bacillus subtilis, Bacillus pumilus such as Bacillus pumilus QST 2808 or Bacillus pumilus GB34, Rhizobium spp strains, especially Rhizobium tropici SP25, Pseudomonas fluorescens, Pseudomonas chloropsis, Penicilium bilaii, Rhizobium japonicum, Purnate Varroacide, Chenopodioum quinoa saponins, Agrobacterium radiobacter, Bacillus cereus, Bacillus thuringiensis, Beauveria bassiana, Beauveria brongniartii, Burkholderia spp., Chromobacterium subtsugae, Flavobacterium spp., Heterorhabditis bacteriophora, Isaria fumosorosea, Lecanicillium longisporum, Lecanicillium muscarium, Metarhizium anisopliaze, Metarhizium anisopliae var. anisopliae, Metarhizium anisopliae var. acridum, Nomuraea rileyi; Paecilomyces lilacinus, Paenibacillus popilliae, Pasteuria spp., Pasteuria nishizawae, Pasteuria penetrans, Pasteuria ramosa, Pasteuria thornea, Pasteuria usage, Steinernema carpocapsae, Steinernema feltiae, Steinernema kraussei, Streptomyces galbus, Streptomyces microflavus, a recombinant exosporium-producing Bacillus cell, such as a Bacillus species cell, including a Bacillus thuringienses cell (such as B. thuringiensis BT013A) that expresses a fusion protein comprising:

1) an endoglucanase, such as an endoglucanase having at least 70, 80, 90, or 95% sequence identity to SEQ ID NO: 107 of WO 2016/044529, a phosphoplipase, such as a phospholipase having at least 70, 80, 90, or 95% sequence identity to SEQ ID NO. 108 of WO 2016/044529, an acetoin reductase, an indole-3-acetamide hydrolase, a tryptophan monooxygenase, an acetolactate synthetase, an a-acetolactate decarboxylase, a pyruvate decarboxylase, a diacetyl reductase, a butanediol dehydrogenase, an aminotransferase, a tryptophan decarboxylase, an amine oxidase, an indole-3-pyruvate decarboxylase, an indole-3-acetaldehyde dehydrogenase, a tryptophan side chain oxidase, a nitrile hydrolase, a nitrilase, a peptidase, a protease, an adenosine phosphate isopentenyltransferase, a phosphatase, an adenosine kinase, an adenine phosphoribosyltransferase, a CYP735A, a 5′ribonucleotide phosphohydrolase, an adenosine nucleosidase, a zeatin cis-trans isomerase, a zeatin O-glucosyltransferase, a β-glucosidase, a cis-hydroxylase, a CK cis-hydroxylase, a CK N-glucosyltransferase, a 2, 5-ribonucleotide phosphohydrolase, an adenosine nucleosidase, a purine nucleoside phosphorylase, a zeatin reductase, a hydroxylamine reductase, a 2-oxoglutarate dioxygenase, a gibberellic 2B/3B hydrolase, a gibberellin 3-oxidase, a gibberellin 20-oxidase, a chitosinase, a chitinase, a β-1,3-glucanase, a -1,4-glucanase, a β-I,ó-glucanase, an aminocyclopropane-1-carboxylic acid deaminase, or an enzyme involved in producing a nod factor, or an enzyme that degrades or modifies a bacterial, fungal, or plant nutrient source selected from the group consisting of a cellulase, a lipase, a lignin oxidase, a glycoside hydrolase, a phosphatase, a nitrogenase, a nuclease, an amidase, a nitrate reductase, a nitrite reductase, an amylase, an ammonia oxidase, a ligninase, a glucosidase, a phospholipase, a phytase, a pectinase, a glucanase, a sulfatase, a urease, a xylanase, or a siderophore and

2) a targeting sequence that localizes the fusion protein to the exosporium of the Bacillus cell, such as the targeting sequence comprising an amino acid sequence having at least about 43% identity with amino acids 20-35 of SEQ ID NO: 1 of WO 2016/044529, wherein the identity with amino acids 25-35 is at least about 54%; a targeting sequence comprising amino acids 1-35 of SEQ ID NO: 1 of WO 2016/044529; a targeting sequence comprising amino acids 20-35 of SEQ ID NO: 1 of WO 2016/044529; a targeting sequence comprising amino acids 22-31 of SEQ ID NO: 1 of WO 2016/044529; a targeting sequence comprising amino acids 22-33 of SEQ ID NO: 1 of WO 2016/044529; a targeting sequence comprising amino acids 20-31 of SEQ ID NO: 1 of WO 2016/044529; a targeting sequence comprising SEQ ID NO: 1 of WO 2016/044529; or an exosporium protein comprising an amino acid sequence having at least 85% identity with SEQ ID NO: 2 of WO 2016/044529.

In one embodiment, biologicals and biologically active ingredients for seed treatment of the seeds comprising the elite event of the invention are selected from the group named SBCA2 consisting of:

Bacilus firmus, especially Bacillus firmus GB126, Bacillus subtilis, Bacillus pumilus such as Bacillus pumilus QST 2808 or Bacillus pumilus GB34, the above-described recombinant exosporium-producing Bacillus, such as a Bacillus thuringienses cell expressing the above-described fusion protein.

In another more preferred embodiment, preferred active ingredients for seed treatment of seeds comprising the elite event of the invention are selected from the group named SAI1 consisting of:

ß-Cyfluthrin, Bradyrhizobium japonicum, Carbendazim, Carboxin Clothianidin, Cypermethrin, Deltamethrin, Difenoconazole, Ethoxysulfuron, Fenamidon, Fenoxaprop-P-Ethyl, Flubendiamide, Fluopicolide, Fluopyram, Fluoxastrobin, Fluquinconazole, Fosetyl-A1, Fipronil, Prochloraz, Propineb, Prothioconazole, Pseudomonas Fluorescens, Spirotetramat, Tebuconazole, Tembotrione, Thiacloprid, Thiodicarb, Thiram, Triadimenol, Trifloxystrobin, Triflumuron, Penflufen, Imidacloprid, Metalaxyl, Mefonoxam, Fludioxinil, Pryaclostrobin, Azoxystrobin, Sedaxane, Ipconazole, Picoxystrobin, Cyantraniliprole, Chlorantraniliprole, Tetraniliprole, Penthiopyrad, Pasteuria nishizawae, such as Pasteuria nishizawae Pn1, Burkholderia spp., such as strain A396, Bacillus amyloliquefaciens, such as Bacillus amyloliquefaciens strain PTA-4838, Bacillus amyloliquefaciens strain D747 or Bacillus amyloliquefaciens TJ1000, Harpin protein, Thiamethoxam, Flupyradifurone, Bacillus firmus, such as Bacillus firmus GB126, Fluoxapyrad, Ethoboxam, Thiophanate-methyl, Pydiflumetofen, and Thiabendazole.

Particularly preferred combinations of compounds and/or biological agents for seed treatment of seeds comprising the elite event of the invention used in the context of the present invention are selected from the group named SC1 consisting of:

combination of Clothianidin and Bacillus firmus (such as B. firmus GB126), combination of Clothianidin, Bacillus thuringiensis (such as B. thuringiensis strain EX297512) and Bacillus firmus (such as B. firmus GB126), combination of Imidacloprid and Thiodicarb, combination of Imidacloprid and Prothioconazole, combination of Clothianidin and Carboxin and Metalaxyl and Trifloxystrobin, combination of Metalaxyl and Prothioconazole and Tebuconazole, combination of Clothianidin and beta-Cyfluthrin, combination of Prothioconazole and Tebuconazole, combination of Clothianidin and Imidacloprid and Prothioconazole and Tebuconazole, combination of Carbendazim and Thiram, combination of Imidacloprid and Methiocarb, combination of Metalaxyl, Penflufen and Prothioconazole, combination of Metalaxyl, Penflufen, Prothioconazole and imidacloprid, combination of Metalaxyl, Penflufen, Prothioconazole and Clothianidin, combination of Metalaxyl, Penflufen, Prothioconazole, Clothianidin and Bacillus firmus (such as B. firmus GB126), combination of Metalaxyl, Penflufen, Prothioconazole, Clothianidin, Bacillus firmus (such as B. firmus GB126) and Bacillus thuringiensis (such as B. thuringiensis strain EX297512), combination of Metalaxyl, Penflufen, Prothioconazole, Clothianidin, Bacillus firmus (such as B. firmus GB126) and fluopyram, combination of Metalaxyl, Penflufen, Prothioconazole, Clothianidin, Bacillus firmus (such as B. firmus GB126), fluopyram and Bacillus thuringiensis (such as B. thuringiensis strain EX297512), combination of Metalaxyl, Penflufen, Prothioconazole, imidacloprid and fluopyram, combination of Metalaxyl, Penflufen, Prothioconazole, imidacloprid, fluopyram and Bacillus firmus (such as B. firmus GB126), combination of Metalaxyl, Penflufen, Prothioconazole and Clothianidin, combination of Metalaxyl, Penflufen, Prothioconazole, Clothianidin and Bacillus firmus (such as B. firmus GB126), combination of Metalaxyl, Penflufen, Prothioconazole, Clothianidin, Bacillus firmus (such as B. firmus GB126) and fluopyram, combination of Imidacloprid and Prothioconazole, combination of Imidacloprid and Tefluthrin, combination of Imidacloprid and Pencycuron, combination of Imidacloprid and Penflufen, combination of Fluoxastrobin, Prothioconazole and Tebuconazole, combination of Fluoxastrobin, Prothioconazole and metalaxyl, combination of Fluoxastrobin, Prothioconazole, metalaxyl and imdiacloprid, combination of Fluoxastrobin, Prothioconazole, metalaxyl, imdiacloprid and fluopyram, combination of Fluoxastrobin, Prothioconazole, metalaxyl, imdiacloprid, fluopyram and Bacillus firmus (such as B. firmus GB126), combination of Fluoxastrobin, Prothioconazole, metalaxyl, imdiacloprid, fluopyram, Bacillus firmus (such as B. firmus GB126), and Bacillus thuringiensis (such as B. thuringiensis strain EX297512, combination of Fluoxastrobin, Prothioconazole, metalaxyl, clothianidin and fluopyram, combination of Fluoxastrobin, Prothioconazole, metalaxyl, clothianidin and Bacillus firmus (such as B. firmus GB126), combination of Fluoxastrobin, Prothioconazole, metalaxyl, clothianidin and Bacillus firmus (such as B. firmus GB126), and Bacillus thuringiensis (such as B. thuringiensis strain EX297512), combination of Fluoxastrobin, Prothioconazole, metalaxyl, clothianidin and Bacillus firmus (such as B. firmus GB126), combination of Fluoxastrobin, Prothioconazole, metalaxyl, clothianidin, Bacillus firmus (such as B. firmus GB126), and Bacillus thuringiensis (such as B. thuringiensis strain EX297512), combination of Metalaxyl and Trifloxystrobin, combination of Penflufen and Trifloxystrobin, combination of Prothioconazole and Tebuconazole, combination of Fluoxastrobin and Prothioconazole and Tebuconazole and Triazoxide, combination of Imidacloprid and Methiocarb and Thiram, combination of Clothianidin and beta-Cyfluthrin, combination of Clothianidin and Fluoxastrobin and Prothioconazole and Tebuconazole, combination of Fluopyram and Fluoxastrobin and Triadimenol, combination of Metalaxyl and Trifloxystrobin, combination of Imidacloprid and Ipconacole, combination of Difenoconazol and Fludioxonil and Tebuconazole, combination of Imidacloprid and Tebuconazole, combination of Imidacloprid, Prothioconazole and Tebuconazole, combination of Metalaxyl, Prothioconazole and Tebuconazole, combination of fluopyram and Bacillus firmus (such as B. firmus GB126), combination of fluopyram, Bacillus firmus (such as B. firmus GB126), and Bacillus thuringiensis (such as B. thuringiensis strain EX297512), Combination of Pasteria nishazawae (such as P. nishizawae Pn1), thiamethoxam, sedexane, fludioxinil and mefonaxam, Combination of thiamethoxam, sedexane, fludioninil and mefonaxam, Combination of thiamethoxam, fludioxinil and mefonaxam, Combination of fludioxinil and mefonaxam, Combination of pyraclostrobin and fluoxayprad, Combination of abamectin and thiamethoxam, Combination of Burkholderia spp. strain (such as strain A396) and imidacloprid, Combination of Bacillus amyloliquefaciens (such as B. amyloliquefaciens strain PTA-4838) and clothianidin, Combination of tioxazafen, imidacloprid, prothioconazole, fluoxastrobin and metalaxyl, Combination of tioxazafen, clothianidin, prothioconazole, fluoxastroin and metalaxyl, Combination of tioxazafen, clothianidin, Bacillus firmus (such as B. firmus GB126), prothioconazole, fluoxastrobin and metalaxyl, Combination of tioxazafen, clothianidin, Bacillus firmus (such as B. firmus GB126), Bacillus thuringiensis (such as B. thuringiensis strain EX297512), prothioconazole, fluoxastrobin and metalaxyl, combination of tioxazafen, prothioconazole, fluoxastrobin and metalaxyl, combination of tioxazafen, pyraclostrobin, fluoxyprad, metalaxyl and imidacloprid, combination of clothianidin, fluopyram and Bacillus firmus (such as B. firmus GB126), combination of clothianidin, fluopyram, tioxazafen and Bacillus firmus (such as B. firmus GB126); combination of clothianidin, fluopyram, Bacillus firmus (such as B. firmus GB126) and Bacillus thuringiensis (such as B. thuringiensis strain EX297512), combination of clothianidin, fluopyram, tioxazafen, Bacillus firmus (such as B. firmus GB126) and Bacillus thuringiensis (such as B. thuringiensis strain EX297512).

In one embodiment of the invention, seeds comprising EE-GM4 of the invention are treated with a combination of prothioconazole, penfluten and metalaxyl, or with a combination of prothioconazole, penfluten, metalaxyl and clothianidine, wherein said seeds or plants also contain one or more soybean SCN resistance genes from PI 548402, PI 209332 or PI 437654, or one or more of the soybean SCN resistance loci or genes selected from the group consisting of: rhg1, rhg1-b, rhg2, rhg3, Rhg4, Rhg5, qSCN11, cqSCN-003, cqSCN-005, cqSCN-006, and cqSCN-007.

In one embodiment of the invention, a combination of active ingredients for seed treatment is selected from the group named SC2 consisting of:

combination of Clothianidin and Bacillus firmus (such as B. firmus GB126), combination of Imidacloprid and Thiodicarb, combination of Imidacloprid and Prothioconazole, combination of Clothianidin Carboxin, Metalaxyl, Trifloxystrobin, combination of Metalaxyl, Prothioconazole and Tebuconazole, combination of Clothianidin and beta-Cyfluthrin, combination of Prothioconazole and Tebuconazole, combination of fluopyram, Bacillus firmus (such as Bacillus firmus GB126), combination of Pasteria nishazawe (such as P. nishizawae Pn1), thiamethoxam, sedexane, fludioxinil and mefonaxam, combination of abamectin and thiamethoxam, combination of penflufen, prothioconazole and metalaxyl, combination of penflufen and trifloxystrobin.

Preferred agents for use in seed treatment in accordance with this invention, are one or more of the nematicidal agents of group NC1, NC2 or N3, or one or more of the biological control agents of group BCA8, BCA9 or BCA10, or a combination of one of more of such nematicidal agents and biological control agents.

### Formulations

The present invention further relates to formulations and use forms for the above-mentioned compounds and/or biological control agents and/or mixtures, for example drench, drip and spray liquors, for application to the plants or seeds of the invention, or for application to the soil wherein the plants or seeds of the invention were planted, or for application to the soil wherein the plants or seeds of the invention are to be planted (followed by planting of the plants or sowing of the seeds of the invention). In some cases, the use forms comprise further pesticides and/or adjuvants which improve action, such as penetrants, e.g. vegetable oils, for example rapeseed oil, sunflower oil, mineral oils, for example paraffin oils, alkyl esters of vegetable fatty acids, for example rapeseed oil methyl ester or soya oil methyl ester, or alkanol alkoxylates and/or spreaders, for example alkylsiloxanes and/or salts, for example organic or inorganic ammonium or phosphonium salts, for example ammonium sulphate or diammonium hydrogenphosphate and/or retention promoters, for example dioctyl sulphosuccinate or hydroxypropyl guar polymers and/or humectants, for example glycerol and/or fertilizers, for example ammonium-, potassium- or phosphorus-containing fertilizers.

Customary formulations are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations, in addition to one or more of the above compounds, optionally comprise further agrochemically active compounds.

These are preferably formulations or use forms which comprise auxiliaries, for example extenders, solvents, spontaneity promoters, carriers, emulsifiers, dispersants, frost protectants, biocides, thickeners and/or further auxiliaries, for example adjuvants. An adjuvant in this context is a component which enhances the biological effect of the formulation, without the component itself having any biological effect. Examples of adjuvants are agents which promote retention, spreading, attachment to the leaf surface or penetration.

These formulations are prepared in a known way, for example by mixing the compounds with auxiliaries such as, for example, extenders, solvents and/or solid carriers and/or other auxiliaries such as, for example, surfactants. The formulations are prepared either in suitable facilities or else before or during application.

The auxiliaries used may be substances suitable for imparting special properties, such as certain physical, technical and/or biological properties, to the formulation of the compounds, or to the use forms prepared from these formulations (for example ready-to-use pesticides such as spray liquors or seed dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), the esters (including fats and oils) and (poly)ethers, the unsubstituted and substituted amines, amides, lactams (such as N-alkylpyrrolidones) and lactones, the sulphones and sulphoxides (such as dimethyl sulphoxide), the carbonates and the nitriles.

If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, carbonates such as propylene carbonate, butylene carbonate, diethyl carbonate or dibutyl carbonate, or nitriles such as acetonitrile or propanenitrile.

In principle, it is possible to use all suitable solvents. Examples of suitable solvents are aromatic hydrocarbons, such as xylene, toluene or alkylnaphthalenes, chlorinated aromatic or chlorinated aliphatic hydrocarbons, such as chlorobenzene, chloroethylene or methylene chloride, aliphatic hydrocarbons, such as cyclohexane, paraffins, petroleum fractions, mineral and vegetable oils, alcohols, such as methanol, ethanol, isopropanol, butanol or glycol and their ethers and esters, ketones such as acetone, methyl ethyl ketone, methyl isobutyl ketone or cyclohexanone, strongly polar solvents, such as dimethyl sulphoxide, carbonates such as propylene carbonate, butylene carbonate, diethyl carbonate or dibutyl carbonate, nitriles such as acetonitrile or propanenitrile, and also water.

In principle, it is possible to use all suitable carriers. Useful carriers include especially: for example ammonium salts and ground natural minerals such as kaolins, clays, talc, chalk, quartz, attapulgite, montmorillonite or diatomaceous earth, and ground synthetic materials such as finely divided silica, alumina and natural or synthetic silicates, resins, waxes and/or solid fertilizers. Mixtures of such carriers can likewise be used. Useful carriers for granules include: for example crushed and fractionated natural rocks such as calcite, marble, pumice, sepiolite, dolomite, and synthetic granules of inorganic and organic meals, and also granules of organic material such as sawdust, paper, coconut shells, corn cobs and tobacco stalks.

Liquefied gaseous extenders or solvents can also be used. Particularly suitable extenders or carriers are those which are gaseous at ambient temperature and under atmospheric pressure, for example aerosol propellant gases, such as halohydrocarbons, and also butane, propane, nitrogen and carbon dioxide.

Examples of emulsifiers and/or foam-formers, dispersants or wetting agents with ionic or nonionic properties, or mixtures of these surfactants, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyl taurates), isethionate derivatives, phosphoric esters of polyethoxylated alcohols or phenols, fatty esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is advantageous if one of the pesticides and/or one of the inert carriers is insoluble in water and when the application takes place in water.

It is possible to use colorants such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc as further auxiliaries in the formulations and the use forms derived therefrom.

Additional components may be stabilizers, such as low-temperature stabilizers, preservatives, antioxidants, light stabilizers or other agents which improve chemical and/or physical stability. Foam formers or antifoams may also be present.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids may also be present as additional auxiliaries in the formulations and the use forms derived therefrom. Further possible auxiliaries are mineral and vegetable oils.

Optionally, further auxiliaries may be present in the formulations and the use forms derived therefrom. Examples of such additives include fragrances, protective colloids, binders, adhesives, thickeners, thixotropic agents, penetrants, retention promoters, stabilizers, sequestrants, complexing agents, humectants, spreaders. In general, the compounds can be combined with any solid or liquid additive commonly used for formulation purposes.

Useful retention promoters include all those substances which reduce the dynamic surface tension, for example dioctyl sulphosuccinate, or increase the viscoelasticity, for example hydroxypropylguar polymers.

Suitable penetrants in the present context are all those substances which are usually used for improving the penetration of agrochemical active compounds into plants. Penetrants are defined in this context by their ability to penetrate from the (generally aqueous) application liquor and/or from the spray coating into the cuticle of the plant and thereby increase the mobility of active compounds in the cuticle. The method described in the literature (Baur et al., 1997, Pesticide Science 51, 131-152) can be used to determine this property. Examples include alcohol alkoxylates such as coconut fatty ethoxylate (10) or isotridecyl ethoxylate (12), fatty acid esters, for example rapeseed oil methyl ester or soya oil methyl ester, fatty amine alkoxylates, for example tallowamine ethoxylate (15), or ammonium and/or phosphonium salts, for example ammonium sulphate or diammonium hydrogenphosphate.

The formulations preferably comprise between 0.00000001 and 98% by weight of the compound or, with particular preference, between 0.01% and 95% by weight of the compound, more preferably between 0.5% and 90% by weight of the compound, based on the weight of the formulation.

The content of the compound in the use forms prepared from the formulations (in particular pesticides) may vary within wide ranges. The concentration of the compound in the use forms is usually between 0.00000001 and 95% by weight of the compound, preferably between 0.00001 and 1% by weight, based on the weight of the use form. The compounds are employed in a customary manner appropriate for the use forms.

### Mixtures

The compounds mentioned herein may also be employed as a mixture with one or more suitable fungicides, bactericides, acaricides, molluscicides, nematicides, insecticides, microbiologicals, beneficial species, herbicides, fertilizers, bird repellents, phytotonics, sterilants, safeners, semiochemicals and/or plant growth regulators, e.g., to broaden the spectrum of action, to prolong the duration of action, to increase the rate of action, to prevent repulsion or prevent evolution of resistance. In addition, such active compound combinations may improve plant growth and/or tolerance to abiotic factors, for example high or low temperatures, to drought or to elevated water content or soil salinity. It is also possible to improve flowering and fruiting performance, optimize germination capacity and root development, facilitate harvesting and improve yields, influence maturation, improve the quality and/or the nutritional value of the harvested products, prolong storage life and/or improve the processability of the harvested products.

Furthermore, the compounds can be present in a mixture with other active compounds or semiochemicals such as attractants and/or bird repellants and/or plant activators and/or growth regulators and/or fertilizers. Likewise, the compounds can be used to improve plant properties such as, for example, growth, yield and quality of the harvested material.

In a particular embodiment according to the invention, the compounds are present in formulations or the use forms prepared from these formulations in a mixture with further compounds, preferably those as described herein.

If one of the compounds mentioned herein can occur in different tautomeric forms, these forms are also included even if not explicitly mentioned in each case. Further, all named mixing partners can, if their functional groups enable this, optionally form salts with suitable bases or acids.

In one embodiment at least one active ingredient selected from group H1 is used on a plant comprising EE-GM4 or plant parts thereof (such as a seed), preferably for increasing yield.

In one embodiment at least one active ingredient selected from group H2 is used on a plant comprising EE-GM4 or plant parts thereof (such as a seed), preferably for increasing yield.

In one embodiment at least one active ingredient selected from group H3 is used on a plant comprising EE-GM4 or plant parts thereof (such as a seed), preferably for increasing yield.

In one embodiment at least one active ingredient mixture selected from group H4 is used on a plant comprising EE-GM4 or plant parts thereof (such as a seed), preferably for increasing yield. In one embodiment at least one active ingredient mixture selected from group H5 is used on a plant comprising EE-GM4 or plant parts thereof (such as a seed), preferably for increasing yield.

In one embodiment at least one active ingredient selected from group IAN1 is used on a plant comprising EE-GM4 or plant parts thereof (such as a seed), preferably for increasing yield.

In one embodiment at least one active ingredient selected from group IAN2 is used on a plant comprising EE-GM4 or plant parts thereof (such as a seed), preferably for increasing yield.

In one embodiment at least one active ingredient selected from group IAN3 is used on a plant comprising EE-GM4 or plant parts thereof (such as a seed), preferably for increasing yield.

In one embodiment at least one active ingredient selected from group IAN4 is used on a plant comprising EE-GM4 or plant parts thereof (such as a seed), preferably for increasing yield.

In one embodiment at least one active ingredient selected from group IAN5 is used on a plant comprising EE-GM4 or plant parts thereof (such as a seed), preferably for increasing yield.

In one embodiment at least one active ingredient selected from group IAN6 is used on a plant comprising EE-GM4 or plant parts thereof (such as a seed), preferably for increasing yield.

In one embodiment at least one active ingredient selected from group IAN7 is used on a plant comprising EE-GM4 or plant parts thereof (such as a seed), preferably for increasing yield.

In one embodiment at least one active ingredient selected from group IAN8 is used on a plant comprising EE-GM4 or plant parts thereof (such as a seed), preferably for increasing yield.

In one embodiment at least one active ingredient selected from group IAN9 is used on a plant comprising EE-GM4 or plant parts thereof (such as a seed), preferably for increasing yield.

In one embodiment at least one active ingredient selected from group IAN9 is used on a plant comprising EE-GM4 or plant parts thereof (such as a seed), preferably for increasing yield.

In one embodiment at least one active ingredient selected from group IAN10 is used on a plant comprising EE-GM4 or plant parts thereof (such as a seed), preferably for increasing yield.

In one embodiment at least one active ingredient selected from group IAN11 is used on a plant comprising EE-GM4 or plant parts thereof (such as a seed), preferably for increasing yield.

In one embodiment at least one active ingredient selected from group IAN12 is used on a plant comprising EE-GM4 or plant parts thereof (such as a seed), preferably for increasing yield.

In one embodiment at least one active ingredient selected from group IAN13 is used on a plant comprising EE-GM4 or plant parts thereof (such as a seed), preferably for increasing yield.

In one embodiment at least one active ingredient selected from group IAN14 is used on a plant comprising EE-GM4 or plant parts thereof (such as a seed), preferably for increasing yield.

In one embodiment at least one active ingredient selected from group IAN15 is used on a plant comprising EE-GM4 or plant parts thereof (such as a seed), preferably for increasing yield.

In one embodiment at least one active ingredient selected from group IAN16 is used on a plant comprising EE-GM4 or plant parts thereof (such as a seed), preferably for increasing yield.

In one embodiment at least one active ingredient selected from group IAN17 is used on a plant comprising EE-GM4 or plant parts thereof (such as a seed), preferably for increasing yield.

In one embodiment at least one active ingredient selected from group IAN18 is used on a plant comprising EE-GM4 or plant parts thereof (such as a seed), preferably for increasing yield.

In one embodiment at least one active ingredient selected from group IAN19 is used on a plant comprising EE-GM4 or plant parts thereof (such as a seed), preferably for increasing yield.

In one embodiment at least one active ingredient selected from group IAN20 is used on a plant comprising EE-GM4 or plant parts thereof (such as a seed), preferably for increasing yield.

In one embodiment at least one active ingredient selected from group IAN21 is used on a plant comprising EE-GM4 or plant parts thereof (such as a seed), preferably for increasing yield.

In one embodiment at least one active ingredient selected from group IAN22 is used on a plant comprising EE-GM4 or plant parts thereof (such as a seed), preferably for increasing yield.

In one embodiment at least one active ingredient selected from group IAN23 is used on a plant comprising EE-GM4 or plant parts thereof (such as a seed), preferably for increasing yield.

In one embodiment at least one active ingredient selected from group IAN24 is used on a plant comprising EE-GM4 or plant parts thereof (such as a seed), preferably for increasing yield.

In one embodiment at least one active ingredient selected from group IAN25 is used on a plant comprising EE-GM4 or plant parts thereof (such as a seed), preferably for increasing yield.

In one embodiment at least one active ingredient selected from group IAN26 is used on a plant comprising EE-GM4 or plant parts thereof (such as a seed), preferably for increasing yield.

In one embodiment at least one active ingredient selected from group IAN27 is used on a plant comprising EE-GM4 or plant parts thereof (such as a seed), preferably for increasing yield.

In one embodiment at least one active ingredient selected from group IAN28 is used on a plant comprising EE-GM4 or plant parts thereof (such as a seed), preferably for increasing yield.

In one embodiment at least one active ingredient selected from group IAN29 is used on a plant comprising EE-GM4 or plant parts thereof (such as a seed), preferably for increasing yield.

In one embodiment at least one active ingredient selected from group IAN30 is used on a plant comprising EE-GM4 or plant parts thereof (such as a seed), preferably for increasing yield.

In one embodiment at least one active ingredient selected from group SIAN1 is used on a plant comprising EE-GM4 or plant parts thereof (such as a seed), preferably for increasing yield.

In one embodiment at least one active ingredient selected from group F1 is used on a plant comprising EE-GM4 or plant parts thereof (such as a seed), preferably for increasing yield.

In one embodiment at least one active ingredient selected from group F2 is used on a plant comprising EE-GM4 or plant parts thereof (such as a seed), preferably for increasing yield.

In one embodiment at least one active ingredient selected from group F3 is used on a plant comprising EE-GM4 or plant parts thereof (such as a seed), preferably for increasing yield.

In one embodiment at least one active ingredient selected from group F4 is used on a plant comprising EE-GM4 or plant parts thereof (such as a seed), preferably for increasing yield.

In one embodiment at least one active ingredient selected from group F5 is used on a plant comprising EE-GM4 or plant parts thereof (such as a seed), preferably for increasing yield.

In one embodiment at least one active ingredient selected from group F6 is used on a plant comprising EE-GM4 or plant parts thereof (such as a seed), preferably for increasing yield.

In one embodiment at least one active ingredient selected from group F7 is used on a plant comprising EE-GM4 or plant parts thereof (such as a seed), preferably for increasing yield.

In one embodiment at least one active ingredient selected from group F8 is used on a plant comprising EE-GM4 or plant parts thereof (such as a seed), preferably for increasing yield.

In one embodiment at least one active ingredient selected from group F9 is used on a plant comprising EE-GM4 or plant parts thereof (such as a seed), preferably for increasing yield.

In one embodiment at least one active ingredient selected from group F10 is used on a plant comprising EE-GM4 or plant parts thereof (such as a seed), preferably for increasing yield.

In one embodiment at least one active ingredient selected from group F11 is used on a plant comprising EE-GM4 or plant parts thereof (such as a seed), preferably for increasing yield.

In one embodiment at least one active ingredient selected from group F12 is used on a plant comprising EE-GM4 or plant parts thereof (such as a seed), preferably for increasing yield.

In one embodiment at least one active ingredient selected from group F13 is used on a plant comprising EE-GM4 or plant parts thereof (such as a seed), preferably for increasing yield.

In one embodiment at least one active ingredient selected from group F14 is used on a plant comprising EE-GM4 or plant parts thereof (such as a seed), preferably for increasing yield.

In one embodiment at least one active ingredient selected from group F15 is used on a plant comprising EE-GM4 or plant parts thereof (such as a seed), preferably for increasing yield.

In one embodiment at least one active ingredient selected from group SF1 is used on a plant comprising EE-GM4 or plant parts thereof (such as a seed), preferably for increasing yield.

In one embodiment at least one active ingredient mixture selected from group F16 is used on a plant comprising EE-GM4 or plant parts thereof (such as a seed), preferably for increasing yield.

In one embodiment at least one active ingredient selected from group P1 is used on a plant comprising EE-GM4 or plant parts thereof (such as a seed), preferably for increasing yield.

In one embodiment at least one active ingredient selected from group BCA1 is used on a plant comprising EE-GM4 or plant parts thereof (such as a seed), preferably for increasing yield.

In one embodiment at least one active ingredient selected from group BCA2 is used on a plant comprising EE-GM4 or plant parts thereof (such as a seed), preferably for increasing yield.

In one embodiment at least one active ingredient selected from group BCA3 is used on a plant comprising EE-GM4 or plant parts thereof (such as a seed), preferably for increasing yield.

In one embodiment at least one active ingredient selected from group BCA4 is used on a plant comprising EE-GM4 or plant parts thereof (such as a seed), preferably for increasing yield.

In one embodiment at least one active ingredient selected from group BCA5 is used on a plant comprising EE-GM4 or plant parts thereof (such as a seed), preferably for increasing yield.

In one embodiment at least one active ingredient selected from group BCA6 is used on a plant comprising EE-GM4 or plant parts thereof (such as a seed), preferably for increasing yield.

In one embodiment at least one active ingredient selected from group BCA7 is used on a plant comprising EE-GM4 or plant parts thereof (such as a seed), preferably for increasing yield.

In one embodiment at least one active ingredient selected from group BCA8 is used on a plant comprising EE-GM4 or plant parts thereof (such as a seed), preferably for increasing yield.

In one embodiment at least one active ingredient selected from group BCA9 is used on a plant comprising EE-GM4 or plant parts thereof (such as a seed), preferably for increasing yield.

In one embodiment at least one active ingredient selected from group BCA10 is used on a plant comprising EE-GM4 or plant parts thereof (such as a seed), preferably for increasing yield.

In one embodiment at least one active ingredient selected from group SBCA1 is used on a plant comprising EE-GM4 or plant parts thereof (such as a seed), preferably for increasing yield.

In one embodiment at least one active ingredient combination selected from group SAI1 is used on a plant comprising EE-GM4 or plant parts thereof (such as a seed), preferably for increasing yield.

In one embodiment at least one active ingredient combination selected from group SC1 is used on a plant comprising EE-GM4 or plant parts thereof (such as a seed), preferably for increasing yield.

In one embodiment at least one active ingredient combination selected from group SC2 is used on a plant comprising EE-GM4 or plant parts thereof (such as a seed), preferably for increasing yield.

In one embodiment at least one active ingredient selected from group NC is used on a plant comprising EE-GM4 or plant parts thereof (such as a seed), preferably for increasing yield.

In one embodiment at least one active ingredient selected from group NC2 is used on a plant comprising EE-GM4 or plant parts thereof (such as a seed), preferably for increasing yield.

In one embodiment at least one active ingredient combination selected from group NC3 is used on a plant comprising EE-GM4 or plant parts thereof (such as a seed), preferably for increasing yield.

In the context of the present invention, “increasing yield” means a significant increase in yield by compared with the untreated plant, preferably a significant increase by at least 1% such as by 1% to 3%, compared with the untreated plant (100% yield), i.e. the yield of the treated plants is at least 101% compared to the yield (100%) of the untreated plant; more preferably, the yield is even more increased by at least 2%, more preferably at least 5%, even more preferably by at least 10% such as by 2% to 5% (yield from 102% to 105%), by 2 to 10% (yield from 102% to 110%), by 5% to 20% (yield of from 105% to 120%), or by 10 to 30% (yield from 110% to 130%). The yield increase may be achieved by curative treatment, i.e. for treatment of already infected plants, or by protective treatment, for protection of plants which have not yet been infected.

In the context of the present invention, “to treat with” means to contact a plant or part of a plant with an effective amount of an active ingredient or a combination thereof or to coat a seed with an active ingredient or a combination thereof.

“Active ingredient” refers to compounds or biological control agents used in agriculture. Active ingredients according to the invention are not applied to humans or animals as a medical or therapeutic treatment.

In the context of the present invention the active ingredients according to the invention or to be used according to the invention may be a composition (i. e. a physical mixture) comprising at least one active ingredient. It may also be a combination of active ingredients composed from separate formulations of a single active ingredient component being active ingredient (tank-mix). Another example of a combination of active ingredients according to the invention is that the active ingredients are not present together in the same formulation, but packaged separately (combipack), i.e., not jointly preformulated. As such, combipacks include one or more separate containers such as vials, cans, bottles, pouches, bags or canisters, each container containing a separate component for an agrochemical composition, here at least one active ingredient. One example is a two-component combipack. Accordingly the present invention also relates to a two-component combipack, comprising a first component which in turn comprises an active ingredient, a liquid or solid carrier and, if appropriate, at least one surfactant and/or at least one customary auxiliary, and a second component which in turn comprises another active ingredient, a liquid or solid carrier and, if appropriate, at least one surfactant and/or at least one customary auxiliary. More details, e.g. as to suitable liquid and solid carriers, surfactants and customary auxiliaries are described below. A mixture or combination according to the invention shall mean/encompass a tank mix or a combipack.

In one embodiment, besides protection against nematodes, the compound(s) and/or biological control agent(s) as described herein are used to also protect soybean plants, parts or seeds from the following pests, bacterial diseases or fungi: hairy caterpillar, Spilarctia obliqua (Walker); leaf roller, Lamprosema indicata F; common cutworm, Spodoptera litura F; pod borer, Armyworms, especially Spodoptera exigua and S. praefica, Helicoverpa armigera (Hubner); stem fly, Ophiomyiaphaseoli (Tryon) and white fly; Bemisia tabaci Genn., Pseudomonas syringae, Xanthomonas campestris, Septoria glycines, Macrophomina phaseolina, Cucumber beetles, especially Acalymma vittata or Diabrotica undecimpunctata, Downy mildew (Peronospora manshurica), Frogeye leaf spot (Cercospora sojina), Mexican Bean beetle (Epilachna varivestis), Phytophthora megasperma, Rhizoctonia solani, Rust (Phakopsora pachyrhizi), Sclerotinia sclerotiorum.

The following examples describe the development and identification of elite event EE-GM4, the development of different soybean lines comprising this event, and the development of tools for the specific identification of elite event EE-GM4 in biological samples.

Unless stated otherwise in the Examples, all recombinant techniques are carried out according to standard protocols as described in “Sambrook J and Russell D W (eds.) (2001) Molecular Cloning: A Laboratory Manual, 3rd Edition, Cold Spring Harbor Laboratory Press, New York” and in “Ausubel F A, Brent R, Kingston R E, Moore D D, Seidman J G, Smith J A and Struhl K (eds.) (2006) Current Protocols in Molecular Biology. John Wiley & Sons, New York”.

Standard materials and references are described in “Croy RDD (ed.) (1993) Plant Molecular Biology LabFax, BIOS Scientific Publishers Ltd., Oxford and Blackwell Scientific Publications, Oxford” and in “Brown TA, (1998) Molecular Biology LabFax, 2nd Edition, Academic Press, San Diego”. Standard materials and methods for polymerase chain reactions (PCR) can be found in “McPherson M J and Møller S G (2000) PCR (The Basics), BIOS Scientific Publishers Ltd., Oxford” and in “PCR Applications Manual, 3rd Edition (2006), Roche Diagnostics GmbH, Mannheim or on the world wide web at roche-applied-science.com”.

It should be understood that a number of parameters in any lab protocol such as the PCR protocols in the below Examples may need to be adjusted to specific laboratory conditions, and may be modified slightly to obtain similar results. For instance, use of a different method for preparation of DNA or the selection of other primers in a PCR method may dictate other optimal conditions for the PCR protocol. These adjustments will however be apparent to a person skilled in the art, and are furthermore detailed in current PCR application manuals.

In the description and examples, reference is made to the following sequences in the enclosed Sequence Listing:

SEQ ID No. 1: 5′ junction EE-GM4

SEQ ID No. 2: 3′ junction EE-GM4

SEQ ID No. 3: EE-GM4 5′ junction

SEQ ID No. 4: EE-GM4 3′ junction

SEQ ID No. 5: EE-GM4 5′ region

SEQ ID No. 6: EE-GM4 3′ region

SEQ ID No. 7: cry14Ab-1.b coding sequence

SEQ ID No. 8: Cry14Ab-1 protein amino acid sequence

SEQ ID No. 9: hppdPf-4 Pa coding sequence

SEQ ID No. 10: HPPD-4 protein amino acid sequence

SEQ ID No. 11: transforming plasmid pSZ8832-sequence between T-DNA borders

SEQ ID No. 12: primer PRIMO937

SEQ ID No. 13: primer PRIMO938

SEQ ID No. 14: probe TM1734

SEQ ID No. 15: primer SHA071

SEQ ID No. 16: primer SHA072

SEQ ID No. 17: probe TM1428

SEQ ID No. 18: primer PRIM1652

SEQ ID No. 19: probe TM2084

SEQ ID No. 20: primer PRIM0939

SEQ ID No. 21: primer PRIM0940

SEQ ID No. 22: probe TM1735

SEQ ID No. 23: soybean event EE-GM4

SEQ ID No. 24: EE-GM4 5′ junction sequence

SEQ ID No. 25: EE-GM4 3′ junction sequence

SEQ ID No. 26: primer GLPB173

SEQ ID No. 27: primer GLPB175

SEQ ID No. 28: primer GLPB167

SEQ ID No. 29: primer GLPB170

SEQ ID No. 30: pre-insertion locus sequence

### EXAMPLES

1. Transformation of Glycine max with a Nematode Resistance and an Herbicide Tolerance Gene
1.1. Description of the Inserted T-DNA Comprising the cry14Ab-1.b and hppdPf-4 Pa Chimeric Genes

EE-GM4 soybean was developed through Agrobacterium-mediated transformation using the vector pSZ8832 containing hppdPf-4 Pa and cry4Ab-1.b expression cassettes:

(i) The mutant hppdPf-4 Pa gene that encodes for the HPPD-4 protein. The hppdPf-4 Pa coding sequence was developed by introducing point mutations at position 335 (substitution of Glu by Pro), at position 336 (substitution of Gly by Trp), at position 339 (substitution of Lys by Ala) and at position 340 (substitution of Ala by Gln) in a DNA encoding the HPPD protein derived from Pseudomonas fluorescens strain A32. Expression of the HPPD-4 protein confers tolerance to HPPD inhibitor herbicides, such as isoxaflutole or mesotrione.

(ii) The cry14Ab-1.b gene encodes for the Cry14Ab-1 protein. Expression of the Cry14Ab-1 protein confers resistance to nematodes such as the soybean cyst nematode Heterodera glycines.

Plasmid pSZ8832 is a plant transformation vector which contains a chimeric cry4Ab-1.b gene and a chimeric hppdPf-4 Pa gene located between the right T-DNA border (RB) and the left T-DNA border (LB). A description of the genetic elements comprised in the T-DNA between the right and the left T-DNA border is given in Table 2 below. Confirmatory sequencing of the T-DNA (between the T-DNA borders) of this plasmid resulted in the sequence of SEQ ID No. 11. The nucleotide sequence of the cry4Ab-1.b and hppdPf-4 Pa coding sequences (showing the coding strand) is represented in SEQ ID No. 7 and 9, respectively.

TABLE 2
Description of the genetic elements between the T-DNA borders in pSZ8832, and
nucleotide positions in SEQ ID No. 11.

| Position in | | |
|---|---|---|
| SEQ ID No. 11 | Orientation | Description |
| 1-130 | | Polylinker sequence: sequence used in cloning |
| 131-400 | Counter | sequence including the 3′ untranslated region of the 35S |
| | clockwise | transcript of the Cauliflower Mosaic Virus (Sanfaçon et al., |
| | | 1991, Genes & development, 5(1), 141-149) |
| 401-411 | | Polylinker sequence: sequence used in cloning |
| 412-3969 | Counter | cry14Ab-1.b: coding sequence of the delta-endotoxin gene of |
| | clockwise | Bacillus thuringiensis |
| 3970-5276 | Counter | Pubi10At: sequence including the promoter region of |
| | clockwise | ubiquitin-10 gene of Arabidopsis thaliana (Grefen et al., |
| | | 2010, The Plant journal, 64(2), 355-365) |
| 5277-5381 | | Polylinker sequence: sequence used in cloning |
| 5382-5576 | Counter | sequence including the 3′ untranslated region of the 35S |
| | clockwise | transcript of the Cauliflower Mosaic Virus (Sanfaçon et al., |
| | | 1991, Genes & development, 5(1), 141-149) |
| 5577-5588 | | Polylinker sequence: sequence used in cloning |
| 5589-6665 | Counter | hppdPf-4Pa: sequence encoding a variant 4- |
| | clockwise | hydroxyphenylpyruvate dioxygenase derived from |
| | | Pseudomonas fluorescens |
| 6666-7037 | Counter | TPotpY-1Pf: coding sequence of an optimized transit peptide |
| | clockwise | derivative (position 55 changed into Tyr), containing |
| | | sequence of the RuBisCO small subunit genes of Zea mays |
| | | and Helianthus annuus (U.S. Pat. No. 5,510,471) |
| 7038-7058 | | Polylinker sequence: sequence used in cloning |
| 7059-7185 | Counter | sequence including the leader sequence of the Tobacco Etch |
| | clockwise | Virus genomic RNA (Allison et al., 1985, Virology, 147(2), |
| | | 309-316) |
| 7186-7191 | | Polylinker sequence: sequence used in cloning |
| 7192-7941 | Counter | sequence including the double enhanced promoter region of |
| | clockwise | the Cauliflower Mosaic Virus 35S genome transcript (Kay et |
| | | al., 1987, Science, 236(4806), 1299-1302) |
| 7942-8068 | | Polylinker sequence: sequence used in cloning |

### 1.2. Event EE-GM4

The T-DNA vector pSZ8832 was introduced into Agrobacterium tumefaciens and transformed soybean plants (var. Thorne) were selected using HPPD inhibitor tolerance according to methods known in the art. The surviving plants were then self-pollinated to generate T1 seed. Subsequent generations were produced through self-pollination, or through crossing into other soybean germplasm.

### 1.2.1 Identification of Elite Event EE-GM4

Elite event EE-GM4 was selected based on an extensive selection procedure (based on parameters including but not limited to trait efficacy in the greenhouse and the field, molecular characteristics, and agronomic characteristics) from a wide range of different transformation events obtained using the same chimeric genes. Soybean plants containing EE-GM4 were found to have an insertion of the transgenes at a single locus in the soybean plant genome, to have overall agronomy similar to the parent plants used for transformation, to cause no yield penalty by the insertion of the transforming DNA (as compared to a corresponding isogenic line without the event, such as a “null” plant line obtained from a transformed plant in which the transgenes segregated out), to result in a significant reduction of adult females infesting the roots in a standard SCN greenhouse assay, and to have improved yield under high SCN nematode pressure in the field compared to the isogenic null line not containing EE-GM4. Additionally, tolerance to HPPD inhibitor herbicide application was measured in field trials, but herbicide tolerance was not a selection criterion for elite event selection.

### 1.2.1.1 Molecular Analysis of the Event

Southern blot results showed that EE-GM4 contains a single transgenic locus which contains a single copy of the cry14Ab-1.b chimeric gene and a single copy of the hppdPf-4 Pa chimeric gene. EE-GM4 is missing a part of the 35S promoter of the hppdPf-4 Pa chimeric gene (indicating that not the entire T-DNA of SEQ ID No. 11 was inserted in the soybean genome during transformation). No PCR fragments were obtained upon PCR analysis using primers targeting vector backbone sequences that are flanking the left and right border of the T-DNA as well as the aadA sequence. Also, the presence of identical EE-GM4 integration fragments in multiple generations of EE-GM4 demonstrates the structural stability of the event.

### 1.2.1.2 Inheritance of the Event

Inheritance of the inserted T-DNA insert in subsequent generations by testing the genotype of hppdPf-4 Pa and cry4Ab-1.b genes by PCR analysis shows that the hppdPf-4 Pa and cry4Ab-1.b genes contained within the EE-GM4 insert are inherited in a predictable manner and as expected for a single insertion. These data are consistent with Mendelian principles and support the conclusion that the EE-GM4 event consists of a single insert integrated into a single chromosomal locus within the soybean nuclear genome.

Also, analysis of the segregation patterns of EE-GM4 in subsequent generations upon introgression of EE-GM4 into 5 elite soybean lines confirmed normal Mendelian segregation. Table 3 shows the observed segregation of EE-GM4 in different segregating populations.

TABLE 3
Segregation analysis EE-GM4

| | | Observed | | | | Statistics | | |
|---|---|---|---|---|---|---|---|---|
| | | | | | | Chi- | P | |
| Parent | Generation | HH | Hemi | null | Total | Square | value | sign |
| Parent 1 | BC2F2 | 437 | 863 | 457 | 1757 | 1,00 | 0,61 | ns |
| Parent 1 | BC3F2 | 101 | 201 | 96 | 398 | 0,17 | 0,92 | ns |
| Parent 2 | BC2F2 | 52 | 127 | 70 | 249 | 2,70 | 0,26 | ns |
| Parent 2 | BC3F2 | 14 | 41 | 28 | 83 | 4,73 | 0,09 | ns |
| Parent 3 | BC2F2 | 41 | 76 | 32 | 149 | 1,15 | 0,56 | ns |
| Parent 3 | BC3F2 | 21 | 31 | 20 | 72 | 1,42 | 0,49 | ns |
| Parent 4 | F2 | 185 | 393 | 203 | 781 | 0,86 | 0,65 | ns |
| Parent 5 | BC2F2 | 63 | 143 | 87 | 293 | 4,10 | 0,13 | ns |

In Table 3, “HH” stands for homozogous plants, “Hemi” for hemizygous plants, and “null” for null-segregants having lost EE-GM4, and “ns” means not statistically significant (as to any variation from normal/expected segregation). In these trials, Parent 1 was a MG VI line with Rhg1 and Rhg4 SCN resistance, Parent 2 was a MG VI line susceptible to SCN, Parent 3 was a MG IX line susceptible to SCN, Parent 4 was a MG III line with Rhg1 SCN resistance, and Parent 5 was a MG I line susceptible to SCN.

### 1.2.1.3 Stability of Protein Expression

Protein expression levels of HPPD-4 and Cry14Ab-1 proteins in greenhouse-grown plants were determined by sandwich enzyme-linked immunosorbent assay (ELISA) in leaf, root and seed samples collected from different generations (e.g., T4, T6 and BC2F3) of EE-GM4 soybean. HPPD-4 and Cry14Ab-1 proteins exhibit similar mean expression levels in leaf, root and seed across all generations tested. Any differences observed in Cry14Ab-1 and HPPD-4 concentrations were attributed to natural plant-to-plant variability.

### 1.2.1.4 Agronomic Performance and Tolerance to HPPD Inhibitor Herbicides

In agronomic equivalency trials, plants comprising EE-GM4 in the original transformation background (Thorne) were compared to segregating nulls (lacking EE-GM4) and to wild-type Thorne plants when grown in the absence of SCN. Plots were not treated with HPPD herbicides but were maintained as weed free through the use of conventional herbicides and hand weeding where necessary. No differences impacting agronomic performance in a biologically significant way were observed between the plants containing the event and the segregating nulls (lacking EE-GM4) when grown in comparable trials at different locations when checking for qualitative plant characteristics such as flower color, pod color, seed color and pubescence and for quantitative characteristics like yield, height, lodging, stand, and days to maturity. Hence, plants comprising EE-GM4 showed normal agronomic characteristics comparable to the corresponding non-transgenic plants.

Additional trials with EE-GM4 in the original Thorne transformation background were conducted in 2017. Preliminary trials wherein EE-GM4 was in elite MG1 and MG3 genetic backgrounds were also established at a limited number of locations in 2017. When checking for qualitative plant characteristics such as flower color, pod color, seed color and pubescence and for quantitative characteristics like yield, height, lodging, stand, test weight, and days to maturity, a small delay (0.8 days) in maturity was found for plants with EE-GM4, but no agronomically meaningful differences were observed between the plants containing the event and the segregating nulls (lacking EE-GM4) in any of the three genetic backgrounds, confirming that plants comprising EE-GM4 showed normal agronomic characteristics.

Tolerance of plants comprising EE-GM4 to HPPD inhibitor herbicides was tested at different locations in the field over 2 years. In these trials, it was found that plants with EE-GM4 had commercially relevant tolerance to isoxaflutole (IFT) when applied pre-emergence, but crop damage was a bit higher for the IFT post-emergence application. These trials also showed that plants containing event EE-GM4 had commercially relevant tolerance to mesotrione (MST) when applied pre-emergence or when applied post-emergence. All post-emergence treatments were at the V2-V3 stage, with adjuvants crop oil concentrate and ammonium sulfate added to increase herbicide activity.

FIG. 5 shows the average of the maximum phytotoxicity data (plant damage) recorded for herbicide treatment in several field trials across 2 years, for soybean plants containing event EE-GM4 as compared to untransformed/conventional soybean plants. Control untransformed Thorne plants showed average maximum phytotoxicity values of about 80 to 90% in these same trials, showing these HPPD inhibitors herbicides are not tolerated by (non-GM) soybean. The “maximum phytotoxicity” as used herein is the highest phytotoxicity rating recorded at any observation during the duration of a trial (with 3 to 4 observations per trial). In existing weed control applications, a normal (1×) dose for isoxaflutole (IFT) in pre- or post-emergence application and for MST in post-emergence application is 105 gr/ha, and a normal (1×) dose for mesotrione in pre-emergence application is 210 gr/ha. Hence, in these trials reported in FIG. 5, the applications used in pre-emergence in FIG. 5 (420 gr/ha for IFT, 840 gr/ha for mesotrione) were at 4 times the normal dose, and in post-emergence (210 gr/ha for each of IFT and mesotrione) were at 2 times the normal dose.

Also, in several field trials across 2 years, soybean plants with event EE-GM4 had good tolerance towards experimental HPPD inhibitor compound 2-methyl-N-(5-methyl-1,3,4-oxadiazol-2-yl)-3-(methylsulfonyl)-4-(trifluoromethyl)benzamide (U.S. Pat. No. 9,101,141) when applied pre-emergence at 400 gr ai/ha or post-emergence at 200 gr ai/ha, respectively (the average maximum phytotoxicity value for each treatment was below 20%). In these trials, soybean plants with event EE-GM4 showed tolerance to experimental compound 2-chloro-3-(methylsulfanyl)-N-(1-methyl-1H-tetrazol-5-yl)-4-(trifluoromethyl)benzamide (U.S. Pat. No. 8,481,749) when applied post-emergence at 100-150 gr ai/ha, but the average maximum phytotoxicity of 30% was somewhat higher than for other HPPD inhibitors. All post-emergence treatments were at the V2-V3 stage, with adjuvants crop oil concentrate and ammonium sulfate added to increase herbicide activity.

The same or very similar average maximum phytotoxicity ratings as those described in FIG. 5 were obtained for IFT when adding the data obtained from a 3rd season of herbicide tolerance field trials, applying isoxaflutole herbicide at the same dosages in pre or post to EE-GM4 but at another geographic location.

### 1.2.1.5 Nematode Resistance

Standard SCN assays in the greenhouse showed a significant reduction of SCN cysts on roots of plants containing EE-GM4 when compared to Thorne wild-type soybean plants. In addition, standard SCN assays measuring female index in the greenhouse also showed that soybean plants containing event EE-GM4 and native SCN resistance showed a significant reduction of SCN cysts on roots compared to SCN resistant elite soybean lines without EE-GM4. When EE-GM4 was introgressed into an elite soybean line with PI88788 soybean resistance (maturity group 3), or into an elite soybean line with Peking soybean resistance (maturity group 6.2), consistently a reduced number of SCN cysts on the roots was seen compared to roots with native resistance alone.

In field trials across 2 years at several locations, soybean plants containing EE-GM4 gave a significant yield increase compared to the isogenic null segregants in naturally SCN-infested fields. FIG. 6 shows the grain yield of EE-GM4 in the original transformant background (Thorne) as tested in 9 different locations throughout Iowa, Illinois, Indiana, Missouri and Tennessee in 2015 and 2016, in SCN infested fields (ranging from low to high SCN infestation). Additional trials with EE-GM4 in the original transformant background (Thorne) were conducted in 2017 at a total of 12 locations with varying (natural) SCN pressure. Across all these 12 trials, plants containing EE-GM4 produced an average of 8% higher yields than the null segregants lacking EE-GM4 (p=0.02). FIG. 7 shows the grain yield of EE-GM4 when introgressed (BC2F3) into an elite MG I (maturity group I) line that is susceptible to SCN and was tested at one location in Minnesota and one location in North Dakota in 2016 (each with high SCN infestation level). The same MG I line was tested at the same two locations (each again with high SCN infestation) and at an additional location site in Wisconsin (the latter having moderate SCN pressure), and grain yield of plants containing EE-GM4 was consistently higher than the corresponding null segregants lacking EE-GM4. Finally, preliminary studies across three locations with moderate to high SCN pressure in Brazil in late planted trials in 2017 show a significant average increase of 29% (p=0.002) in a susceptible elite line for plants with EE-GM4 when compared to the segregating null (lacking EE-GM4). Due to the late planting date, overall yields in these preliminary Brazil trials tended to be low and the variability within one trial was rather high, which may have influenced the magnitude of the yield increase, but a clearly significant and visually observable yield increase was found for plants with EE-GM4. Hence, event EE-GM4 confers a significant yield increase on soybean plants in SCN-infested fields.

In a preliminary study to evaluate the effect of event EE-GM4 on yield when combined with native SCN resistance, a series of F3 populations were developed from the single cross of EE-GM4 with an elite MG III conventional line carrying the rhg1 resistance gene from P88788. In the F3 populations one ‘stacked’ population that is homozygous for both event EE-GM4 and the rhg1 allele, was compared to a population homozygous for just the rhg1 allele (lacking EE-GM4). Preliminary yield trials were established with these populations in 2016 at three locations with moderate to high infestation of SCN. Across all three locations, the ‘stacked’ population (plants homozygous for the EE-GM4 event and the rhg1 allele) produced 8% greater yields than the population carrying only the rhg1 allele (p=0.05). These results provide preliminary indications that adding the event to varieties with conventional SCN resistance can improve yields under moderate to high levels of SCN pressure.

Conducting yield trials under moderate to high SCN infestation are challenging due many factors that have an impact on the results. SCN population densities within fields can vary substantially and so the overall impact of SCN on yield can also vary from one plot to the next (see, e.g., on the world wide web at plantmanagementnetwork.org/pub/php/review/2009/sce08/). Favorable soil types, good fertility and adequate rainfall can mitigate the impact of SCN infestation on the soybean plant and can minimize yield impacts even under high SCN populations. Many fields with very high SCN populations tend to have poor soils and thus lower yield potential, making it difficult to discern statistically significant impacts on yield. Thus, yield data from SCN field trials can be quite variable and one would not expect to see significant improvements in yield in every trial with high SCN populations. The overall trends across trials are the most relevant criteria for judging performance of an event.

SCN field trials that were done with plants containing EE-GM4 were established in field with natural SCN infestation. Experimental units consisted of a field plot containing 2 to 4 rows spaced 0.76 m apart and ranging from 3.8 to 9.1 m long. The number of rows per plot and plot length varied from location to location based on field size and equipment configurations. Plots were seeded at 26 seeds per meter and so each experimental unit contained between 200 and 960 seeds. Plots were randomized in the field using a split-plot or split-split plot design. Split plot designs are well suited to help minimize the effect of high variability in soil type or SCN populations which is common in SCN infested fields. In SCN field trials plants comprising EE-GM4 were planted in a sub plot next to, or very close to, a companion sub plot containing segregating null plants (without EE-GM4). The close proximity of the two plots helps minimize the effect of (SCN) field variability on the estimate of the difference between the plants with and without event EE-GM4. Most trials were replicated four times, but a few were replicated three times and a few were replicated five or six times.

Also, moderate to severe infestations of Sudden Death Syndrome (SDS) were observed at two locations (Indiana and Iowa) in 2016 where plants with EE-GM4 were field-tested. Plots at these two locations were rated for incidence and severity of SDS symptoms and SDS Disease Index (DX) was calculated using the “SIUC Method of SDS Scoring” (on the world wide web at scnresearch.info/462.pdf). DX ratings on plants homozygous for event EE-GM4 were 43% lower at Indiana and 33% lower in Iowa than on the susceptible null segregate (lacking EE-GM4), indicating that the event was providing protection against SDS infection. SDS and SCN are often closely associated in the field and will show some interactions in the plant (see, e.g., on the world wide web at soybeanresearchinfo.com/pdf_docs/sdsupdate.pdf and on the world wide web at apsnet.org/edcenter/intropp/lessons/fungi/ascomycetes/pages/suddendeath.aspx).

In 2017, Iron Deficiency Chlorosis (IDC) scores were gathered on plants with EE-GM4 (and their null segregants) at one trial location in the US (with high SCN infestation) where IDC symptoms were observed. The trial was a split-plot design looking at the effect of event in three different backgrounds. IDC ratings were taken as described by Cianzio et al. (1979) Crop Science 19: 644-646. FIG. 10 shows the averages of IDC scores for plants with event EE-GM4 and those for the corresponding null segregants (lacking EE-GM4) across three genetic backgrounds (1 SCN-resistant (PI88788 resistance), 1 SCN-susceptible, and the SCN-susceptible Thorne background). Lower IDC scores were found for plants containing EE-GM4 compared to their null segregants. Hence, EE-GM4 can reduce the foliar severity of IDC in a field trial where soybean plants are challenged by both SCN and IDC.

Also, non-transformed Thorne and EE-GM4 seeds were geminated and planted in the greenhouse to check for control of the lesion nematode, Pratylenchus brachyurus. Pratylenchus brachyurus nematodes (#1500/plant, different developmental stages) were applied to the plants when 2 weeks old. 30 days after application, Pratylenchus nematodes were extracted from the roots and counted. The average number of nematodes found in the roots of plants containing EE-GM4 were compared with the average number of Pratylenchus nematodes found in the wild-type Thorne plant roots. On average about 80-90% fewer Pratylenchus nematodes were found in roots of plants containing EE-GM4 when compared with the Thorne control roots, indicating significant control of lesion nematodes by soybean event EE-GM4.

FIG. 8 show results from a Pratylenchus brachyurus greenhouse assay in the US, comparing elite lines with EE-GM4 in 5 elite soybean lines (one SCN susceptible (MG 1), one SCN resistant (PI88788, MG 3), one SCN susceptible (MG 6.2), one SCN resistant (Peking, MG 6.2), and one SCN susceptible (MG 9) to SCN-susceptible and SCN-resistant US soybean lines. The soybean plants were grown in small cone pots and kept in greenhouses with temperature varying between 25-32° C. Pratylenchus brachyurus nematodes, obtained from South Carolina and increased in the greenhouse were used to inoculate plants in the V2-V3 development stage. Approximately 1500 eggs+adults were inoculated per plant and each entry had 5 plants. 30 days after infestation, nematodes and eggs were extracted from the roots and counted. Each entry was run in two independent experiments. While SCN-susceptible and SCN-resistant US soybean lines did not show control of Pratylenchus, plants with EE-GM4 showed about 85% control of Pratylenchus.

FIG. 9 shows results from a Pratylenchus brachyurus greenhouse assay in Brazil, comparing soybean plants with EE-GM4 to Brazil soybean lines with no resistance and 1 low Rf line, and SCN-susceptible and -resistant plants. The soybean lines were grown in small cone pots and kept in greenhouses with temperature varying between 25-32° C. Pratylenchus brachyurus nematodes, obtained from Brazil fields and increased in the greenhouse were used to inoculate plants in the V2-V3 development stage. Approximately 1000 eggs+adults were inoculated per plant and each entry had 5 plants. 30 days after infestation, nematodes and eggs were extracted from the roots and counted. Results shown are from a single experiment. One Brazilian soybean line (BRS 7380), labeled as having a low reproductive factor for Pratylenchus, showed about 89% reduction of Pratylenchus. Plants with EE-GM4 gave 97% control of Pratylenchus. Soybean lines that carry native resistance to SCN (rhg1+Rhg4) do not control Pratylenchus brachyurus.

FIG. 11 shows the results from a Pratylenchus brachyurus field trial in Brazil, comparing soybean plants with EE-GM4 to soybean lines without transgenes and the wild-type parent (an elite maturity group IX soybean line, not known to have any native tolerance or resistance to Pratylenchus). These Pratylenchus brachyurus field trials were conducted with soybean plants containing EE-GM4 in fields naturally infested with P. brachyurus. The experimental units were four 5 m-long rows spaced 0.5 m apart. Plots were seeded at approximately 20 seeds per meter. Plots were randomized in the field using a split-plot design to help minimize the spatial variability among homozygous and null segregant comparison treatments. Each experimental location contained six replications. Efficacy of the elite event was assessed approximately 90 days after planting by collecting two subsamples from each plot. Each subsample contained the whole root systems of three plants. Root system samples were taken to the lab where juvenile and adult P. brachyurus were extracted and counted. Efficacy against P. brachyurus was determined based on the difference in the total number of juvenile and adult P. brachyurus per plant between soybean plants homozygous for EE-GM4 and null segregants. Root samples were processed for P. brachyurus according to the methods of Coolen and D'Herde (a 1972 book, entitled: A method for the quantitative extraction of nematodes from plant tissue. Belgium: State Nematology and Entomology Research Station (Ghent, (on the world wide web at cabdirect.org/cabdirect/abstract/19722001202) and Jenkins (1964, Plant Disease Report 48: 692). In these field trials, soybean plants containing EE-GM4 showed a significant reduction in the total number of Pratylenchus brachyurus (adult and juvenile) nematodes in soybean roots, compared to the null segregants lacking the event (see FIG. 11).

Also, plants containing EE-GM4 can be used to control root-knot nematodes (RKN) such as Meloidogyne incognita. Even though the population of Meloidogyne incognita does not infest Thorne wild-type soybean very well, Thorne plants with EE-GM4 show a further reduction in the number of RKN eggs/root mass on average, as compared to untransformed Thorne plants.

### 1.2.2 Identification of the Flanking Regions and Inserted T-DNA of Elite Event EE-GM4

The sequence of the regions flanking the inserted T-DNA and the T-DNA contiguous therewith in the EE-GM4 elite event are shown in the enclosed Sequence Listing.

### 1.2.2.1 5′ T-DNA Flanking Region

A fragment identified as comprising the 5′ T-DNA flanking region of EE-GM4 was sequenced and its nucleotide sequence is represented in SEQ ID No. 5, nucleotides 1-227. This 5′ T-DNA flanking region is made up of soybean genomic sequences corresponding to the pre-insertion locus sequence (SEQ ID No. 5, nucleotides 1-227). The 5′ junction region comprising part of the inserted T-DNA sequence and part of the T-DNA 5′ flanking sequence contiguous therewith is represented in SEQ ID No. 1 and 3.

### 1.2.2.2 3′ T-DNA Flanking Region

A fragment identified as comprising the 3′ T-DNA flanking region of EE-GM4 was sequenced and its nucleotide sequence is represented in SEQ ID No. 6, nucleotides 254-501. This 3′ T-DNA flanking region is made up of soybean genomic sequences corresponding to the pre-insertion locus sequence (SEQ ID No. 6, nucleotides 254-501). The 3′ junction region comprising part of the inserted T-DNA sequence and part of the T-DNA 3′ flanking sequence contiguous therewith is represented in SEQ ID No. 2 and 4.

### 1.2.2.3 Inserted T-DNA of EE-GM4

The inserted T-DNA contiguous with the above 5′ T-DNA flanking sequence was sequenced and its nucleotide sequence is represented in SEQ ID No. 5, nucleotides 228-398. Also, the inserted T-DNA contiguous with the above 3′ T-DNA flanking sequence was sequenced and its nucleotide sequence is represented in SEQ ID No. 6, nucleotides 1-253. During transformation, 970 bp of genomic DNA were deleted at the pre-insertion locus sequence, and these were replaced by the inserted T-DNA.

Sequencing of the T-DNA region in transformation plasmid pSZ8832 (the part between the T-DNA borders) resulted in the sequence reported in SEQ ID No. 11. The chimeric cry4Ab-1.b gene sequence (comprising the Ubi10 promoter and the 35S 3′ untranslated region) is represented in SEQ ID No. 11 from nucleotides 131-5276 (counterclockwise). The inserted T-DNA sequence at the 5′ flanking region in SEQ ID No. 5 (nucleotide 228-398) is identical to the nucleotide sequence in SEQ ID No. 11 from nucleotide 17 to nucleotide 187, and the inserted T-DNA sequence at the 3′ flanking region in SEQ ID No. 6 (nucleotide 1-253) is identical to the nucleotide sequence in SEQ ID No. 11 from nucleotide 7369 to nucleotide 7621. Hence, the 5′ end of the T-DNA inserted in EE-GM4 corresponds to nucleotide 17 in the transformation plasmid sequence of SEQ ID No. 11 and the 3′ end of the T-DNA inserted in EE-GM4 corresponds to nucleotide 7621 in the transformation plasmid sequence of SEQ ID No. 11. The T-DNA inserted in EE-GM4 between the sequence of SEQ ID No. 5 and the sequence of SEQ ID No. 6 is contained in the seed deposited at the ATCC under accession number PTA-123624, and has a sequence essentially similar or identical to the sequence of SEQ ID No. 11 from nucleotide 188 to nucleotide 7368.

The pre-insertion locus for event EE-GM4 can be determined from wild-type soybean var. Thorne based on the 5′ and 3′ T-DNA flanking sequences provided herein (SEQ ID No. 5 from nt 1 to nt 227 and SEQ ID No. 6 from nt 254 to nt 501) by methods known in the art. The pre-insertion locus sequence in the soybean genome corresponds to the following sequences in order: nucleotide position 1 to nucleotide position 227 in SEQ ID No. 5, a 970 nt deletion, and nucleotide position 254 to nucleotide position 501 in SEQ ID No. 6. The complete pre-insertion locus sequence is given in SEQ ID No. 30, wherein nt 1-1000 are 5′ flanking genomic sequences, nt 1001-1970 are the target site deletion, and 1971-2970 are 3′ flanking genomic sequences.

### 1.2.3. Confirmation of the Flanking Regions and Inserted T-DNA of Elite Event EE-GM4

PCR amplification using primers targeted to the plant DNA upstream and downstream of the inserted T-DNA and to the inserted T-DNA in EE-GM4, confirmed and extended the 5′ and 3′ flanking sequences.

### 1.2.3.1. 5′ Junction Sequence EE-GM4-Specific Reaction

Two primers, GLPB173 and GLPB167, were designed to amplify an amplicon of approximately 5059 bp spanning the junction region of the 5′ T-DNA flanking sequence with the T-DNA insertion fragment for event EE-GM4. The sequence of primer GLPB173 originates from the soybean reference sequence of Glycine max Williams 82.a2.vl.
Forward primer targeted to the EE-GM4 T-DNA 5′ flanking sequence:

| (SEQ ID No. 26) | |
|---|---|
| GLPB173 | 5′-CTTCATCTCCCCgTTAAAgTg-3′ |

Reverse primer targeted to the EE-GM4 inserted T-DNA sequence:

| (SEQ ID No. 28) | |
|---|---|
| GLPB167 | 5′-TACAACgTgCTCgCTATTCC-3′ |

Composition of the reaction mixture for the 5′ junction sequence reaction:

| | | |
|---|---|---|
| 5 | μl | AccuPrime ™ PCR Buffer II (Thermo Scientific) |
| 2 | μl | forward primer (10 pmol/μl) |
| 2 | μl | reverse primer (10 pmol/μl) |
| 0.75 | μl | AccuPrime ™ Taq DNA Polymerase High |
| | | Fidelity (2 U/μL; Thermo Scientific) |
| 50 | ng | template DNA |

Water up to 50 μl

Thermocycling conditions for the 5′ junction sequence reaction:

| | Time | Temperature |
|---|---|---|
| Followed by: | 1 min. | 94° C. |
| | 30 sec. | 94° C. |
| | 30 sec. | 57° C. |
| | 7 min. | 68° C. |
| | For 30 cycles | |
| Followed by: | 10 min. | 4° C. |
| | Forever | 10° C. |

The sequence of the extended T-DNA 5′ flanking sequence that was obtained and that is contiguous with and upstream of part of the inserted T-DNA as shown in SEQ ID No. 5 is shown in SEQ ID No. 24.

### 1.2.3.2. 3′ Junction Sequence EE-GM4-Specific Reaction

Two primers, GLPB170 and GLPB175, were designed to amplify an amplicon of approximately 5141 bp spanning the junction region of the T-DNA insertion fragment for event EE-GM4 with the 3′ T-DNA flanking sequence. The sequence of primer GLPB175 originates from the reference sequence of Glycine max Williams 82.a2.vl.
Forward primer targeted to the EE-GM4 inserted T-DNA sequence:

| (SEQ ID No. 29) | |
|---|---|
| GLPB170 | 5′-TCTCggTATCAgCgTTCTTg-3′ |

Reverse primer targeted to the EE-GM4 T-DNA 3′ flanking sequence:

| (SEQ ID No. 27) | |
|---|---|
| GLPB175 | 5′-gTTgTCAACAATgACCAgAAg-3′ |

Composition of the reaction mixture for the 3′ junction sequence reaction:

| | | |
|---|---|---|
| 5 | μl | AccuPrime ™ PCR Buffer II (Thermo Scientific) |
| 2 | μl | forward primer (10 pmol/μl) |
| 2 | μl | reverse primer (10 pmol/μl) |
| 0.75 | μl | AccuPrime ™ Taq DNA Polymerase High |
| | | Fidelity (2 U/μL; Thermo Scientific) |
| 50 | ng | template DNA |

Water up to 50 μl

Thermocycling conditions for the 3′ junction sequence reaction:

| | Time | Temperature |
|---|---|---|
| Followed by: | 1 min. | 94° C. |
| | 30 sec. | 94° C. |
| | 30 sec. | 57° C. |
| | 7 min. | 68° C. |
| | For 30 cycles | |
| Followed by: | 10 min. | 4° C. |
| | Forever | 10° C. |

The sequence of the extended T-DNA 3′ flanking sequence that was obtained and is contiguous with and downstream of part of the inserted T-DNA as shown in SEQ ID No. 6 is shown in SEQ ID No. 25.

Since the resulting amplicons in the above 2 reactions overlapped, this allowed a reconstruction of the sequence of the EE-GM4 inserted T-DNA and the extended 5′ and 3′ flanking sequences, which is shown in SEQ ID No. 23. The 5′ T-DNA flanking sequence in SEQ ID No. 23 is from nucleotide position 1 to nucleotide position 1058 (corresponding to pre-insertion locus genomic sequences), the inserted T-DNA sequence is from nucleotide position 1059 to nucleotide position 8663 and the 3′ T-DNA flanking sequence in SEQ ID No. 23 is from nucleotide position 8664 to nucleotide position 9749 (corresponding to pre-insertion locus genomic sequences).

### 2. Development of Identification Protocols for EE-GM4

### 2.1. End-Point Method for EE-GM4 Identity Analysis

This method describes a polymerase chain reaction detection method to analyze the presence of event EE-GM4-specific DNA sequences in DNA samples obtained from biological samples, such as plant materials (e.g., leaf or seed) using standard DNA extraction procedures.

The method description outlines the method design, including the oligonucleotide primer and probe sequences, the composition of the reaction mixture, the thermocycling conditions required to perform the reaction, and the fluorescent reader settings found appropriate for amplicon detection. It also provides general recommendations on the nature and use of control samples. In addition, guidance is provided for data analysis and interpretation, including an example of a method result taking into account the recommendations on the use of control materials and the guidance for data analysis.

### 2.1.1. Method Design

The method uses the Taqman chemistry to amplify and detect two target sequences: a EE-GM4 specific reaction determines the presence of the event, a taxon-specific reaction validates negative results for the event-specific reaction.

### 2.1.1.1. EE-GM4-Specific Reaction

Two primers, PRIM0937 and PRIM0938, were designed to amplify an amplicon of 126 bp spanning the junction region of the 3′ flanking sequence with the T-DNA insertion fragment for event EE-GM4.

A probe, TM1734 using FAM as fluorescent label and BHQ1 as quencher was designed to detected the amplified sequence.

Forward primer targeted to the EE-GM4 T-DNA sequence:

PRIM0937
(SEQ ID No. 12)
5′- gAgACTgTATCTTTgATATTTTTggAgTAgA -3′

Reverse primer targeted to the EE-GM4 T-DNA 3′ flanking sequence:

PRIM0938
(SEQ ID No. 13)
5′- CTgAgTCgATCAAAACCAATCAAT -3′

Probe targeted to the junction of the EE-GM4 T-DNA and its' 3′ flanking sequence:

TM1734
(SEQ ID No. 14)
FAM 5′- AAgTgTgTCgTgCTCCACCAgTTATCACA -3′ BHQ1

2.1.1.2. Taxon-specific specific reaction Two primers, SHA071 and SHA072, were designed to amplify an amplicon of 74 bp of the soybean endogenous lectin1 gene sequence.

A probe, TM1428 using JOE as fluorescent label and BHQ1 as was designed to detected the amplified sequence
Forward primer targeted to the endogenous Lectin 1 gene sequence:

SHA071
(SEQ ID No. 15)
5′- CCAgCTTCgCCgCTTCCTTC -3′

Reverse primer targeted to the endogenous Lectin 1 gene sequence:

SHA072
(SEQ ID No. 16)
5′- gAAggCAAgCCCATCTgCAAgCC -3′

Probe targeted to the endogenous Lectin 1 gene sequence:

TM1428
(SEQ ID No. 17)
JOE 5′- CTTCACCTTCTATgCCCCTgACAC -3′ BHQ1

### 2.1.2. Composition of the Reaction Mixture

| | | |
|---|---|---|
| 5.0 | μl | 2× PerfeCta qPCR FastMix II, ROX |
| 0.5 | μl | PRIM0937 [10 pmol/μl] |
| 0.5 | μl | PRIM0938 [10 pmol/μl] |
| 0.5 | μl | SHA071 [10 pmol/μl] |
| 0.5 | μl | SHA072 [10 pmol/μl] |
| 0.1 | μl | TM1734 [10 pmol/μl] |
| 0.1 | μl | TM1428 [10 pmol/μl] |
| x | μl | template DNA (20 ng*) |

Water up to 10 μl
Notes:
The 2× PerfeCta qPCR FastMix II, ROX was supplied by Quanta Bioscience. Other enzyme buffers may be used but performance should be verified.
Primers and labeled probes were ordered with Integrated DNA Technologies
*The amount of template DNA per reaction may vary but should be verified

### 2.1.3. Thermocycling Conditions

| | Time | Temperature |
|---|---|---|
| Followed by: | 5 min. | 95° C. |
| | 3 sec. | 95° C. |
| | 30 sec. | 60° C. |
| | For 35 cycles | |
| Followed by: | Forever | 10° C. |

Notes:
The thermocycling conditions were validated for use on a BIORAD C1000 thermal cycler. Other equipment may be used but performance should be verified

### 2.1.4. Wavelength and Bandwidth Settings

| | Excitation | Emission |
|---|---|---|
| FAM | 495 nm ± 5 nm | 517 nm ± 5 nm |
| JOE | 530 nm ± 5 nm | 555 nm ± 5 nm |
| ROX | 581 nm ± 5 nm | 607 nm ± 5 nm |

Notes:
Wavelength and bandwidth settings were validated for use on a Tecan M1000 plate reader. Other equipment and settings may be used but performance should be verified

### 2.1.5. Control Samples

Following control samples should be included in the experiment to validate the results of test samples:
Positive control: a DNA sample containing the target and endogenous sequences
Negative control: a DNA sample containing only the endogenous sequence
No template control: a water sample (no DNA)

### 2.1.6. Data Analysis

For all samples, fluorescent Signal to Background ratio's (S/B) are calculated for both the target and endogenous reaction.
Control samples should give the expected result, i.e.:
   The positive control should be scored “detected”
   The negative control should be scored “not detected”
   The no template control should only show fluorescent background levels
A sample is scored as follows:
   Detected: the target S/B and the endogenous S/B exceeds an acceptable threshold ratio, e.g. 2
   Not-detected: the target S/B is below an acceptable threshold ratio, e.g. 1.3, and, in addition, the endogenous S/B exceeds an acceptable threshold ratio, e.g. 2
   Inconclusive: the target and endogenous S/B are below an acceptable threshold, e.g. 1.3

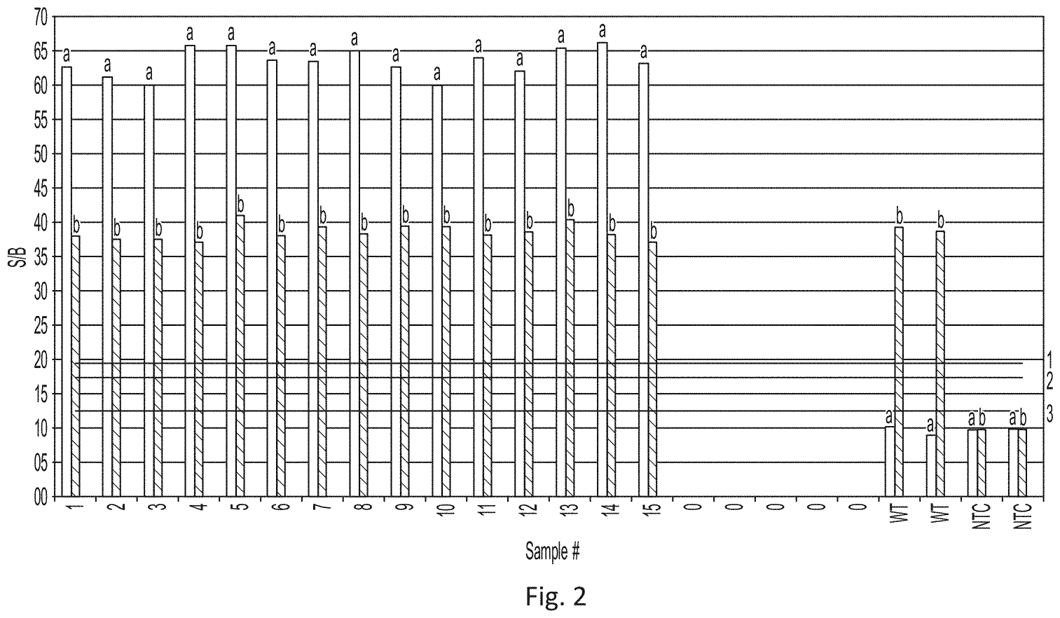

FIG. 2 shows an example of the result of the method for a series of soybean samples containing EE-GM4 and conventional soybean samples. For each sample the S/B ratios for both the EE-GM4 specific reaction and the endogenous reaction are displayed.

### 2.2. End-Point Method for EE-GM4 Identity and Zygosity Analysis

This method describes a polymerase chain reaction detection method to analyze the presence and the zygosity status of event EE-GM4-specific DNA sequences in DNA samples obtained from biological samples, such as plant materials (e.g., leaf or seed) using standard DNA extraction procedures.

The method description outlines the reaction reagents, the oligonucleotide primer and probe sequences, the thermocycling conditions required to perform the reaction, and the fluorescent reader settings found appropriate for amplicon detection. It also provides general recommendations on the nature and use of control samples. In addition, guidance is provided for data analysis and interpretation, including an example of a method result taking into account the recommendations on the use of control materials and the guidance for data analysis.

It is noted that the method performance for zygosity analysis may be variety dependent due to the nature of the pre-insertion locus sequence. Therefore, performance verification is required for each variety in which the event is introgressed. For cases of inadequate performance, an alternative Real-Time PCR method based on copy number analysis can be used. E.g., such a copy number analysis can use the Taqman chemistry and principles of Real-Time PCR to quantify the relative copy number of a EE-GM4 specific sequence. The alternative method will typically include a EE-GM4 specific reaction to quantify the EE-GM4 copy number, and a taxon-specific reaction for normalization of the EE-GM4 copy number. Samples containing the EE-GM4 insertion sequence in a homozygous state will have a relative copy number that is two-fold higher than hemizygous samples. Azygous samples will not amplify the EE-GM4 sequence in such method.
2.2.1. Method design
   The method uses the Taqman chemistry to amplify and detect two target sequences: a EE-GM4 specific reaction determines the presence of the event, a pre-insertion locus-specific reaction determines the presence of the pre-insertion locus of the event.
   Detection of only the EE-GM4 specific sequence indicates the presence of event EE-GM4 in a homozygous zygosity state.
   Detection of the EE-GM4 specific and pre-insertion locus specific sequence indicates the presence of event EE-GM4 in a hemizygous zygosity state.
   Detection of only the pre-insertion locus specific sequence indicates the absence of event EE-GM4.

2.2.1.1. EE-GM4-Specific Reaction
Two primers, PRIM0937 and PRIM0938, were designed to amplify an amplicon of 126 bp spanning the junction region of the T-DNA 3′ flanking sequence with the T-DNA insertion fragment for event EE-GM4.
A probe, TM1734 using FAM as fluorescent label and BHQ1 as quencher, was designed to detected the amplified sequence.
Forward primer targeted to the EE-GM4 T-DNA sequence:

PRIM0937
(SEQ ID No. 12)
5′- gAgACTgTATCTTTgATATTTTTggAgTAgA -3′

Reverse primer targeted to the EE-GM4 T-DNA 3′ flanking sequence:

PRIM0938
(SEQ ID No. 13)
5′- CTgAgTCgATCAAAACCAATCAAT -3′

Probe targeted to the junction of the EE-GM4 T-DNA and its' 3′ flanking sequence:

TM1734
(SEQ ID No. 14)
FAM 5′- AAgTgTgTCgTgCTCCACCAgTTATCACA -3′ BHQ1

2.2.1.2. Pre-Insertion Locus Specific Reaction
Two primers, PRIM1652 and PRIM0938, are designed to amplify an amplicon of 108 bp spanning the junction of the pre-insertion locus and the 3′flanking sequence of the EE-GM4 pre-insertion locus.
A MGB probe, TM2084 using VIC as fluorescent label and the MGB-NFQ as quencher, was designed to detect the amplified sequence
Forward primer targeted to the EE-GM4 pre-insertion locus sequence:

PRIM1652
(SEQ ID No. 18)
5′- gAgAAgTTTCAATACTAATAgTATCAATACTCAgAAT -3′

Reverse primer targeted to the EE-GM4 T-DNA 3′ flanking sequence:

PRIM0938
(SEQ ID No. 13)
5′- CTgAgTCgATCAAAACCAATCAAT -3′

Wild type probe targeted to the junction of the pre-insertion locus and 3′ flanking sequence:

TM2084
(SEQ ID No. 19)
VIC 5′- CgAgTATTAgCCATATTTA -3′ MGB-NFQ

### 2.2.2. Composition of the Reaction Mixture

| | | |
|---|---|---|
| 5.0 | μl | 2× PerfeCta qPCR FastMix II, ROX |
| 0.4 | μl | PRIM1652 [10 pmol/μl] |
| 0.4 | μl | PRIM0938 [10 pmol/μl] |
| 0.1 | μl | PRIM0937 [10 pmol/μl] |
| 0.2 | μl | TM2084 [10 pmol/μl] |
| 0.05 | μl | TM1734 [10 pmol/μl] |
| x | μl | template DNA (20 ng*) |

Water up to 10 μl
Notes:
The 2× PerfeCta qPCR FastMix II, ROX was supplied by Quanta Bioscience. Other enzyme buffers may be used but performance should be verified.
Primers and labeled probes were ordered with Integrated DNA Technologies
*The amount of template DNA per reaction may vary but should be verified

### 2.2.3. Thermocycling Conditions

| | Time | Temperature |
|---|---|---|
| Followed by: | 5 min. | 95° C. |
| | 3 sec. | 95° C. |
| | 30 sec. | 60° C. |
| | For 35 cycles | |
| Followed by: | Forever | 10° C. |

Notes:
The thermocycling conditions were validated for use on a BIORAD C1000 thermal cycler. Other equipment may be used but performance should be verified

### 2.2.4. Wavelength and Bandwidth Settings

| | Excitation | Emission |
|---|---|---|
| FAM | 495 nm ± 5 nm | 517 nm ± 5 nm |
| JOE | 530 nm ± 5 nm | 555 nm ± 5 nm |
| ROX | 581 nm ± 5 nm | 607 nm ± 5 nm |

Notes:
Wavelength and bandwidth settings were validated for use on a Tecan M1000 plate reader. Other equipment and settings may be used but performance should be verified

### 2.2.5. Control Samples

Following control samples should be included in the experiment to validate the results of test samples:
Homozygous control: a DNA sample containing the target sequence in a homozygous state
Hemizygous control: a DNA sample containing the target sequence in a hemizygous state
Wild type control: a DNA sample not containing the target sequence
No template control: a water sample

### 2.2.6. Data Analysis

For all samples, fluorescent Signal to Background ratio's (S/B) are calculated for both the target and pre-insertion locus reaction.
Control samples should give the expected result, i.e.:
   The homozygous control should be scored “homozygous”
   The hemizygous control should be scored “hemizygous”
   The wild type control should be scored “wild type”
   The no template control should only show fluorescent background levels
A sample is scored as follows:
   homozygous: the target S/B exceeds an acceptable threshold ratio, e.g. 2, and the pre-insertion locus S/B is below an acceptable threshold ratio, e.g. 1
   hemizygous: both the target and pre-insertion locus S/B exceeds an acceptable threshold ratio, e.g. 2
   wild type: the target S/B is below an acceptable threshold ratio, e.g. 1, and the pre-insertion locus S/B exceeds an acceptable threshold ratio, e.g. 2
   Inconclusive: the target and pre-insertion locus S/B are below an acceptable threshold, e.g. 1

FIG. 3 shows an example of the result of the method for a series of soybean samples containing EE-GM4 in a homozygous state, soybean samples containing EE-GM4 in a hemizygous state and conventional soybean samples.

### 2.3. Real-Time PCR Method for EE-GM4 Low Level Presence Analysis

The method describes a detection method to analyze the Low Level Presence of event EE-GM4 DNA sequences obtained from bulked plant materials (e.g., leaf or seed) or processed materials (e.g., food or feed products produced from processed soybean grain, containing EE-GM4 DNA) using standard DNA extraction procedures.

The method description outlines the reaction reagents, the oligonucleotide primer and probe sequences, and the thermocycling conditions required to perform the reaction. It also provides general recommendations on the concurrent use of a taxon-specific method to support data analysis and result interpretation. In addition, recommendations are provided on the nature and use of control samples.

It is noted that alternative methods may be available for the intended purpose, including but not limited to digital droplet PCR methods. Digital droplet PCR methods use End-Point methods for event identity analysis, as described in section 1.1, in combination with principles of subsampling on the extracted DNA sample. In this method the low level presence of the event is determined based on the ratio of DNA subsamples found positive and negative for the event sequence.
2.3.1. Method Design
   The method uses the Taqman chemistry and principles of Real-Time PCR to detect or quantify low levels of EE-GM4 in a DNA sample.
   Two primers, PRIM0939 and PRM0940, are designed to amplify an amplicon of 90 bp spanning the junction region of the T-DNA 5′ flanking sequence with the T-DNA insertion fragment for event EE-GM4.
   A probe, TM1735 using FAM as fluorescent label and BHQ1 as quencher, is designed to quantify the amplified sequence.
      Forward primer targeted to the EE-GM4 T-DNA sequence:

PRIM0939
(SEQ ID No. 20)
5′- CCATTgTgCTgAATAggTTTATAgCT -3′

Reverse primer targeted to the EE-GM4 T-DNA 5′ flanking sequence:

PRIM0940
(SEQ ID No. 21)
5′- gACAAATACTACTTTgTTAAgTTTAgACCCC -3′

Probe targeted to the junction of the EE-GM4 T-DNA and its' 5′ flanking sequence:

TM1735
(SEQ ID No. 22)
FAM 5′- TgATAgAgCgCCTgggCCTAACTTTCTAAA -3′ BHQ-1

### 2.3.2. Composition of the Reaction Mixture

| | | |
|---|---|---|
| 10.0 | μl | 2× PerfeCta qPCR Fastmix II, Low ROX |
| 0.5 | μl | PRIM0939 [10 pmol/μl] |
| 0.5 | μl | PRIM0940 [10 pmol/μl] |
| 0.5 | μl | TM1735 [10 pmol/μl] |
| x | μl | template DNA (200 ng*) |

Water up to 20 μl
Notes:
The 2× PerfeCta qPCR FastMix II, LOW ROX was supplied by Quanta Bioscience. Other enzyme buffers may be used but performance should be verified.
Primer and labeled probes were ordered with Integrated DNA Technologies
*The amount of template DNA per reaction may vary but should be verified

### 2.3.3. Thermocycling Conditions

| | Time | Temperature |
|---|---|---|
| Followed by: | 5 min. | 95° C. |
| | 3 sec. | 95° C. |
| | 30 sec. | 60° C. ** |
| | For 40 cycles | |

Notes:
** Fluorescent read-out is performed at each cycle, upon finalization of the primer elongation step at 60° C.
The thermocycling conditions were validated for use on a ViiA7 and Quantstudio 7 Real-Time PCR apparatus. Other equipment may be used but performance should be verified

### 2.3.4. Taxon Specific Method

A Real-Time PCR detection method targeting an endogenous sequence should be performed concurrently on an identical amount of template DNA as used in the target specific Real-Time PCR method. The outcome of the taxon specific method should be used to support data analysis and interpretation, i.e. to normalize the amount of input DNA and to validate any negative results for the target specific reaction.

### 2.3.5. Test Samples, Calibration Samples and Control Samples

It is recommended that all test samples are analyzed in duplicate.
A set of calibration samples is included in the experiment to generate standard curves for both the target and taxon specific method.
In addition the following control samples are included:
   Positive control: a DNA sample containing the target sequence at the level of the Limit Of Detection,
   Negative control: a DNA sample containing only the endogenous sequence (no EE-GM4)
   No template control: a water sample

### 2.3.6. Data Analysis

For all samples threshold cycle values (i.e., Ct values) are determined for both the target and taxon specific method. A threshold cycle is defined as the cycling number at which the amplification plot for a given sample reaches a defined signal threshold (see figure below)
Standard curve formulas are calculated for both the target and taxon specific method using the Ct values and the amount of genome copies of the calibration samples
The standard curve parameters should fulfill acceptance criteria for slope and linearity (R²), e.g.
   −3.2<slope<−3.6
   R²>0.98
For all samples, the genome copy number for the target and endogenous method is calculated using linear regression analysis.
The amount of low level presence relative to the total amount of taxon specific DNA is determined by calculating the % ratio of the genome copy numbers for the target and taxon specific method.
Control samples should give the expected result, i.e.:
   The positive control should be scored “detected”
   The negative control should be scored “not detected”
   The no template control should only show fluorescent background levels
A sample is scored as follows:
   Detected: the low level presence is above the limit of detection for all replicates, taking into account the measurement uncertainty of the method
   Not-detected: the low level presence is below the limit of detection for all replicates, taking into account the measurement uncertainty of the method
   Inconclusive: replicated samples give inconsistent scores

FIG. 4 shows an example of the result of the method performed on the calibration samples.

3. Introgression of EE-GM4 into Preferred Cultivars

Elite event EE-GM4 was introduced by repeated back-crossing into six different elite soybean lines. The lines were selected to represent a range of maturities: two lines from MG I, one line from MG III, two lines from MG VI and one line from MG IX. One of the MG I lines and the MG III line contained the Rhg1 native resistance allele from PI 88788, and one of the MG VI lines carried the Rhg1 and Rhg4 native resistance alleles from PI 437654. The other three lines were susceptible to SCN.

Also, in initial testing, in several experiments, no biologically significant differences were observed for Cry14Ab-1 or HPPD-4 protein expression levels measured in leaves of greenhouse-grown plants (as measured with ELISA or western blot (only normal assay variation was seen)), and no significant differences were seen in the standard greenhouse SCN assay results (measuring % reduction in SCN cysts vs. the Thorne control), when event EE-GM4 was introgressed from Thorne background into other soybean germplasm backgrounds (of different maturity, at different stages of introgression), compared to what was found in the Thorne background. While in an initial experiment, the EE-GM4 event in a BC2F3 soybean plant background of maturity 9.1 showed a lower expression of Cry14Ab-1 protein, no biologically significant differences in Cry14Ab-1 protein expression with respect to EE-GM4 in Thorne background was found when this experiment was repeated with a larger number of plants of that maturity.

Introgression of the elite event EE-GM4 into other soybean cultivars does not significantly influence any of the desirable phenotypic or agrnomic characteristics of these cultivars (no linkage drag) while expression of the transgenes meets commercially acceptable levels. This confirms the status of event EE-GM4 as an elite event.

Furthermore, elite event EE-GM4 is advantageously combined with other soybean elite transformation events. Particularly useful plants according to the invention are plants containing EE-GM4 combined with another soybean transformation event, or a combination of more than one other soybean transformation event, such as those listed in the databases of various national or regional regulatory agencies, including but not limited to Event MON87751 (aka MON-87751-7, described in WO2014201235 and USDA-APHIS Petition 13-337-01p), Event pDAB8264.42.32.1 (described in WO2013010094), Event DAS-81419-2 (aka Conkesta™ Soybean, described in WO2013016527 and USDA-APHIS Petition 12-272-01p), Event EE-GM3 (aka FG-072, MST-FG072-3, described in WO2011063411, USDA-APHIS Petition 09-328-01p), Event SYHTOH2 (aka 0H2, SYN-ØØØH2-5, described in WO2012/082548 and 12-215-01p), Event DAS-68416-4 (aka Enlist Soybean, described in WO2011/066384 and WO2011/066360, USDA-APHIS Petition 09-349-01p), Event DAS-81615-9 (described in WO2014004458), Event DAS-444Ø6-6 (aka Enlist E3, DAS-444Ø6-6, described in WO2012/075426 and USDA-APHIS 11-234-01p), Event MON87708 (Xtend Soybeans, described in WO2011/034704 and USDA-APHIS Petition 10-188-01p), Event MON89788 (aka Genuity Roundup Ready 2 Yield, described in WO2006/130436 and USDA-APHIS Petition 06-178-01p), Event DAS-14536-7 (described in WO2012/075429), Event 40-3-2 (aka RoundUp Ready, MON-Ø4Ø32-6, described in USDA-APHIS Petition 93-258-01), Event A2704-12 (aka LL27, ACS-GMØØ5-3, described in WO2006108674 and USDA-APHIS Petition 96-068-01p), Event 127 (aka BPS-CV127-9, described in WO2010/080829), Event A5547-127 (aka LL55, ACS-GMØØ6-4, described in WO2006108675 and in USDA-APHIS Petition 96-068-01p), Event MON87754 (aka Vistive III, MON-87754-1, described in WO2010/024976), Event HOS (aka DP-3Ø5423-1, Plenish High Oleic Soybean, described in WO2008054747), Event MON87701 (aka MON-877Ø1-2, described in WO2009064652 and USDA-APHIS Petition 09-082-01p), Event MON 87705 (aka MON-877Ø5-6, described in and USDA-APHIS Petition 09-201-01p), Event MON87712 (aka MON-87712-4, described in WO2012/051199, USDA-APHIS Petition 11-202-01p), Event pDAB4472-1606 (aka Event 1606, described in WO2012/033794), Event 3560.4.3.5 (aka DP-356043-5, described in WO2008/002872), Event MON87769 (aka MON-87769-7, described in WO2009102873 and in USDA-APHIS Petition 09-183-01p), event IND-ØØ41Ø-5 (aka HB4 Soybean, US FDA BNF No. 000155, USDA-APHIS Docket No. APHIS-2017-0075, www.aphis.usda.gov/brs/aphisdocs/17_22301p.pdf), event DP305423 (aka DP-3Ø5423-1, published PCT patent application, USDA-APHIS Petition 06-354-01p), or any combination of EE-GM4 with several of these other transgenic soybean events, such as a combination of EE-GM4 with any one of the following combinations. Event MON89788 x MON87708 (aka Roundup Ready 2 Xtend Soybeans, MON-877Ø8-9×MON-89788-1), Event HOS×Event 40-3-2 (aka Plenish High Oleic Soybeans×Roundup Ready Soybeans), Event EE-GM3×EE-GM2 (aka FG-072×LL55, described in WO2011063413), Event MON 87701×MON 89788 (aka Intacta RR2 Pro Soybean, MON-877Ø1-2×MON-89788-1), DAS-81419-2×DAS-44406-6 (aka Conkesta™ Enlist E3™ Soybean, DAS-81419-2×DAS-444Ø6-6), Event DAS-81419-2×Event DAS-68416-4 (described in WO2013016516), Event DAS-68416-4×Event MON 89788 (aka Enlist™ RoundUp Ready® 2 Soybean, DAS-68416-4 X MON-89788-1), MON 87705×MON 89788 (aka Vistive Gold, MON-877Ø5-6×MON-89788-1), MON 87769 x MON 89788 (aka Omega-3×Genuity Roundup Ready 2 Yield Soybeans, MON-87769-7 x MON-89788-1), Event DP305423×Event 40-3-2 (DP-3Ø5423-1×MON-Ø4Ø32-6), Event DP305423×MON87708 (DP-3Ø5423-1×MON-877Ø8-9), Event DP305423×MON87708 x Event MON89788 (DP-3Ø5423-1×MON-877Ø8-9×MON-89788-1), Event DP305423×Event MON89788 (DP-3Ø5423-1×MON-89788-1), Event MON87705×MON87708 (MON-877Ø5-6 x MON-877Ø8-9), Event MON87705×MON87708×MON89788 (MON-877Ø5-6×MON-87708-9×MON-89788-1), Event MON89788×MON87708×A5547-127 (MON-89788-1 x MON-877Ø8-9×ACS-GMØØ6-4), Event MON87751×MON87701×MON87708 x MON89788 (MON-87751-7×MON-877Ø1-2×MON-877Ø8-9×MON-89788-1).

As used in the claims below, unless otherwise clearly indicated, the term “plant” is intended to encompass plant tissues, at any stage of maturity, as well as any cells, tissues, or organs taken from or derived from any such plant, including without limitation, any seeds, leaves, stems, flowers, roots, single cells, gametes, cell cultures, tissue cultures or protoplasts.

Reference seed comprising elite event EE-GM4 was deposited at the ATCC (10801 University Blvd., Manassas, Va. 20110-2209) on Nov. 9, 2016, under ATCC accession number PTA-123624, and the viability thereof was confirmed. Alternative names for EE-GM4 are event GMB471 or BCS-GM471-2.

The above description of the invention is intended to be illustrative and not limiting.

Various changes or modifications in the embodiments described may occur to those skilled in the art. These can be made without departing from the spirit or scope of the invention.

1. A soybean plant, cell, plant part, or seed, comprising any of the nucleic acid molecules: a) a nucleic acid molecule comprising a nucleotide sequence essentially similar to the sequence of any one of SEQ ID No. 1, 3 or 5 or to the sequence of any one of SEQ ID No. 2, 4, or 6, or the complement of said sequences, b) a nucleic acid molecule comprising a nucleotide sequence with at least 99% sequence identity to the nucleotide sequence of SEQ ID No. 3, 4, 5, 6, 24 or 25, or the complement thereof, c) a nucleic acid molecule comprising the nucleotide sequence of any one of SEQ ID No. 1 or 3 or SEQ ID No. 2 or 4, or the complement of said sequences, or comprising the nucleotide sequence of SEQ ID No. 1 or 3 and SEQ ID No. 2 or 4, or the complement thereof, d) a nucleic acid molecule comprising the nucleotide sequence of any one of SEQ ID No. 1 or 3, or SEQ ID No. 2 or 4, or the complement of said sequences, and the nucleotide sequence of SEQ ID No. 7 and 9, or the complement thereof, or comprising the nucleotide sequence of SEQ ID No. 1 or 3 and SEQ ID No. 2 or 4, or the complement of said sequences, and the nucleotide sequence of SEQ ID No. 7 and 9, or the complement thereof, e) a nucleic acid molecule comprising the nucleotide sequence of SEQ ID No. 11 from nucleotide position 188 to nucleotide position 7368, or a nucleotide sequence having at least 98%, or least 99%, or at least 99.5% or at least 99.9% sequence identity thereto, f) a nucleic acid molecule comprising the nucleotide sequence of SEQ ID No. 11 from nucleotide position 188 to nucleotide position 7101, which comprises the nucleotide sequence of SEQ ID No. 5 or 24 and SEQ ID No. 6 or 24, or the complement thereof, g) a nucleic acid molecule obtainable from the seed deposited at the ATCC under accession number PTA-123624, wherein said nucleic acid molecule comprises the nucleotide sequence of any one of SEQ ID No. 1, 3, or 5 and the nucleotide sequence of any one of SEQ ID No. 2, 4, or 6, wherein said soybean plant, cell, plant part, seed or progeny thereof, or the soil in which they are grown or are intended to be grown, is treated with one or more of the compounds and/or biological control agents or mixtures thereof, as described herein, such as a compound and/or biological control agent or mixture from any one of H1, H2, H3, H4, H5, IAN1, IAN2, IAN3, IAN4, IAN5, IAN6, IAN7, IAN8, IAN9, IAN10, IAN11, IAN12, IAN13, IAN14, IAN15, IAN16, IAN17, IAN18, IAN19, IAN20, IAN21, IAN22, IAN23, IAN24, IAN25, IAN26, IAN27, IAN28, IAN29, IAN30, SIAN1, F1, F2, F3, F4, F5, F6, F7, F8, F9, F9, F10, F11, F12, F13, F14, F15, F16, SF1, P1, BCA1, BCA2, BCA3, BCA4, BCA5, BCA6, BCA7, BCA8; BCA9, BCA10, NC1, NC2, NC3, SBCA1, SAI1, SC1 and SC2.

2. A soybean plant, cell, plant part, or seed, each comprising in its genome elite event EE-GM4, wherein said elite event is the genetic locus comprising an inserted T-DNA containing a chimeric HPPD-4 protein-encoding gene and a chimeric Cry14Ab-1 protein-encoding gene, and 5′ and 3′ flanking sequences immediately surrounding said inserted T-DNA, as found in reference seed deposited at the ATCC under deposit number PTA-123624, such as said plant, cell, part or seed which also comprises the nucleotide sequence of SEQ ID No. 3 and the nucleotide sequence of SEQ ID No. 4, wherein said soybean plant, cell, plant part, or seed, or the soil in which they are grown or are intended to be grown, are treated with one or more of the compounds and/or biological control agents or mixtures thereof as described herein.

3. The plant, plant part or seed of claim 1 or 2, wherein the soil in which they are grown or are intended to be grown, is treated with one or more of said compounds and/or biological control agents or mixtures thereof, and wherein said plant, plant part or seed is planted or sown in the soil treated with one or more of the compounds and/or biological control agents or mixtures thereof as described herein.

4. The plant, cell, plant part, or seed of the any one of claim 1 top 3 wherein said plant, cell, plant part or seed also comprises native soybean SCN resistance loci or genes, such as any one of the SCN resistance genes or loci from the resistance sources PI 548316, PI 567305, PI 437654, PI 90763, PI404198B, PI 88788, PI 468916, PI 567516C, PI 209332, PI 438489B, PI 89772, Peking, PI 548402, PI 404198A, PI 561389B, PI 629013, PI 507471, PI 633736, PI 507354, PI 404166, PI 437655, PI 467312, PI 567328, PI 22897, or PI 494182, or any one of the SCN resistance genes or loci from the resistance sources of Table 1, or any one of the SCN resistance genes or loci from the resistance sources PI 88788, PI 548402, PI 209332, PI 437654, or a combination thereof.

5. A process for weed control, comprising treating a field in which the soybean seeds of any of claims 1 to 4 were sown with an HPPD inhibitor herbicide, and with a nematicidal agent, before the soybean plants emerge but after the seeds are sown.

6. A process for protecting emerging soybean plants of any of claims 1 to 4 from competition by weeds, comprising treating a field to be sown or planted with said soybean seeds or plants with an HPPD inhibitor herbicide and a nematicidal agent, before the soybean plants are planted or the seeds are sown, followed by planting or sowing of said soybean plants or seeds in said field that has been treated.

7. The process of claim 5 or 6, wherein said nematicidal agent is or contains a nematicidal compound or a nematicidal biological control agent (such as Bacillus firmus, Pasteuria nishizawae, Bacillus subtilis, Bacillus licheniformis, Burkholderia rinojensis or Bacillus amyloliquefaciens) as described herein.

8. A process for producing a soybean plant resistant to SCN and/or tolerant to HPPD inhibitor herbicides, comprising introducing resistance to SCN and/or tolerance to HPPD inhibitor herbicides into the genome of a soybean plant by crossing a first soybean plant, such as a soybean plant lacking a Cry14Ab-1-encoding gene and lacking an HPPD-4-encoding gene, with a second soybean plant comprising elite event EE-GM4, and selecting seed of a progeny plant comprising elite event EE-GM4, which method includes the step of treating said seed with one or more of the compound(s) and/or biological control agent(s) or mixtures comprising them, as described herein, wherein said elite event comprises a nucleic acid molecule as described in claim 1, or wherein said elite event is the genetic locus comprising an inserted T-DNA containing a chimeric HPPD-4 protein-encoding gene and a chimeric Cry14Ab-1 protein-encoding gene, and 5′ and 3′ flanking sequences immediately surrounding said inserted T-DNA, as found in reference seed deposited at the ATCC under deposit number PTA-123624, such as said plant which also comprises the nucleotide sequence of SEQ ID No. 3 and the nucleotide sequence of SEQ ID No. 4.

9. Use of the plant, seed, part, or cell of any of claims 1 to 4, and said compound and/or biological control agent or a mixture, to produce soybean seed.

10. Use of the treated soybean plant or seed of any of claims 1 to 4 to grow a nematode-resistant and/or HPPD inhibitor herbicide-tolerant plant.

11. A process for increasing yield of soybean plants when planted in a field that contains, contained or is expected to contain nematodes or nematode eggs such as SCN, RKN, Pratylenchus or reniform nematodes, comprising the step of 1) obtaining soybean plants or seed comprising elite event EE-GM4, and 2) planting or sowing of said plants or seeds in said field, wherein reference seed comprising said elite event is deposited at the at the ATCC under deposit number PTA-123624, which method comprises the step of treating said plant or seed with one or more of the compound(s) and/or biological control agent(s) or mixtures comprising them, as described herein.

12. A process to increase yield of soybean plants in SCN-containing fields infested with Sudden Death Syndrome or in SCN-containing fields causing Iron Deficiency Chlorosis in soybean, which method comprises sowing treated seeds comprising elite event EE-GM4, wherein reference seed comprising said elite event is deposited at the at the ATCC under deposit number PTA-123624, wherein said seeds are treated with one or more of the compound(s) and/or biological control agent(s) or mixtures comprising them, as described herein, such as a nematicidal, insecticidal or fungicidal compound(s) and/or biological control agent(s), or a combination of a nematicidal, insecticidal and fungicidal compound(s) and/or biological control agent.

13. The plant, cell, plant part or seed of any one of claims 1 to 4, or the soil in which they are grown or are intended to be grown, treated with any of the following nematicidal agents: alanycarb, aldicarb, carbofuran, carbosulfan, fosthiazate, cadusafos, oxamyl, thiodicarb, dimethoate, ethoprophos, terbufos, abamectin, methyl bromide and other alkyl halides, methyl isocyanate generators selected from diazomet and metam, fluazaindolizine, fluensulfone, fluopyram, tioxazafen, N-[1-(2,6-difluorophenyl)-1H-pyrazol-3-yl]-2-trifluoromethylbenzamide, cis-Jasmone, harpin, Azadirachta indica oil, or Azadirachtin.

14. The plant, cell, plant part or seed of any one of claims 1 to 4, or the soil in which they are grown or are intended to be grown, treated with any of the following nematicidal agents: fosthiazate, cadusafos, thiodicarb, abamectin, fluazaindolizine, fluopyram, tioxazafen, N-[1-(2,6-difluorophenyl)-1H-pyrazol-3-yl]-2-trifluoromethylbenzamide, cis-Jasmone, harpin, Azadirachta indica oil, or Azadirachtin.

15. The plant, cell, plant part or seed of claim 14, treated with a combination selected from the group consisting of: fosthiazate and cadusafos, fosthiazate and thiodicarb, fosthiazate and abamectin, fosthiazate and fluazaindolizine, fosthiazate and fluopyram, fosthiazate and tioxazafen, fosthiazate and N-[1-(2,6-difluorophenyl)-1H-pyrazol-3-yl]-2-trifluoromethylbenzamide, fosthiazate and cis-Jasmone, fosthiazate and harpin, fosthiazate and Azadirachta indica oil, fosthiazate and Azadirachtin, cadusafos and fosthiazate, cadusafos and thiodicarb, cadusafos and abamectin, cadusafos and fluazaindolizine, cadusafos and fluopyram, cadusafos and tioxazafen, cadusafos and N-[1-(2,6-difluorophenyl)-1H-pyrazol-3-yl]-2-trifluoromethylbenzamide, cadusafos and cis-Jasmone, cadusafos and harpin, cadusafos and Azadirachta indica oil, cadusafos and Azadirachtin, thiodicarb and fosthiazate, thiodicarb and cadusafos, thiodicarb and abamectin, thiodicarb and fluazaindolizine, thiodicarb and fluopyram, thiodicarb and tioxazafen, thiodicarb and N-[1-(2,6-difluorophenyl)-1H-pyrazol-3-yl]-2-trifluoromethylbenzamide, thiodicarb and cis-Jasmone, thiodicarb and harpin, thiodicarb and Azadirachta indica oil, abamectin and cadusafos, abamectin and thiodicarb, abamectin and fluazaindolizine, abamectin and fluopyram, abamectin and tioxazafen, abamectin and N-[1-(2,6-difluorophenyl)-1H-pyrazol-3-yl]-2-trifluoromethylbenzamide, abamectin and cis-Jasmone, abamectin and harpin, abamectin and Azadirachta indica oil, abamectin and Azadirachtin, fluazaindolizine and abamectin, fluazaindolizine and fluopyram, fluazaindolizine and tioxazafen, fluazaindolizine and N-[1-(2,6-difluorophenyl)-1H-pyrazol-3-yl]-2-trifluoromethylbenzamide, fluazaindolizine and cis-Jasmone, fluazaindolizine and harpin, fluazaindolizine and Azadirachta indica oil, fluazaindolizine and Azadirachtin, fluopyram and fluazaindolizine, fluopyram and tioxazafen, fluopyram and N-[1-(2,6-difluorophenyl)-1H-pyrazol-3-yl]-2-trifluoromethylbenzamide, fluopyram and cis-Jasmone, fluopyram and harpin, fluopyram and Azadirachta indica oil, fluopyram and Azadirachtin, tioxazafen and N-[1-(2,6-difluorophenyl)-1H-pyrazol-3-yl]-2-trifluoromethylbenzamide, tioxazafen and cis-Jasmone, tioxazafen and harpin, tioxazafen and Azadirachta indica oil, tioxazafen and Azadirachtin, N-[1-(2,6-difluorophenyl)-1H-pyrazol-3-yl]-2-trifluoromethylbenzamide and cis-Jasmone, N-[1-(2,6-difluorophenyl)-1H-pyrazol-3-yl]-2-trifluoromethylbenzamide and harpin, N-[1-(2,6-difluorophenyl)-1H-pyrazol-3-yl]-2-trifluoromethylbenzamide and Azadirachta indica oil, N-[1-(2,6-difluorophenyl)-1H-pyrazol-3-yl]-2-trifluoromethylbenzamide and Azadirachtin, cis-Jasmone and harpin, cis-Jasmone and Azadirachta indica oil, cis-Jasmone and Azadirachtin, harpin and Azadirachta indica oil, and harpin and Azadirachtin.

16. The plant, cell, plant part or seed of any one of claims 1 to 4 or 13 to 15, or the soil in which they are grown or are intended to be grown, treated with a biological control agent selected from the group consisting of: a Bacillus species strain, a Brevibacillus species strain, a Burkholderia species strain, a Lysobacter species strain, a Pasteuria species strain, an Arthrobotrys species strain, a Nematoctonus species strain, a Myrothecium species strain, a Paecilomyces species strain, a Trichoderma species strain, and a Tsukamurella species strain.

17. The plant, cell, plant part or seed of claim 16, or the soil in which they are grown or are intended to be grown, treated with a biological control agent selected from the group consisting of: Bacillus amyloliquefaciens, Bacillus firmus, Bacillus laterosporus, Bacillus lentus, Bacillus lichenformis, Bacillus nematocida, Bacillus pumilus, Bacillus subtilis, Bacillus penetrans, Bacillus thuringiensis, Brevibacillus laterosporus, Burkholderia rinojensis, Lysobacter antibioticus, Lysobacter enzymogenes, Pasteuria nishizawae, Pasteuria penetrans, Pasteuria ramosa, Pasteuria reniformis, Pasteuria thornei, Pasteuria usage, Arthrobotrys dactyloides, Arthrobotrys oligospora, Arthrobotrys superba, Nematoctonus geogenius, Nematoctonus leiosporus, Myrothecium verrucaria, Paecilomyces lilacinus, Paecilomyces variotii, Trichoderma asperellum, Trichoderma harzianum, Trichoderma viride, Trichoderma harzianum rifai, and Tsukamurella paurometabola.

18. The plant, cell, plant part or seed of claim 17, or the soil in which they are grown or are intended to be grown, treated with a biological control agent selected from the group consisting of: Bacillus amyloliquefaciens strain IN937a, Bacillus amyloliquefaciens strain FZB42, Bacillus amyloliquefaciens strain FZB24, Bacillus amyloliquefaciens strain NRRL B-50349, Bacillus amyloliquefaciens strain ABI01, Bacillus amyloliquefaciens strain B3, Bacillus amyloliquefaciens strain D747, Bacillus amyloliquefaciens strain APM-1, Bacillus amyloliquefaciens strain TJ1000, Bacillus amyloliquefaciens strain AP-136, Bacillus amyloliquefaciens strain AP-188, Bacillus amyloliquefaciens strain AP-218, Bacillus amyloliquefaciens strain AP-219, Bacillus amyloliquefaciens strain AP-295, Bacillus amyloliquefaciens strain MBI 600, Bacillus amyloliquefaciens strain PTA-4838, Bacillus amyloliquefaciens strain F727, Bacillus firmus strain I-1582, Bacillus firmus strain NRRL B-67003, Bacillus firmus strain NRRL B-67518, Bacillus firmus strain GB126, Bacillus laterosporus strain ATCC PTA-3952, Bacillus laterosporus strain ATCC PTA-3593, Bacillus licheniformis strain ATCC PTA-6175, Bacillus licheniformis SB3086, licheniformis CH200, licheniformis RTI 184, a combination of licheniformis CH200 and licheniformis RTI 184, Bacillus subtilis var. amyloliquefaciens strain FZB24, Bacillus thuringiensis strain EX297512, Bacillus thuringiensis strain CR-371, or Bacillus thuringiensis strain AQ52, Brevibacillus laterosporus strain ATCC 64, Brevibacillus laterosporus strain NRS 1111, Brevibacillus laterosporus strain NRS 1645, Brevibacillus laterosporus strain NRS 1647, Brevibacillus laterosporus strain BPM3, Brevibacillus laterosporus strain G4, Brevibacillus laterosporus strain NCIMB 41419, Burkholderia rinojensis, Burkholderia rinojensis strain A396, Lysobacter antibioticus strain 13-1, Lysobacter enzymogenes strain C3, Myrothecium verrucaria strain AARC-0255, Paecilomyces lilacinus strain 251, Paecilomyces variotii strain Q-09, Pasteuria nishizawae strain Pn1, Trichoderma asperellum strain ICC 012, Trichoderma asperellum strain SKT-1, Trichoderma asperellum strain T34, Trichoderma asperellum strain T25, Trichoderma asperellum strain SF04, Trichoderma asperellum strain TV1, Trichoderma asperellum strain T 11, Trichoderma harzianum strain ICC012, Trichoderma harzianum rifai T39, Trichoderma harzianum rifai strain KRL-AG2, Trichoderma viride strain TV1, Trichoderma viride strain TV25, Trichoderma atroviride strain CNCM I-1237, Trichoderma atroviride strain CNCM I-1237, Trichoderma atroviride strain NMI No. V08/002387, Trichoderma atroviride strain NMI No. V08/002388, Trichoderma atroviride strain NMI No. V08/002389, Trichoderma atroviride strain NMI No. V08/002390, Trichoderma atroviride strain ATCC 20476, Trichoderma atroviride strain T11, Trichoderma atroviride strain LC52, Trichoderma atroviride strain SC1, Trichoderma atroviride strain SKT-1, Trichoderma atroviride strain SKT-2, Trichoderma atroviride strain SKT-3, and Tsukamurella paurometabola strain C-92.

19. The plant, cell, plant part or seed of any one of claims 1 to 4 or any one of claims 13 to 18, wherein said treatment is a seed treatment.

20. The plant, cell, plant part or seed of any one of claims 1 to 4 or any one of claims 13 to 19, or the soil in which they are grown or are intended to be grown, treated with a combination of a nematicidal agent and a biological control agent selected from: a Bacillus species strain and alanycarb; a Bacillus species strain and aldicarb; a Bacillus species strain and carbofuran; a Bacillus species strain and carbosulfan; a Bacillus species strain and fosthiazate; a Bacillus species strain and cadusafos; a Bacillus species strain and oxamyl; a Bacillus species strain and thiodicarb; a Bacillus species strain and dimethoate; a Bacillus species strain and ethoprophos; a Bacillus species strain and terbufos; a Bacillus species strain and abamectin; a Bacillus species strain and methyl bromide and other alkyl halides; a Bacillus species strain and methyl isocyanate generators selected from diazomet and metam; a Bacillus species strain and fluazaindolizine; a Bacillus species strain and fluensulfone; a Bacillus species strain and fluopyram; a Bacillus species strain and tioxazafen; a Bacillus species strain and N-[1-(2,6-difluorophenyl)-1H-pyrazol-3-yl]-2-trifluoromethylbenzamide; a Bacillus species strain and cis-Jasmone; a Bacillus species strain and harpin; a Bacillus species strain and Azadirachta indica oil; a Bacillus species strain and Azadirachtin; a Brevibacillus species strain and alanycarb; a Brevibacillus species strain and aldicarb; a Brevibacillus species strain and carbofuran; a Brevibacillus species strain and carbosulfan; a Brevibacillus species strain and fosthiazate; a Brevibacillus species strain and cadusafos; a Brevibacillus species strain and oxamyl; a Brevibacillus species strain and thiodicarb; a Brevibacillus species strain and dimethoate; a Brevibacillus species strain and ethoprophos; a Brevibacillus species strain and terbufos; a Brevibacillus species strain and abamectin; a Brevibacillus species strain and methyl bromide and other alkyl halides; a Brevibacillus species strain and methyl isocyanate generators selected from diazomet and metam; a Brevibacillus species strain and fluazaindolizine; a Brevibacillus species strain and fluensulfone; a Brevibacillus species strain and fluopyram; a Brevibacillus species strain and tioxazafen; a Brevibacillus species strain and N-[1-(2,6-difluorophenyl)-1H-pyrazol-3-yl]-2-trifluoromethylbenzamide; a Brevibacillus species strain and cis-Jasmone; a Brevibacillus species strain and harpin; a Brevibacillus species strain and Azadirachta indica oil; a Brevibacillus species strain and Azadirachtin; a Burkholderia species strain and alanycarb; a Burkholderia species strain and aldicarb; a Burkholderia species strain and carbofuran; a Burkholderia species strain and carbosulfan; a Burkholderia species strain and fosthiazate; a Burkholderia species strain and cadusafos; a Burkholderia species strain and oxamyl; a Burkholderia species strain and thiodicarb; a Burkholderia species strain and dimethoate; a Burkholderia species strain and ethoprophos; a Burkholderia species strain and terbufos; a Burkholderia species strain and abamectin; a Burkholderia species strain and methyl bromide and other alkyl halides; a Burkholderia species strain and methyl isocyanate generators selected from diazomet and metam; a Burkholderia species strain and fluazaindolizine; a Burkholderia species strain and fluensulfone; a Burkholderia species strain and fluopyram; a Burkholderia species strain and tioxazafen; a Burkholderia species strain and N-[1-(2,6-difluorophenyl)-1H-pyrazol-3-yl]-2-trifluoromethylbenzamide; a Burkholderia species strain and cis-Jasmone; a Burkholderia species strain and harpin; a Burkholderia species strain and Azadirachta indica oil; a Burkholderia species strain and Azadirachtin; a Lysobacter species strain and alanycarb; a Lysobacter species strain and aldicarb; a Lysobacter species strain and carbofuran; a Lysobacter species strain and carbosulfan; a Lysobacter species strain and fosthiazate; a Lysobacter species strain and cadusafos; a Lysobacter species strain and oxamyl; a Lysobacter species strain and thiodicarb; a Lysobacter species strain and dimethoate; a Lysobacter species strain and ethoprophos; a Lysobacter species strain and terbufos; a Lysobacter species strain and abamectin; a Lysobacter species strain and methyl bromide and other alkyl halides; a Lysobacter species strain and methyl isocyanate generators selected from diazomet and metam; a Lysobacter species strain and fluazaindolizine; a Lysobacter species strain and fluensulfone; a Lysobacter species strain and fluopyram; a Lysobacter species strain and tioxazafen; a Lysobacter species strain and N-[1-(2,6-difluorophenyl)-1H-pyrazol-3-yl]-2-trifluoromethylbenzamide; a Lysobacter species strain and cis-Jasmone; a Lysobacter species strain and harpin; a Lysobacter species strain and Azadirachta indica oil; a Lysobacter species strain and Azadirachtin; a Pasteuria species strain and alanycarb; a Pasteuria species strain and aldicarb; a Pasteuria species strain and carbofuran; a Pasteuria species strain and carbosulfan; a Pasteuria species strain and fosthiazate; a Pasteuria species strain and cadusafos; a Pasteuria species strain and oxamyl; a Pasteuria species strain and thiodicarb; a Pasteuria species strain and dimethoate; a Pasteuria species strain and ethoprophos; a Pasteuria species strain and terbufos; a Pasteuria species strain and abamectin; a Pasteuria species strain and methyl bromide and other alkyl halides; a Pasteuria species strain and methyl isocyanate generators selected from diazomet and metam; a Pasteuria species strain and fluazaindolizine; a Pasteuria species strain and fluensulfone; a Pasteuria species strain and fluopyram; a Pasteuria species strain and tioxazafen; a Pasteuria species strain and N-[1-(2,6-difluorophenyl)-1H-pyrazol-3-yl]-2-trifluoromethylbenzamide; a Pasteuria species strain and cis-Jasmone; a Pasteuria species strain and harpin; a Pasteuria species strain and Azadirachta indica oil; a Pasteuria species strain and Azadirachtin; an Arthrobotrys species strain and alanycarb; an Arthrobotrys species strain and aldicarb; an Arthrobotrys species strain and carbofuran; an Arthrobotrys species strain and carbosulfan; an Arthrobotrys species strain and fosthiazate; an Arthrobotrys species strain and cadusafos; an Arthrobotrys species strain and oxamyl; an Arthrobotrys species strain and thiodicarb; an Arthrobotrys species strain and dimethoate; an Arthrobotrys species strain and ethoprophos; an Arthrobotrys species strain and terbufos; an Arthrobotrys species strain and abamectin; an Arthrobotrys species strain and methyl bromide and other alkyl halides; an Arthrobotrys species strain and methyl isocyanate generators selected from diazomet and metam; an Arthrobotrys species strain and fluazaindolizine; an Arthrobotrys species strain and fluensulfone; an Arthrobotrys species strain and fluopyram; an Arthrobotrys species strain and tioxazafen; an Arthrobotrys species strain and N-[1-(2,6-difluorophenyl)-1H-pyrazol-3-yl]-2-trifluoromethylbenzamide; an Arthrobotrys species strain and cis-Jasmone; an Arthrobotrys species strain and harpin; an Arthrobotrys species strain and Azadirachta indica oil; an Arthrobotrys species strain and Azadirachtin; a Nematoctonus species strain and alanycarb; a Nematoctonus species strain and aldicarb; a Nematoctonus species strain and carbofuran; a Nematoctonus species strain and carbosulfan; a Nematoctonus species strain and fosthiazate; a Nematoctonus species strain and cadusafos; a Nematoctonus species strain and oxamyl; a Nematoctonus species strain and thiodicarb; a Nematoctonus species strain and dimethoate; a Nematoctonus species strain and ethoprophos; a Nematoctonus species strain and terbufos; a Nematoctonus species strain and abamectin; a Nematoctonus species strain and methyl bromide and other alkyl halides; a Nematoctonus species strain and methyl isocyanate generators selected from diazomet and metam; a Nematoctonus species strain and fluazaindolizine; a Nematoctonus species strain and fluensulfone; a Nematoctonus species strain and fluopyram; a Nematoctonus species strain and tioxazafen; a Nematoctonus species strain and N-[1-(2,6-difluorophenyl)-1H-pyrazol-3-yl]-2-trifluoromethylbenzamide; a Nematoctonus species strain and cis-Jasmone; a Nematoctonus species strain and harpin; a Nematoctonus species strain and Azadirachta indica oil; a Nematoctonus species strain and Azadirachtin; a Myrothecium species strain and alanycarb; a Myrothecium species strain and aldicarb; a Myrothecium species strain and carbofuran; a Myrothecium species strain and carbosulfan; a Myrothecium species strain and fosthiazate; a Myrothecium species strain and cadusafos; a Myrothecium species strain and oxamyl; a Myrothecium species strain and thiodicarb; a Myrothecium species strain and dimethoate; a Myrothecium species strain and ethoprophos; a Myrothecium species strain and terbufos; a Myrothecium species strain and abamectin; a Myrothecium species strain and methyl bromide and other alkyl halides; a Myrothecium species strain and methyl isocyanate generators selected from diazomet and metam; a Myrothecium species strain and fluazaindolizine; a Myrothecium species strain and fluensulfone; a Myrothecium species strain and fluopyram; a Myrothecium species strain and tioxazafen; a Myrothecium species strain and N-[1-(2,6-difluorophenyl)-1H-pyrazol-3-yl]-2-trifluoromethylbenzamide; a Myrothecium species strain and cis-Jasmone; a Myrothecium species strain and harpin; a Myrothecium species strain and Azadirachta indica oil; a Myrothecium species strain and Azadirachtin; a Paecilomyces species strain and alanycarb; a Paecilomyces species strain and aldicarb; a Paecilomyces species strain and carbofuran; a Paecilomyces species strain and carbosulfan; a Paecilomyces species strain and fosthiazate; a Paecilomyces species strain and cadusafos; a Paecilomyces species strain and oxamyl; a Paecilomyces species strain and thiodicarb; a Paecilomyces species strain and dimethoate; a Paecilomyces species strain and ethoprophos; a Paecilomyces species strain and terbufos; a Paecilomyces species strain and abamectin; a Paecilomyces species strain and methyl bromide and other alkyl halides; a Paecilomyces species strain and methyl isocyanate generators selected from diazomet and metam; a Paecilomyces species strain and fluazaindolizine; a Paecilomyces species strain and fluensulfone; a Paecilomyces species strain and fluopyram; a Paecilomyces species strain and tioxazafen; a Paecilomyces species strain and N-[1-(2,6-difluorophenyl)-1H-pyrazol-3-yl]-2-trifluoromethylbenzamide; a Paecilomyces species strain and cis-Jasmone; a Paecilomyces species strain and harpin; a Paecilomyces species strain and Azadirachta indica oil; a Paecilomyces species strain and Azadirachtin; a Trichoderma species strain and alanycarb; a Trichoderma species strain and aldicarb; a Trichoderma species strain and carbofuran; a Trichoderma species strain and carbosulfan; a Trichoderma species strain and fosthiazate; a Trichoderma species strain and cadusafos; a Trichoderma species strain and oxamyl; a Trichoderma species strain and thiodicarb; a Trichoderma species strain and dimethoate; a Trichoderma species strain and ethoprophos; a Trichoderma species strain and terbufos; a Trichoderma species strain and abamectin; a Trichoderma species strain and methyl bromide and other alkyl halides; a Trichoderma species strain and methyl isocyanate generators selected from diazomet and metam; a Trichoderma species strain and fluazaindolizine; a Trichoderma species strain and fluensulfone; a Trichoderma species strain and fluopyram; a Trichoderma species strain and tioxazafen; a Trichoderma species strain and N-[1-(2,6-difluorophenyl)-1H-pyrazol-3-yl]-2-trifluoromethylbenzamide; a Trichoderma species strain and cis-Jasmone; a Trichoderma species strain and harpin; a Trichoderma species strain and Azadirachta indica oil; a Trichoderma species strain and Azadirachtin; a Tsukamurella species strain and alanycarb; a Tsukamurella species strain and aldicarb; a Tsukamurella species strain and carbofuran; a Tsukamurella species strain and carbosulfan; a Tsukamurella species strain and fosthiazate; a Tsukamurella species strain and cadusafos; a Tsukamurella species strain and oxamyl; a Tsukamurella species strain and thiodicarb; a Tsukamurella species strain and dimethoate; a Tsukamurella species strain and ethoprophos; a Tsukamurella species strain and terbufos; a Tsukamurella species strain and abamectin; a Tsukamurella species strain and methyl bromide and other alkyl halides; a Tsukamurella species strain and methyl isocyanate generators selected from diazomet and metam; a Tsukamurella species strain and fluazaindolizine; a Tsukamurella species strain and fluensulfone; a Tsukamurella species strain and fluopyram; a Tsukamurella species strain and tioxazafen; a Tsukamurella species strain and N-[1-(2,6-difluorophenyl)-1H-pyrazol-3-yl]-2-trifluoromethylbenzamide; a Tsukamurella species strain and cis-Jasmone; a Tsukamurella species strain and harpin; a Tsukamurella species strain and Azadirachta indica oil; and a Tsukamurella species strain and Azadirachtin.

21. The plant, cell, plant part or seed of any one of claims 1 to 4 or any one of claims 13 to 20, or the soil in which they are grown or are intended to be grown, wherein said plant, cell, plant part or seed is further treated with an insecticide selected from IAN1, IAN2, IAN3, IAN4, IAN5, IAN6, IAN7, IAN8, IAN9, IAN10, IAN11, IAN12, IAN13, IAN14, IAN15, IAN16, IAN17, IAN18, IAN19, IAN20, IAN21, IAN22, IAN23, IAN24, IAN25, IAN26, IAN27, IAN28, IAN29, IAN30, or SIAN1.

22. The plant, cell, plant part or seed of any one of claims 1 to 4 or any one of claims 13 to 21, or the soil in which they are grown or are intended to be grown, wherein said plant, cell, plant part or seed is further treated with a fungicide selected from F1, F2, F3, F4, F5, F6, F7, F8, F9, F9, F10, F11, F12, F13, F14, F15, F16, or SF1.

23. The plant, cell, plant part or seed of any one of claims 1 to 4 or any one of claims 13 to 22, or the soil in which they are grown or are intended to be grown, treated with a combination selected from the group consisting of: combination of Clothianidin and Bacillus firmus (such as B. firmus GB126), combination of Clothianidin, Bacillus thuringiensis (such as B. thuringiensis strain EX297512) and Bacillus firmus (such as B. firmus GB126), combination of Imidacloprid and Thiodicarb, combination of Imidacloprid and Prothioconazole, combination of Clothianidin and Carboxin and Metalaxyl and Trifloxystrobin, combination of Metalaxyl and Prothioconazole and Tebuconazole, combination of Clothianidin and beta-Cyfluthrin, combination of Prothioconazole and Tebuconazole, combination of Clothianidin and Imidacloprid and Prothioconazole and Tebuconazole, combination of Carbendazim and Thiram, combination of Imidacloprid and Methiocarb, combination of Metalaxyl, Penflufen and Prothioconazole, combination of Metalaxyl, Penflufen, Prothioconazole and imidacloprid, combination of Metalaxyl, Penflufen, Prothioconazole, imidacloprid and fluopyram, combination of Metalaxyl, Penflufen, Prothioconazole, imidacloprid, fluopyram and Bacillus firmus (such as B. firmus GB126), combination of Metalaxyl, Penflufen, Prothioconazole, imidacloprid, fluopyram, Bacillus firmus (such as B. firmus GB126) and Bacillus thuringiensis (such as B. thuringiensis strain EX297512), combination of Metalaxyl, Penflufen, Prothioconazole and Clothianidin, combination of Metalaxyl, Penflufen, Prothioconazole, Clothianidin and Bacillus firmus (such as B. firmus GB126), combination of Metalaxyl, Penflufen, Prothioconazole, Clothianidin, Bacillus firmus (such as B. firmus GB126) and Bacillus thuringiensis (such as B. thuringiensis strain EX297512), combination of Metalaxyl, Penflufen, Prothioconazole, Clothianidin, Bacillus firmus (such as B. firmus GB126) and fluopyram, combination of Metalaxyl, Penflufen, Prothioconazole, Clothianidin, Bacillus firmus (such as B. firmus GB126), fluopyram and Bacillus thuringiensis (such as B. thuringiensis strain EX297512), combination of Imidacloprid and Prothioconazole, combination of Imidacloprid and Tefluthrin, combination of Imidacloprid and Pencycuron, combination of Imidacloprid and Penflufen, combination of Fluoxastrobin, Prothioconazole and Tebuconazole, combination of Fluoxastrobin, Prothioconazole and metalaxyl, combination of Fluoxastrobin, Prothioconazole, metalaxyl and imdiacloprid, combination of Fluoxastrobin, Prothioconazole, metalaxyl, imdiacloprid and fluopyram, combination of Fluoxastrobin, Prothioconazole, metalaxyl, imdiacloprid, fluopyram and Bacillus firmus (such as B. firmus GB126), and combination of Fluoxastrobin, Prothioconazole, metalaxyl, imdiacloprid, fluopyram, Bacillus firmus (such as B. firmus GB126), and Bacillus thuringiensis (such as B. thuringiensis strain EX297512), combination of Fluoxastrobin, Prothioconazole, metalaxyl, clothianidin and fluopyram, combination of Fluoxastrobin, Prothioconazole, metalaxyl, clothianidin and Bacillus firmus (such as B. firmus GB126), combination of Fluoxastrobin, Prothioconazole, metalaxyl, clothianidin, Bacillus firmus (such as B. firmus GB126), and Bacillus thuringiensis (such as B. thuringiensis strain EX297512), combination of Fluoxastrobin, Prothioconazole, metalaxyl, clothianidin and Bacillus firmus (such as B. firmus GB126), combination of Fluoxastrobin, Prothioconazole, metalaxyl, clothianidin, Bacillus firmus (such as B. firmus GB126), and Bacillus thuringiensis (such as B. thuringiensis strain EX297512), combination of Metalaxyl and Trifloxystrobin, combination of Penflufen and Trifloxystrobin, combination of Prothioconazole and Tebuconazole, combination of Fluoxastrobin and Prothioconazole and Tebuconazole and Triazoxide, combination of Imidacloprid and Methiocarb and Thiram, combination of Clothianidin and beta-Cyfluthrin, combination of Clothianidin and Fluoxastrobin and Prothioconazole and Tebuconazole, combination of Fluopyram and Fluoxastrobin and Triadimenol, combination of Metalaxyl and Trifloxystrobin, combination of Imidacloprid and Ipconacole, combination of Difenoconazol and Fludioxonil and Tebuconazole, combination of Imidacloprid and Tebuconazole, combination of Imidacloprid, Prothioconazole and Tebuconazole, combination of Metalaxyl, Prothioconazole and Tebuconazole, combination of fluopyram and Bacillus firmus (such as B. firmus GB126), combination of fluopyram, Bacillus firmus (such as B. firmus GB126), and Bacillus thuringiensis (such as B. thuringiensis strain EX297512), Combination of Pasteria nishazawae (such as P. nishizawae Pn1), thiamethoxam, sedexane, fludioxinil and mefonaxam, Combination of thiamethoxam, sedexane, fludioninil and mefonaxam, Combination of thiamethoxam, fludioxinil and mefonaxam, Combination of fludioxinil and mefonaxam, Combination of pyraclostrobin and fluoxayprad, Combination of abamectin and thiamethoxam, Combination of Burkholderia spp. strain (such as strain A396) and imidacloprid, Combination of Bacillus amyloliquefaciens (such as B. amyloliquefaciens strain PTA-4838) and clothianidin, Combination of tioxazafen, imidacloprid, prothioconazole, fluoxastrobin and metalaxyl, Combination of tioxazafen, clothiandiin, prothioconazole, fluoxastroin and metalaxyl, Combination of tioxazafen, clothianidin, Bacillus firmus (such as B. firmus GB126), prothioconazole, fluoxastrobin and metalaxyl, Combination of tioxazafen, clothianidin, Bacillus firmus (such as B. firmus GB126), Bacillus thuringiensis (such as B. thuringiensis strain EX297512), prothioconazole, fluoxastrobin and metalaxyl, combination of tioxazafen, prothioconazole, fluoxastrobin and metalaxyl, combination of tioxazafen, pyraclostrobin, fluoxyprad, metalaxyl and imidacloprid, a combination of Sedaxane, Thiamethoxam, Fludioxonil, and Mefenoxam, and a combination of Pasteuria nishizawae, Sedaxane, Thiamethoxam, Fludioxonil, and Mefenoxam, combination of clothianidin, fluopyram and Bacillus firmus (such as B. firmus GB126), combination of clothianidin, fluopyram, tioxazafen and Bacillus firmus (such as B. firmus GB126); combination of clothianidin, fluopyram, Bacillus firmus (such as B. firmus GB126) and Bacillus thuringiensis (such as B. thuringiensis strain EX297512), combination of clothianidin, fluopyram, tioxazafen, Bacillus firmus (such as B. firmus GB126) and Bacillus thuringiensis (such as B. thuringiensis strain EX297512).

24. The plant, cell, plant part or seed of any one of claims 1 to 4 or any one of claims 13 to 23, also comprising another soybean transformation event such as a soybean transformation event providing tolerance to additional herbicides, a soybean transformation event providing tolerance to nematodes via a different mode of action, or a soybean transformation event providing insect control, or any one of the following soybean transformation events: Event MON87751, Event pDAB8264.42.32.1, Event DAS-81419-2, Event FG-072, Event SYHTOH2, Event DAS-68416-4, Event DAS-81615-9, Event DAS-44406-6, Event MON87708, Event MON89788, Event DAS-14536-7, Event GTS 40-3-2, Event A2704-12, Event BPS-CV127-9, Event A5547-127, Event MON87754, Event DP-3Ø5423-1, Event MON87701, Event MON87705, Event MON87712, Event pDAB4472-1606, Event DP-356043-5, Event MON87769, Event IND-ØØ41Ø-5, Event DP305423, or any of the following Event combinations: MON89788×MON87708, HOS×GTS 40-3-2, FG-072×A5547-127, MON87701×MON 89788, DAS-81419-2×DAS-44406-6, DAS-81419-2×DAS-68416-4, DAS-68416-4×MON 89788, MON 87705×MON 89788, MON 87769 x MON 89788, DP305423×GTS 40-3-2, DP305423×MON87708, Event DP305423 x MON87708×Event MON89788, DP305423×MON89788, MON87705×MON87708, MON87705×MON87708×MON89788, MON89788×MON87708×A5547-127, MON87751 x MON87701×MON87708×MON89788, SYHTOH2×MON89788, SYHTOH2×GTS 40-3-2, SYHTOH2×MON89788×MON87708.

25. The plant, cell, plant part or seed of any one of claims 1 to 4 or any one of claims 13 to 24, wherein said plant, plant part, or seed are planted or sown in the soil treated with said compounds, agents or mixtures.
